(19) United States
(12) Patent Application Publication
(10) Pub. No.: US 2016/0235837 A1
(43) Pub. Date: Aug. 18, 2016

(54) THERAPIES, VACCINES, AND PREDICTIVE METHODS FOR MIDDLE EAST RESPIRATORY SYNDROME VIRUS (MERS CoV)

(71) Applicants: Samuel BOGOCH, New York, NY (US); Elenore S. BOGOCH, New York, NY (US); Anne-Elenore Bogoch BORSANYI, New York, NY (US); Samuel Winston BOGOCH, Oakland, CA (US)

(72) Inventors: Samuel BOGOCH, New York, NY (US); Elenore S. BOGOCH, New York, NY (US); Anne-Elenore Bogoch BORSANYI, New York, NY (US); Samuel Winston BOGOCH, Oakland, CA (US)

(21) Appl. No.: 15/029,169

(22) PCT Filed: Oct. 16, 2014
(86) PCT No.: PCT/US14/60906

§ 371 Date: Apr. 13, 2016

Related U.S. Application Data
(60) Provisional application No. 61/891,677, filed on Oct. 16, 2013.
(60) Provisional application No. 61/993,332, filed on May 15, 2014.

(51) Int. Cl. A61K 39/215; C07K 14/005; C12N 7/00; C07K 16/10

(52) U.S. Cl. CPC A61K 39/215; C07K 16/10; C07K 14/005; C12N 7/00; C12N 2770/20034; C12N 2770/20022; C07K 2317/34

(57) ABSTRACT

The present invention provides an isolated or synthesized protein fragment or peptide comprising at least one peptide sequence that is at least 50% homologous with at least one Replikin peptide sequence identified in a MERS CoV. The at least one peptide sequence may be at least 80% homologous with at least one Replikin peptide sequence identified in a MERS CoV. The at least one peptide sequence may be at least 46% homologous with at least one Replikin peptide sequence identified in a MERS CoV. The at least one peptide sequence may be at least 53% homologous with at least one Replikin peptide sequence.

This application claims priority to U.S. Provisional Appln. Ser. No. 61/993,332, filed May 15, 2014, and U.S. Provisional Appln. Ser. No. 61/891,677, filed Oct. 16, 2013.

This application incorporates by reference the following applications in their entireties: PCT/US14/34692, filed Apr. 18, 2014, U.S. Provisional Appln. Ser. No. 61/975,674, filed Apr. 4, 2014, PCT/US14/25053, filed Mar. 12, 2014, PCT/US13/69310, filed Feb. 13, 2014, U.S. Provisional Application Ser. No. 61/907,802, filed Nov. 22, 2013, PCT/US13/69310, filed Nov. 8, 2013, U.S. Provisional Application Ser. No. 61/891,677, filed Oct. 16, 2013, PCT/US2013/039111, filed May 1, 2013, U.S. Provisional Application Ser. No. 61/813,889, filed Apr. 19, 2013, U.S. Provisional Application Ser. No. 61/779,324, filed Mar. 13, 2013, U.S. Provisional Application Ser. No. 61/609,074, filed Mar. 9, 2012, U.S. Provisional Appln. Ser. No. 617/65,106, filed Feb. 15, 2013, U.S. Provisional Appln. Ser. No. 61/724,538, filed Nov. 9, 2012, U.S. application Ser. No. 13/553,137, filed Jul. 19, 2012, PCT/US2012/047451, filed Jul. 19, 2012, U.S. Provisional Appln. Ser. No. 61/509,896, filed Jul. 20, 2011, U.S. application Ser. No. 12/581,112, filed Oct. 16, 2009, U.S. Provisional Appln. Ser. No. 61/246,006, filed Sep. 25, 2009, U.S. application Ser. No. 12/538,027, filed Aug. 7, 2009, U.S. Provisional Appln. Ser. No. 61/185,160, filed Jun. 8, 2009, U.S. Provisional Appln. Ser. No. 61/179,686, filed May 19, 2009, U.S. Provisional Appln. Ser. No. 61/172,115, filed Apr. 23, 2009, U.S. application Ser. No. 12/429,044, filed Apr. 23, 2009, and PCT/US09/41565, filed Apr. 23, 2009, U.S. Provisional Appln. Ser. No. 61/143,618, filed Jan. 9, 2009, U.S. Provisional Appln. Ser. No. 61/087,354, filed Aug. 8, 2008, U.S. Provisional Appln. Ser. No. 61/054,010, filed May 16, 2008, U.S. application Ser. No. 12/108,458, filed Apr. 23, 2008, PCT/US2008/61336, filed Apr. 23, 2008, U.S. application Ser. No. 12/010,027, filed Jan. 18, 2008, U.S. Provisional Appln. Ser. No. 60/991,676, filed Nov. 30, 2007, U.S. application Ser. No. 11/923,559, filed Oct. 24, 2007, now U.S. Pat. No. 8,050,871, U.S. Provisional Appln. Ser. No. 60/982,336, filed Oct. 24, 2007, U.S. Provisional Appln. Ser. No. 60/982,333, filed Oct. 24, 2007, U.S. Provisional Appln. Ser. No. 60/982,338, filed Oct. 24, 2007, U.S. Provisional Appln. Ser. No. 60/935,816, filed Aug. 31, 2007, U.S. Provisional Appln. Ser. No. 60/935,499 filed Aug. 16, 2007, U.S. Provisional Appln. Ser. No. 60/954,743, filed Aug. 8, 2007, U.S. application Ser. No. 11/755,597, filed May 30, 2007, U.S. Provisional Appln. Ser. No. 60/898,097, filed Jan. 30, 2007, U.S. Provisional Appln. Ser. No. 60/880,966, filed Jan. 18, 2007, U.S. Provisional Appln. Ser. No. 60/853,744, filed Oct. 24, 2006, U.S. application Ser. No. 11/355,120, filed Feb. 16, 2006, U.S. application Ser. No. 11/116,203, filed Apr. 28, 2005, U.S. application Ser. No. 10/860,050, filed Jun. 4, 2004, now U.S. Pat. No. 7,442,761, U.S. application Ser. No. 10/189,437, filed Jul. 8, 2002, now U.S. Pat. No. 7,452,963, U.S. application Ser. No. 10/105,232, filed Mar. 26, 2002, now U.S. Pat. No. 7,189,800, U.S. application Ser. No. 09/984,057, filed Oct. 26, 2001, now U.S. Pat. No. 7,420,028, and U.S. application Ser. No. 09/984,056, filed Oct. 26, 2001, now U.S. Pat. No. 7,176,275.

### SEQUENCE LISTING

The instant application contains a Sequence Listing, which has been submitted electronically in ASCII format and is hereby incorporated by reference in its entirety. Said ASCII copy, created on Oct. 16, 2014, is named 13795-48876_SL.txt and is 174,231 bytes in size.

### FIELD OF THE INVENTION

The present invention relates to therapies for preventing and treating Middle East Respiratory Syndrome Virus (MERS CoV), methods of differentiating lethality of MERS CoV and of predicting outbreaks of lethal MERS CoV, and compounds for diagnostic, therapeutic, and/or preventive purposes in MERS CoV.

### BACKGROUND OF THE INVENTION

Middle East Respiratory Syndrome (MERS) is a viral respiratory illness first reported in Saudi Arabia in 2012. The disease is caused by a coronavirus known as MERS-CoV. Most confirmed MERS-CoV infections manifest as severe acute respiratory illness with fever, cough, and shortness of breath. Mortality rate in confirmed cases is around fifty percent. At the present time, confirmed cases are linked to four countries in or near the Arabian Peninsula including, Saudi Arabia, Jordan, and Qatar. Spread of the virus from ill people to others through close contact has reportedly been observed in a few cases. Nevertheless, at present the virus has not been observed to spread in a sustained way in human communities.

MERS CoV is a novel coronavirus belonging to the genus Betacoronavirus. Severe Acute Respiratory Coronavirus (SARS-CoV) also belongs to this genus. MERS CoV has been referred to as a SARS-like virus but is nevertheless more closely related to the bat coronaviruses HKU4 and HKU5 (lineage 2C) than it is to SARS-CoV (lineage 2B) and shares more than 90% sequence identity in closest relationships with HKU4 and HKU5.

MERS CoV was first reported Sep. 24, 2012 from Saudi Arabia. The virus was isolated and identified from the lungs of a 60-year-old male patient with acute pneumonia and acute renal failure.

MERS CoV is a single-stranded RNA Group IV virus. It is positive sense. The virus is categorized in Order Nidovirales, Family Coronaviridae, Subfamily Coronavirinae, and Genus Betacoronavirus. Species within the genus include Betacoronavirus 1 (commonly called Human coronavirus OC43), Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, and MERS-CoV. The MERS CoV genome has been reported to contain the following gene segments: N, M, S, ORF4b, ORF1ab, ORF1a, ORF1b, NS3C, and N3.

In April 2012, six hospital workers were diagnosed with acute respiratory failure of unknown origin. Of the six, two died. All the cases were reported to the European Centre for Disease Prevention and Control (ECDC). Following publication of the MERS CoV strain, a trace back was undertaken. Epidemiologists discovered the Jordan cases. Using stored laboratory samples for all six, it was found that samples from the two patients who had died tested positive for MERS CoV.

On May 14, 2014, Bloomberg news reported that at least 365 cases of MERS had been reported to the World Health Organization since Mar. 27, 2014, which was reported as more than double the number of infections since the start of the outbreak. It reported that contact with camels during the spring, when females wean their young, was thought to have contributed to the surge in new cases, and this was thought to have been amplified by poor infection control measures in hospitals. An analysis of 128 cases in the Saudi city of Jeddah had found that more than 60 percent were infected while in hospital. Bloomberg News, Simeon Bennett, 9:25 a.m.

There is a need in the art for quantitative methods of predicting further progression and additional outbreaks or waning of MERS CoV. There is likewise a need in the art for methods of preventing, and treating MERS CoV infections and outbreaks and a need for therapies against MERS CoV including prophylactic therapies, such as vaccines, and treatments, such as therapies for providing passive immunity and other methods of blocking progression or transmission of MERS CoV and related viruses.

Replikin peptides are a family of small peptides that have been correlated with the phenomenon of rapid replication in SARS, influenza, malaria, West Nile virus, foot and mouth disease, and many other pathogens. See, e.g., WO 2008/143717. Replikin peptides have likewise been generally correlated with the phenomenon of rapid replication in viruses, organisms, and malignancies.

Identification of Replikin peptides has provided targets for detection and treatment of pathogens, including vaccine development against virulent pathogens such as influenza virus, malaria, West Nile virus, and foot and mouth disease virus. See, e.g., WO 2008/143717. In general, knowledge of and identification of this family of peptides enables development of effective therapies and vaccines for any pathogen that harbors Replikins. The phenomenon of the association of Replikins with rapid replication and virulence has been fully described in U.S. Pat. No. 7,189,800; U.S. Pat. No. 7,176,275; U.S. Pat. No. 7,442,761; U.S. Pat. No. 7,894,999, U.S. Pat. No. 8,050,871, and U.S. application Ser. No. 12/108,458. Both Replikin concentration (number of Replikins per 100 amino acids) and Replikin composition have been correlated with the functional phenomenon of rapid replication.

There is a continuing need for monitoring Replikin sequences in MERS CoV to identify compounds for therapies that target MERS CoV. There is also a need to develop Replikin-based therapies that are effective across strains and within strains as they mutate over time.

In response to these continuing needs and despite extensive efforts to understand infectivity and lethality in MERS CoV and to track and predict outbreaks of MERS CoV, applicants have now surprisingly applied their previous discovery of Replikin chemistry in the virus genome structure to methods of predicting outbreaks of MERS CoV. They have likewise now surprisingly provided methods of identifying conserved targets in emerging MERS CoV against which vaccines are provided and likewise may be provided prior to or at the outset of any further outbreak. Such vaccine development can be undertaken in as few as seven days.

### SUMMARY OF THE INVENTION

The present invention provides compounds for diagnostic, therapeutic, and/or preventive purposes against MERS CoV and methods of predicting outbreaks of MERS CoV.

A first non-limiting aspect of the present invention provides an isolated or synthesized protein fragment or peptide comprising at least one peptide sequence that is at least 50% homologous with at least one Replikin peptide sequence identified in a MERS CoV. In a non-limiting embodiment, the at least one peptide sequence may be at least 80% homologous with at least one Replikin peptide sequence identified in a MERS CoV. In a non-limiting embodiment, the at least one peptide sequence may be at least 46% homologous with at least one Replikin peptide sequence identified in a MERS CoV. In a non-limiting embodiment, the at least one peptide sequence may be at least 53% homologous with at least one Replikin peptide sequence identified in a MERS CoV. In a non-limiting embodiment, the at least one peptide sequence may be at least 60%, 70%, 80%, 90%, or 95% homologous with at least one Replikin peptide sequence identified in a MERS CoV. In another non-limiting embodiment, the isolated or synthesized protein fragment or peptide may comprise at least one Replikin peptide sequence identified in an MERS CoV or at least one homologue of the at least one Replikin sequence identified in an MERS CoV.

In a non-limiting embodiment, an isolated or synthesized protein fragment or peptide consists of at least one peptide sequence that is at least 50% homologous with at least one Replikin peptide sequence identified in a MERS CoV. In a non-limiting embodiment, an isolated or synthesized protein fragment or peptide consists of at least one peptide sequence that is at least 46%, 50%, 53%, 60%, 70%, 80%, 90%, or 95% homologous with at least one Replikin peptide sequence identified in a MERS CoV. In a non-limiting embodiment, an isolated or synthesized protein fragment or peptide consists of at least one Replikin peptide sequence identified in a MERS CoV. In a non-limiting embodiment, the at least one Replikin peptide sequence identified in a MERS CoV is at least one of SEQ ID NO(s): 1-28, 30-172, 174-190, 192-209, 211-222, 224-235, and 237-239. In a non-limiting embodiment, the isolated or synthesized protein fragment or peptide consists of up to 50, up to 75, up to 100, up to 150, or up to 200 or more amino acid residues.

In a non-limiting embodiment, an isolated or synthesized protein fragment or peptide consists of at least one peptide sequence that is at least 80% homologous with at least one of SEQ ID NO(s): 1-28, 30-172, 174-190, 192-209, 211-222, 224-235, and 237-239. In a non-limiting embodiment, the isolated or synthesized protein fragment or peptide consists essentially of at least one of SEQ ID NO(s): 1-28, 30-172, 174-190, 192-209, 211-222, 224-235, and 237-239. In a non-limiting embodiment, the isolated or synthesized protein fragment or peptide consists of at least one of SEQ ID NO(s): 1-28, 30-172, 174-190, 192-209, 211-222, 224-235, and 237-239. In a non-limiting embodiment, the isolated or synthesized protein fragment or peptide consists of at least one of SEQ ID NO(s): 1-28. In a non-limiting embodiment, the isolated or synthesized protein fragment or peptide of consists of at least one of SEQ ID NO(s): 1-9. In a non-limiting embodiment, the isolated or synthesized protein fragment or peptide of consists of at least one of SEQ ID NO(s): 5, 18, 22, and 23. In a non-limiting embodiment, the isolated or synthesized protein fragment or peptide consists of at least one of SEQ ID NO(s): 6, 11, 12, 13, 14, 18, 19, 20, 26, 27, and 28.

In a non-limiting embodiment an isolated or synthesized protein fragment or peptide consists of up to 200 amino acid residues. In a non-limiting embodiment, an isolated or synthesized protein fragment or peptide consists of up to 150 amino acid residues. In a non-limiting embodiment, an isolated or synthesized protein fragment or peptide consists of up to 100 amino acid residues. In a non-limiting embodiment, an isolated or synthesized protein fragment or peptide consists of up to 75 amino acid residues. In a non-limiting embodiment, an isolated or synthesized protein fragment or peptide consists of up to 50 amino acid residues. In a non-limiting embodiment, an isolated or synthesized protein fragment or peptide consists of up to 25 amino acid residues.

An isolated or synthesized protein fragment or peptide may consist essentially of at least one Replikin peptide sequence identified in an MERS CoV or at least one homologue of said at least one Replikin peptide sequence identified in an MERS CoV. The isolated or synthesized protein fragment or peptide may consist of at least one Replikin peptide sequence identified in an MERS CoV or at least one homologue of said at least one Replikin peptide sequence identified in an MERS CoV. Another non-limiting embodiment provides an isolated or synthesized peptide sequence comprising at least one functional fragment of a Replikin sequence identified in MERS CoV. In a non-limiting embodiment, the protein fragment or peptide may be isolated or derived from one of the gene segments of the genome of MERS CoV. A non-limiting gene segment may be N, M, S, ORF4b, ORF1ab, ORF1a, ORF1b, NS3C, N3, or any other gene segment.

In a non-limiting embodiment, the isolated or synthesized protein fragment or peptide may comprise at least one Replikin peptide sequence of KQKAPKH (SEQ ID NO: 1), KSAGHPFNK (SEQ ID NO: 2), KRSHSPTKK (SEQ ID NO: 3), HSPTKKLRYVK (SEQ ID NO: 4), KARKRSHSPTK (SEQ ID NO: 5), KHVEVFTDGK (SEQ ID NO: 6), HKWKMVVCDK (SEQ ID NO: 7), KKHVEVFTDGK (SEQ ID NO: 8), KDAAAAKNKMRHK (SEQ ID NO: 9), KSVVRHLGVTK (SEQ ID NO: 10), KFYQHVINGCK (SEQ ID NO: 11), KQVHQVQLTDK (SEQ ID NO: 12), KGDSCSSNCKH (SEQ ID NO: 13), HARLKGGLILK (SEQ ID NO: 14), KAMLLKKEPLLYVPIRLAGH (SEQ ID NO: 15), KHLVPLMHK (SEQ ID NO: 16), KYYAFLNKH (SEQ ID NO: 17), KAAVHKWK (SEQ ID NO: 18), KLNPSEDFIKH (SEQ ID NO: 19), KFCDHMVK (SEQ ID NO: 20), KPGHAMPSLFK (SEQ ID NO: 21), KNKMRHK (SEQ ID NO: 22), KRSHSPTK (SEQ ID NO: 23), KDAAAAKNKMRH (SEQ ID NO: 24), KMRHKRTSTK (SEQ ID NO: 25), HVERKDVPYPK (SEQ ID NO: 26), KGMQLLHTK (SEQ ID NO: 27), or KEGSSVTLKH (SEQ ID NO: 28), or at least one homologue of SEQ ID NO(s): 1-28. In a non-limiting embodiment, the isolated or synthesized protein fragment or peptide may consists essentially of at least one Replikin peptide sequence of SEQ ID NO(s): 1-28 or at least one homologue of SEQ ID NO(s): 1-28. In another non-limiting embodiment, the isolated or synthesized protein fragment or peptide may consist of at least one Replikin peptide sequence of SEQ ID NO(s): 1-28 or at least one homologue of SEQ ID NO(s): 1-28. Another non-limiting embodiment provides an isolated or synthesized peptide sequence comprising at least one functional fragment of at least one Replikin peptide sequence of SEQ ID NO(s): 1-28. Another non-limiting embodiment provides an isolated or synthesized peptide sequence comprising at least one functional fragment of at least one Replikin peptide sequence of SEQ ID NO(s): 1-28, 30-172, 174-190, 192-209, 211-222, 224-235, 237, or 238.

A non-limiting embodiment of the first aspect of the invention provides an isolated or synthesized protein, protein fragment, polypeptide, or peptide comprising at least one Replikin peptide of an MERS CoV virus. A further embodiment of the first aspect of the invention provides an isolated or synthesized protein, protein fragment, polypeptide, or peptide comprising at least one peptide sequence that is at least 30%, 40%, 50%, 60%, 70%, 80%, 90% 95%, or 100%, homologous with at least one Replikin peptide sequence identified in a MERS CoV virus. In a non-limiting embodiment, the at least one sequence is one of SEQ ID NO(s): 1-28.

In a further non-limiting embodiment of the first aspect of the present invention, the isolated or synthesized protein, protein fragment, polypeptide, or peptide consists of 7 to about 50 amino acid residues comprising at least one peptide A, wherein said peptide A is at least 30%, 40%, 50%, 60%, 70%, 80%, 90%, 95%, or 100%, homologous with at least one Replikin peptide sequence identified in an MERS CoV. In one non-limiting embodiment, said at least one Replikin peptide sequence identified in MERS CoV is at least one peptide sequence of SEQ ID NO(s): 1-28, 30-172, 174-190, 192-209, 211-222, 224-235, and 237-239, or SEQ ID NO(s): 1-28, SEQ ID NO(s): 1-9, SEQ ID NO(s): 5, 18, 22, and 23, or SEQ ID NO(s): 6, 11, 12, 13, 14, 18, 19, 20, 26, 27, and 28.

In a further non-limiting embodiment of the first aspect of the present invention, the isolated or synthesized protein, protein fragment, polypeptide, or peptide consists essentially of a Replikin peptide identified in MERS CoV. In a further non-limiting embodiment, the Replikin peptide sequence identified in an MERS CoV is at least one peptide sequence of SEQ ID NO(s): 1-28, 30-172, 174-190, 192-209, 211-222, 224-235, and 237-239.

In a non-limiting embodiment, the Replikin sequence is shared among isolates of MERS CoV isolated from camel and human. In a further non-limiting embodiment, the Replikin sequences shared among human and camel isolates are identical. In a further non-limiting embodiment, the lysine and histidine residues that make the Replikin sequences are identical among human and camel isolates while one or more other amino acid residues are not identical. In non-limiting embodiment, the Replikin sequences are conserved in camels and humans over one, two, three, or more years.

Another non-limiting embodiment of the first aspect of the invention provides a biosynthetic composition comprising the protein, protein fragment, polypeptide, or peptide of an aspect of the invention. In a further non-limiting embodiment, the biosynthetic composition consists essentially of a Replikin peptide of MERS CoV or consists of a Replikin peptide of MERS CoV. In a non-limiting embodiment, an isolated protein, protein fragment, polypeptide, or peptide is chemically synthesized by solid phase methods.

A second non-limiting aspect of the present invention provides an immunogenic and/or blocking composition comprising at least one protein, protein fragment, polypeptide, or peptide of any one of the above-listed proteins, protein fragments, polypeptides, or peptides including and not limited to comprising at least one Replikin peptide sequence identified in MERS CoV or at least one homologue of said at least one Replikin peptide identified in MERS CoV or at least one functional fragment of at least one Replikin peptide sequence identified in MERS CoV. In a non-limiting embodiment of the second aspect of the present invention, the immunogenic and/or blocking composition comprises at least one peptide sequence of SEQ ID NO(s): 1-28. In a further non-limiting embodiment, the immunogenic and/or blocking composition comprises at least one peptide consisting essentially of any one of SEQ ID NO(s): 1-28. In further non-limiting embodiment, the immunogenic and/or blocking composition comprises at least one peptide consisting of any one of SEQ ID NO(s): 1-28 or at least one functional fragment of any one of SEQ ID NO(s): 1-28. In a non-limiting embodiment, the Replikin sequence is shared among isolates isolated from camel and human. In a further non-limiting embodiment, the Replikin sequences are identical. In non-limiting embodiment, the Replikin sequences are conserved in camels and humans over one, two, three, or more years.

A third non-limiting aspect of the present invention provides a vaccine comprising at least one protein, protein fragment, polypeptide, or peptide comprising or consisting of a Replikin sequence or homologue thereof including any one of the above-listed proteins, protein fragments, polypeptides, or peptides. In a non-limiting embodiment, a Replikin sequence is shared among isolates isolated from camel and human. In a further non-limiting embodiment, the Replikin sequences are identical. In a further non-limiting embodiment, the lysine and histidine residues that make the Replikin sequences are identical among human and camel isolates while one or more other amino acid residues are not identical. In a non-limiting embodiment, the Replikin sequences are conserved in camels and humans over one, two, three, or more years.

In a non-limiting embodiment of the third aspect of the present invention, the vaccine comprises at least one peptide sequence of any one of SEQ ID NO(s): 1-9; 1-28; 5, 18, 22, and 23; or SEQ ID NO(s): 6, 11, 12, 13, 14, 18, 19, 20, 26, 27, and 28, comprises at least one peptide sequence consisting essentially of any one of SEQ ID NO(s): 1-9; 1-28; 5, 18, 22, and 23; or SEQ ID NO(s): 6, 11, 12, 13, 14, 18, 19, 20, 26, 27, and 28, and/or comprises at least one peptide sequence consisting of any one of SEQ ID NO(s): 1-9; 1-28; 5, 18, 22, and 23; or SEQ ID NO(s): 6, 11, 12, 13, 14, 18, 19, 20, 26, 27, and 28, or at least one functional fragment of any one of SEQ ID NO(s): 1-9; 1-28; 5, 18, 22, and 23; or SEQ ID NO(s): 6, 11, 12, 13, 14, 18, 19, 20, 26, 27, and 28, at least one functional fragment of a Replikin peptide sequence identified in MERS CoV, or at least one functional fragment of SEQ ID NO(s): 1-9; 1-28; 5, 18, 22, and 23; or SEQ ID NO(s): 6, 11, 12, 13, 14, 18, 19, 20, 26, 27, and 28.

In a further non-limiting embodiment of the third aspect of the present invention, the vaccine comprises a mixture of a plurality peptides of different sequences, wherein each of said peptides of different sequences is at least 80% homologous with at least one Replikin sequence identified in MERS CoV. In a non-limiting embodiment, the vaccine comprises a mixture of a plurality of peptides of different sequences, wherein at least one peptide of the mixture of a plurality of peptides of different sequences is at least 80% homologous with each of SEQ ID NO(s): 1-9, each of SEQ ID NO(s): 1-28, each of SEQ ID NO(s): 5, 18, 22, and 23, or each of SEQ ID NO(s): 6, 11, 12, 13, 14, 18, 19, 20, 26, 27, and 28. In a non-limiting embodiment, the vaccine comprises a mixture of a plurality of peptides of different sequences where at least one peptide of the mixture of a plurality of peptides of different sequences consists of each of SEQ ID NO(s): 1-9, each of SEQ ID NO(s): 1-28, each of SEQ ID NO(s): 5, 18, 22, and 23, or each of SEQ ID NO(s): 6, 11, 12, 13, 14, 18, 19, 20, 26, 27, and 28. In a non-limiting embodiment, a vaccine comprises at least one functional fragment of any one of SEQ ID NO(s): 1-28, 30-172, 174-190, 192-209, 211-222, 224-235, and 237-239. In a non-limiting embodiment, a vaccine comprises a pharmaceutically-acceptable carrier and/or adjuvant.

In a non-limiting embodiment, a vaccine comprises a mixture of a plurality of peptide sequences of any of SEQ ID NO(s): 1-9; 1-28; 5, 18, 22, and 23; or SEQ ID NO(s): 6, 11, 12, 13, 14, 18, 19, 20, 26, 27, and 28 and/or a mixture of a plurality of homologues of peptide sequences of any of SEQ ID NO(s): 1-9; 1-28; 5, 18, 22, and 23; or SEQ ID NO(s): 6, 11, 12, 13, 14, 18, 19, 20, 26, 27, and 28. In a further non-limiting embodiment, the vaccine comprises a mixture of a plurality of peptide sequences consisting essentially of any one or more of SEQ ID NO(s): 1-9; 1-28; 5, 18, 22, and 23; or SEQ ID NO(s): 6, 11, 12, 13, 14, 18, 19, 20, 26, 27, and 28. In a further non-limiting embodiment, the vaccine comprises a mixture of a plurality of peptide sequences consisting of any one or more of SEQ ID NO(s): 1-9; 1-28; 5, 18, 22, and 23; or SEQ ID NO(s): 6, 11, 12, 13, 14, 18, 19, 20, 26, 27, and 28. In a further non-limiting embodiment, the vaccine comprises a mixture of a plurality of peptides consisting of each of SEQ ID NO(s): 1-9; 1-28; 5, 18, 22, and 23; or SEQ ID NO(s): 6, 11, 12, 13, 14, 18, 19, 20, 26, 27, and 28, or any combination of the listed sequences.

In another non-limiting embodiment of the third aspect of the invention, the vaccine comprises a mixture of Replikin peptides. In a non-limiting embodiment, the vaccine comprises an approximately equal molar mixture of the isolated or synthesized peptides of SEQ ID NO(s): 1-9; 1-28; 5, 18, 22, and 23; or SEQ ID NO(s): 6, 11, 12, 13, 14, 18, 19, 20, 26, 27, and 28. In a further non-limiting embodiment, the vaccine comprises approximately equal weight of the isolated or synthesized peptides of SEQ ID NO(s): 1-9; 1-28; 5, 18, 22, and 23; or SEQ ID NO(s): 6, 11, 12, 13, 14, 18, 19, 20, 26, 27, and 28. In a further non-limiting embodiment, the vaccine comprises a pharmaceutically-acceptable carrier and/or adjuvant. In a further non-limiting embodiment, the vaccine comprises peptides in sterile water. In a further non-limiting embodiment, the vaccine comprises freeze-dried peptides in sterile water. In a further non-limiting embodiment, the vaccine is for the treatment or prevention of MERS CoV infection.

A fourth non-limiting aspect of the invention provides an isolated or purified antibody, antibody fragment, or binding agent that specifically binds to at least a portion of a protein fragment or peptide comprising at least one peptide sequence that is at least 80% homologous with at least one Replikin peptide sequence identified in a MERS CoV. In a non-limiting embodiment, the isolate or purified antibody, antibody fragment, or binding agent specifically binds at least a portion of any one of SEQ ID NO(s): 1-28, 30-172, 174-190, 192-209, 211-222, 224-235, and 237-239. In a non-limiting embodiment, the isolate or purified antibody, antibody fragment, or binding agent specifically binds at least a portion of any one of SEQ ID NO(s): 1-28. In a non-limiting embodiment, the isolate or purified antibody, antibody fragment, or binding agent specifically binds at least a portion of any one of SEQ ID NO(s): 1-9. In a non-limiting embodiment, the isolate or purified antibody, antibody fragment, or binding agent specifically binds at least a portion of any one of SEQ ID NO(s): 5, 18, 22, and 23 or SEQ ID NO(s): 6, 11, 12, 13, 14, 18, 19, 20, 26, 27, and 28.

A fourth non-limiting aspect of the invention provides an isolated or purified antibody, antibody fragment, or binding agent that binds to at least a portion of an amino acid sequence of at least one protein, protein fragment, polypeptide, or peptide comprising a peptide sequence that is 30%, 40%, 50%, 60%, 70%, 80%, 90%, or 95% or more homologous with at least one Replikin peptide sequence identified in MERS COV. In a further embodiment, the at least one Replikin peptide sequence identified in MERS COV is at least one peptide sequence of SEQ ID NO(s): 1-9; 1-28; 5, 18, 22, and 23; or 6, 11, 12, 13, 14, 18, 19, 20, 26, 27, and 28.

A fifth non-limiting aspect of the present invention provides a method of making a vaccine comprising: isolating or synthesizing a protein fragment or peptide comprising at least one peptide sequence that is at least 80% homologous with at least one Replikin peptide sequence identified in a MERS CoV as a component of a vaccine; and making the vaccine with the component.

A non-limiting embodiment comprises: selecting at least one isolated or synthesized protein, protein fragment, polypeptide, or peptide comprising at least one peptide sequence that is at least 30%, 40%, 50%, 60%, 70%, 80%, 90% or 95%, or 100% homologous with at least one Replikin peptide sequence identified in MERS CoV as a component of a vaccine; and making said vaccine. In a non-limiting embodiment, the method of making a vaccine comprises selecting at least one isolated or synthesized peptide of SEQ ID NO(s): 1-9; 1-28; 5, 18, 22, and 23; or 6, 11, 12, 13, 14, 18, 19, 20, 26, 27, and 28 as at least one component and making said vaccine with the at least one component.

In another non-limiting embodiment, the method of making a vaccine comprises selecting at least two, three, four, five, six, seven, eight, nine, ten, eleven, twelve, thirteen, fourteen, fifteen, sixteen, seventeen, eighteen, or up to 28 or more isolated or synthesized Replikin peptide sequences identified in MERS CoV and/or isolated or synthesized functional fragments of Replikin peptide sequences identified in MERS CoV and/or homologous of Replikin peptide sequences identified in MERS CoV. In a further embodiment, the isolated or synthesized Replikin peptide sequences, functional fragments of Replikin peptide sequences identified in MERS CoV, or homologues of Replikin peptide sequences identified in MERS CoV comprise at least one peptide sequence of SEQ ID NO(s): 1-28, at least one functional fragment of at least one peptide sequence of SEQ ID NO(s): 1-28, at least one functional fragment of at least one Replikin peptide sequence identified in MERS CoV, or at least one homologues of at least one Replikin peptide sequence identified in MERS CoV. In another non-limiting embodiment, the at least one isolated or synthesized protein, protein fragment, polypeptide, or peptide has the same amino acid sequence as at least one protein, protein fragment, polypeptide or peptide identified in a relatively lethal strain of MERS CoV up to seven days, one month, six months, one year, two years, or three years prior to making said vaccine.

A sixth non-limiting aspect of the present invention provides a method for preventing or treating MERS COV infection comprising administering at least one isolated or synthesized protein, protein fragment, polypeptide, or peptide comprising at least one peptide sequence to an animal or human, where the peptide sequence is at least 30%, 40%, 50%, 60%, 70%, 80%, 90% or 95%, or 100%, homologous with at least one Replikin peptide identified in MERS CoV. In a further non-limiting embodiment, the Replikin peptide sequence is at least one peptide sequence of SEQ ID NO(s): 1-9; 1-28; 5, 18, 22, and 23; or 6, 11, 12, 13, 14, 18, 19, 20, 26, 27, and 28. In a further non-limiting embodiment, the Replikin peptide sequence is at least one peptide sequence of SEQ ID NO(s): 1-28, 30-172, 174-190, 192-209, 211-222, 224-235, and 237-239. In a non-limiting embodiment, the at least one isolated or synthesized protein fragment, polypeptide, or peptide consists of at least one peptide sequence that is at least 30%, 40%, 50%, 60%, 70%, 80%, 90%, or 95% or more homologous with at least one of the peptide sequences of SEQ ID NO(s): 1-28. In another non-limiting embodiment, the at least one isolated or synthesized peptide of SEQ ID NO(s): 1-28 is administered to a human or other animal. In a further non-limiting embodiment the at least one Replikin peptide sequence is at least one peptide sequence of SEQ ID NO(s): 1-28.

A non-limiting embodiment of the sixth aspect provides use of at least one Replikin peptide sequence identified in MERS CoV, at least one homologue of at least one Replikin peptide sequence identified in MERS CoV, or at least one functional fragment of at least one Replikin peptide sequence identified in MERS CoV in the manufacture of a medicament for preventing or treating MERS CoV infection. In a non-limiting embodiment, the Replikin peptide sequence is any one of SEQ ID NO(s): 1-28, 30-172, 174-190, 192-209, 211-222, 224-235, and 237-239. A non-limiting embodiment provides use of a Replikin sequence, a homologue of a Replikin sequence, or a functional fragment of a Replikin sequence of MERS CoV for the prevention or treatment of MERS CoV.

A seventh non-limiting aspect of the present invention provides a method of predicting expansion or retraction of a population of MERS CoV comprising, respectively, identifying an increase in the percentage of isolates of MERS CoV having a Replikin concentration (number of Replikin sequences per 100 amino acid residues) in at least one portion of the genome greater than 4.0 between two time periods or identifying a decrease in the percentage of isolates of MERS CoV having a Replikin concentration (number of Replikin sequences per 100 amino acid residues) in at least one portion of the genome greater than 4.0 between two time periods. In a non-limiting embodiment, the more than two time periods are compared and the percentage of isolates in the more than two time periods shows a pattern of increase or decrease. In a non-limiting embodiment, the portion of the genome reflects encoding of an expressed protein. In a non-limiting embodiment, the portion of the genome is N, M, S, ORF4b, ORF1ab, ORF1a, ORF1b, NS3C, N3, or any other gene segment of the genome.

A non-limiting embodiment of the seventh aspect of the present invention provides a method of differentiating between relatively more lethal and relatively less lethal forms of MERS CoV. A non-limiting embodiment provides a method of identifying and/or diagnosing a relatively more lethal form of MERS CoV comprising determining the Replikin concentration of at least one portion of at least one protein of at least one isolate of MERS CoV or at least one portion of at least one gene that expresses at least one protein of the at least one isolate of MERS CoV and comparing the Replikin concentration of the at least one isolate of MERS CoV to a comparable Replikin concentration in at least one other isolate of MERS CoV. In a further non-limiting embodiment, the at least one portion of at least one protein comprises the entirety of at least one protein expressed in MERS CoV and the comparable Replikin concentration is the Replikin concentration of the entirety of the same protein expressed in MERS CoV from the at least one other isolate of MERS CoV. In a non-limiting embodiment, the Replikin concentration of the at least one isolate of MERS CoV is a mean of Replikin concentrations determined in a plurality of isolates of MERS CoV. In a further non-limiting embodiment, the Replikin concentration of the at least one other isolate of MERS CoV is a mean of Replikin concentrations determined in a plurality of other isolates of MERS CoV. In a further non-limiting embodiment, the plurality of isolates of MERS CoV is a collection of isolates isolated in a given year and the plurality of other isolates of MERS COV is a collection of isolates isolated in a different year. In a further non-limiting embodiment, the Replikin concentration of the more lethal isolate of MERS COV is 3.0 or greater, 4.0 or greater, or 5.0 or greater per 100 amino acid residues. In a further non-limiting embodiment, the Replikin concentration of the more lethal isolate of MERS COV is 4.0 or greater per 100 amino acid residues. In a further non-limiting embodiment, the Replikin concentration of the more lethal isolate of MERS COV is 4.6 per 100 amino acid residues or greater. In a further non-limiting embodiment, a vaccine is manufactured following the differentiation between relatively more lethal and relatively less lethal forms of MERS CoV. In a further non-limiting embodiment, the vaccine comprises at least one structure of the isolate of MERS CoV differentiated as relatively more lethal. In a further non-limiting embodiment, the vaccine comprises at least one Replikin peptide sequence identified in the isolate of MERS CoV differentiated as relatively more lethal.

In a further non-limiting embodiment of the seventh aspect of the present invention, the at least one portion of at least one gene expressing at least one protein is at least one portion of the ORF4b gene region.

In a further non-limiting embodiment of the seventh aspect of the present invention, the Replikin concentration of the at least one isolate of MERS CoV is greater than the Replikin concentration of the at least one other isolate of MERS CoV. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

Another non-limiting embodiment of the seventh aspect of the invention provides a method of determining an increased probability of an outbreak of MERS CoV within about one year following an increase in Replikin concentration in an isolate of MERS CoV comprising identifying an increase in the concentration of Replikin sequences in at least one first isolate of MERS CoV as compared to at least one other isolate of MERS CoV wherein said at least one first isolate is isolated at a later time period than said one other isolate and wherein said increase in the concentration of Replikin sequences signifies the increased probability of the outbreak of MERS CoV within about one year following the increase in the concentration of Replikin sequences.

In a non-limiting embodiment, a method of prediction comprises: (1) obtaining a plurality of isolates of MERS CoV wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an outbreak of MERS CoV within about one month to about three years following said identified increase in the concentration of Replikin sequences. In another embodiment of the invention, the outbreak of MERS CoV is predicted within about six months. In a further embodiment of the invention, the outbreak of MERS CoV is predicted within about one year to about three years. In a further non-limiting embodiment, the method of prediction further comprises processing at least one step of the method on a computer.

In a further non-limiting embodiment of the seventh aspect of the invention, the method of prediction further comprises comparison of the standard deviation from the mean of Replikin concentrations of isolates of MERS CoV from a given time period, such as a given month, a given year, or any other given time period. In a further non-limiting embodiment, the Replikin concentration is a mean Replikin concentration of a plurality of isolates with standard deviation from the mean and the standard deviation from the mean is greater than the standard deviation from the mean Replikin concentration of a plurality of other isolates.

A further non-limiting embodiment provides a computer readable medium having stored thereon instructions which, when executed, cause a processor to perform a method of predicting an expansion of a strain of MERS CoV or an increase in virulence, morbidity, and/or mortality of MERS CoV. In a further embodiment, the processor reports a prediction to a display, user, researcher, or other machine or person. In a further embodiment, the processor identifies to a display, user, researcher, or other machine or person, a portion of a pathogen predicted to be an expanding MERS CoV or predicted to increase in virulence, morbidity, and/or mortality, wherein said portion may be employed as a therapeutic or diagnostic compound. Said portion may be a Replikin peptide or plurality of Replikin peptides or any other structure or portion of said genome of said pathogen including a Replikin Peak Gene.

Another non-limiting embodiment of the seventh aspect of the invention provides a computer system, including a processor coupled to a network and a memory coupled to the processor, the memory containing a plurality of instructions to perform a method of predicting an increase in virulence, morbidity, or mortality as compared to at least one second malignancy.

Another non-limiting embodiment of the seventh aspect of the invention provides a machine-readable storage medium having stored thereon executable instructions that, when executed by a processor, cause the processor to provide sufficient data to a user, a display, or a printout such that said user or a user of said display or said printout may predict an increase in virulence, morbidity, or mortality of MERS CoV based on regression analysis. Another non-limiting embodiment provides a computer system, comprising: a processor coupled to a network; a memory coupled to the processor, the memory containing a plurality of instructions to perform the method of predicting an increase in virulence, morbidity, or mortality of MERS CoV based on the regression analysis.

A non-limiting embodiment provides an electromagnetic signal carrying computer-readable instructions for performing a method of predicting an outbreak of MERS CoV or the relative lethality or virulence of MERS CoV. In a non-limiting embodiment, the signal is non-transient.

### BRIEF DESCRIPTION OF THE DRAWINGS

FIG. 1 illustrates percent of publicly available genomic sequences of isolates of MERS CoV for given time periods analyzed as having Replikin sequences per 100 amino acid residues (Replikin Count) of greater than 4.0. The graph reflects analysis of the genomic or proteomic information publicly available for isolates at the PubMed website of the National Center for Biotechnology Information. Data are illustrated from April through June of 2012, May through June of 2012, September 2012, October 2012, and February through June 2013. The data show a rise in the percentage of MERS CoV isolates having a Replikin concentration (number of Replikin sequences per 100 amino acid residues) of greater than 4.0 from April through June 2012 to September 2012 followed by a decline in percentage of isolates of MERS having a Replikin concentration of greater than 4.0 from September 2012 through February through June 2013. The percentage of isolates of MERS CoV having a Replikin concentration of greater than 4.0 is around 16 percent in April through June 2012, rises to around 35 percent in September 2012, and falls back to around 17 percent in February through June of 2013. Observation of this fall in percent of isolates having Replikin concentration of greater than 4.0 predicts the MERS CoV outbreak of 2012 and 2013 will continue to diminish.

FIG. 2 illustrates an increase in Replikin concentration (Replikin sequences per 100 amino acid residues) in spike and nucleocapsid coronavirus proteins preceding the SARS coronavirus epidemic of 2003. The x-axis indicates the year and the y-axis indicates the Replikin concentration. The appearance of the SARS outbreak and the eight countries involved in the outbreak is shown by the conical shaded area. The solid black symbols represent the mean Replikin concentration for spike coronavirus proteins and the vertical black bars represent the standard deviation of the mean. Replikin concentration rose between 1995 and 2002, consistent with the SARS coronavirus outbreak, which emerged at the end of 2002 and persisted into 2003. The decline in Replikin concentration correctly signaled the end of the SARS outbreak and had already begun its return to pre-outbreak levels when the outbreak emerged.

FIG. 3 illustrates Replikin concentrations (Replikin sequences per 100 amino acid residues) for genomic or proteomic information publicly available for isolates of H1N1 at the PubMed website of the National Center for Biotechnology Information for hemagglutinin protein sequences (Infectivity Gene) and for pB1 protein sequences (Lethality Gene) from 2001 through April 2010. Gray bars illustrate mean Replikin concentration of hemagglutinin proteins with standard deviation bars above and black bars illustrate mean Replikin concentration of pB1 proteins with standard deviation bars above. The graph illustrates an increase in Replikin concentration (with a p value of less than 0.001) in Human H1N1 Influenza Virus Infectivity Gene from 2002 through April 2008, which prospectively predicted the global H1N1 influenza epidemic of 2009, followed by persistence in the elevation of Replikin concentration of the Infectivity Gene through April 2010. The persistence predicted that a clinical recurrence was likely, which was observed in December 2010. The data in FIG. 3 illustrate mean Replikin concentration determinations annually from 2001 through 2008 and then mean Replikin concentration determinations every 2 to 3 days, then weekly, from Apr. 30, 2009.

FIG. 4 illustrates a high-level block diagram of a computer system incorporating a system and method for identifying Replikin patterns in amino acid sequences.

FIG. 5 illustrates a simple flow chart illustrating a general method for locating a Replikin pattern in a sequence of amino acids.

FIG. 6 illustrates a flow chart illustrating a generalized method for locating a plurality of Replikin-like patterns in a sequence of amino acids.

### DETAILED DESCRIPTION OF THE INVENTION

### Definitions

A “protein fragment” as used in this specification is any fragment of an expressed whole protein, which is any portion of an expressed whole protein where a “portion” of a protein is less than an expressed whole protein. A protein fragment reflects an expressed whole protein with one or more amino acids removed from the amino acid sequence of the expressed whole protein. A protein fragment may also reflect an amino acid sequence that is at least 30%, 40%, 50%, 60%, 70%, 80%, 90%, or 95% or greater homologous with any portion of an expressed whole protein. A “polypeptide,” as used in this specification, is any portion of a protein fragment and is less than an expressed whole protein.

A “whole protein” or an “expressed whole protein” as used in this specification reflect a protein that is expressible from an intact gene of MERS CoV from a start codon to a stop codon. A whole protein or expressed whole protein may also reflect a whole protein or expressed whole protein that has been subject to cellular processing to create a protein that is capable of functioning within the virus replication system in a proper manner for virus replication. A protein fragment, polypeptide, or peptide “partially matches” the amino acid sequence of an expressed whole protein when the protein fragment, polypeptide, or peptide shares substantial homology with at least a portion of the expressed whole protein.

A “functional fragment” of a Replikin sequence as described herein is a fragment, variant, analog, or chemical derivative of a Replikin sequence that retains at least a portion of the immunological cross reactivity with an antibody specific for the Replikin sequence. A fragment of the Replikin sequence refers to any subset of the molecule. Variant peptides of the sequence may be made by direct chemical synthesis, for example, using methods well known in the art. An analog of a Replikin sequence to a non-natural protein or polypeptide is substantially similar to either the Replikin sequence of the protein or a fragment thereof. Chemical derivatives of a Replikin sequence contain additional chemical moieties.

As used herein, the term “preferentially binds” or “specifically binds” and related terms referencing the interaction of a binding molecule such as, for example, an antibody, and the structure to which it binds (antigen) means that the binding molecule preferentially recognizes the structure to which it binds even when present among other molecules (such as in a mixture of molecules). Specific or preferential binding of a binding molecule to a binding structure or an immunogenic portion of a binding structure is specific and preferential when the binding molecule binds to the structure or portion thereof and does not bind with the same level of affinity to other structures. Binding affinity may be determined by one of ordinary skill in the art using, for example, BIACORE, enzyme-linked immunosorbent assays, or radioimmuno assays. A binding molecule may cross-react with related antigens and preferably does not cross-react with affinity to unrelated antigens. Binding between a binding molecule and the structure to which it binds may be mediated by covalent or non-covalent attachment, or both.

As used herein a “vaccine” is any substance, compound, composition, mixture, or other therapeutic substance that, when administered to a human or animal via any method of administration known to the skilled artisan now or hereafter, produces an immune response, an antibody response, a blocking response, or a protective effect in the human or animal.

As used herein, a “Replikin sequence” is an amino acid sequence of 7 to 50 amino acid residues comprising (1) a first lysine residue located six to ten residues from a second lysine residue; (2) at least one histidine residue; and (3) at least 6% lysine residues, where the sequence is the shortest sequence comprising the first and second lysine residues of element (1) and the at least one histidine of element (2). A Replikin sequence may comprise more than two lysine residues and more than one histidine residue so long as at least two of the lysine residues and at least one histidine residue reflect the requirements of the definition of a Replikin sequence. A Replikin sequence may be targeted by the immune system within a peptide sequence that comprises more than the shortest sequence comprising the lysine and histidine residues required for a Replikin sequence.

The term “Replikin sequence” can also refer to a nucleic acid sequence encoding an amino acid sequence having 7 to about 50 amino acids comprising:
(1) at least one lysine residue located six to ten amino acid residues from a second lysine residue;
(2) at least one histidine residue; and
(3) at least 6% lysine residues,
   where the sequence is the shortest sequence encoding the first and second lysine residues of element (1) and the at least one histidine of element (2).

In polypeptides, peptides, proteins, and protein fragments, the carboxyl group of one amino acid is attached to an amino group of another amino acid via a peptide bond forming a compound that is a chain of amino acid residues.

As used herein, a “synthesized” peptide may be synthesized by organic chemical methods and may be synthesized by biosynthetic methods. An “isolated” peptide may refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques. A protein or peptide that has been isolated in silico from nucleic acid or amino acid sequences that are available through public or private databases or sequence collections may be synthesized or isolated as compounds. An isolated peptide may be synthesized by biosynthetic or organic chemical methods.

Proteins, protein fragments, polypeptides, or peptides in this specification may be chemically synthesized by any method known to one of skill in the art now and hereafter. For example, isolated proteins, protein fragments, polypeptides, or peptides may be synthesized by solid phase synthesis. The production of these materials by chemical synthesis avoids the inclusion of (or the need to remove by purification) materials that are byproducts of other production methods such as recombinant expression or isolation from biological material. Such byproducts may include, for example, avian proteins associated with vaccines produced using birds' eggs, bacterial proteins associated with recombinant production in bacteria, or proteins or contaminants associated with any recombinant activity such as with productions of proteins or other sequences in insect cells.

An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well known in the art that, due to redundancy in the genetic code, individual nucleotides can be readily exchanged in a codon and still result in an identical amino acid sequence. As will be understood by one of ordinary skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

“Homologous” or “homology” or “sequence identity” as used in this specification indicate that an amino acid sequence or nucleic acid sequence exhibits substantial structural equivalence with another sequence, namely, any Replikin peptide sequence (including SEQ ID NO(s): 1-28) identified in an isolate of MERS CoV or any nucleotide sequence encoding a Replikin peptide sequence in an isolate of MERS CoV (a redundancy in a coding sequence may be considered identical to a sequence encoding the same amino acid). To determine the percent identity or percent homology of an identified sequence, a sequence is aligned for optimal comparison purposes with any one of possible basis sequences. For purposes of this paragraph, a basis sequence is a Replikin sequence identified in an isolate of MERS CoV. Where gaps are necessary to provide optimal alignment, gaps may be introduced in the identified sequence or in the basis sequence. When a position in the identified sequence is occupied by the same amino acid residue or same nucleotide as the corresponding position in the basis sequence, the molecules are considered identical at that position (as used herein amino acid or nucleic acid “identity” is equivalent to amino acid or nucleic acid “homology”). To determine percent homology, the amino acid residues or nucleotides at corresponding amino acid positions or nucleotide positions are compared between the identified (reference) sequence and the basis sequence. The total number of amino acid residues or nucleotides in the identified sequence that are identical with amino acid residues or nucleotides in the basis sequence is divided by the total number of residues or nucleotides in the basis sequence (if the number of residues or nucleotides in the basis sequence is greater than the total number of residues or nucleotides in the identified sequence) with gaps included or by the total number of amino acid residues or nucleotides in the identified sequence (if the number of residues or nucleotides in the identified sequence is greater than the total number of residues or nucleotides in the basis sequence) with gaps included. The final number is determined as a percentage. As such, the percent identity between the two sequences is a function of the number of identical positions shared by the sequences, taking into account the number of gaps (where a gap must be introduced for optimal alignment of the two sequences) and the length of each gap. Any structural or functional differences between sequences having sequence identity or homology will not affect the ability of the sequence to function as indicated in the desired application.

For example, SEQ ID NO: 6 (KHVEVFTDGK) is considered more than 90% homologous with SEQ ID NO: 8 (KKHVEVFTDGK). The more than 90% homology between SEQ ID NO: 6 and SEQ ID NO: 8 is determined as follows: SEQ ID NO: 6 is the identified sequence. SEQ ID NO: 8 is the basis sequence. Upon alignment, SEQ ID NO: 6 is identical to SEQ ID NO: 8 in all 11 residues of SEQ ID NO: 8 with the exception of the first lysine in SEQ ID NO: 8. To determine percent homology, then, the 10 aligned identical residues are divided by the total number of residues in SEQ ID NO: 8, namely 11 residues, giving 0.909 or greater than 90% homology.

In a further example, SEQ ID NO: 24 (KDAAAAKNKMRH) is more than 46% homologous with SEQ ID NO: 22 (KNKMRHK). SEQ ID NO: 24 is the basis sequence and has 12 residues. SEQ ID NO: 22 is the identified (or reference) sequence and has 7 residues. Six of the seven residues in SEQ ID NO: 22 are in the same position as six residues in SEQ ID NO: 24 when SEQ ID NO: 22 and SEQ ID NO: 24 are optimally aligned. A gap of one residue on the C-terminal end of SEQ ID NO: 24 must be added to encompass the lysine at the C-terminus of SEQ ID NO: 22. Including the gap of one residue on the C-terminal end, SEQ ID NO: 24 has a length of 13 residues. To determine percent homology, then, the 6 aligned identical residues are divided by the total number of residues in SEQ ID NO: 24 (with gap include), namely, 13 residues, giving 0.461 or more than 46% homology.

In another examples, SEQ ID NO: 22 (KNKMRHK) is more than 53% homologous with SEQ ID NO: 9 (KDAAAAKNKMRHK) because the seven residues of SEQ ID NO: 22 align exactly with seven residues of SEQ ID NO: 9 and SEQ ID NO: 9 has 13 total residues (giving greater than 53% homology).

Concerning gaps, the number of gaps in either the basis sequence or the identified sequence should be limited to the number of gaps allowable without significantly compromising the function of the identified sequence as compared to the basis sequence. In general, many gaps in the sequence of the basis peptide or in the sequence of the identified peptide are allowed based on homology as defined herein. Relatively more gaps are allowed if the lysines and histidines that create the definition of the Replikin peptide are identically shared between the basis peptide and the identified peptide. Relatively more gaps are also allowed if the lysines and histidines that create the definition of the Replikin peptide are shared at least in close position (for example within ten, nine, eight, seven, six, five, four, three, two, or one amino acid residue). If some of the lysine residues and histidine residues that create the definition of the Replikin peptide are not present in the identified peptide, fewer gaps may be allowed. Nevertheless, if the identified peptide functions similarly to the basis peptide, any number of gaps is allowed. In general, three or more gaps are allowed in the sequence of the basis peptide or in the sequence of the identified peptide within ten amino acid residues of the basis peptide if no lysines or histidines are present in the identified peptide. Two or more gaps or one or more gaps are also allowed. Nevertheless, if the identified sequence provides the same or a similar function to the basis sequence, more gaps are allowed up to the number of gaps that will provide a homology of 30%, 40%, 50%, 60%, 70%, 80%, 90%, 95%, or more homology. Additionally, where the lysines and histidines of the Replikin definition are present in both the identified peptide and the basis peptide, there should be no limit on how many gaps are allowed.

The presence of lysines and histidines providing for the Replikin definition in an identified peptide requires significantly less homology because the lysines and the histidines of the Replikin definition provide for conservation of Replikin function. For example, in Table 8 and the description thereof in columns 62 and 63 in U.S. Pat. No. 7,442,761, a highly-mutable tat protein in HIV is described and analyzed. As may be seen from Table 8 in U.S. Pat. No. 7,442,761, in tat protein of HIV, which is essential for replication in the virus, lysines and histidines that are essential to maintaining the Replikin definition within a key Replikin peptide in the protein are observed to be 100% conserved, while substitutions in amino acid residues that are not essential to maintaining the Replikin definition are commonly substituted. The conservation of the key amino acids for maintaining the Replikin definition is understood to provide a specific survival function for HIV. The same phenomenon is seen in influenza. See U.S. Pat. No. 7,442,761, column 62, lines 42-45.

As used herein, “Replikin Count” or “Replikin concentration” refers to the number of Replikin sequences per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence, wherein a Replikin sequence is a peptide of 7 to 50 amino acid residues comprising (1) a first lysine residue six to ten residues from a second lysine residue, (2) at least one histidine residue, (3) and 6% or more lysine residues where the Replikin sequence is the shortest sequence comprising the first and second lysine residues of element (1) and the at least one histidine residue of element (2). A Replikin sequence may comprise more than two lysine residues and more than one histidine residue so long as there is at least one lysine residue six to ten residues from a second lysine residue and at least one histidine residue. A Replikin sequence for the purpose of determining Replikin concentration as described in this paragraph may also be a nucleic acid that encodes a Replikin peptide sequence defined according to this paragraph.

### Quantitative Real-Time Analysis of Replikin Sequences in MERS CoV

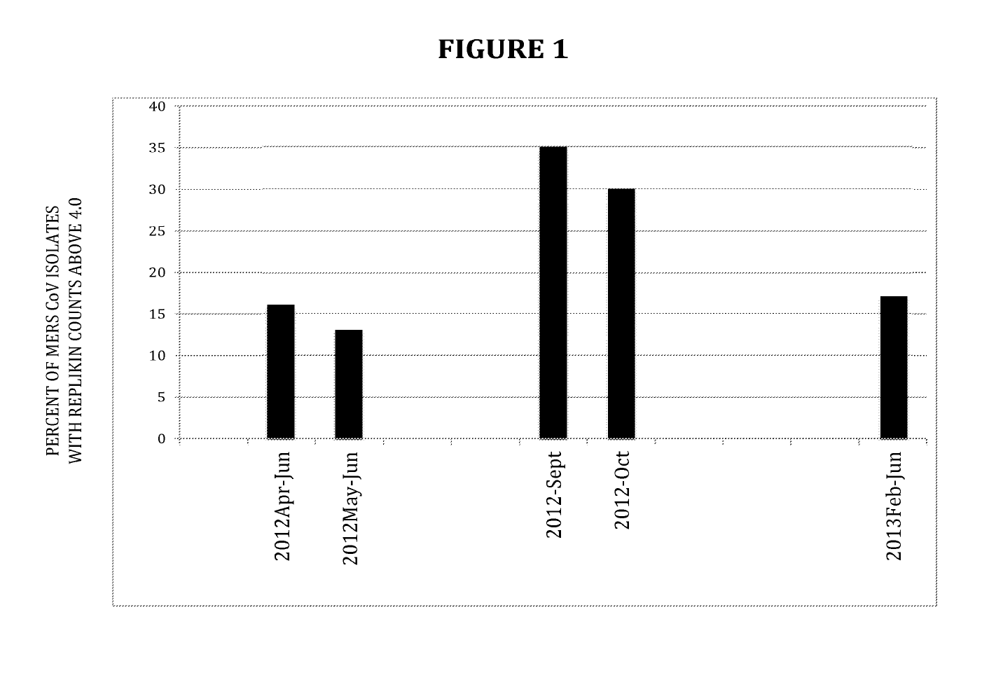

Quantitative Real-Time Replikins Analysis of the virus genome of lethal Middle Eastern Respiratory Syndrome Coronavirus (MERS CoV) demonstrates in FIG. 1 that the percent of the virus' genomic Replikins concentrations greater than 4.0 per 100 amino acids, an indication of virulence and infectivity, has dropped from its peak of 35% in 2012 to 17% on specimens collected up to June 2013.

The Figure summarizes all of the data listed in at the NCBI PubMed database. No data were available for large intervals of up to four months. Further, many sequences were published only up to 4 months after specimen collection.

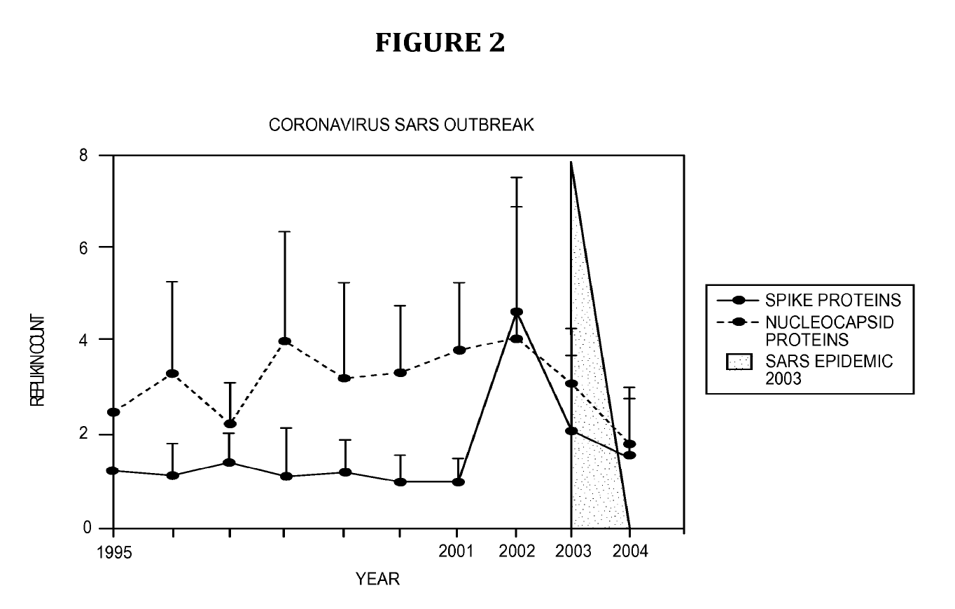

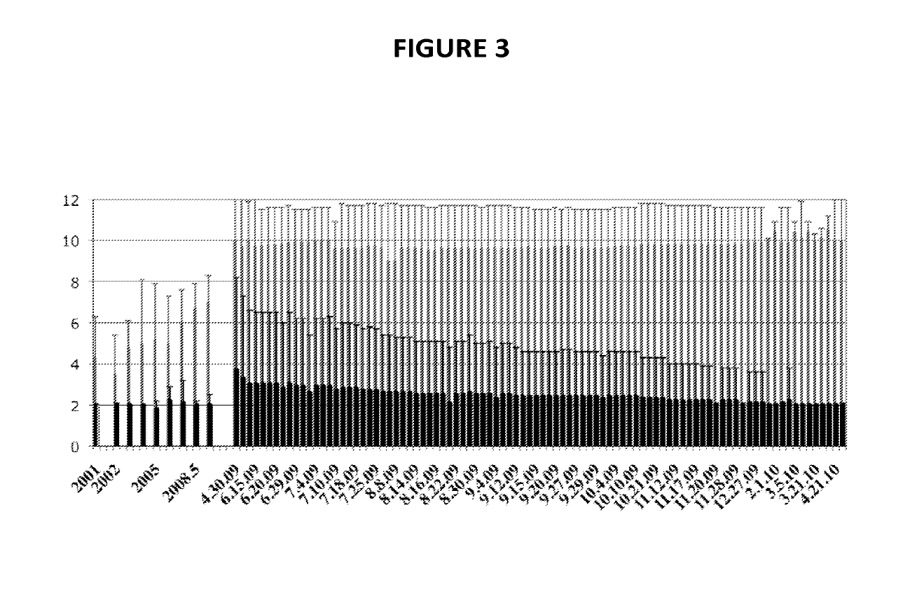

The rapid quantitative decline in MERS CoV gene Replikin sequences reflected in FIG. 1 resembles that previously found by Replikins Bioradar UK Ltd in the outbreak of SARS in 2002. See FIG. 2. SARS is a coronavirus related to MERS CoV. After an abrupt rise in gene Replikin Count in 2002, SARS Replikins began their immediate decline in concentration as the clinical outbreak occurred and quickly terminated in 2003. In another example, the occurrence of the H1N1 pandemic of 2009 was predicted one year in advance, its termination in 2009 (see FIG. 3) and its recurrence in 2010, all were predicted by the Replikin Count. However, the course of MERS CoV appears to resemble that of its coronavirus SARS relative more than the course of the influenza virus H1N1 in the 2009 pandemic. Real-time quantitative gene analysis is changing the surveillance of emerging virus diseases and offers the possibility of pandemic prevention.

Because genomic Replikin changes can occur rapidly, and these correlate very closely with clinical outcomes, it is important that the genomic sequences be published as soon as possible after the specimens are collected. Ideal surveillance, attainable now, would have sequences published within a few days of specimen collection and Replikin analysis performed within an additional 24 hours. Testing completely synthetic Replikins vaccines based on this technology can be available quickly, in days to weeks.

### Methods of Predicting Outbreaks of MERS CoV

One non-limiting aspect of the present invention provides a method of predicting expansion or retraction of a population of MERS CoV comprising, respectively, identifying an increase in the percentage of isolates of MERS CoV having a Replikin concentration (number of Replikin sequences per 100 amino acid residues) greater than 4.0 between two time periods or identifying a decrease in the percentage of isolates of MERS CoV having a Replikin concentration (number of Replikin sequences per 100 amino acid residues) greater than 4.0 between two time periods. In a non-limiting embodiment, the more than two time periods are compared and the percentage of isolates in the more than two time periods shows a pattern of increase or decrease.

In a further non-limiting embodiment, a plurality of isolates in a given time period may be analyzed for Replikin concentration. The percentage of isolates having a Replikin concentration of greater than 4.0 Replikin sequences per 100 amino acid residues in a first time period may be compared to the percentage of isolates having a Replikin concentration of greater than 4.0 Replikin sequences per 100 amino acid residue in a second time period. If the later timer period has a higher percentage of isolates having a Replikin concentration of greater than 4.0 then it is predicted that the MERS population will expand and an outbreak is more likely. If the later time period has a lower percentage of isolates having a Replikin concentration of greater than 4.0 then it is predicted that the MERS populations will retract and an outbreak is expected to decrease in severity.

For example, FIG. 1 provides data demonstrating a marked increase in percent of isolates having a Replikin concentration of greater than 4.0 between April and September 2012 (predicting an outbreak) and a marked decrease in percent of isolates having a Replikin concentration of greater than 4.0 between September 2012 and June 2013 (predicting a retraction in the population).

One non-limiting aspect of the present invention provides a method of determining an increased probability of an outbreak of MERS CoV within about six months to about three years following an increase in Replikin concentration in an isolate of MERS CoV comprising identifying an increase in the concentration of Replikin sequences in at least one first isolate of MERS CoV as compared to at least one other isolate of MERS CoV wherein said at least one first isolate is isolated later than said at least one other isolate is isolated, and wherein said increase in the concentration of Replikin sequences signifies the increased probability of the outbreak of MERS CoV within about six months to about three years following the increase in the concentration of Replikin sequences. In a non-limiting embodiment, the first isolate of MERS CoV is isolated at least about six months later than the at least one other isolate.

In a non-limiting embodiment, a method of prediction comprises: (1) obtaining a plurality of isolates of MERS CoV, wherein at least one of said isolates is isolated later (less than six months later or about six months to about 3 years later) than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period (said period may be one day, one week, one month, or six months or greater); and (5) predicting an outbreak of MERS CoV following said identified increase in the concentration of Replikin sequences. In one embodiment of the invention, the outbreak of MERS CoV is predicted within about six months to about five years. In a further embodiment of the invention, the outbreak of MERS CoV is predicted within about one year to about three years.

In a further non-limiting embodiment of the aspect of the invention, the method of prediction further comprises comparison of the standard deviation of the mean of Replikin concentrations of isolates of MERS CoV from a given time period, such as a given month, a given year, or any other given time period. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

### Computer Methods for Determining Expansion, Retraction, and Outbreaks of MERS CoV

A prediction of expansion or retraction of a MERS CoV population may be performed by a processor. A prediction may be output to a user or display. Likewise, a particular Replikin peptide or Replikin Peak Gene within an isolate or population of isolates of MERS CoV predicted to be expanding or retracting in replication or lethality may be output to a user or display. A machine-readable storage medium may contain executable instructions that, when executed by a processor, cause the processor to provide sufficient data to a user, a printout, or a display such that the user or a user of the printout or display may predict expansion or retraction of population of MERS CoV. A process for prediction may comprise: comparing a Replikin Count of at least one first isolate of MERS CoV with a Replikin Count of at least one second isolate of MERS CoV; and predicting the population of the first isolate to be expanding if the Replikin Count of the first isolate is greater than the Replikin Count of the second isolate. In another embodiment, predicting the population of the first isolate to be expanding if the Replikin Count of the first isolate is greater than four Replikin sequences per 100 amino acid residues.

A computer system may include a processor coupled to a network, and a memory coupled to a processor, wherein the memory contains a plurality of instruction to perform the methods of prediction discussed herein.

A user of outputted data from a processor, storage medium, machine-readable medium, signal, or computer system may include any person or any machine that records or analyzes the outputted data. A display or printout may include any mechanism by which data is outputted so that any person or any machine may record or analyze the outputted data, including a printed document, a visual impulse, an aural impulse, a signal, or any other perceivable impulse, a computer monitor, a set of numbers, or any other display or printout of data including a digital recording medium.

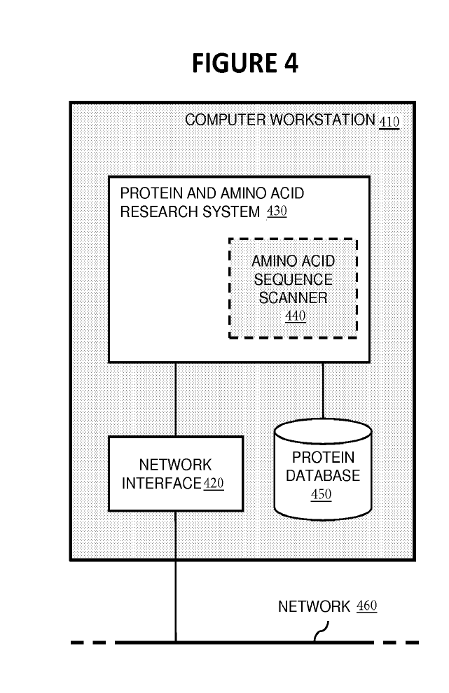

Concerning a non-limiting computer system that may implement methods disclosed in the application, FIG. 4 is a high-level block diagram of a computer system incorporating a system and method for identifying Replikin patterns in amino acid sequences. As shown in FIG. 4, computer workstation 410 may be a computer having a processor and a memory configured to permit a researcher to search protein databases and to scan protein descriptions for selected amino acid patterns. To accomplish these functions, computer workstation 410 may include protein and amino acid research system 430, which may receive instructions from a user/researcher to conduct protein searching and amino acid scanning operations. Protein and amino acid research system 430 may further include amino acid sequence scanner 440 that scans and searches retrieved protein and amino acid sequences for specific patterns of amino acids, including Replikin patterns. Protein and amino acid research system 430 may communicate with network interface 420 to obtain protein sequences and amino acid sequences from resources on network 460, which may include the Internet. Alternatively, protein and amino acid research system 430 may obtain protein sequences and amino acid sequences from a local protein database 450. In addition, protein and amino acid research system 430 may obtain protein sequences and amino acid sequences directly from other input means, such as keyboard input. Protein and amino acid research system 430 may also communicate with network interface 420 to transmit results to other computers on network 460.

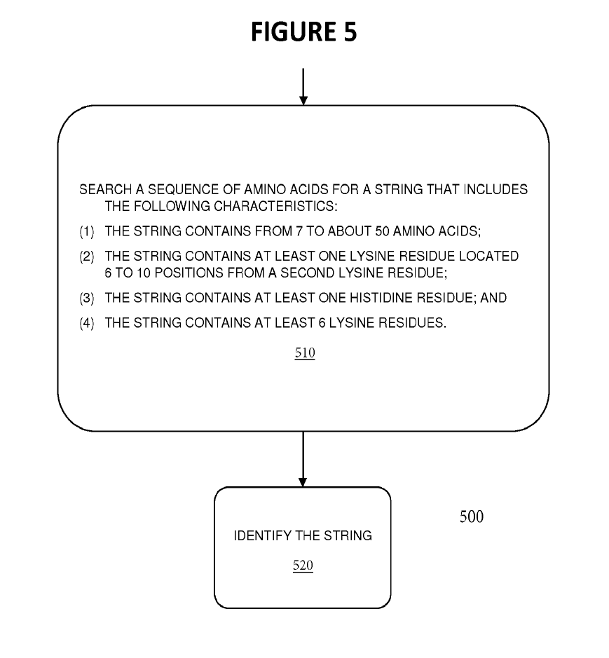

FIG. 5 is a simple flow chart illustrating a general method for locating a Replikin pattern in a sequence of amino acids. The method 500 may begin after a sequence of amino acids has been obtained. Typically, the sequence of amino acids may be represented by three alphabetic characters. However, other encodings are envisioned by the present invention as well. Referring to FIG. 5, once a sequence of amino acids has been obtained, the sequence is searched for a Replikin pattern (510), which comprises a subsequence (or string) of amino acids that includes the following characteristics:
(1) the string contains from 7 to about 50 amino acids;
(2) the string contains at least one lysine residue located 6 to 10 positions from a second lysine residue;
(3) the string contains at least one histidine residue; and
(4) the string contains at least 6% lysine residues.

Once a string of amino acids is found to match the Replikin pattern, the string may be identified or marked (520) accordingly.

A given sequence of amino acids may contain many subsequences or strings that match the Replikin pattern. Additionally, Replikin patterns may overlap each other. Thus, to locate and identify all possible Replikin patterns in a sequence of amino acids, method 500 may be invoked iteratively for each subsequence of amino acids contained within the original sequence of amino acids.

When method 500 is invoked iteratively to identify and locate all possible Replikin patterns in an amino acid sequence, the number of resulting Replikin patterns may be counted. A Replikin count may be reported as an absolute number. Additionally, a ratio of the number of Replikins per N amino acids in the sequence may be calculated. For example, it may be determined that a given protein contains a ratio of 6 Replikin sequences for every 100 amino acid residues. Replikin ratios have been shown by laboratory experiment and by epidemiological evidence to correlate directly to the rate that a given virus replicates. Rapid replication in pathogens is an indication of disease. For example, the presence of relatively high ratios of Replikin patterns has been correlated to epidemics of influenza. Similarly, an increase in the count of Replikin patterns observed in a protein over time indicates a future disease caused by the organism from which the protein was obtained.

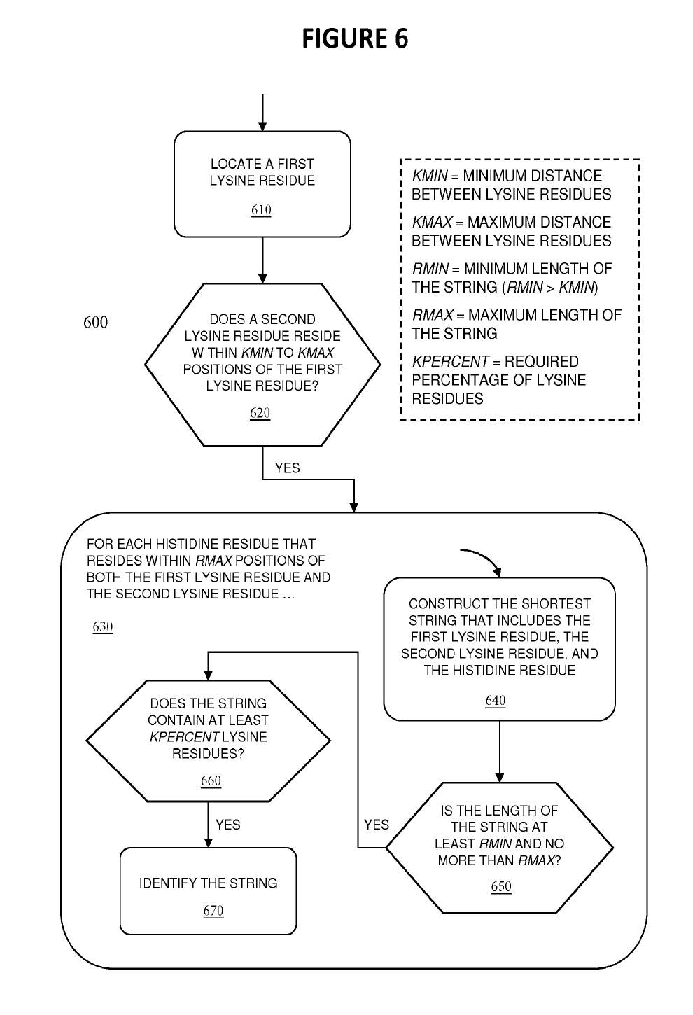

FIG. 6 is a flow chart illustrating a generalized method 600 for locating a plurality of Replikin patterns in a given sequence of amino acids. The method 600 begins by locating a first lysine residue in the given sequence (610). Then, the method 600 may determine whether a second lysine residue resides within kmin to kmax positions of the first lysine residue (620). As indicated in FIG. 6, kmin and kmax define the limits on the distance between the first and second lysine residues. For a typical Replikin pattern, kmin will equal 6 and kmax will equal 10. However, these values may be varied by a researcher interested in discovering other similar patterns.

Once method 600 has identified two lysine residues that are close enough to each other (620), the method 600 may examine every histidine residue that resides within rmax positions of both the first and second lysine residues (630). When method 600 is employed to identify and locate typical Replikin patterns, rmax will be set to equal 50. For every histidine residue that resides within rmax positions of the two lysine residues identified in steps (610) and (620), method 600 will construct the shortest string of amino acid residues that includes the first lysine residue, the second lysine residue, and the identified histidine residue (640). Then, method 600 will determine whether the length of that shortest string is within the desired range—that is, whether it contains at least rmin amino acid residues and no more than rmax amino acid residues (650). Finally, if the identified string of amino acids also contains at least kpercent of lysine residues (660), the string will be identified as matching the desired Replikin pattern (670).

Still referring to FIG. 6, it is apparent that method 600 may identify several Replikin patterns from a single given amino acid sequence. This may happen because method 600 may examine more than one histidine residue that resides within rmax positions of the two identified lysine residues. Each identified histidine residue may, in combination with the two lysine residues, match the desired Replikin-like pattern.

### Methods of Determining Relative Lethality of Isolates of MERS CoV

One non-limiting aspect of the present invention provides methods of predicting expansion or retraction of outbreaks of MERS CoV. Compounds for diagnostic, therapeutic, and/or preventive purposes in MERS CoV and therapies for the prevention and treatment of MERS CoV are provided based on the disclosed methods of prediction.

### Replikin Peptide Sequences Available for Therapies in MERS CoV Across Strains and in Different Countries

An aspect of the present invention provides compounds for diagnostic, therapeutic, and/or preventive purposes in MERS CoV, methods of differentiating relative lethality between one or more isolates of MERS COV, and methods of designing therapies against MERS CoV based on compounds of the invention and differentiation of lethality among isolates of MERS CoV.

Compounds of one aspect of the invention comprise Replikin peptides and homologues of Replikin peptides identified in and isolated from different strains of MERS CoV and include Replikin peptides conserved over time in the same and different strains of MERS CoV. These Replikin peptides are useful when comprised in immunogenic and blocking compounds to provide a protective effect against MERS CoV infection including antagonism of the lethality of strains of MERS CoV. Replikin peptides that are conserved within strains of MERS CoV over time or across different strains of MERS CoV at conserved positions in the different strains of MERS CoV provide the ordinary skilled artisan with an expectation that the functionality of these peptides share commonality among various strains of MERS CoV and among various isolates of the same strain of MERS CoV at different times.

Four peptides provided in an aspect of this invention were identified as conserved in isolates of MERS CoV. The four peptides are combined in a vaccine for administration against challenge by MERS CoV. The vaccine is designed to generate therapeutic blocking response and an immune response that antagonizes infectivity, replication, and transmission of MERS CoV. Any virus that is not blocked on entry will be blocked intracellularly.

The four peptides were surprisingly identified as conserved in MERS CoV across regions and time. The ORF4b gene area of MERS CoV has been shown to be a Replikin Peak Gene area of the virus and Replikin Peak Gene areas of other viruses and pathogens have been demonstrated to be associated with lethality. See, e.g., WO 2008/143717, FIGS. 10-13, 16, 17, and 19.

The peptides and their homologues described herein are, among other things, antigenic, common to various strains of MERS CoV in both position and function, and conserved in various strains of MERS CoV over time. One of ordinary skill in the art expects the Replikin peptides and their homologues described herein to be useful in immunogenic and blocking compounds for therapies against MERS CoV within strains, across strains, and across time.

Shared and Conserved Replikin Peptide Sequences and their Homologues

Replikin sequences and their homologues provided by an aspect of the invention may be identified in strains of MERS CoV including any strain of MERS CoV known now or identified or known hereafter. Compounds of the invention may be conserved within strains of MERS CoV, across types within strains of MERS CoV, and across strains of MERS CoV. Compounds of the invention may be conserved in humans and camels and may be preserved over one, two, or more years in these hosts. In a non-limiting embodiment, the Replikin sequences are shared among isolates isolated from camels and humans. In a non-limiting embodiment, the Replikin sequences are identical isolates isolated from camels and humans. In a non-limiting embodiment the Replikin sequences are identical and shared among camels and humans and are conserved among camels and humans. The compounds, because they are Replikin sequences, related to Replikin sequences, derived from Replikin sequences, identified as comprising Replikin sequences, or designed to comprise Replikin sequences, are related to rapid replication, virulence, and lethality in MERS CoV and comprise necessary structure for antigenicity. These characteristics of Replikin sequences have been previously established in other viruses and organisms (see, e.g., U.S. Pat. No. 7,894,999, U.S. Pat. No. 7,758,863 and WO 2008/143717) but have not previously been disclosed in MERS CoV; and the surprisingly effective utility of the Replikin sequence in predictions and therapies in MERS CoV is established herein. Compounds of the invention, including conserved Replikin peptides, are useful as immunogenic compounds to stimulate the immune system of a subject to produce an immune response, which may include production of antibodies or other binding molecules. Compounds of the invention are also useful in therapies such as vaccines. Compounds of the invention are likewise useful in producing antibodies, antibody fragments, or other binding or antagonizing agents, which may be used, among other things, for diagnostic and therapeutic purposes, including passive immunity. Conservation between camel reservoirs and human hosts allows for targeting of the Replikin structures in both hosts.

The immunogenic compounds, antibodies (and other binding or antagonizing agents) and vaccines of the invention are useful against any strain of MERS CoV and are likewise useful against other coronaviruses. The compounds of the invention are also useful for diagnostic purposes, including identifying rapidly replicating, virulent, or lethal strains of virus.

Information on the conservation of homologous sequences across various strains of MERS CoV and in different regions provides sequences that offer immunogenic compounds for antagonism of all strains comprising these homologues across all regions having strains comprising these homologues. As a result, a vaccine is provided herein that offers cross-strain protection for a variety of strains of MERS CoV.

Replikin peptides in general are seen to be conserved across strains of MERS CoV. The key amino acid residues that provide for the Replikin sequence structure are the lysine and histidine residues wherein a Replikin sequence has at least one lysine on one terminus and at least one lysine or one histidine on the other terminus, at least one lysine that is six to ten residues from at least one other lysine, at least one histidine, and at least six percent lysines in total between the terminal lysine and the terminal lysine or histidine.

As may be seen in FIG. 10 of WO 2005/104754, when conserved homologous Replikin sequences are aligned one on top of the other over time, it is most apparent that fixed and conserved portions of the structure of Replikin sequences align in a series of posts or girders that illustrate, like the structure of a building, how key conserved amino acids provide constancy for the survival of a virus such as MERS CoV over time as it mutates to avoid immune recognition in its prospective host but maintains key functional genetic structures that provide for continued replication of the virus. These key functional genetic structures provide targets antagonized by Replikin-based therapies.

### Compounds and Compositions Comprising Peptides Homologous to MERS COV Replikin Peptides

One aspect of the present invention provides a protein, a protein fragment, a polypeptide, or a peptide that comprises at least one peptide A homologous with at least one Replikin peptide identified in an isolate of MERS CoV. The Replikin peptide may be any Replikin peptide identified in an isolate of MERS COV. The Replikin peptide may further be a Replikin peptide identified as conserved across strains or across regions in isolates of MERS COV or any homologue of a Replikin peptide identified as conserved across strains or across regions in isolates of MERS COV. For example, the Replikin peptide may be any one of SEQ ID NO(s): 1-28 or any homologue of any one of SEQ ID NO(s): 1-28.

Peptide A of the protein, protein fragment, polypeptide, or peptide may be 30%, 40%, 50%, 60%, 70%, 80%, 90%, or 95% or more homologous or 100% homologous with a Replikin peptide, including any of the peptides of SEQ ID NO(s): 1-28. A protein fragment or peptide may likewise be a peptide that consists of a peptide A that is homologous with a Replikin peptide of MERS COV, including any of SEQ ID NO(s): 1-28. A peptide consisting essentially of or consisting of a Replikin peptide of MERS COV, including any one of SEQ ID NO(s): 1-28, is also provided.

The amino acid sequence of the provided isolated or synthesized protein, protein fragment, polypeptide, or peptide may partially match an amino acid sequence of an expressed whole protein. At least one, five, ten, twenty, thirty, forty, fifty, one hundred, two hundred, three hundred, four hundred, five hundred, five hundred and fifty or more amino acid residues of the amino acid sequence of the expressed whole protein may not be present in the protein, protein fragment, polypeptide, or peptide. The amino acid sequence of an isolated or synthesized protein fragment, polypeptide, or peptide may also partially match the amino acid sequence of an expressed whole protein where at least one, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of at least one terminus of the amino acid sequence of the expressed whole protein is (are) not present at at least one terminus of said protein fragment, polypeptide, or peptide. Any additional number of amino acids may be situated on one or the other terminus or on both termini of the protein fragment, polypeptide, or peptide.

Because a Replikin peptide, such as SEQ ID NO(s): 1-28, is associated with rapid replication, infectivity, and/or lethality, in functional proteins in MERS CoV and because a Replikin peptide such as any one of SEQ ID NO(s): 1-28 is antigenic, inclusion of any Replikin peptide in a protein, protein fragment, polypeptide, or peptide does not negate the functional nature of the Replikin peptide. As such, antagonism of at least one Replikin peptide, including at least one of SEQ ID NO(s): 1-28 or a homologue of SEQ ID NO(s): 1-28 (with homology of 30% or greater) within a protein, protein fragment, polypeptide, or peptide would be expected to antagonize the replication, infectivity, and/or lethality of the protein fragment, polypeptide, or peptide.

A provided peptide may further be a peptide B of 7 to about 50 amino acid residues where peptide B contains a peptide A that is 30%, 40%, 50%, 60%, 70%, 80%, 90%, or 95% or more homologous or 100% homologous with any Replikin peptide, including one of SEQ ID NO(s): 1-28. A non-limiting peptide may further be a peptide A that is a Replikin peptide.

An isolated or synthesized protein, protein fragment, polypeptide, or peptide may consist of a peptide that is 30%, 40%, 50%, 60%, 70%, 80%, 90%, 95%, or 100% homologous with at least one of SEQ ID NO(s): 1-28 where the length of the peptide is no more than one, five, ten, twenty, thirty, forty, or fifty amino acid residues longer than a Replikin peptide of MERS COV including the sequence of SEQ ID NO(s): 1-28 with which it is homologous. An isolated or synthesized protein fragment, polypeptide, or peptide may likewise be no more than one, two, three, four, five, six, seven, eight, nine, or ten amino acid residues longer than the Replikin sequence with which it is homologous (including, for example, SEQ ID NO(s): 1-28).

Another non-limiting embodiment provides a biosynthetic composition of the invention. The biosynthetic composition may comprise the isolated or synthesized protein, protein fragment, polypeptide, or peptide of an aspect of the invention disclosed herein. The biosynthetic composition may further consist essentially of a Replikin peptide of a MERS CoV. In a further embodiment, the Replikin peptide of a MERS CoV is at least one peptide having a sequence of SEQ ID NO(s): 1-28. A further non-limiting embodiment provides a biosynthetic composition consisting of a Replikin peptide of MERS CoV. In a non-limiting embodiment, the isolated protein fragment, polypeptide, or peptide of an aspect of the invention is chemically synthesized by solid phase methods.

An isolated or synthesized polypeptide or peptide may comprise a peptide A that has about the same number of amino acid residues as a peptide B, where peptide B is one of the peptides of SEQ ID NO: 1-28 and where the lysine residues and histidine residues in peptide A are conserved as compared to the lysine residues and histidine residues in peptide B. An isolated or synthesized polypeptide or peptide comprising peptide A may have up to 100 additional amino acid residues as compared to peptide B. Some or all of the up to 100 additional amino acid residues may be positioned toward the amino-terminus and/or carboxy-terminus of the lysine or histidine termini of peptide A. Some of the additional amino acid residues may be positioned within the lysine or histidine termini of peptide A so long as a level of homology is maintained between peptide A and peptide B that retains at least some of the functionality of the Replikin peptide of peptide B. Functionality may include, but is not limited to, antigenicity, rate of replication, antagonizability of a protein containing said peptide A or said peptide B, binding capacity of binding agents to peptides A or B, etc.

An isolated or synthesized polypeptide or peptide may also comprise up to about 90, about 80, about 70, about 60, about 50, about 40, about 30, about 20, about 10, about 5, about 4, about 3, about 2, or about 1 additional amino acid residues. The residues may be entirely outside of the Replikin structure or entirely within the Replikin structure or partially within and partially outside the Replikin structure. A level of homology should be maintained between peptides B and A when additional residues are present or are added. Residues outside of the Replikin structure are those residues on the amino-terminus or carboxy-terminus of the polypeptide or peptide as compared to the lysine or histidine termini of peptide A. Residues within the Replikin structure are those residues that are between the lysine or histidine termini of peptide A. An isolated or synthesized polypeptide or peptide may also consist of peptide A and peptide A may consist of peptide B.

The at least one isolated or synthesized protein, protein fragment, or peptide may also comprise at least one peptide A and at least one peptide C where peptide A is at least 30%, 40%, 50%, 60%, 70%, 80%, 90%, 95%, or 100% homologous with at least one Replikin peptide of MERS CoV, which may include a peptide of SEQ ID NO(s): 1-28, and where peptide C is at least 30%, 40%, 50%, 60%, 70%, 80%, 90%, 95%, or 100% homologous with at least one other Replikin peptide of MERS CoV, which also may include a peptide of SEQ ID NO(s): 1-28. Peptide C may be homologous with a different Replikin peptide than the peptide that peptide A is homologous with. The at least one isolated or synthesized protein, protein fragment, or peptide may comprise three or more peptides homologous with at least three different Replikin peptides.

All of the above-discussed proteins, protein fragments, polypeptides, and peptides comprise the functional unit of a homologue of a Replikin peptide present in or isolated from an MERS CoV. The Replikin peptide may be any one of SEQ ID NO(s): 1-28 or any one of SEQ ID NO(s): 30-172, 174-190, 192-209, 211-222, 224-235, and 237-239. Antagonism of any of the homologues of a Replikin peptide will antagonize replication in MERS CoV. As a result, the proteins, protein fragments, polypeptides, and peptides are useful as immunogenic compounds, therapeutic compounds, vaccines, and for other therapies directed to antagonizing the replication and/or lethality of a strain of MERS CoV. When comprised in a vaccine, disclosed proteins, protein fragments, polypeptides, and peptides are expected to be capable of limiting the excretion or shedding of MERS CoV such that the virus is limited in its spread from host to host or from host to reservoir to host, etc. As such, disclosed compounds are effective at limiting sources of MERS CoV infection. Likewise, any binding agent that binds one of the proteins, protein fragments, polypeptides, and peptides discussed above will antagonize the replication and/or lethality of a strain of MERS CoV and limit sources of MERS COV infection such as transmission from host to host or from host to reservoir to host.

### Immunogenic Compositions Comprising Peptides Homologous to MERS CoV Replikin Peptides

A protein, protein fragment, or peptide comprising a Replikin peptide present or identified in an isolate of MERS CoV may be comprised in an immunogenic compound. The proteins, protein fragment, polypeptides, and peptides provided by an aspect of the invention comprise at least a portion that is homologous with a Replikin peptide or homologous with one of the MERS CoV Replikin peptides of SEQ ID NO(s): 1-28. These homologues are expected by one of ordinary skill in the art to stimulate the immune system of a subject upon sufficient exposure to produce antibodies against at least the homologous portion of the protein, protein fragment, polypeptide, or peptide and/or to produce a protective effect against MERS CoV. One of ordinary skill in the art would expect that antibodies or other binding agents arrayed against a protein or protein fragment comprising one of the antigenic homologues disclosed herein would be antagonistic to the protein or protein fragment.

One of ordinary skill would also expect an antagonist of one of these homologues to antagonize any MERS CoV that comprises a homologue of a Replikin peptide. Because homologues of SEQ ID NO(s): 1-28 have been shown to be conserved across strains and time, one of ordinary skill would expect antagonism of such homologues to result in antagonism of MERS CoV replication. One of ordinary skill would further expect particular antagonism of the lethality mechanisms of MERS CoV when an immune system is stimulated against a homologue of any one or more of SEQ ID NO(s): 1-28.

As a result, one aspect of the present invention is a method of stimulating the immune system of a subject with at least one compound comprising at least one Replikin sequence identified in MERS CoV or at least one isolated or synthesized homologue or functional fragment of at least one Replikin sequence identified in MERS CoV. The at least one Replikin sequence of the compound reflects an immunogenic target against which the immune system of the subject responds. Because at least a functional portion of the immunogenic structure of the target is maintained in a functional fragment of the at least one Replikin sequence, a functional fragment of the Replikin sequence is likewise a target against which the immune system of the subject responds. The compound may comprise a protein comprising the at least one Replikin sequence or functional fragment thereof, a protein fragment, a polypeptide, or a peptide comprising the at least one Replikin sequence or functional fragment thereof. The compound may comprise more than one protein, protein fragment, polypeptide or peptide. The compound may further be a composition of a plurality of synthesized or isolated Replikin sequences.

### Vaccines Comprising Peptides Homologous to MERS COV Replikin Peptides

An immunogenic compound provided as an aspect of the invention may be used as a component of a non-limiting vaccine against any strain of MERS CoV. A vaccine comprising one or more homologues of a Replikin peptide of MERS CoV may be used against MERS CoV. The vaccine may comprise one or more homologues of SEQ ID NO(s): 1-28 or 30-172, 174-190, 192-209, 211-222, 224-235, and 237-239. Likewise, a vaccine comprising one or more homologues of a Replikin peptide may be used against MERS CoV and may antagonize the replication and/or lethality of an MERS CoV infection. Further, mixtures of homologues of SEQ ID NO(s): 1-28 are provided as vaccines to antagonize the replication and/or lethality of an MERS CoV infection. Such vaccines are useful for antagonizing replication, lethality, and excretion or spread of MERS CoV.

One vaccine may comprise at least one protein, protein fragment, polypeptide, or peptide of any one or more of the proteins, protein fragments, polypeptides, or peptides of an aspect of the invention. The vaccine may further comprise at least one Replikin peptide of MERS CoV. One such Replikin peptide may be any one of SEQ ID NO(s): 1-28. A vaccine may comprise at least one peptide consisting essentially of any one of SEQ ID NO(s): 1-28 or at least one peptide consisting of any one of SEQ ID NO(s): 1-28. A vaccine may comprise a mixture of Replikin peptides of MERS CoV. The vaccine may comprise a plurality of peptides such as two, three, or four peptides of SEQ ID NO(s): 1-28. The peptide may consist essentially of any one or more SEQ ID NO(s): 1-28 or may consist of any one or more of SEQ ID NO(s): 1-28.

In another non-limiting embodiment, the vaccine comprises a mixture of peptides, wherein the mixture comprises isolated or synthesized peptides of SEQ ID NO(s): 1-9, SEQ ID NO(s):1-28, SEQ ID NO(s): 5, 18, 22, and 23, or SEQ ID NO(s): 6, 11, 12, 13, 14, 18, 19, 20, 26, 27, and 28. In a non-limiting embodiment, the vaccine comprises an approximately equal molar mixture of the isolated or synthesized peptides of SEQ ID NO(s): 1-9, SEQ ID NO(s):1-28, SEQ ID NO(s): 5, 18, 22, and 23, or SEQ ID NO(s): 6, 11, 12, 13, 14, 18, 19, 20, 26, 27, and 28. In a further non-limiting embodiment, the vaccine comprises approximately equal weight of the isolated or synthesized peptides of SEQ ID NO(s): 1-9, SEQ ID NO(s):1-28, SEQ ID NO(s): 5, 18, 22, and 23, or SEQ ID NO(s): 6, 11, 12, 13, 14, 18, 19, 20, 26, 27, and 28.

A vaccine may comprise a plurality of the shortest Replikin peptides from any region of the genome of MERS CoV including the ORF 4b gene area. A vaccine may comprise the shortest Replikin peptides from a gene area identified in a MERS CoV isolate or a plurality of MERS CoV isolates predicted to have a greater lethality than at least one other isolate of MERS CoV.

A vaccine may further comprise a plurality of the longest Replikin peptides from any gene area of the virus including the ORF4b gene area identified in an MERS CoV isolate or a plurality of MERS CoV isolates. A vaccine may also comprise a mixture of the shortest and longest Replikin peptides identified in the ORF4b gene area or any other gene area.

A vaccine may be formulated with a pharmaceutically acceptable excipient, carrier, or adjuvant. One pharmaceutically acceptable carrier or excipient is sterile water. A vaccine may comprise freeze-dried Replikin peptides in sterile water. Excipients, carriers, or adjuvants may include, but are not limited to, excipients, carriers and adjuvants known to those of skill in the art now or hereafter.

A non-limiting acceptable carrier or adjuvant may include sterile water, oil and water emulsion, or keyhole limpet hemocyanin, A non-limiting carrier and/or adjuvant may include a sterile diluent such as water (for dermal, nasal, or ocular application, spraying, or injection), saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as hydrochloric acid or sodium hydroxide. Preparations may be enclosed in ampoules, disposable syringes or multiple dose vials made of glass or plastic.

Pharmaceutical compositions suitable for use typically include sterile aqueous solutions (water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In general, a relevant carrier can be a solvent or dispersion medium containing, for example, water, ethanol, polyol (for example, glycerol, propylene glycol, and liquid polyetheylene glycol, and the like), and suitable mixtures thereof

In a further non-limiting embodiment, one adjuvant is a UTOPE. A UTOPE may be covalently attached to an isolated or synthesized peptide at the C-terminus, the N-terminus, or both termini. A UTOPE is a peptide sequence of 6 to 10 residues comprising one histidine residue with all other residues being lysine residues. A UTOPE may be included in a vaccine for the treatment or prevention of MERS CoV infection.

Peptides may be synthesized using L- or D-amino acids and may be synthesized with chemical moieties to discourage breaking of peptide bonds in biological systems.

A composition of the invention may be formulated for delivery by any available route including, but not limited to parenteral (e.g., intravenous), intradermal, subcutaneous, oral, nasal, bronchial, ophthalmic, transdermal (topical), transmucosal or any other routes. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Administration of the vaccine via any method may produce an immune response in an animal or human, it may further produce an antibody response in the animal or human. In a further non-limiting embodiment, the vaccine may produce a protective effect in the animal or human.

Generally, the dosage of peptides is in the range of from about 0.01 μg to about 500 mg, from about 0.05 μg to about 200 mg, about 0.075 μg to about 30 mg, about 0.09 μg to about 20 mg, about 0.1 μg to about 10 mg, about 10 μg to about 1 mg, and about 50 μg to about 500 μg. The skilled practitioner can readily determine the dosage and number of dosages needed to produce an effective immune response and/or blocking effect.

### Compositions Comprising any of SEQ ID NO(s): 1-28

A non-limiting composition is provided comprising one or more isolated or synthesized peptides that are 30%, 40%, 50%, 60%, 70%, 80%, 90%, 95% or more homologous with at least one of the peptides of SEQ ID NO(s): 1-28. A composition is provided comprising one or more isolated or synthesized peptides consisting essentially of or consisting of at least one peptide of SEQ ID NO(s): 1-28. A composition is further provided comprising two, three, four five, six, seven, eight, nine, ten, eleven, twelve, thirteen, fourteen, fifteen, sixteen, seventeen, eighteen, or up to twenty-eight isolated or synthesized peptides of SEQ ID NO(s): 1-28.

A composition comprising a mixture of peptides is provided wherein the mixture comprises at least each of the isolated or synthesized peptides of SEQ ID NO(s): 1-9; 1-28; 5, 18, 22, and 23; or 6, 11, 12, 13, 14, 18, 19, 20, 26, 27, and 28. A mixture is provided that is equimolar. A mixture is also provided that is equal by weight.

A composition of an aspect of the invention is a therapeutic composition. The therapeutic composition may provide, upon administration to a subject, a therapeutic effect against MERS COV. The therapeutic composition may be administered to any animal susceptible to infection from MERS CoV. The therapeutic composition may be administered to any animal susceptible to MERS CoV, including a human or a camel.

### Conserved Replikin Peptides Across MERS CoV and Peptide Homologies

Identification of conserved Replikin peptides across strains of MERS CoV in different countries has provided for the development of vaccines that may be directed across strains of MERS CoV in different countries. Identification of conserved Replikin peptides in isolates of MERS CoV of any strain may be accomplished in any way known to one of skill in the art now or hereafter. One method is by review of in silico sequences provided at the PubMed website of the National Center for Biotechnology Information. Peptides that share exact identity or 100% homology with earlier identified Replikin peptides may be tracked using computer-searching methods. Peptides that share 30%, 40%, 50%, 60%, 70%, 80%, 90%, or 95% or more homology with an earlier identified Replikin peptide may also be tracked by computer methods.

For example, vaccines have now been developed for prevention and treatment of infection from MERS CoV. See, e.g., Examples 1-3 below. The sequences that are used in the vaccine in Example 1 are identically shared among human and camel hosts and conserved from 2012 to 2014. The vaccine in Example 2 likewise comprises sequences shared and conserved. The sequences that are used in the vaccine in Example 3 have now been identified as conserved across countries and strains of MERS CoV. All of these sequences, homologues of these sequences, and proteins, protein fragments, polypeptides, and peptides comprising, consisting essentially of, or consisting of these sequences or their homologues or functional fragments are useful in identifying lethal strains of MERS COV, treating infections from MERS CoV, and developing prophylactic therapies, such as vaccines, against infection from MERS CoV.

These proteins, fragment, polypeptides and peptides including any one of SEQ ID NO(s): 1-28 and/or homologues of any one of SEQ ID NO(s): 1-28 and/or functional fragments of SEQ ID NO(s): 1-28 are expected by one of ordinary skill in the art to provide antigenicity, antagonism of replication, and blocking that is comparable to any one of SEQ ID NO(s): 1-28.

Homology that is sufficient to produce a useful target for antagonism includes peptides that are 30%, 40%, 50%, 60%, 70%, 80%, 90%, 95%, or up to 100% homologous with any of SEQ ID NO(s): 1-28. Homology may be determined with peptides wherein gaps exists in the sequence that is being compared to any one of SEQ ID NO(s): 1-28 between amino acids that are identical to those of the peptide chosen from SEQ ID NO(s): 1-28.

Sequences that are conserved across strains of MERS CoV are excellent targets for controlling infectivity and lethality.

### Methods of Designing Vaccines

On aspect of the invention also provides methods of designing and making vaccines. For example, the invention provides a method of making a vaccine comprising selecting at least one or more isolated or synthesized Replikin peptides present or identified in an isolate of MERS CoV. Such peptides may include any one or more of SEQ ID NO(s): 1-28 as a component of a vaccine and making said vaccine. The method may comprise selecting from 1 to up to 4 or more isolated or synthesized MERS CoV Replikin peptides as a component of a vaccine. The peptides may be identified in the ORF4b gene area of the virus or may be identified in any area of the genome of MERS CoV. The method may comprise identifying one or more Replikin peptides in an emerging strain of MERS CoV up to about three years before the vaccine is administered, up to about one year before the vaccine is administered, up to about six months before the vaccine is administered, or up to about seven days before the vaccine is made.

An emerging strain may be any strain of MERS CoV identified by one of skill in the art as predicted to expand in a population in hosts, or predicted to increase in virulence, morbidity, and/or mortality (lethality) in its hosts. An emerging strain may likewise be a strain of MERS CoV wherein Replikin concentration is observed to be increasing over time. An emerging strain may likewise be a strain of MERS CoV identified within a rising portion of Replikin cycle, following a peak in a Replikin cycle, following a step-wise rise in a Replikin cycle, or identified by a Replikin Count Virus Expansion Index as an emerging strain of virus. See WO 2009/132209, the contents of which are incorporated herein by reference.

A method of making a vaccine is also provided comprising: selecting at least one isolated or synthesized protein, protein fragment, polypeptide, or peptide comprising a homologue of a Replikin peptide (including, for example, SEQ ID NO(s): 1-28) as a component of a vaccine; and making said vaccine. An isolated or synthesized protein, protein fragment, polypeptide, or peptide may comprise a peptide that is 30%, 40%, 50%, 60%, 70%, 80%, 90% or 95%, or 100%, homologous with at least one Replikin peptide. At least two, three, four, five, six, seven, eight, nine, ten, eleven, twelve, or more homologues of Replikin peptides may be selected. Also, at least two, three, four, five, six, seven, eight, nine, ten, eleven, twelve, or more Replikin peptides may be selected. The isolated or synthesized protein, protein fragment, polypeptide, or peptide may have the same amino acid sequence as at least one protein, protein fragment, polypeptide or peptide identified in an emerging strain of MERS COV up to one, two, or three or more years prior to making said vaccine. The at least one protein, protein fragment, polypeptide or peptide may be identified in an emerging strain of MERS COV one week, one month, two months, three months, four months, five months, or six months prior to making said vaccine.

The invention also provides a kit for making a vaccine where the kit includes at least one isolated or synthesized Replikin peptide of MERS CoV (including, for example, at least one peptide of SEQ ID NO(s): 1-28 or homologue of SEQ ID NO(s): 1-28). The kit may also include two, three, four, and up to twenty-eight or more peptides of SEQ ID NO(s): 1-28 or homologues of SEQ ID NO(s): 1-28.

### Antibodies as Diagnostics and Therapies for Identified Replikin Sequences

In another aspect of the invention, isolated Replikin peptides may be used to generate antibodies, antibody fragments, or to generate or identify other binding agents, which may be used, for example, for diagnostic purposes or to provide passive immunity in an individual. See, e.g., U.S. application Ser. No. 11/355,120, filed Feb. 16, 2006 and U.S. application Ser. No. 12/010,027, filed Jan. 18, 2008 (each incorporated herein by reference in their entirety).

Various procedures known in the art may be used for the production of antibodies to Replikin sequences or to proteins, protein fragments, polypeptides, or peptides comprising Replikin sequences. Such antibodies include, but are not limited to, polyclonal, monoclonal, chimeric, humanized, single chain, Fab fragments and fragments produced by a Fab expression library. Antibodies that are linked to a cytotoxic agent or signaling moiety may also be generated. Antibodies may also be administered in combination with an antiviral agent. Furthermore, combinations of antibodies to different Replikins may be administered as an antibody cocktail.

For the production of antibodies, various host animals may be immunized by injection with a Replikin peptide or a combination of Replikin peptides, including, but not limited to rabbits, mice, rats, and larger mammals. Monoclonal antibodies to Replikins may be prepared using any technique that provides for the production of antibody molecules. These include but are not limited to the hybridoma technique originally described by Kohler and Milstein, (Nature, 1975, 256:495-497), the human B-cell hybridoma technique (Kosbor et al., 1983, Immunology Today, 4:72), and the EBV hybridoma technique (Cole et al., Monoclonal Antibodies and Cancer Therapy, Alan R. Liss, Inc., pp. 77-96). In addition, techniques developed for the production of chimeric antibodies (Morrison et al., 1984, Proc. Nat. Acad. Sci USA, 81:6851-6855) or other techniques may be used. Alternatively, techniques described for the production of single chain antibodies (U.S. Pat. No. 4,946,778) can be adapted to produce Replikin-specific single chain antibodies. Antibody fragments that contain binding sites for a Replikin may be generated by known techniques. For example, such fragments include but are not limited to F(ab′)2 fragments which can be produced by pepsin digestion of the antibody molecules and or fragments that can be generated by reducing the disulfide bridges of F(ab′)2 fragments. Alternatively, Fab expression libraries can be generated (Huse et al., 1989, Science, 246:1275-1281) to allow rapid and easy identification of monoclonal Fab fragments with the desired specificity.

Binding agents are provided including an antibody, antibody fragment, or binding agent that binds to at least a portion of an amino acid sequence of at least one protein, protein fragment, polypeptide, or peptide comprising at least one peptide A, where peptide A is at least 30%, 40%, 50%, 60%, 70%, 80%, 90% or 95%, or 100%, homologous with at least one Replikin peptide of MERS CoV, which may include, for example, at least one Replikin peptide of SEQ ID NO(s): 1-28. The amino acid sequence of a protein fragment, polypeptide, or peptide may partially match the amino acid sequence of an expressed whole protein where at least one, five, ten, twenty, thirty, forty, fifty, one hundred, two hundred, three hundred, four hundred, five hundred or more amino acid residues of the amino acid sequence of the expressed whole protein are not present in the protein fragment, polypeptide, or peptide. The amino acid sequence of the protein fragment, polypeptide, or peptide may also partially match the amino acid sequence of an expressed whole protein where at least one, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of the amino acid sequence of at least one terminus of the expressed whole protein are not present at least one terminus of said protein fragment, polypeptide, or peptide.

Binding agents are also provided including an antibody, antibody fragment, or binding agent that binds to at least a portion of an amino acid sequence that is 30%, 40%, 50%, 60%, 70%, 80%, 90%, or 95% or more homologous with at least one Replikin peptide of MERS CoV. In a non-limiting embodiment, the length of peptide A may be no more than one, five, ten, twenty, thirty, forty, or fifty amino acid residues longer than the identified Replikin sequence with which it is homologous. Binding agents are also provided that bind to at least a portion of an amino acid sequence of at least one of SEQ ID NO(s): 1-28.

Binding agents may specifically or preferentially bind to the target protein, protein fragment, polypeptide, or peptide. Binding agents may specifically or preferentially bind to a homologue of at least one of SEQ ID NO(s): 1-28. Binding agents may likewise specifically or preferentially bind to a peptide consisting of any one of SEQ ID NO(s): 1-28. Binding agents may also specifically or preferentially bind to a portion of a peptide consisting of any one of SEQ ID NO(s): 1-28 including a single amino acid within a homologue of SEQ ID NO(s): 1-28, two amino acids, three amino acids, four amino acids, five amino acids, or any number of amino acids spread within or outside a homologue.

### Nucleic Acids and Compositions of Nucleic Acids

An isolated or synthesized nucleic acid sequence is also provided that encodes a protein, protein fragment, polypeptide, or peptide comprising at least one peptide A, where peptide A is at least 30%, 40%, 50%, 60%, 70%, 80%, 90% or 95%, or 100%, homologous with at least one Replikin peptide of MERS CoV. The at least one Replikin peptide may be any peptide of SEQ ID NO(s): 1-28. A nucleic acid sequence may also encode a protein, a protein fragment, a polypeptide, or a peptide where the amino acid sequence of the protein, protein fragment, polypeptide, or peptide partially matches the amino acid sequence of an expressed whole protein and at least one, two, three, four, five, ten, twenty, thirty, forty, fifty, one hundred, two hundred, three hundred, four hundred, five hundred or more amino acid residues of the amino acid sequence of the expressed whole protein are not present in the protein fragment, polypeptide, or peptide. Further, the amino acid sequence of the protein, protein fragment, polypeptide, or peptide may partially match the amino acid sequence of an expressed whole protein where at least one, two, three, four, five, ten, twenty, thirty, forty, fifty, sixty, seventy, eighty, ninety, one hundred, one hundred fifty, two hundred, two hundred fifty, three hundred, three hundred fifty, four hundred, four hundred fifty, five hundred, five hundred fifty or more amino acid residues of the amino acid sequence of at least one terminus of the expressed whole protein may not be present at least one terminus of the protein, protein fragment, polypeptide, or peptide

An isolated or synthesized nucleic acid sequence may also encode a peptide consisting of 7 to about 50 amino acid residues comprising at least one Replikin peptide, which may be one of the peptide sequences of SEQ ID NO(s): 1-28. It may also encode a peptide that is at least 30%, 40%, 50%, 60%, 70%, 80%, 90%, or 95% or more homologous with at least one of the peptide sequences of SEQ ID NO(s): 1-28. It may also encode a peptide consisting essentially of or consisting of at least one of the peptide sequences of SEQ ID NO(s): 1-28.

One aspect of the invention further provides an immunogenic composition that comprises an isolated or synthesized nucleic acid provided above. One aspect of the invention further provides a vaccine against MERS CoV comprising an isolated or synthesized nucleic acid provided above.

Anti-Sense Nucleic Acids and siRNA

One aspect of the invention further provides a nucleic acid sequence that is antisense to a nucleic acid that encodes for any Replikin peptide present in or identified in an MERS CoV isolate. This may include one of SEQ ID NO(s): 1-28 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide of MERS CoV including, for example, any one of SEQ ID NO(s): 1-28 or is 30%, 40%, 50%, 60%, 70%, 80%, 90%, 95% or more homologous with a nucleic acid that is antisense to a nucleic acid that encodes for any one of SEQ ID NO(s): 1-28.

The nucleotide sequence of the invention may be used in hybridization assays of biopsied tissue or blood, e.g., Southern or Northern analysis, including in situ hybridization assays, to diagnose the presence of a particular MERS CoV strain in a tissue sample or an environmental sample, for example. The present invention also provides kits containing antibodies specific for particular Replikins that are present in a particular isolate of MERS CoV, or containing nucleic acid molecules (sense or antisense) that hybridize specifically to a particular Replikin sequence, and optionally, various buffers and/or reagents needed for diagnosis.

Also within the scope of an aspect of the invention are oligoribonucleotide sequences that include antisense RNA and DNA molecules and ribozymes that function to inhibit the translation of Replikin-containing mRNA. Both antisense RNA and DNA molecules and ribozymes may be prepared by any method known in the art. The antisense molecules can be incorporated into a wide variety of vectors for delivery to a subject. The skilled practitioner can readily determine the best route of delivery, although generally intravenous or intramuscular delivery is routine. The dosage amount is also readily ascertainable.

An aspect of the invention further provides antisense nucleic acid molecules that are complementary to a nucleic acid of the invention, wherein the antisense nucleic acid molecule is complementary to a nucleotide sequence encoding a peptide of the invention. In particular the nucleic acid sequence may be anti-sense to a nucleic acid sequence that has been demonstrated to be conserved over a period of six months to one or more years and/or which are present in a strain of MERS CoV shown to have an increase in concentration of Replikin sequences relative to Replikin concentration in other MERS CoV strains.

An aspect of the invention also provides compositions comprising RNAi-inducing entities used to inhibit or reduce MERS CoV infection or replication including small interfering RNA, which is a class of about 10 to about 50 and often about 20 to about 25 nucleotide-long double-stranded RNA molecules. siRNA is involved in the RNA interference pathway, where it interferes with the expression of a specific gene such as the ORF4b gene area of MERS CoV. siRNAs also act in RNAi-related pathways, e.g., as an antiviral mechanism.

An effective amount of an RNAi-inducing entity is delivered to a cell or organism prior to, simultaneously with, or after exposure to MERS CoV. A dosage may be sufficient to reduce or delay one or more symptoms of MERS CoV infection. Compositions of the invention may comprise a single siRNA species targeted to a target transcript or may comprise a plurality of different siRNA species targeting one or more target transcripts.

One aspect of the invention provides a small interfering nucleic acid sequence that is about 10 to about 50 nucleic acids in length and is 30%, 40%, 50%, 60%, 70%, 80%, 90%, or 95% or more homologous with a nucleic acid that encodes for any portion of an MERS CoV Replikin peptide including, for example, any portion of SEQ ID NO(s): 1-28 or is 30%, 40%, 50%, 60%, 70%, 80%, 90%, or 95% or more homologous with a nucleic acid that is antisense to a nucleic acid that encodes for any portion of a Replikin peptide, including, for example, a portion of one of SEQ ID NO(s): 1-28. In a further non-limiting embodiment, the nucleic acid sequence is about 15 to about 30 nucleic acids. In a further non-limiting embodiment, the nucleic acid sequence is about 20 to about 25 nucleic acids. In a further non-limiting embodiment, the nucleic acid sequence is about 21 nucleic acids.

### Advance Replikin-Based Information on Pathogenic Outbreaks Provides for Rapid Production of Vaccines

Advance information concerning Replikin peptides in expanding strains of pathogen allows for the rapid production of specific effective synthetic vaccines using one, or a combination, of Replikin peptides. Such synthetic vaccines have been demonstrated in rabbits, chickens, and shrimp. See, e.g., Examples 1-3 herein, Examples 6 and 7 of U.S. application Ser. No. 11/355,120, filed Feb. 16, 2006, and Example 2 of U.S. application Ser. No. 12/108,458, filed Apr. 23, 2008. For example, a mixture of Replikin peptides administered orally to shrimp provided up to a 91% protective effect for shrimp challenged with taura syndrome virus. Taura syndrome virus is an often-lethal rapidly replicating pathogen that has a significant negative impact on the shrimp industry.

Synthetic Replikin vaccines have also been demonstrated in the H5N1 strain of influenza virus in chickens. For example, in a test of chickens administered a mixture of twelve H5N1 Replikin peptides from the hemagglutinin and pB1 gene areas intranasally, intraocularly, and by spray inhalation and challenged with low pathogenic H5N1 influenza isolated from a black duck in the state of North Carolina in the United States, a protective effect was observed at both the entry site of influenza (diminished antibody production in the serum was observed as compared to a control) and at excretion sites of influenza (influenza virus was not observed excreted in feces or saliva from treated chickens as compared to a control). See Example 1 of U.S. application Ser. No. 12/581,112, filed Oct. 16, 2009 (incorporated herein by reference).

Administration of Replikin peptides in both shrimp and chickens appears to have provided a notable measure of mucosal immunity. For example, in Example 2 of U.S. application Ser. No. 12/108,458, a mixture of Replikin peptides was administered by mouth to shrimp later challenged with taura syndrome virus. The 91% protective effect of the vaccine is expected to have been a result, at least in part, of a mucosal immune-like response in the gut of the shrimp.

Likewise, in chickens, the administration of a mixture of Replikin peptides provided a protective effect against entry of the H5N1 virus. For example, as may be seen in Example 1 of U.S. application Ser. No. 12/581,112, filed Oct. 16, 2009 (incorporated herein by reference), three of six vaccinated chickens, when inoculated with H5N1 virus, produced no measurable amount of antibodies against H5N1 in their serum. Instead, the virus was apparently blocked by mucosal immunity from even entering the chickens' system. Some virus apparently entered the system of the chickens but was then blocked intracellularly. While the applicants do not wish to be bound by theory, the virus may have been blocked in its intracellular transport to the RNA or in synthesis of virus on RNA or in transport from the RNA to excretion. Wherever the block occurs, the fact is that the examination of the excreta of the chicken showed complete absence of virus. For those three chickens in which a serum immune response was measured (that is, virus did enter their system), the vaccine additionally provided a protective effect against replication of the virus in the chickens' system (no virus was excreted in the feces or saliva of the chickens). As such, mucosal immunity, in addition to other immunities, is an important aspect of the immunity imparted by Replikin-based vaccines.

### Example 1

### Synthetic Replikin Vaccine Against MERS CoV Using Selected Peptides Identically-Shared Among Camels and Humans

A synthetic Replikin vaccine containing an approximately equal-parts-by-weight mixture of nine MERS CoV Replikin peptides was designed for use against relatively-lethal isolates of MERS CoV. The sequences were found to be identical, shared Replikin structures in MERS CoV in dromedary camels (Camelus dromedaries) and humans. The vaccine was designed as a best-fit synthetic vaccine against MERS CoV for identified isolates from 2012 to 2014 shared among these two host species. The vaccine makes possible immediate MERS-CoV Replikins Synthetic Vaccine™ tests in camels and rapid interruption of the spread of the MERS-CoV coronavirus, more lethal to humans than SARS 2003.

The vaccine was engineered to inhibit the lethality of relatively-lethal strains of MERS CoV and to attack the camel reservoir of the virus as well as protect humans.

The vaccine comprises a mixture of the following nine Replikin peptides in sterile water:

| (SEQ ID NO: 1) | |
|---|---|
| (1) | KQKAPKH |
| (SEQ ID NO: 2) | |
| (2) | KSAGHPFNK |
| (SEQ ID NO: 3) | |
| (3) | KRSHSPTKK |
| (SEQ ID NO: 4) | |
| (4) | HSPTKKLRYVK |
| (SEQ ID NO: 5) | |
| (5) | KARKRSHSPTK |
| (SEQ ID NO: 6) | |
| (6) | KHVEVFTDGK |
| (SEQ ID NO: 7) | |
| (7) | HKWKMVVCDK |
| (SEQ ID NO: 8) | |
| (8) | KKHVEVFTDGK |
| (SEQ ID NO: 9) | |
| (9) | KDAAAAKNKMRHK |

The vaccine is administered to a camel and an immune response is detected. In camels where some antibody is detected prior to administration of the vaccine, administration of the vaccine produces an increase in antibody response and an increase in immune protection. Challenge of the camel with MERS-CoV isolates results in a protective effect against the challenge including reduction in entrance of virus into camel at inoculation, reduction in replication of virus in camel system, and blocking of excretion of virus from camel in body fluids. The camel is protected from infection. The vaccine is likewise tested in humans, providing an immune response, a blocking effect, and a protective effect.

The vaccine may be administered to an animal or human susceptible, exposed to, or suffering from infection of MERS CoV. The blocking mechanism of the vaccine provides a therapeutic and prophylactic effect. The immune response generated by the vaccine provides a prophylactic effect. Because each of the sequences comprised in the vaccine is related to rapid replication and lethality of the virus and provides an immune and a blocking response upon administration to a subject at sufficient volume and concentration, each sequence may be used as an individual active component in a vaccine against MERS-CoV; a mixture of peptides is not necessary to provide an effective vaccine.

### Example 2

### Synthetic Replikin Vaccine Against MERS CoV Using Further Selected Peptides Shared Among Camels and Humans

A synthetic Replikin vaccine containing an approximately equal-parts-by-weight mixture of twenty-eight MERS CoV Replikin peptides was designed for use against relatively lethal isolates of MERS CoV. The sequences were found to be shared Replikin structures in MERS CoV in dromedary camels (Camelus dromedaries) and humans. The vaccine was designed as a synthetic vaccine against MERS CoV for identified isolates from 2012 to 2014 shared among these two host species. The vaccine makes possible immediate MERS-CoV Replikins Synthetic Vaccine™ tests in camels and rapid interruption of the spread of the MERS-CoV coronavirus, more lethal to humans than SARS 2003.

The vaccine was engineered to inhibit the lethality of relatively-lethal strains of MERS CoV.

The vaccine comprises a mixture of the following twenty-eight Replikin peptides in sterile water:

(SEQ ID NO: 1)
KQKAPKH
(SEQ ID NO: 2)
KSAGHPFNK
(SEQ ID NO: 3)
KRSHSPTKK
(SEQ ID NO: 4)
HSPTKKLRYVK
(SEQ ID NO: 5)
KARKRSHSPTK
(SEQ ID NO: 6)
KHVEVFTDGK
(SEQ ID NO: 7)
HKWKMVVCDK
(SEQ ID NO: 8)
KKHVEVFTDGK
(SEQ ID NO: 9)
KDAAAAKNKMRHK
(SEQ ID NO: 10)
KSVVRHLGVTK
(SEQ ID NO: 11)
KFYQHVINGCK
(SEQ ID NO: 12)
KQVHQVQLTDK
(SEQ ID NO: 13)
KGDSCSSNCKH
(SEQ ID NO: 14)
HARLKGGLILK
(SEQ ID NO: 15)
KAMLLKKEPLLYVPIRLAGH
(SEQ ID NO: 16)
KHLVPLMHK
(SEQ ID NO: 17)
KYYAFLNKH
(SEQ ID NO: 18)
KAAVHKWK
(SEQ ID NO: 19)
KLNPSEDFIKH
(SEQ ID NO: 20)
KFCDHMVK
(SEQ ID NO: 21)
KPGHAMPSLFK
(SEQ ID NO: 22)
KNKMRHK
(SEQ ID NO: 23)
KRSHSPTK
(SEQ ID NO: 24)
KDAAAAKNKMRH
(SEQ ID NO: 25)
KMRHKRTSTK
(SEQ ID NO: 26)
HVERKDVPYPK
(SEQ ID NO: 27)
KGMQLLHTK
(SEQ ID NO: 28)
KEGSSVTLKH.

The vaccine is administered to a camel and an immune response is detected. Challenge of the camel with MERS-CoV isolates results in a protective effect against the challenge including reduction in entrance of virus into camel at inoculation, reduction in replication of virus in camel system, and blocking of excretion of virus from camel in body fluids. The camel is protected from infection. The vaccine is likewise tested in humans, providing an immune response, a blocking effect, and a protective effect.

The vaccine may be administered to an animal or human susceptible, exposed to, or suffering from infection of MERS CoV. The blocking mechanism of the vaccine provides a therapeutic and prophylactic effect. The immune response generated by the vaccine provides a prophylactic effect. Because each of the sequences comprised in the vaccine is related to rapid replication and lethality of the virus and provides an immune and a blocking response upon administration to a subject at sufficient volume and concentration, each sequences may be used as an individual active component in a vaccine against MERS-CoV; a mixture of peptides is not necessary to provide an effective vaccine.

### Example 3

### Synthetic Replikin Vaccine Against MERS CoV

A synthetic Replikin vaccine containing an approximately equal-parts-by-weight mixture of four MERS CoV Replikin peptides was designed for use against relatively lethal isolates of MERS CoV. The vaccine was engineered from sequences confirmed to be conserved across regions (countries) and across time. Conservation was particularly noted in the key amino acid residues of the Replikin sequence, namely, lysine and histidine amino acid residues. The vaccine was engineered to inhibit the lethality of relatively lethal strains of MERS CoV.

The vaccine comprises a mixture of the following four Replikin peptides in sterile water:

| (SEQ ID NO: 22) | |
|---|---|
| (1) | KNKMRHK; |
| (SEQ ID NO: 18) | |
| (2) | KAAVHKWK; |
| (SEQ ID NO: 23) | |
| (3) | KRSHSPTK; |
| and | |
| (SEQ ID NO: 5) | |
| (4) | KARKRSHSPTK. |

The vaccine may be administered to an animal or human susceptible, exposed to, or suffering from infection of MERS CoV. The blocking mechanism of the vaccine provides a therapeutic and prophylactic effect. The immune response generated by the vaccine provides a prophylactic effect.

### Example 4

### Analysis of Accession No. AHX71944 for Replikin Sequences Conserved in Camel Host and Shared with Isolates in Human Hosts

PubMed Code:
AHX71944
Description:
Isolation and Characterization of MERS Coronavirus from a Dromedary Camel, Qatar, 2014
Direct Submission
Isolated:
2014 in Qatar
Source:
Middle East respiratory syndrome coronavirus (MERS-CoV)
Strain:
MERS-CoV
(SEQ ID NO: 29)
m¹ s² f³ v⁴ a⁵ g⁶ v⁷ t⁸ a⁹ q¹⁰ g¹¹ a¹² r¹³ g¹⁴ t¹⁵ y¹⁶ r¹⁷ a¹⁸ a¹⁹ l²⁰ n²¹ s²² e²³ k²⁴ h²⁵ q²⁶ d²⁷ h²⁸
v²⁹ s³⁰ l³¹ t³² v³³ p³⁴ l³⁵ e³⁶ g³⁷ s³⁸ g³⁹ n⁴⁰ l⁴¹ v⁴² e⁴³ k⁴⁴ l⁴⁵ s⁴⁶ p⁴⁷ w⁴⁸ f⁴⁹ m⁵⁰ d⁵¹ g⁵² e⁵³ n⁵⁴
a⁵⁵ y⁵⁶ e⁵⁷ v⁵⁸ v⁵⁹ k⁶⁰ a⁶¹ m⁶² l⁶³ l⁶⁴ k⁶⁵ k⁶⁶ e⁶⁷ p⁶⁸ k⁶⁹ l⁷⁰ y⁷¹ v⁷² p⁷³ i⁷⁴ r⁷⁵ k⁷⁶ a⁷⁷ g⁷⁸ h⁷⁹ t⁸⁰
r⁸¹ h⁸² k⁸³ p⁸⁴ g⁸⁵ p⁸⁶ r⁸⁷ v⁸⁸ y⁸⁹ k⁹⁰ v⁹¹ e⁹² r⁹³ l⁹⁴ i⁹⁵ a⁹⁶ e⁹⁷ e⁹⁸ n⁹⁹ p¹⁰⁰ f¹⁰¹ m¹⁰² e¹⁰³ n¹⁰⁴ q¹⁰⁵
¹⁰⁶ a¹⁰⁷ y¹⁰⁸ s¹⁰⁹ s¹¹⁰ s¹¹¹ a¹¹² n¹¹³ g¹¹⁴ s¹¹⁵ l¹¹⁶ v¹¹⁷ g¹¹⁸ t¹¹⁹ t¹²⁰ l¹²¹ q¹²² g¹²³ k¹²⁴ p¹²⁵ i¹²⁶ g¹²⁷
m¹²⁸ f¹²⁹ f¹³⁰ p¹³¹ y¹³² d¹³³ i¹³⁴ e¹³⁵ l¹³⁶ e¹³⁷ t¹³⁸ g¹³⁹ k¹⁴⁰ q¹⁴¹ n¹⁴² i¹⁴³ l¹⁴⁴ l¹⁴⁵ r¹⁴⁶ k¹⁴⁷ y¹⁴⁸ g¹⁴⁹
r¹⁵⁰ g¹⁵¹ g¹⁵² y¹⁵³ h¹⁵⁴ y¹⁵⁵ t¹⁵⁶ p¹⁵⁷ f¹⁵⁸ h¹⁵⁹ y¹⁶⁰ e¹⁶¹ r¹⁶² d¹⁶³ n¹⁶⁴ t¹⁶⁵ s¹⁶⁶ e¹⁶⁷ p¹⁶⁸ e¹⁶⁹ w¹⁷⁰ m¹⁷¹
d¹⁷² d¹⁷³ f¹⁷⁴ e¹⁷⁵ a¹⁷⁶ d¹⁷⁷ p¹⁷⁸ k¹⁷⁹ g¹⁸⁰ k¹⁸¹ y¹⁸² a¹⁸³ q¹⁸⁴ n¹⁸⁵ l¹⁸⁶ l¹⁸⁷ k¹⁸⁸ k¹⁸⁹ l¹⁹⁰ i¹⁹¹ g¹⁹² g¹⁹³
d¹⁹⁴ e¹⁹⁵ t¹⁹⁶ p¹⁹⁷ e¹⁹⁸ d¹⁹⁹ q²⁰⁰ y²⁰¹ m²⁰² c²⁰³ g²⁰⁴ v²⁰⁵ d²⁰⁶ g²⁰⁷ k²⁰⁸ p²⁰⁹ i²¹⁰ s²¹¹ a²¹² y²¹³ a²¹⁴ f²¹⁵
l²¹⁶ m²¹⁷ a²¹⁸ k²¹⁹ d²²⁰ g²²¹ i²²² t²²³ k²²⁴ l²²⁵ a²²⁶ d²²⁷ v²²⁸ e²²⁹ a²³⁰ d²³¹ v²³² a²³³ a²³⁴ r²³⁵ a²³⁶ d²³⁷
d²³⁸ e²³⁹ g²⁴⁰ f²⁴¹ i²⁴² t²⁴³ l²⁴⁴ k²⁴⁵ n²⁴⁶ n²⁴⁷ l²⁴⁸ y²⁴⁹ r²⁵⁰ l²⁵¹ v²⁵² w²⁵³ h²⁵⁴ v²⁵⁵ e²⁵⁶ r²⁵⁷ k²⁵⁸ d²⁵⁹
v²⁶⁰ p²⁶¹ y²⁶² p²⁶³ k²⁶⁴ q²⁶⁵ s²⁶⁶ i²⁶⁷ f²⁶⁸ t²⁶⁹ i²⁷⁰ n²⁷¹ s²⁷² v²⁷³ v²⁷⁴ q²⁷⁵ k²⁷⁶ d²⁷⁷ g²⁷⁸ v²⁷⁹ e²⁸⁰ n²⁸¹
t²⁸² p²⁸³ p²⁸⁴ h²⁸⁵ y²⁸⁶ f²⁸⁷ t²⁸⁸ l²⁸⁹ g²⁹⁰ e²⁹¹ k²⁹² i²⁹³ l²⁹⁴ t²⁹⁵ l²⁹⁶ t²⁹⁷ p²⁹⁸ r²⁹⁹ n³⁰⁰ k³⁰¹ w³⁰² s³⁰³
g³⁰⁴ v³⁰⁵ s³⁰⁶ d³⁰⁷ l³⁰⁸ s³⁰⁹ l³¹⁰ k³¹¹ q³¹² k³¹³ l³¹⁴ l³¹⁵ y³¹⁶ t³¹⁷ f³¹⁸ y³¹⁹ g³²⁰ k³²¹ e³²² s³²³ l³²⁴ e³²⁵
n³²⁶ p³²⁷ t³²⁸ y³²⁹ i³³⁰ y³³¹ h³³² s³³³ a³³⁴ f³³⁵ i³³⁶ e³³⁷ e³³⁸ g³³⁹ s³⁴⁰ e³⁴¹ g³⁴² n³⁴³ d³⁴⁴ s³⁴⁵ w³⁴⁶ l³⁴⁷
t³⁴⁸ g³⁴⁹ n³⁵⁰ a³⁵¹ i³⁵² q³⁵³ g³⁵⁴ f³⁵⁵ a³⁵⁶ e³⁵⁷ g³⁵⁸ e³⁵⁹ g³⁶⁰ a³⁶¹ s³⁶² y³⁶³ t³⁶⁴ a³⁶⁵ n³⁶⁶ d³⁶⁷ v³⁶⁸ e³⁶⁹
e³⁷⁰ q³⁷¹ s³⁷² s³⁷³ g³⁷⁴ m³⁷⁵ i³⁷⁶ k³⁷⁷ p³⁷⁸ n³⁷⁹ a³⁸⁰ l³⁸¹ l³⁸² e³⁸³ a³⁸⁴ t³⁸⁵ e³⁸⁶ p³⁸⁷ f³⁸⁸ a³⁸⁹ k³⁹⁰ g³⁹¹
d³⁹² s³⁹³ e³⁹⁴ s³⁹⁵ s³⁹⁶ n³⁹⁷ e³⁹⁸ k³⁹⁹ h⁴⁰⁰ s⁴⁰¹ v⁴⁰² a⁴⁰³ q⁴⁰⁴ l⁴⁰⁵ v⁴⁰⁶ s⁴⁰⁷ y⁴⁰⁸ l⁴⁰⁹ s⁴¹⁰ e⁴¹¹ r⁴¹² e⁴¹³
n⁴¹⁴ v⁴¹⁵ i⁴¹⁶ a⁴¹⁷ d⁴¹⁸ s⁴¹⁹ k⁴²⁰ s⁴²¹ f⁴²² t⁴²³ l⁴²⁴ i⁴²⁵ f⁴²⁶ g⁴²⁷ g⁴²⁸ v⁴²⁹ a⁴³⁰ y⁴³¹ a⁴³² y⁴³³ f⁴³⁴ g⁴³⁵
e⁴³⁶ e⁴³⁷ e⁴³⁸ g⁴³⁹ t⁴⁴⁰ m⁴⁴¹ y⁴⁴² f⁴⁴³ e⁴⁴⁴ p⁴⁴⁵ r⁴⁴⁶ a⁴⁴⁷ k⁴⁴⁸ s⁴⁴⁹ e⁴⁵⁰ v⁴⁵¹ s⁴⁵² r⁴⁵³ i⁴⁵⁴ g⁴⁵⁵ d⁴⁵⁶ s⁴⁵⁷
i⁴⁵⁸ f⁴⁵⁹ t⁴⁶⁰ g⁴⁶¹ e⁴⁶² t⁴⁶³ g⁴⁶⁴ s⁴⁶⁵ w⁴⁶⁶ p⁴⁶⁷ k⁴⁶⁸ v⁴⁶⁹ t⁴⁷⁰ q⁴⁷¹ i⁴⁷² a⁴⁷³ n⁴⁷⁴ m⁴⁷⁵ f⁴⁷⁶ l⁴⁷⁷ e⁴⁷⁸ q⁴⁷⁹
t⁴⁸⁰ q⁴⁸¹ h⁴⁸² s⁴⁸³ l⁴⁸⁴ n⁴⁸⁵ f⁴⁸⁶ v⁴⁸⁷ g⁴⁸⁸ e⁴⁸⁹ f⁴⁹⁰ v⁴⁹¹ v⁴⁹² n⁴⁹³ d⁴⁹⁴ v⁴⁹⁵ v⁴⁹⁶ l⁴⁹⁷ a⁴⁹⁸ i⁴⁹⁹ l⁵⁰⁰ a⁵⁰¹
t⁵⁰² t⁵⁰³ t⁵⁰⁴ t⁵⁰⁵ n⁵⁰⁶ v⁵⁰⁷ d⁵⁰⁸ k⁵⁰⁹ i⁵¹⁰ r⁵¹¹ q⁵¹² l⁵¹³ l⁵¹⁴ k⁵¹⁵ g⁵¹⁶ v⁵¹⁷ t⁵¹⁸ l⁵¹⁹ d⁵²⁰ k⁵²¹ l⁵²² r⁵²³
d⁵²⁴ y⁵²⁵ l⁵²⁶ a⁵²⁷ d⁵²⁸ y⁵²⁹ d⁵³⁰ v⁵³¹ a⁵³² v⁵³³ t⁵³⁴ a⁵³⁵ g⁵³⁶ p⁵³⁷ f⁵³⁸ m⁵³⁹ d⁵⁴⁰ n⁵⁴¹ a⁵⁴² i⁵⁴³ n⁵⁴⁴ v⁵⁴⁵
g⁵⁴⁶ g⁵⁴⁷ t⁵⁴⁸ g⁵⁴⁹ l⁵⁵⁰ q⁵⁵¹ y⁵⁵² a⁵⁵³ a⁵⁵⁴ i⁵⁵⁵ t⁵⁵⁶ a⁵⁵⁷ p⁵⁵⁸ y⁵⁵⁹ e⁵⁶⁰ v⁵⁶¹ l⁵⁶² t⁵⁶³ g⁵⁶⁴ l⁵⁶⁵ g⁵⁶⁶ e⁵⁶⁷
s⁵⁶⁸ f⁵⁶⁹ k⁵⁷⁰ k⁵⁷¹ v⁵⁷² a⁵⁷³ t⁵⁷⁴ i⁵⁷⁵ p⁵⁷⁶ y⁵⁷⁷ k⁵⁷⁸ v⁵⁷⁹ e⁵⁸⁰ n⁵⁸¹ s⁵⁸² v⁵⁸³ k⁵⁸⁴ d⁵⁸⁵ t⁵⁸⁶ l⁵⁸⁷ t⁵⁸⁸ y⁵⁸⁹
y⁵⁹⁰ a⁵⁹¹ h⁵⁹² s⁵⁹³ v⁵⁹⁴ l⁵⁹⁵ y⁵⁹⁶ r⁵⁹⁷ v⁵⁹⁸ f⁵⁹⁹ p⁶⁰⁰ y⁶⁰¹ d⁶⁰² m⁶⁰³ d⁶⁰⁴ s⁶⁰⁵ g⁶⁰⁶ v⁶⁰⁷ s⁶⁰⁸ s⁶⁰⁹ f⁶¹⁰ s⁶¹¹
e⁶¹² l⁶¹³ l⁶¹⁴ f⁶¹⁵ d⁶¹⁶ e⁶¹⁷ v⁶¹⁸ d⁶¹⁹ l⁶²⁰ s⁶²¹ v⁶²² a⁶²³ s⁶²⁴ t⁶²⁵ y⁶²⁶ f⁶²⁷ l⁶²⁸ v⁶²⁹ r⁶³⁰ l⁶³¹ l⁶³² q⁶³³
d⁶³⁴ k⁶³⁵ t⁶³⁶ g⁶³⁷ d⁶³⁸ f⁶³⁹ m⁶⁴⁰ s⁶⁴¹ t⁶⁴² i⁶⁴³ i⁶⁴⁴ t⁶⁴⁵ s⁶⁴⁶ e⁶⁴⁷ q⁶⁴⁸ t⁶⁴⁹ a⁶⁵⁰ v⁶⁵¹ s⁶⁵² k⁶⁵³ l⁶⁵⁴ l⁶⁵⁵
d⁶⁵⁶ t⁶⁵⁷ e⁶⁵⁸ f⁶⁵⁹ e⁶⁶⁰ a⁶⁶¹ t⁶⁶² e⁶⁶³ a⁶⁶⁴ t⁶⁶⁵ f⁶⁶⁶ n⁶⁶⁷ f⁶⁶⁸ l⁶⁶⁹ l⁶⁷⁰ d⁶⁷¹ l⁶⁷² a⁶⁷³ g⁶⁷⁴ l⁶⁷⁵ f⁶⁷⁶ r⁶⁷⁷
i⁶⁷⁸ f⁶⁷⁹ l⁶⁸⁰ r⁶⁸¹ n⁶⁸² a⁶⁸³ y⁶⁸⁴ v⁶⁸⁵ y⁶⁸⁶ t⁶⁸⁷ s⁶⁸⁸ q⁶⁸⁹ g⁶⁹⁰ f⁶⁹¹ v⁶⁹² v⁶⁹³ v⁶⁹⁴ n⁶⁹⁵ g⁶⁹⁶ k⁶⁹⁷ v⁶⁹⁸ s⁶⁹⁹
t⁷⁰⁰ l⁷⁰¹ v⁷⁰² k⁷⁰³ q⁷⁰⁴ v⁷⁰⁵ l⁷⁰⁶ d⁷⁰⁷ l⁷⁰⁸ l⁷⁰⁹ n⁷¹⁰ k⁷¹¹ g⁷¹² m⁷¹³ q⁷¹⁴ l⁷¹⁵ l⁷¹⁶ h⁷¹⁷ t⁷¹⁸ k⁷¹⁹ e⁷²⁰ s⁷²¹
w⁷²² a⁷²³ g⁷²⁴ s⁷²⁵ n⁷²⁶ i⁷²⁷ s⁷²⁸ a⁷²⁹ e⁷³⁰ i⁷³¹ y⁷³² s⁷³³ g⁷³⁴ r⁷³⁵ e⁷³⁶ s⁷³⁷ l⁷³⁸ i⁷³⁹ f⁷⁴⁰ p⁷⁴¹ s⁷⁴² g⁷⁴³
t⁷⁴⁴ y⁷⁴⁵ y⁷⁴⁶ c⁷⁴⁷ v⁷⁴⁸ t⁷⁴⁹ t⁷⁵⁰ k⁷⁵¹ a⁷⁵² k⁷⁵³ s⁷⁵⁴ v⁷⁵⁵ q⁷⁵⁶ q⁷⁵⁷ d⁷⁵⁸ l⁷⁵⁹ d⁷⁶⁰ v⁷⁶¹ i⁷⁶² l⁷⁶³ p⁷⁶⁴ g⁷⁶⁵
e⁷⁶⁶ f⁷⁶⁷ s⁷⁶⁸ k⁷⁶⁹ k⁷⁷⁰ q⁷⁷¹ l⁷⁷² g⁷⁷³ l⁷⁷⁴ l⁷⁷⁵ q⁷⁷⁶ p⁷⁷⁷ t⁷⁷⁸ d⁷⁷⁹ n⁷⁸⁰ s⁷⁸¹ t⁷⁸² t⁷⁸³ v⁷⁸⁴ s⁷⁸⁵ v⁷⁸⁶ t⁷⁸⁷
v⁷⁸⁸ s⁷⁸⁹ s⁷⁹⁰ n⁷⁹¹ m⁷⁹² v⁷⁹³ e⁷⁹⁴ t⁷⁹⁵ v⁷⁹⁶ v⁷⁹⁷ g⁷⁹⁸ q⁷⁹⁹ l⁸⁰⁰ e⁸⁰¹ q⁸⁰² t⁸⁰³ n⁸⁰⁴ m⁸⁰⁵ h⁸⁰⁶ s⁸⁰⁷ p⁸⁰⁸ d⁸⁰⁹
e⁸¹⁰ i⁸¹¹ v⁸¹² g⁸¹³ d⁸¹⁴ y⁸¹⁵ v⁸¹⁶ i⁸¹⁷ i⁸¹⁸ s⁸¹⁹ e⁸²⁰ k⁸²¹ l⁸²² f⁸²³ v⁸²⁴ r⁸²⁵ s⁸²⁶ k⁸²⁷ e⁸²⁸ e⁸²⁹ d⁸³⁰ g⁸³¹
f⁸³² a⁸³³ f⁸³⁴ y⁸³⁵ p⁸³⁶ a⁸³⁷ e⁸³⁸ t⁸³⁹ n⁸⁴⁰ g⁸⁴¹ h⁸⁴² a⁸⁴³ v⁸⁴⁴ p⁸⁴⁵ t⁸⁴⁶ l⁸⁴⁷ f⁸⁴⁸ r⁸⁴⁹ l⁸⁵⁰ k⁸⁵¹ g⁸⁵² g⁸⁵³
a⁸⁵⁴ p⁸⁵⁵ v⁸⁵⁶ k⁸⁵⁷ k⁸⁵⁸ v⁸⁵⁹ a⁸⁶⁰ f⁸⁶¹ g⁸⁶² g⁸⁶³ d⁸⁶⁴ q⁸⁶⁵ v⁸⁶⁶ h⁸⁶⁷ e⁸⁶⁸ v⁸⁶⁹ a⁸⁷⁰ a⁸⁷¹ v⁸⁷² r⁸⁷³ s⁸⁷⁴ v⁸⁷⁵
t⁸⁷⁶ v⁸⁷⁷ e⁸⁷⁸ y⁸⁷⁹ n⁸⁸⁰ i⁸⁸¹ h⁸⁸² a⁸⁸³ v⁸⁸⁴ l⁸⁸⁵ d⁸⁸⁶ t⁸⁸⁷ l⁸⁸⁸ l⁸⁸⁹ a⁸⁹⁰ s⁸⁹¹ s⁸⁹² s⁸⁹³ l⁸⁹⁴ r⁸⁹⁵ t⁸⁹⁶ f⁸⁹⁷
v⁸⁹⁸ v⁸⁹⁹ d⁹⁰⁰ k⁹⁰¹ s⁹⁰² l⁹⁰³ s⁹⁰⁴ i⁹⁰⁵ e⁹⁰⁶ e⁹⁰⁷ f⁹⁰⁸ a⁹⁰⁹ d⁹¹⁰ v⁹¹¹ v⁹¹² k⁹¹³ e⁹¹⁴ q⁹¹⁵ v⁹¹⁶ s⁹¹⁷ d⁹¹⁸ l⁹¹⁹
l⁹²⁰ v⁹²¹ k⁹²² l⁹²³ l⁹²⁴ r⁹²⁵ g⁹²⁶ m⁹²⁷ p⁹²⁸ i⁹²⁹ p⁹³⁰ d⁹³¹ f⁹³² d⁹³³ l⁹³⁴ d⁹³⁵ d⁹³⁶ f⁹³⁷ i⁹³⁸ d⁹³⁹ a⁹⁴⁰ p⁹⁴¹
e⁹⁴² y⁹⁴³ e⁹⁴⁴ f⁹⁴⁵ p⁹⁴⁶ a⁹⁴⁷ e⁹⁴⁸ g⁹⁴⁹ d⁹⁵⁰ a⁹⁵¹ s⁹⁵² w⁹⁵³ s⁹⁵⁴ s⁹⁵⁵ t⁹⁵⁶ m⁹⁵⁷ i⁹⁵⁸ f⁹⁵⁹ s⁹⁶⁰ l⁹⁶¹ h⁹⁶² p⁹⁶³
e⁹⁶⁴ e⁹⁶⁵ e⁹⁶⁶ d⁹⁶⁷ e⁹⁶⁸ e⁹⁶⁹ e⁹⁷⁰ s⁹⁷¹ e⁹⁷² v⁹⁷³ e⁹⁷⁴ a⁹⁷⁵ s⁹⁷⁶ d⁹⁷⁷ l⁹⁷⁸ e⁹⁷⁹ e⁹⁸⁰ g⁹⁸¹ e⁹⁸² s⁹⁸³ e⁹⁸⁴ c⁹⁸⁵
i⁹⁸⁶ s⁹⁸⁷ e⁹⁸⁸ t⁹⁸⁹ s⁹⁹⁰ t⁹⁹¹ e⁹⁹² q⁹⁹³ v⁹⁹⁴ d⁹⁹⁵ v⁹⁹⁶ s⁹⁹⁷ h⁹⁹⁸ e⁹⁹⁹ i¹⁰⁰⁰ s¹⁰⁰¹ d¹⁰⁰² d¹⁰⁰³ e¹⁰⁰⁴ w¹⁰⁰⁵ a¹⁰⁰⁶
a¹⁰⁰⁷ a¹⁰⁰⁸ v¹⁰⁰⁹ d¹⁰¹⁰ e¹⁰¹¹ a¹⁰¹² f¹⁰¹³ p¹⁰¹⁴ l¹⁰¹⁵ d¹⁰¹⁶ e¹⁰¹⁷ a¹⁰¹⁸ e¹⁰¹⁹ d¹⁰²⁰ e¹⁰²¹ t¹⁰²² e¹⁰²³ s¹⁰²⁴ e¹⁰²⁵
q¹⁰²⁶ e¹⁰²⁷ e¹⁰²⁸ a¹⁰²⁹ q¹⁰³⁰ p¹⁰³¹ e¹⁰³² e¹⁰³³ e¹⁰³⁴ p¹⁰³⁵ e¹⁰³⁶ e¹⁰³⁷ d¹⁰³⁸ i¹⁰³⁹ a¹⁰⁴⁰ q¹⁰⁴¹ e¹⁰⁴² e¹⁰⁴³ i¹⁰⁴⁴
a¹⁰⁴⁵ d¹⁰⁴⁶ t¹⁰⁴⁷ l¹⁰⁴⁸ q¹⁰⁴⁹ e¹⁰⁵⁰ t¹⁰⁵¹ p¹⁰⁵² e¹⁰⁵³ e¹⁰⁵⁴ 5¹⁰⁵⁵ d¹⁰⁵⁶ t¹⁰⁵⁷ e¹⁰⁵⁸ e¹⁰⁵⁹ v¹⁰⁶⁰ p¹⁰⁶¹ p¹⁰⁶² q¹⁰⁶³
e¹⁰⁶⁴ e¹⁰⁶⁵ k¹⁰⁶⁶ l¹⁰⁶⁷ p¹⁰⁶⁸ s¹⁰⁶⁹ e¹⁰⁷⁰ p¹⁰⁷¹ q¹⁰⁷² t¹⁰⁷³ i¹⁰⁷⁴ q¹⁰⁷⁵ p¹⁰⁷⁶ e¹⁰⁷⁷ e¹⁰⁷⁸ k¹⁰⁷⁹ e¹⁰⁸⁰ e¹⁰⁸¹ a¹⁰⁸²
p¹⁰⁸³ e¹⁰⁸⁴ y¹⁰⁸⁵ e¹⁰⁸⁶ a¹⁰⁸⁷ d¹⁰⁸⁸ t¹⁰⁸⁹ e¹⁰⁹⁰ q¹⁰⁹¹ t¹⁰⁹² q¹⁰⁹³ s¹⁰⁹⁴ e¹⁰⁹⁵ t¹⁰⁹⁶ e¹⁰⁹⁷ k¹⁰⁹⁸ p¹⁰⁹⁹ k¹¹⁰⁰ r¹¹⁰¹
l¹¹⁰² r¹¹⁰³ k¹¹⁰⁴ k¹¹⁰⁵ r¹¹⁰⁶ n¹¹⁰⁷ e¹¹⁰⁸ d¹¹⁰⁹ p¹¹¹⁰ l¹¹¹¹ s¹¹¹² n¹¹¹³ f¹¹¹⁴ e¹¹¹⁵ h¹¹¹⁶ k¹¹¹⁷ v¹¹¹⁸ i¹¹¹⁹ t¹¹²⁰
e¹¹²¹ c¹¹²² v¹¹²³ t¹¹²⁴ i¹¹²⁵ v¹¹²⁶ l¹¹²⁷ g¹¹²⁸ d¹¹²⁹ a¹¹³⁰ i¹¹³¹ q¹¹²¹ v¹¹³³ a¹¹³⁴ k¹¹³⁵ c¹¹³⁶ y¹¹³⁷ g¹¹³⁸ e¹¹³⁹
s¹¹⁴⁰ v¹¹⁴¹ l¹¹⁴² v¹¹⁴³ n¹¹⁴⁴ a¹¹⁴⁵ a¹¹⁴⁶ n¹¹⁴⁷ t¹¹⁴⁸ h^{l149} l¹¹⁵⁰ k¹¹⁵¹ h¹¹⁵² g¹¹⁵³ g¹¹⁵⁴ g¹¹⁵⁵ i¹¹⁵⁶ a¹¹⁵⁷ g¹¹⁵⁸
a¹¹⁵⁹ i¹¹⁶⁰ n¹¹⁶¹ a¹¹⁶² a¹¹⁶³ s¹¹⁶⁴ k¹¹⁶⁵ g¹¹⁶⁶ a¹¹⁶⁷ v¹¹⁶⁸ q¹¹⁶⁹ k¹¹⁷⁰ e¹¹⁷¹ s¹¹⁷² d¹¹⁷³ e¹¹⁷⁴ y¹¹⁷⁵ i¹¹⁷⁶ l¹¹⁷⁷
a¹¹⁷⁸ k¹¹⁷⁹ g¹¹⁸⁰ p¹¹⁸¹ l¹¹⁸² q^{ll83} v¹¹⁸⁴ g¹¹⁸⁵ d¹¹⁸⁶ s¹¹⁸⁷ v¹¹⁸⁸ l¹¹⁸⁹ l¹¹⁹⁰ q¹¹⁹¹ g¹¹⁹² h¹¹⁹³ s¹¹⁹⁴ l¹¹⁹⁵ a¹¹⁹⁶
k¹¹⁹⁷ n¹¹⁹⁸ i¹¹⁹⁹ l¹²⁰⁰ h¹²⁰¹ v¹²⁰² v¹²⁰³ g¹²⁰⁴ p¹²⁰⁵ d¹²⁰⁶ a¹²⁰⁷ r¹²⁰⁸ a¹²⁰⁹ k¹²¹⁰ q¹²¹¹ d¹²¹² v¹²¹³ s¹²¹⁴ l¹²¹⁵
l¹²¹⁶ s¹²¹⁷ k¹²¹⁸ c¹²¹⁹ y¹²²⁰ k¹²²¹ a¹²²² m¹²²³ n¹²²⁴ a¹²²⁵ y¹²²⁶ p¹²²⁷ l¹²²⁸ v¹²²⁹ v¹²³⁰ t¹²³¹ p¹²³² p¹²³³ v¹²³⁴
s¹²³⁵ a¹²³⁶ g¹²³⁷ i¹²³⁸ f¹²³⁹ g¹²⁴⁰ v¹²⁴¹ k¹²⁴² p¹²⁴³ g¹²⁴⁴ v¹²⁴⁵ s¹²⁴⁶ f¹²⁴⁷ d¹²⁴⁸ y¹²⁴⁹ l¹²⁵⁰ i¹²⁵¹ r¹²⁵² e¹²⁵³
a¹²⁵⁴ k¹²⁵⁵ t¹²⁵⁶ r¹²⁵⁷ v¹²⁵⁸ l¹²⁵⁹ v¹²⁶⁰ v¹²⁶¹ v¹²⁶² n¹²⁶³ s¹²⁶⁴ q¹²⁶⁵ d¹²⁶⁶ v¹²⁶⁷ y¹²⁶⁸ k¹²⁶⁹ s¹²⁷⁰ l¹²⁷¹ l¹²⁷²
i¹²⁷³ v¹²⁷⁴ d¹²⁷⁵ i¹²⁷⁶ p¹²⁷⁷ q¹²⁷⁸ s¹²⁷⁹ l¹²⁸⁰ t¹²⁸¹ f¹²⁸² s¹²⁸³ y¹²⁸⁴ d¹²⁸⁵ g¹²⁸⁶ l¹²⁸⁷ r¹²⁸⁸ g¹²⁸⁹ a¹²⁹⁰ i¹²⁹¹
r¹²⁹² k¹²⁹³ a¹²⁹⁴ k¹²⁹⁵ d¹²⁹⁶ y¹²⁹⁷ g¹²⁹⁸ f¹²⁹⁹ t¹³⁰⁰ v¹³⁰¹ f¹³⁰² v¹³⁰³ c¹³⁰⁴ t¹³⁰⁵ d¹³⁰⁶ n¹³⁰⁷ s¹³⁰⁸ a¹³⁰⁹ n¹³¹⁰
t¹³¹¹ k¹³¹² v¹³¹³ l¹³¹⁴ r¹³¹⁵ n¹³¹⁶ k¹³¹⁷ g¹³¹⁸ v¹³¹⁹ d¹³²⁰ y¹³²¹ t¹³²² k¹³²³ k¹³²⁴ f¹³²⁵ l¹³²⁶ t¹³²⁷ v¹³²⁸ d¹³²⁹
g¹³³⁰ v¹³³¹ q¹³³² y¹³³³ y¹³³⁴ c¹³³⁵ y¹³³⁶ t¹³³⁷ s¹³³⁸ k¹³³⁹ d¹³⁴⁰ t¹³⁴¹ l¹³⁴² d¹³⁴³ d¹³⁴⁴ i¹³⁴⁵ l¹³⁴⁶ q¹³⁴⁷ q¹³⁴⁸
a¹³⁴⁹ n¹³⁵⁰ k¹³⁵¹ s¹³⁵² v¹³⁵³ g¹³⁵⁴ i¹³⁵⁵ i¹³⁵⁶ s¹³⁵⁷ m¹³⁵⁸ p¹³⁵⁹ l¹³⁶⁰ g¹³⁶¹ y¹³⁶² v¹³⁶³ s¹³⁶⁴ h¹³⁶⁵ g¹³⁶⁶ l¹³⁶⁷
f¹³⁶⁸ l¹³⁶⁹ i¹³⁷⁰ q¹³⁷¹ a¹³⁷² g¹³⁷³ s¹³⁷⁴ v¹³⁷⁵ v¹³⁷⁶ r¹³⁷⁷ r¹³⁷⁸ v¹³⁷⁹ n¹³⁸⁰ v¹³⁸¹ p¹³⁸² y¹³⁸³ v¹³⁸⁴ c¹³⁸⁵ l¹³⁸⁶
l¹³⁸⁷ a¹³⁸⁸ n¹³⁸⁹ k¹³⁹⁰ e¹³⁹¹ q¹³⁹² e¹³⁹³ a¹³⁹⁴ i¹³⁹⁵ l¹³⁹⁶ m¹³⁹⁷ s¹³⁹⁸ e¹³⁹⁹ d¹⁴⁰⁰ v¹⁴⁰¹ k¹⁴⁰² l¹⁴⁰³ n¹⁴⁰⁴ p¹⁴⁰⁵
s¹⁴⁰⁶ e¹⁴⁰⁷ d¹⁴⁰⁸ f¹⁴⁰⁹ i¹⁴¹⁰ k¹⁴¹¹ h¹⁴¹² v¹⁴¹³ r¹⁴¹⁴ t¹⁴¹⁵ n¹⁴¹⁶ g¹⁴¹⁷ g¹⁴¹⁸ y¹⁴¹⁹ n¹⁴²⁰ s¹⁴²¹ w¹⁴²² h¹⁴²³ l¹⁴²⁴
v¹⁴²⁵ e¹⁴²⁶ g¹⁴²⁷ e¹⁴²⁸ l¹⁴²⁹ l¹⁴³⁰ v¹⁴³¹ q¹⁴³² d¹⁴³³ l¹⁴³⁴ r¹⁴³⁵ l¹⁴³⁶ n¹⁴³⁷ k¹⁴³⁸ l¹⁴³⁹ l¹⁴⁴⁰ h¹⁴⁴¹ w¹⁴⁴² s¹⁴⁴³
d¹⁴⁴⁴ q¹⁴⁴⁵ t¹⁴⁴⁶ i¹⁴⁴⁷ c¹⁴⁴⁸ y¹⁴⁴⁹ k¹⁴⁵⁰ d¹⁴⁵¹ s¹⁴⁵² v¹⁴⁵³ f¹⁴⁵⁴ y¹⁴⁵⁵ v¹⁴⁵⁶ v¹⁴⁵⁷ k¹⁴⁵⁸ n¹⁴⁵⁹ s¹⁴⁶⁰ t¹⁴⁶¹ a¹⁴⁶²
f¹⁴⁶³ p¹⁴⁶⁴ f¹⁴⁶⁵ e¹⁴⁶⁶ t¹⁴⁶⁷ l¹⁴⁶⁸ s¹⁴⁶⁹ a¹⁴⁷⁰ c¹⁴⁷¹ r¹⁴⁷² a¹⁴⁷³ y¹⁴⁷⁴ l¹⁴⁷⁵ d¹⁴⁷⁶ s¹⁴⁷⁷ r¹⁴⁷⁸ t¹⁴⁷⁹ t¹⁴⁸⁰ q¹⁴⁸¹
q¹⁴⁸² l¹⁴⁸³ t¹⁴⁸⁴ i¹⁴⁸⁵ e¹⁴⁸⁶ v¹⁴⁸⁷ l¹⁴⁸⁸ v¹⁴⁸⁹ t¹⁴⁹⁰ v¹⁴⁹¹ d¹⁴⁹² g¹⁴⁹³ v¹⁴⁹⁴ n¹⁴⁹⁵ f¹⁴⁹⁶ r¹⁴⁹⁷ t¹⁴⁹⁸ v¹⁴⁹⁹ v¹⁵⁰⁰
l¹⁵⁰¹ n¹⁵⁰² n¹⁵⁰³ k¹⁵⁰⁴ n¹⁵⁰⁵ t¹⁵⁰⁶ y¹⁵⁰⁷ r¹⁵⁰⁸ s¹⁵⁰⁹ q¹⁵¹⁰ l¹⁵¹¹ g¹⁵¹² c¹⁵¹³ v¹⁵¹⁴ f¹⁵¹⁵ f¹⁵¹⁶ n¹⁵¹⁷ g¹⁵¹⁸ a¹⁵¹⁹
d¹⁵²⁰ i¹⁵²¹ s¹⁵²² d¹⁵²³ t¹⁵²⁴ i¹⁵²⁵ p¹⁵²⁶ d¹⁵²⁷ e¹⁵²⁸ k¹⁵²⁹ q¹⁵³⁰ n¹⁵³¹ g¹⁵³² h¹⁵³³ g¹⁵³⁴ l¹⁵³⁵ y¹⁵³⁶ l¹⁵³⁷ a¹⁵³⁸
d¹⁵³⁹ n¹⁵⁴⁰ l¹⁵⁴¹ t¹⁵⁴² a¹⁵⁴³ d¹⁵⁴⁴ e¹⁵⁴⁵ t¹⁵⁴⁶ k¹⁵⁴⁷ a¹⁵⁴⁸ l¹⁵⁴⁹ k¹⁵⁵⁰ e¹⁵⁵¹ l¹⁵⁵² y¹⁵⁵³ g¹⁵⁵⁴ p¹⁵⁵⁵ v¹⁵⁵⁶ d¹⁵⁵⁷
p¹⁵⁵⁸ t¹⁵⁵⁹ f¹⁵⁶⁰ l¹⁵⁶¹ h¹⁵⁶² r¹⁵⁶³ f¹⁵⁶⁴ y¹⁵⁶⁵ s¹⁵⁶⁶ l¹⁵⁶⁷ k¹⁵⁶⁸ a¹⁵⁶⁹ a¹⁵⁷⁰ v¹⁵⁷¹ h¹⁵⁷² k¹⁵⁷³ w¹⁵⁷⁴ k¹⁵⁷⁵ m¹⁵⁷⁶
v¹⁵⁷⁷ v¹⁵⁷⁸ c¹⁵⁷⁹ d¹⁵⁸⁰ k¹⁵⁸¹ v¹⁵⁸² r¹⁵⁸³ s¹⁵⁸⁴ l¹⁵⁸⁵ k¹⁵⁸⁶ l¹⁵⁸⁷ s¹⁵⁸⁸ d¹⁵⁸⁹ n¹⁵⁹⁰ n¹⁵⁹¹ c¹⁵⁹² y¹⁵⁹³ l¹⁵⁹⁴ n¹⁵⁹⁵
a¹⁵⁹⁶ v¹⁵⁹⁷ i¹⁵⁹⁸ m¹⁵⁹⁹ t¹⁶⁰⁰ l¹⁶⁰¹ d¹⁶⁰² l¹⁶⁰³ l¹⁶⁰⁴ k¹⁶⁰⁵ d¹⁶⁰⁶ i¹⁶⁰⁷ k¹⁶⁰⁸ f¹⁶⁰⁹ v¹⁶¹⁰ i¹⁶¹¹ p¹⁶¹² a¹⁶¹³ l¹⁶¹⁴
q¹⁶¹⁵ h¹⁶¹⁶ a¹⁶¹⁷ f¹⁶¹⁸ m¹⁶¹⁹ k¹⁶²⁰ h¹⁶²¹ k¹⁶²² g¹⁶²³ g¹⁶²⁴ d¹⁶²⁵ g¹⁶²⁶ t¹⁶²⁷ d¹⁶²⁸ f¹⁶²⁹ i¹⁶³⁰ a¹⁶³¹ l¹⁶³² i¹⁶³³
m¹⁶³⁴ a¹⁶³⁵ y¹⁶³⁶ g¹⁶³⁷ n¹⁶³⁸ c¹⁶³⁹ t¹⁶⁴⁰ f¹⁶⁴¹ g¹⁶⁴² a¹⁶⁴³ p¹⁶⁴⁴ d¹⁶⁴⁵ d¹⁶⁴⁶ a¹⁶⁴⁷ s¹⁶⁴⁸ r¹⁶⁴⁹ l¹⁶⁵⁰ l¹⁶⁵¹ h¹⁶⁵²
t¹⁶⁵³ v¹⁶⁵⁴ l¹⁶⁵⁵ a¹⁶⁵⁶ k¹⁶⁵⁷ a¹⁶⁵⁸ e¹⁶⁵⁹ 1¹⁶⁶⁰ c¹⁶⁶¹ c¹⁶⁶² s¹⁶⁶³ a¹⁶⁶⁴ r¹⁶⁶⁵ m¹⁶⁶⁶ v¹⁶⁶⁷ w¹⁶⁶⁸ r¹⁶⁶⁹ e¹⁶⁷⁰ w¹⁶⁷¹
c¹⁶⁷² n¹⁶⁷³ v¹⁶⁷⁴ c¹⁶⁷⁵ g¹⁶⁷⁶ i¹⁶⁷⁷ k¹⁶⁷⁸ d¹⁶⁷⁹ v¹⁶⁸⁰ v¹⁶⁸¹ l¹⁶⁸² q¹⁶⁸³ g¹⁶⁸⁴ l¹⁶⁸⁵ k¹⁶⁸⁶ a¹⁶⁸⁷ c¹⁶⁸⁸ c¹⁶⁸⁹ y¹⁶⁹⁰
v¹⁶⁹¹ g¹⁶⁹² v¹⁶⁹³ q¹⁶⁹⁴ t¹⁶⁹⁵ v¹⁶⁹⁶ e¹⁶⁹⁷ d¹⁶⁹⁸ l¹⁶⁹⁹ r¹⁷⁰⁰ a¹⁷⁰¹ r¹⁷⁰² m¹⁷⁰³ t¹⁷⁰⁴ y¹⁷⁰⁵ v¹⁷⁰⁶ c¹⁷⁰⁷ q¹⁷⁰⁸ c¹⁷⁰⁹
g¹⁷¹⁰ g¹⁷¹¹ e¹⁷¹² r¹⁷¹³ h¹⁷¹⁴ r¹⁷¹⁵ q¹⁷¹⁶ i¹⁷¹⁷ v¹⁷¹⁸ e¹⁷¹⁹ h¹⁷²⁰ t¹⁷²¹ t¹⁷²² p¹⁷²³ w¹⁷²⁴ l¹⁷²⁵ l¹⁷²⁶ l¹⁷²⁷ s¹⁷²⁸
g¹⁷²⁹ t¹⁷³⁰ p¹⁷³¹ n¹⁷³² e¹⁷³³ k¹⁷³⁴ l¹⁷³⁵ v¹⁷³⁶ t¹⁷³⁷ t¹⁷³⁸ s¹⁷³⁹ t¹⁷⁴⁰ a¹⁷⁴¹ p¹⁷⁴² d¹⁷⁴³ f¹⁷⁴⁴ v¹⁷⁴⁵ a¹⁷⁴⁶ f¹⁷⁴⁷
n¹⁷⁴⁸ v¹⁷⁴⁹ f¹⁷⁵⁰ q¹⁷⁵¹ g¹⁷⁵² i¹⁷⁵³ e¹⁷⁵⁴ t¹⁷⁵⁵ a¹⁷⁵⁶ v¹⁷⁵⁷ g¹⁷⁵⁸ j¹⁷⁵⁹ y¹⁷⁶⁰ v¹⁷⁶¹ h¹⁷⁶² a¹⁷⁶³ r¹⁷⁶⁴ l¹⁷⁶⁵ k¹⁷⁶⁶
g¹⁷⁶⁷ g¹⁷⁶⁸ l¹⁷⁶⁹ i¹⁷⁷⁰ l¹⁷⁷¹ k¹⁷⁷² f¹⁷⁷³ d¹⁷⁷⁴ s¹⁷⁷⁵ g¹⁷⁷⁶ t¹⁷⁷⁷ v¹⁷⁷⁸ s¹⁷⁷⁹ k¹⁷⁸⁰ t¹⁷⁸¹ s¹⁷⁸² d¹⁷⁸³ w¹⁷⁸⁴ k¹⁷⁸⁵
c¹⁷⁸⁶ k¹⁷⁸⁷ v¹⁷⁸⁸ t¹⁷⁸⁹ d¹⁷⁹⁰ v¹⁷⁹¹ l¹⁷⁹² f¹⁷⁹³ s¹⁷⁹⁴ g¹⁷⁹⁵ q¹⁷⁹⁶ k¹⁷⁹⁷ y¹⁷⁹⁸ s¹⁷⁹⁹ s¹⁸⁰⁰ d⁸⁰⁰¹ c¹⁸⁰² n¹⁸⁰³ v¹⁸⁰⁴
v¹⁸⁰⁵ r¹⁸⁰⁶ y¹⁸⁰⁷ s¹⁸⁰⁸ l¹⁸⁰⁹ d¹⁸¹⁰ g¹⁸¹¹ n¹⁸¹² f¹⁸¹³ r¹⁸¹⁴ t¹⁸¹⁵ e¹⁸¹⁶ v¹⁸¹⁷ d¹⁸¹⁸ p¹⁸¹⁹ d¹⁸²⁰ l¹⁸²¹ s¹⁸²² a¹⁸²³
f¹⁸²⁴ y¹⁸²⁵ v¹⁸²⁶ k¹⁸²⁷ d¹⁸²⁸ g¹⁸²⁹ k¹⁸³⁰ y¹⁸³¹ f¹⁸³² t¹⁸³³ s¹⁸³⁴ e¹⁸³⁵ p¹⁸³⁶ p¹⁸³⁷ v¹⁸³⁸ t¹⁸³⁹ y¹⁸⁴⁰ s¹⁸⁴¹ p¹⁸⁴²
a¹⁸⁴³ t¹⁸⁴⁴ i¹⁸⁴⁵ l¹⁸⁴⁶ a¹⁸⁴⁷ g¹⁸⁴⁸ s¹⁸⁴⁹ v¹⁸⁵⁰ y¹⁸⁵¹ t¹⁸⁵² n¹⁸⁵³ s¹⁸⁵⁴ c¹⁸⁵⁵ l¹⁸⁵⁶ v¹⁸⁵⁷ s¹⁸⁵⁸ s¹⁸⁵⁹ d¹⁸⁶⁰ g¹⁸⁶¹
q¹⁸⁶² p¹⁸⁶³ g¹⁸⁶⁴ g¹⁸⁶⁵ d¹⁸⁶⁶ a¹⁸⁶⁷ i¹⁸⁶⁸ s¹⁸⁶⁹ l¹⁸⁷⁰ s¹⁸⁷¹ f¹⁸⁷² n¹⁸⁷³ n¹⁸⁷⁴ l¹⁸⁷⁵ l¹⁸⁷⁶ g¹⁸⁷⁷ f¹⁸⁷⁸ d¹⁸⁷⁹ s¹⁸⁸⁰
s¹⁸⁸¹ k¹⁸⁸² p¹⁸⁸³ v¹⁸⁸⁴ t¹⁸⁸⁵ k¹⁸⁸⁶ k¹⁸⁸⁷ y¹⁸⁸⁸ t¹⁸⁸⁹ y¹⁸⁹⁰ s¹⁸⁹¹ f¹⁸⁹² l¹⁸⁹³ p¹⁸⁹⁴ k¹⁸⁹⁵ e¹⁸⁹⁶ d¹⁸⁹⁷ g¹⁸⁹⁸ d¹⁸⁹⁹
v¹⁹⁰⁰ l¹⁹⁰¹ l¹⁹⁰² a¹⁹⁰³ e¹⁹⁰⁴ f¹⁹⁰⁵ d¹⁹⁰⁶ t¹⁹⁰⁷ y¹⁹⁰⁸ d¹⁹⁰⁹ p¹⁹¹⁰ i¹⁹¹¹ y¹⁹¹² k¹⁹¹³ n¹⁹¹⁴ g¹⁹¹⁵ a¹⁹¹⁶ m¹⁹¹⁷ y¹⁹¹⁸
k¹⁹¹⁹ g¹⁹²⁰ k¹⁹²¹ p¹⁹²² i¹⁹²³ l¹⁹²⁴ w¹⁹²⁵ v¹⁹²⁶ n¹⁹²⁷ k¹⁹²⁸ a¹⁹²⁹ s¹⁹³⁰ y¹⁹³¹ d¹⁹³² t¹⁹³³ n¹⁹³⁴ l¹⁹³⁵ n¹⁹³⁶ k¹⁹³⁷
f¹⁹³⁸ n¹⁹³⁹ r¹⁹⁴⁰ a¹⁹⁴¹ s¹⁹⁴² l¹⁹⁴³ r¹⁹⁴⁴ q¹⁹⁴⁵ i¹⁹⁴⁶ f¹⁹⁴⁷ d¹⁹⁴⁸ v¹⁹⁴⁹ a¹⁹⁵⁰ p¹⁹⁵¹ i¹⁹⁵² e¹⁹⁵³ l¹⁹⁵⁴ e¹⁹⁵⁵ n¹⁹⁵⁶
k¹⁹⁵⁷ f¹⁹⁵⁸ t¹⁹⁵⁹ p¹⁹⁶⁰ l¹⁹⁶¹ s¹⁹⁶² v¹⁹⁶³ g¹⁹⁶⁴ s¹⁹⁶⁵ t¹⁹⁶⁶ p¹⁹⁶⁷ v¹⁹⁶⁸ e¹⁹⁶⁹ p¹⁹⁷⁰ p¹⁹⁷¹ t¹⁹⁷² v¹⁹⁷³ d¹⁹⁷⁴ v¹⁹⁷⁵
v¹⁹⁷⁶ a¹⁹⁷⁷ l¹⁹⁷⁸ q¹⁹⁷⁹ q¹⁹⁸⁰ e¹⁹⁸¹ m¹⁹⁸² t¹⁹⁸³ i¹⁹⁸⁴ v¹⁹⁸⁵ k¹⁹⁸⁶ c¹⁹⁸⁷ k¹⁹⁸⁸ g¹⁹⁸⁹ l¹⁹⁹⁰ n¹⁹⁹¹ k¹⁹⁹² p¹⁹⁹³ f¹⁹⁹⁴
v¹⁹⁹⁵ k¹⁹⁹⁶ d¹⁹⁹⁷ n¹⁹⁹⁸ v¹⁹⁹⁹ s²⁰⁰⁰ f²⁰⁰¹ v²⁰⁰² a²⁰⁰³ d²⁰⁰⁴ d²⁰⁰⁵ s²⁰⁰⁶ g²⁰⁰⁷ t²⁰⁰⁸ p²⁰⁰⁹ v²⁰¹⁰ v²⁰¹¹ e²⁰¹² y²⁰¹³
l²⁰¹⁴ s²⁰¹⁵ k²⁰¹⁶ e²⁰¹⁷ d²⁰¹⁸ l²⁰¹⁹ h²⁰²⁰ t²⁰²¹ l²⁰²² y²⁰²³ v²⁰²⁴ d²⁰²⁵ p²⁰²⁶ k²⁰²⁷ y²⁰²⁸ q²⁰²⁹ v²⁰³⁰ i²⁰³¹ v²⁰³²
l²⁰³³ k²⁰³⁴ d²⁰³⁵ n²⁰³⁶ v²⁰³⁷ l²⁰³⁸ s²⁰³⁹ s²⁰⁴⁰ m²⁰⁴¹ l²⁰⁴² r²⁰⁴³ l²⁰⁴⁴ h²⁰⁴⁵ t²⁰⁴⁶ v²⁰⁴⁷ e²⁰⁴⁸ s²⁰⁴⁹ g²⁰⁵⁰ d²⁰⁵¹
i²⁰⁵² n²⁰⁵³ v²⁰⁵⁴ v²⁰⁵⁵ a²⁰⁵⁶ a²⁰⁵⁷ s²⁰⁵⁸ g²⁰⁵⁹ s²⁰⁶⁰ l²⁰⁶¹ t²⁰⁶² r²⁰⁶³ k²⁰⁶⁴ v²⁰⁶⁵ k²⁰⁶⁶ l²⁰⁶⁷ l²⁰⁶⁸ f²⁰⁶⁹ r²⁰⁷⁰
a²⁰⁷¹ s²⁰⁷² f²⁰⁷³ y²⁰⁷⁴ f²⁰⁷⁵ k²⁰⁷⁶ e²⁰⁷⁷ f²⁰⁷⁸ a²⁰⁷⁹ t²⁰⁸⁰ r²⁰⁸¹ t²⁰⁸² f²⁰⁸³ t²⁰⁸⁴ a²⁰⁸⁵ t²⁰⁸⁶ t²⁰⁸⁷ a²⁰⁸⁸ v²⁰⁸⁹
g²⁰⁹⁰ s²⁰⁹¹ c²⁰⁹² i²⁰⁹³ k²⁰⁹⁴ s²⁰⁹⁵ v²⁰⁹⁶ v²⁰⁹⁷ r²⁰⁹⁸ h²⁰⁹⁹ l²¹⁰⁰ g²¹⁰¹ v²¹⁰² t²¹⁰³ k²¹⁰⁴ g²¹⁰⁵ i²¹⁰⁶ l²¹⁰⁷ t²¹⁰⁸
g²¹⁰⁹ c²¹¹⁰ f²¹¹¹ s²¹¹² f²¹¹³ v²¹¹⁴ k²¹¹⁵ m²¹¹⁶ l²¹¹⁷ f²¹¹⁸ m²¹¹⁹ l²¹²⁰ p²¹²¹ l²¹²² a²¹²³ y²¹²⁴ f²¹²⁵ s²¹²⁶ d²¹²⁷
s²¹²⁸ k²¹²⁹ l²¹³⁰ g²¹³¹ t²¹³² t²¹³³ e²¹³⁴ v²¹³⁵ k²¹³⁶ v²¹³⁷ s²¹³⁸ a²¹³⁹ l²¹⁴³ k²¹⁴¹ t²¹⁴² a²¹⁴³ g²¹⁴⁴ v²¹⁴⁵ v²¹⁴⁶
t²¹⁴⁷ g²¹⁴⁸ n²¹⁴⁹ v²¹⁵⁰ v²¹⁵¹ k²¹⁵² q²¹⁵³ c²¹⁵⁴ c²¹⁵⁵ t²¹⁵⁶ a²¹⁵⁷ a²¹⁵⁸ v²¹⁵⁹ d²¹⁶⁰ l²¹⁶¹ s²¹⁶² m²¹⁶³ d²¹⁶⁴ k²¹⁶⁵
l²¹⁶⁶ r²¹⁶⁷ r²¹⁶⁸ v²¹⁶⁹ d²¹⁷⁰ w²¹⁷¹ k²¹⁷² s²¹⁷³ t²¹⁷⁴ l²¹⁷⁵ r²¹⁷⁶ l²¹⁷⁷ l²¹⁷⁸ l²¹⁷⁹ m²¹⁸⁰ l²¹⁸¹ c²¹⁸² t²¹⁸³ t²¹⁸⁴
m²¹⁸⁵ v²¹⁸⁶ l²¹⁸⁷ l²¹⁸⁸ s²¹⁸⁹ s²¹⁹⁰ v²¹⁹¹ y²¹⁹² h²¹⁹³ l²¹⁹⁴ y²¹⁹⁵ v²¹⁹⁶ f²¹⁹⁷ n²¹⁹⁸ q²¹⁹⁹ v²²⁰⁰ l²²⁰¹ s²²⁰² s²²⁰³
d²²⁰⁴ v²²⁰⁵ m²²⁰⁶ f²²⁰⁷ e²²⁰⁸ d²²⁰⁹ a²²¹⁰ q²²¹¹ g²²¹² l²²¹³ k²²¹⁴ k²²¹⁵ f²²¹⁶ y²²¹⁷ k²²¹⁸ e²²¹⁹ v²²²⁰ r²²²¹ a²²²²
y²²²³ l²²²⁴ g²²²⁵ i²²²⁶ s²²²⁷ s²²²⁸ a²²²⁹ c²²³⁰ d²²³¹ g²²³² l²²³³ a²²³⁴ s²²³⁵ a²²³⁶ y²²³⁷ r²²³⁸ a²²³⁹ n²²⁴⁰ s²²⁴¹
f²²⁴² d²²⁴³ v²²⁴⁴ p²²⁴⁵ t²²⁴⁶ f²²⁴⁷ c²²⁴⁸ a²²⁴⁹ n²²⁵⁰ r²²⁵¹ s²²⁵² a²²⁵³ m²²⁵⁴ c²²⁵⁵ n²²⁵⁶ w²²⁵⁷ c²²⁵⁸ l²²⁵⁹ i²²⁶⁰
s²²⁶¹ q²²⁶² d²²⁶³ s²²⁶⁴ i²²⁶⁵ t²²⁶⁶ h²²⁶⁷ y²²⁶⁸ p²²⁶⁹ a²²⁷⁰ l²²⁷¹ k²²⁷² m²²⁷³ v²²⁷⁴ q²²⁷⁵ t²²⁷⁶ h²²⁷⁷ l²²⁷⁸ s²²⁷⁹
h²²⁸⁰ y²²⁸¹ v²²⁸² l²²⁸³ n²²⁸⁴ i²²⁸⁵ d²²⁸⁶ w²²⁸⁷ l²²⁸⁸ w²²⁸⁹ f²²⁹⁰ a²²⁹¹ f²²⁹² e²²⁹³ t²²⁹⁴ g²²⁹⁵ l²²⁹⁶ a²²⁹⁷ y²²⁹⁸
m²²⁹⁹ l²³⁰⁰ y²³⁰¹ t²³⁰² s²³⁰³ a²³⁰⁴ f²³⁰⁵ n²³⁰⁶ w²³⁰⁷ l²³⁰⁸ l²³⁰⁹ l²³¹⁰ a²³¹¹ g²³¹² t²³¹³ l²³¹⁴ h²³¹⁵ y²³¹⁶ f²³¹⁷
f²³¹⁸ a²³¹⁹ q²³²⁰ t²³²¹ s²³²² i²³²³ f²³²⁴ v²³²⁵ d²³²⁶ w²³²⁷ r²³²⁸ s²³²⁹ y²³³⁰ n²³³¹ y²³³² a²³³³ v²³³⁴ s²³³⁵ s²³³⁶
a²³³⁷ f²³³⁸ w²³³⁹ l²³⁴⁰ f²³⁴¹ t²³⁴² j²³⁴³ i²³⁴⁴ p²³⁴⁵ m²³⁴⁶ a²³⁴⁷ g²³⁴⁸ l²³⁴⁹ v²³⁵⁰ r²³⁵¹ m²³⁵² y²³⁵³ n²³⁵⁴ l²³⁵⁵
l²³⁵⁶ a²³⁵⁷ c²³⁵⁸ l²³⁵⁹ w²³⁶⁰ l²³⁶¹ l²³⁶² r²³⁶³ k²³⁶⁴ f²³⁶⁵ y²³⁶⁶ q²³⁶⁷ h²³⁶⁸ v²³⁶⁹ i²³⁷⁰ n²³⁷¹ g²³⁷² c²³⁷³ k²³⁷⁴
d²³⁷⁵ t²³⁷⁶ a²³⁷⁷ c²³⁷⁸ l²³⁷⁹ l²³⁸⁰ c²³⁸¹ y²³⁸² k²³⁸³ r²³⁸⁴ n²³⁸⁵ r²³⁸⁶ l²³⁸⁷ t²³⁸⁸ r²³⁸⁹ v²³⁹⁰ e²³⁹¹ a²³⁹² s²³⁹³
t²³⁹⁴ v²³⁹⁵ v²³⁹⁶ c²³⁹⁷ g²³⁹⁸ g²³⁹⁹ k²⁴⁰⁰ r²⁴⁰¹ t²⁴⁰² f²⁴⁰³ y²⁴⁰⁴ i²⁴⁰⁵ t²⁴⁰⁶ a²⁴⁰⁷ n²⁴⁰⁸ g²⁴⁰⁹ g²⁴¹⁰ i²⁴¹¹ s²⁴¹²
f²⁴¹³ r²⁴¹⁴ r²⁴¹⁵ h²⁴¹⁶ n²⁴¹⁷ n²⁴¹⁸ w²⁴¹⁹ n²⁴²⁰ c²⁴²¹ v²⁴²² d²⁴²³ c²⁴²⁴ d²⁴²⁵ t²⁴²⁶ a²⁴²⁷ g²⁴²⁸ v²⁴²⁹ g²⁴³⁰ n²⁴³¹
t²⁴³² f²⁴³³ i²⁴³⁴ c²⁴³⁵ e²⁴³⁶ e²⁴³⁷ v²⁴³⁸ a²⁴³⁹ n²⁴⁴⁰ d²⁴⁴¹ l²⁴⁴² t²⁴⁴³ t²⁴⁴⁴ a²⁴⁴⁵ l²⁴⁴⁶ r²⁴⁴⁷ r²⁴⁴⁸ p²⁴⁴⁹ i²⁴⁵⁰
n²⁴⁵¹ a²⁴⁵² t²⁴⁵³ d²⁴⁵⁴ r²⁴⁵⁵ s²⁴⁵⁶ h²⁴⁵⁷ y²⁴⁵⁸ y²⁴⁵⁹ v²⁴⁶⁰ d²⁴⁶¹ s²⁴⁶² v²⁴⁶³ t²⁴⁶⁴ v²⁴⁶⁵ k²⁴⁶⁶ e²⁴⁶⁷ t²⁴⁶⁸ v²⁴⁶⁹
v²⁴⁷⁰ q²⁴⁷¹ f²⁴⁷² n²⁴⁷³ y²⁴⁷⁴ r²⁴⁷⁵ r²⁴⁷⁶ d²⁴⁷⁷ g²⁴⁷⁸ q²⁴⁷⁹ p²⁴⁸⁰ f²⁴⁸¹ y²⁴⁸² e²⁴⁸³ r²⁴⁸⁴ f²⁴⁸⁵ p²⁴⁸⁶ l²⁴⁸⁷ c²⁴⁸⁸
a²⁴⁸⁹ f²⁴⁹⁰ t²⁴⁹¹ n²⁴⁹² l²⁴⁹³ d²⁴⁹⁴ k²⁴⁹⁵ l²⁴⁹⁶ k²⁴⁹⁷ f²⁴⁹⁸ k²⁴⁹⁹ e²⁵⁰⁰ v²⁵⁰¹ c²⁵⁰² k²⁵⁰³ t²⁵⁰⁴ t²⁵⁰⁵ t²⁵⁰⁶ g²⁵⁰⁷
i²⁵⁰⁸ p²⁵⁰⁹ e²⁵¹⁰ y²⁵¹¹ n²⁵¹² f²⁵¹³ i²⁵¹⁴ i²⁵¹⁵ y²⁵¹⁶ d²⁵¹⁷ s²⁵¹⁸ s²⁵¹⁹ d²⁵²⁰ r²⁵²¹ g²⁵²² q²⁵²³ e²⁵²⁴ s²⁵²⁵ l²⁵²⁶
a²⁵²⁷ r²⁵²⁸ s²⁵²⁹ a²⁵³⁰ c²⁵³¹ v²⁵³² y²⁵³³ y²⁵³⁴ s²⁵³⁵ q²⁵³⁶ v²⁵³⁷ l²⁵³⁸ c²⁵³⁹ k²⁵⁴⁰ s²⁵⁴¹ i²⁵⁴² l²⁵⁴³ l²⁵⁴⁴ v²⁵⁴⁵
d²⁵⁴⁶ s²⁵⁴⁷ s²⁵⁴⁸ l²⁵⁴⁹ v²⁵⁵⁰ t²⁵⁵¹ s²⁵⁵² v²⁵⁵³ g²⁵⁵⁴ d²⁵⁵⁵ s²⁵⁵⁶ s²⁵⁵⁷ e²⁵⁵⁸ i²⁵⁵⁹ a²⁵⁶⁰ t²⁵⁶¹ k²⁵⁶² m²⁵⁶³ f²⁵⁶⁴
d²⁵⁶⁵ s²⁵⁶⁶ f²⁵⁶⁷ v²⁵⁶⁸ n²⁵⁶⁹ s²⁵⁷⁰ f²⁵⁷¹ v²⁵⁷² s²⁵⁷³ l²⁵⁷⁴ y²⁵⁷⁵ n²⁵⁷⁶ v²⁵⁷⁷ t²⁵⁷⁸ r²⁵⁷⁹ d²⁵⁸⁰ k²⁵⁸¹ l²⁵⁸² e²⁵⁸³
k²⁵⁸⁴ l²⁵⁸⁵ i²⁵⁸⁶ s²⁵⁸⁷ t²⁵⁸⁸ a²⁵⁸⁹ r²⁵⁹⁰ d²⁵⁹¹ v²⁵⁹³ r²⁵⁹⁴ r²⁵⁹⁵ g²⁵⁹⁶ d²⁵⁹⁷ n²⁵⁹⁸ f²⁵⁹⁹ h²⁶⁰⁰ s²⁶⁰¹ v²⁶⁰² l²⁶⁰³
t²⁶⁰⁴ t²⁶⁰⁵ f²⁶⁰⁶ i²⁶⁰⁷ d²⁶⁰⁸ a²⁶⁰⁹ a²⁶¹⁰ r²⁶¹¹ g²⁶¹² p²⁶¹³ a²⁶¹⁴ g²⁶¹⁵ v²⁶¹⁶ e²⁶¹⁷ s²⁶¹⁸ d²⁶¹⁹ v²⁶²⁰ e²⁶²¹ t²⁶²²
n²⁶²³ e²⁶²⁴ i²⁶²⁵ v²⁶²⁶ d²⁶²⁷ s²⁶²⁸ v²⁶²⁹ q²⁶³⁰ y²⁶³¹ a²⁶³² h²⁶³³ k²⁶³⁴ h²⁶³⁵ d²⁶³⁶ i²⁶³⁷ q²⁶³⁸ i²⁶³⁹ t²⁶⁴⁰ n²⁶⁴¹
e²⁶⁴² s²⁶⁴³ y²⁶⁴⁴ n²⁶⁴⁵ n²⁶⁴⁶ y²⁶⁴⁷ v²⁶⁴⁸ p²⁶⁴⁹ s²⁶³⁰ y²⁶⁵¹ v²⁶⁵² k²⁶⁵³ p²⁶⁵⁴ d²⁶⁵⁵ s²⁶⁵⁶ v²⁶⁵⁷ s²⁶⁵⁸ t²⁶⁵⁹ s²⁶⁶⁰
d²⁶⁶¹ l²⁶⁶² g²⁶⁶³ s²⁶⁶⁴ l²⁶⁶⁵ i²⁶⁶⁶ d²⁶⁶⁷ c²⁶⁶⁸ n²⁶⁶⁹ a²⁶⁷⁰ a²⁶⁷¹ s²⁶⁷² v²⁶⁷³ n²⁶⁷⁴ q²⁶⁷⁵ i²⁶⁷⁶ v²⁶⁷⁷ l²⁶⁷⁸ r²⁶⁷⁹
n²⁶⁸⁰ s²⁶⁸¹ n²⁶⁸² g²⁶⁸³ a²⁶⁸⁴ c²⁶⁸⁵ i²⁶⁸⁶ w²⁶⁸⁷ n²⁶⁸⁸ a²⁶⁸⁹ a²⁶⁹⁰ a²⁶⁹¹ y²⁶⁹² m²⁶⁹³ k²⁶⁹⁴ l²⁶⁹⁵ s²⁶⁹⁶ d²⁶⁹⁷ a²⁶⁹⁸
l²⁶⁹⁹ k²⁷⁰⁰ r²⁷⁰¹ q²⁷⁰² i²⁷⁰³ r²⁷⁰⁴ i²⁷⁰⁵ a²⁷⁰⁶ c²⁷⁰⁷ r²⁷⁰⁸ k²⁷⁰⁹ c²⁷¹⁰ n²⁷¹¹ l²⁷¹² a²⁷¹³ f²⁷¹⁴ r²⁷¹⁵ l²⁷¹⁶ t²⁷¹⁷
t²⁷¹⁸ s²⁷¹⁹ k²⁷²⁰ l²⁷²¹ r²⁷²² a²⁷²³ n²⁷²⁴ d²⁷²⁵ n²⁷²⁶ i²⁷²⁷ l²⁷²⁸ s²⁷²⁹ v²⁷³⁰ r²⁷³¹ f²⁷³² t²⁷³³ a²⁷³⁴ n²⁷³⁵ k²⁷³⁶
i²⁷³⁷ v²⁷³⁸ g²⁷³⁹ g²⁷⁴⁰ a²⁷⁴¹ p²⁷⁴² t²⁷⁴³ w²⁷⁴⁴ f²⁷⁴⁵ n²⁷⁴⁶ a²⁷⁴⁷ l²⁷⁴⁸ r²⁷⁴⁹ d²⁷⁵⁰ f²⁷⁵¹ t²⁷⁵² l²⁷⁵³ k²⁷⁵⁴ g²⁷⁵⁵
y²⁷⁵⁶ v²⁷⁵⁷ l²⁷⁵⁸ a²⁷⁵⁹ t²⁷⁶⁰ i²⁷⁶¹ i²⁷⁶² v²⁷⁶³ f²⁷⁶⁴ l²⁷⁶⁵ c²⁷⁶⁶ a²⁷⁶⁷ v²⁷⁶⁸ l²⁷⁶⁹ m²⁷⁷⁰ y²⁷⁷¹ l²⁷⁷² c²⁷⁷³ l²⁷⁷⁴
p²⁷⁷⁵ t²⁷⁷⁶ f²⁷⁷⁷ s²⁷⁷⁸ m²⁷⁷⁹ v²⁷⁸⁰ p²⁷⁸¹ v²⁷⁸² e²⁷⁸³ f²⁷⁸⁴ y²⁷⁸⁵ e²⁷⁸⁶ d²⁷⁸⁷ r²⁷⁸⁸ i²⁷⁸⁹ l²⁷⁹⁰ d²⁷⁹¹ f²⁷⁹² k²⁷⁹³
v²⁷⁹⁴ l²⁷⁹⁵ d²⁷⁹⁶ n²⁷⁹⁷ g²⁷⁹⁸ i²⁷⁹⁹ i²⁸⁰⁰ r²⁸⁰¹ d²⁸⁰² v²⁸⁰³ n²⁸⁰⁴ p²⁸⁰⁵ d²⁸⁰⁶ d²⁸⁰⁷ k²⁸⁰⁸ c²⁸⁰⁹ f²⁸¹⁰ a²⁸¹¹ n²⁸¹²
k²⁸¹³ h²⁸¹⁴ r²⁸¹⁵ s²⁸¹⁶ f²⁸¹⁷ t²⁸¹⁸ q²⁸¹⁹ w²⁸²⁰ y²⁸²¹ h²⁸²² e²⁸²³ h²⁸²⁴ v²⁸²⁵ g²⁸²⁶ g²⁸²⁷ v²⁸²⁸ y²⁸²⁹ d²⁸³⁰ n²⁸³¹
s²⁸³² i²⁸³³ t²⁸³⁴ c²⁸³⁵ p²⁸³⁶ l²⁸³⁷ t²⁸³⁸ v²⁸³⁹ a²⁸⁴⁰ v²⁸⁴¹ i²⁸⁴² a²⁸⁴³ g²⁸⁴⁴ v²⁸⁴⁵ a²⁸⁴⁶ g²⁸⁴⁷ a²⁸⁴⁸ r²⁸⁴⁹ i²⁸⁵⁰
p²⁸⁵¹ d²⁸⁵² v²⁸⁵³ p²⁸⁵⁴ t²⁸⁵⁵ t²⁸⁵⁶ l²⁸⁵⁷ a²⁸⁵⁸ w²⁸⁵⁹ v²⁸⁶⁰ n²⁸⁶¹ n²⁸⁶² q²⁸⁶³ i²⁸⁶⁴ i²⁸⁶⁵ f²⁸⁶⁶ f²⁸⁶⁷ v²⁸⁶⁸ s²⁸⁶⁹
r²⁸⁷⁰ v²⁸⁷¹ f²⁸⁷² a²⁸⁷³ n²⁸⁷⁴ t²⁸⁷⁵ g²⁸⁷⁶ s²⁸⁷⁷ v²⁸⁷⁸ c²⁸⁷⁹ y²⁸⁸⁰ t²⁸⁸¹ p²⁸⁸² i²⁸⁸³ d²⁸⁸⁴ e²⁸⁸⁵ i²⁸⁸⁶ p²⁸⁸⁷ y²⁸⁸⁸
k²⁸⁸⁹ s²⁸⁹⁰ f²⁸⁹¹ s²⁸⁹² d²⁸⁹³ s²⁸⁹⁴ g²⁸⁹⁵ c²⁸⁹⁶ i²⁸⁹⁷ l²⁸⁹⁸ p²⁸⁹⁹ s²⁹⁰⁰ e²⁹⁰¹ c²⁹⁰² t²⁹⁰³ m²⁹⁰⁴ f²⁹⁰⁵ r²⁹⁰⁶ d²⁹⁰⁷
a²⁹⁰⁸ e²⁹⁰⁹ g²⁹¹⁰ r²⁹¹¹ m²⁹¹² t²⁹¹³ p²⁹¹⁴ y²⁹¹⁵ c²⁹¹⁶ h²⁹¹⁷ d²⁹¹⁸ p²⁹¹⁹ t²⁹²⁰ v²⁹²¹ l²⁹²² p²⁹²³ g²⁹²⁴ a²⁹²⁵ f²⁹²⁶
a²⁹²⁷ y²⁹²⁸ s²⁹²⁹ q²⁹³⁰ m²⁹³¹ r²⁹³² p²⁹³³ h²⁹³⁴ v²⁹³⁵ r²⁹³⁶ y²⁹³⁷ d²⁹³⁸ l²⁹³⁹ y²⁹⁴⁰ d²⁹⁴¹ g²⁹⁴² n²⁹⁴³ m²⁹⁴⁴ f²⁹⁴⁵
i²⁹⁴⁶ k²⁹⁴⁷ f²⁹⁴⁸ p²⁹⁴⁹ e²⁹⁵⁰ v²⁹⁵¹ v²⁹⁵² f²⁹⁵³ e²⁹⁵⁴ s²⁹⁵⁵ t²⁹⁵⁶ l²⁹⁵⁷ r²⁹⁵⁸ i²⁹⁵⁹ t²⁹⁶⁰ r²⁹⁶¹ t²⁹⁶² l²⁹⁶³ s²⁹⁶⁴
t²⁹⁶⁵ q²⁹⁶⁶ y²⁹⁶⁷ c²⁹⁶⁸ r²⁹⁶⁹ f²⁹⁷⁰ g²⁹⁷¹ s²⁹⁷² c²⁹⁷³ e²⁹⁷⁴ y²⁹⁷⁵ a²⁹⁷⁶ q²⁹⁷⁷ e²⁹⁷⁸ g²⁹⁷⁹ v²⁹⁸⁰ c²⁹⁸¹ i²⁹⁸² t²⁹⁸³
t²⁹⁸⁴ n²⁹⁸⁵ g²⁹⁸⁶ s²⁹⁸⁷ w²⁹⁸⁸ a²⁹⁸⁹ i²⁹⁹⁰ f²⁹⁹¹ n²⁹⁹² d²⁹⁹³ h²⁹⁹⁴ h²⁹⁹⁵ l²⁹⁹⁶ n²⁹⁹⁷ r²⁹⁹⁸ p²⁹⁹⁹ g³⁰⁰⁰ v³⁰⁰¹ y³⁰⁰²
c³⁰⁰³ g³⁰⁰⁴ s³⁰⁰⁵ d³⁰⁰⁶ f³⁰⁰⁷ i³⁰⁰⁸ d³⁰⁰⁹ i³⁰¹⁰ v³⁰¹¹ r³⁰¹² r³⁰¹³ l³⁰¹⁴ a³⁰¹⁵ v³⁰¹⁶ s³⁰¹⁷ l³⁰¹⁸ f³⁰¹⁹ q³⁰²⁰ p³⁰²¹
i³⁰²² t³⁰²³ y³⁰²⁴ f³⁰²⁵ q³⁰²⁶ l³⁰²⁷ t³⁰²⁸ t³⁰²⁹ s³⁰³⁰ l³⁰³¹ v³⁰³² l³⁰³³ g³⁰³⁴ i³⁰³⁵ g³⁰³⁶ l³⁰³⁷ c³⁰³⁸ a³⁰³⁹ f³⁰⁴⁰
l³⁰⁴¹ t³⁰⁴² l³⁰⁴³ l³⁰⁴⁴ f³⁰⁴⁵ y³⁰⁴⁶ y³⁰⁴⁷ i³⁰⁴⁸ n³⁰⁴⁹ k³⁰⁵⁰ v³⁰⁵¹ k³⁰⁵² r³⁰⁵³ a³⁰⁵⁴ f³⁰⁵⁵ a³⁰⁵⁶ d³⁰⁵⁷ y³⁰⁵⁸ t³⁰⁵⁹
q³⁰⁶⁰ c³⁰⁶¹ a³⁰⁶² v³⁰⁶³ i³⁰⁶⁴ a³⁰⁶⁵ v³⁰⁶⁶ v³⁰⁶⁷ a³⁰⁶⁸ a³⁰⁶⁹ v³⁰⁷⁰ l³⁰⁷¹ n³⁰⁷² s³⁰⁷³ l³⁰⁷⁴ c³⁰⁷⁵ i³⁰⁷⁶ c³⁰⁷⁷ f³⁰⁷⁸
v³⁰⁷⁹ a³⁰⁸⁰ s³⁰⁸¹ i³⁰⁸² p³⁰⁸³ l³⁰⁸⁴ c³⁰⁸⁵ i³⁰⁸⁶ v³⁰⁸⁷ p³⁰⁸⁸ y³⁰⁸⁹ t³⁰⁹⁰ a³⁰⁹¹ l³⁰⁹² y³⁰⁹³ y³⁰⁹⁴ y³⁰⁹⁵ a³⁰⁹⁶ t³⁰⁹⁷
f³⁰⁹⁸ y³⁰⁹⁹ f³¹⁰⁰ t³¹⁰¹ n³¹⁰² e³¹⁰³ p³¹⁰⁴ a³¹⁰⁵ f³¹⁰⁶ i³¹⁰⁷ m³¹⁰⁸ h³¹⁰⁹ v³¹¹⁰ s³¹¹¹ w³¹¹² y³¹¹³ i³¹¹⁴ m³¹¹⁵ f³¹¹⁶
g³¹¹⁷ p³¹¹⁸ i³¹¹⁹ v³¹²⁰ p³¹²¹ i³¹²² w³¹²³ m³¹²⁴ t³¹²⁵ c³¹²⁶ v³¹²⁷ y³¹²⁸ t³¹²⁹ v³¹³⁰ a³¹³¹ m³¹³² c³¹³³ f³¹³⁴ r³¹³⁵
h³¹³⁶ f³¹³⁷ f³¹³⁸ w³¹³⁹ v³¹⁴⁰ l³¹⁴¹ a³¹⁴² y³¹⁴³ f³¹⁴⁴ s³¹⁴⁵ k³¹⁴⁶ k³¹⁴⁷ h³¹⁴⁸ v³¹⁴⁹ e³¹⁵⁰ v³¹⁵¹ f³¹⁵² t³¹⁵³ d³¹⁵⁴
g³¹⁵⁵ k³¹⁵⁶ l³¹⁵⁷ n³¹⁵⁸ c³¹⁵⁹ s³¹⁶⁰ f³¹⁶¹ q³¹⁶² d³¹⁶³ a³¹⁶⁴ a³¹⁶⁵ s³¹⁶⁶ n³¹⁶⁷ i³¹⁶⁸ f³¹⁶⁹ v³¹⁷⁰ i³¹⁷¹ n³¹⁷² k³¹⁷³
d³¹⁷⁴ t³¹⁷⁵ y³¹⁷⁶ a³¹⁷⁷ a³¹⁷⁸ l³¹⁷⁹ r³¹⁸⁰ n³¹⁸¹ s³¹⁸² l³¹⁸³ t³¹⁸⁴ n³¹⁸⁵ d³¹⁸⁶ a³¹⁸⁷ y³¹⁸⁸ s³¹⁸⁹ r³¹⁹⁰ f³¹⁹¹ l³¹⁹²
g³¹⁹³ l³¹⁹⁴ f³¹⁹⁵ n³¹⁹⁶ k³¹⁹⁷ y³¹⁹⁸ k³¹⁹⁹ y³²⁰⁰ f³²⁰¹ s³²⁰² g³²⁰³ a³²⁰⁴ m³²⁰⁵ e³²⁰⁶ t³²⁰⁷ a³²⁰⁸ a³²⁰⁹ y³²¹⁰ r³²¹¹
e³²¹² a³²¹³ a³²¹⁴ a³²¹⁵ c³²¹⁶ h³²¹⁷ l³²¹⁸ a³²¹⁹ k³²²⁰ a³²²¹ l³²²² q³²²³ t³²²⁴ y³²²⁵ s³²²⁶ e³²²⁷ t³²²⁸ g³²²⁹ 5³²³⁰
d³²³¹ l³²³² l³²³³ y³²³⁴ q³²³⁵ p³²³⁶ p³²³⁷ n³²³⁸ c³²³⁹ s³²⁴⁰ i³²⁴¹ t³²⁴² s³²⁴³ g³²⁴⁴ v³²⁴⁵ l³²⁴⁶ q³²⁴⁷ s³²⁴⁸ g³²⁴⁹
l³²⁵⁰ v³²⁵¹ k³²⁵² m³²⁵³ s³²⁵⁴ h³²⁵⁵ p³²⁵⁶ s³²⁵⁷ g³²⁵⁸ d³²⁵⁹ v³²⁶⁰ e³²⁶¹ a³²⁶² c³²⁶³ m³²⁶⁴ v³²⁶⁵ q³²⁶⁶ v³²⁶⁷ t³²⁶⁸
c³²⁶⁹ g³²⁷⁰ s³²⁷¹ m³²⁷² t³²⁷³ l³²⁷⁴ n³²⁷⁵ g³²⁷⁶ l³²⁷⁷ w³²⁷⁸ l³²⁷⁹ d³²⁸⁰ n³²⁸¹ t³²⁸² v³²⁸³ w³²⁸⁴ c³²⁸⁵ p³²⁸⁶ r³²⁸⁷
h³²⁸⁸ v³²⁸⁹ m³²⁹⁰ c³²⁹¹ p³²⁹² a³²⁹³ d³²⁹⁴ q³²⁹⁵ l³²⁹⁶ s³²⁹⁷ d³²⁹⁸ p³²⁹⁹ n³³⁰⁰ y³³⁰¹ d³³⁰² a³³⁰³ l³³⁰⁴ l³³⁰⁵ i³³⁰⁶
s³³⁰⁷ m³³⁰⁸ t³³⁰⁹ n³³¹⁰ h³³¹¹ s³³¹² f³³¹³ s³³¹⁴ v³³¹⁵ q³³¹⁶ k³³¹⁷ h³³¹⁸ i³³¹⁹ g³³²⁰ a³³²¹ p³³²² a³³²³ n³³²⁴ l³³²⁵
r³³²⁶ v³³²⁷ v³³²⁸ g³³²⁹ h³³³⁰ a³³³¹ m³³³² q³³³³ g³³³⁴ t³³³⁵ l³³³⁶ l³³³⁷ k³³³⁸ l³³³⁹ t³³⁴⁰ v³³⁴¹ d³³⁴² v³³⁴³ a³³⁴⁴
n³³⁴⁵ p³³⁴⁶ s³³⁴⁷ t³³⁴⁸ p³³⁴⁹ a³³⁵⁰ y³³⁵¹ t³³⁵² f³³⁵³ t³³⁵⁴ t³³⁵⁵ v³³⁵⁶ k³³⁵⁷ p³³⁵⁸ g³³⁵⁹ a³³⁶⁰ a³³⁶¹ f³³⁶² s³³⁶³
v³³⁶⁴ l³³⁶⁵ a³³⁶⁶ c³³⁶⁷ y³³⁶⁸ n³³⁶⁹ g³³⁷⁰ r³³⁷¹ p³³⁷² t³³⁷³ g³³⁷⁴ t³³⁷⁵ f³³⁷⁶ t³³⁷⁷ v³³⁷⁸ v³³⁷⁹ m³³⁸⁰ r³³⁸¹ p³³⁸²
n³³⁸³ y³³⁸⁴ t³³⁸⁵ i³³⁸⁶ k³³⁸⁷ g³³⁸⁸ s³³⁸⁹ f³³⁹⁰ l³³⁹¹ c³³⁹² g³³⁹³ s³³⁹⁴ c³³⁹⁵ g³³⁹⁶ s³³⁹⁷ v³³⁹⁸ g³³⁹⁹ y³⁴⁰⁰ t³⁴⁰¹
k³⁴⁰² e³⁴⁰³ g³⁴⁰⁴ s³⁴⁰⁵ v³⁴⁰⁶ i³⁴⁰⁷ n³⁴⁰⁸ f³⁴⁰⁹ c³⁴¹⁰ y³⁴¹¹ m³⁴¹² h³⁴¹³ q³⁴¹⁴ m³⁴¹⁵ e³⁴¹⁶ l³⁴¹⁷ a³⁴¹⁸ n³⁴¹⁹ g³⁴²⁰
t³⁴²¹ h³⁴²² t³⁴²³ g³⁴²⁴ s³⁴²⁵ a³⁴²⁶ f³⁴²⁷ d³⁴²⁸ g³⁴²⁹ t³⁴³⁰ m³⁴³¹ y³⁴³² g³⁴³³ a³⁴³⁴ f³⁴³⁵ m³⁴³⁶ d³⁴³⁷ k³⁴³⁸ q³⁴³⁹
v³⁴⁴⁰ h³⁴⁴¹ q³⁴⁴² v³⁴⁴³ q³⁴⁴⁴ l³⁴⁴⁵ t³⁴⁴⁶ d³⁴⁴⁷ k³⁴⁴⁸ y³⁴⁴⁹ c³⁴⁵⁰ s³⁴⁵¹ v³⁴⁵² n³⁴⁵³ v³⁴⁵⁴ v³⁴⁵⁵ a³⁴⁵⁶ w³⁴⁵⁷ l³⁴⁵⁸
y³⁴⁵⁹ a³⁴⁶⁰ a³⁴⁶¹ i³⁴⁶² l³⁴⁶³ n³⁴⁶⁴ g³⁴⁶⁵ c³⁴⁶⁶ a³⁴⁶⁷ w³⁴⁶⁸ f³⁴⁶⁹ v³⁴⁷⁰ k³⁴⁷¹ p³⁴⁷² n³⁴⁷³ r³⁴⁷⁴ t³⁴⁷⁵ s³⁴⁷⁶ v³⁴⁷⁷
v³⁴⁷⁸ s³⁴⁷⁹ f³⁴⁸⁰ n³⁴⁸¹ e³⁴⁸² w³⁴⁸³ a³⁴⁸⁴ l³⁴⁸⁵ a³⁴⁸⁶ n³⁴⁸⁷ q³⁴⁸⁸ f³⁴⁸⁹ t³⁴⁹⁰ e³⁴⁹¹ f³⁴⁹² v³⁴⁹³ g³⁴⁹⁴ t³⁴⁹⁵ q³⁴⁹⁶
s³⁴⁹⁷ v³⁴⁹⁸ d³⁴⁹⁹ m³⁵⁰⁰ l³⁵⁰¹ a³⁵⁰² v³⁵⁰³ k³⁵⁰⁴ t³⁵⁰⁵ g³⁵⁰⁶ v³⁵⁰⁷ a³⁵⁰⁸ i³⁵⁰⁹ e³⁵¹⁰ q³⁵¹¹ l³⁵¹² l³⁵¹³ y³⁵¹⁴ a³⁵¹⁵
i³⁵¹⁶ q³⁵¹⁷ q³⁵¹⁸ l³⁵¹⁹ y³⁵²⁰ t³⁵²¹ g³⁵²² f³⁵²³ q³⁵²⁴ g³⁵²⁵ k³⁵²⁶ q³⁵²⁷ i³⁵²⁸ l³⁵²⁹ g³⁵³⁰ s³⁵³¹ t³⁵³² m³⁵³³ l³⁵³⁴
e³⁵³⁵ d³⁵³⁶ e³⁵³⁷ f³⁵³⁸ t³⁵³⁹ p³⁵⁴⁰ e³⁵⁴¹ d³⁵⁴² v³⁵⁴³ n³⁵⁴⁴ m³⁵⁴⁵ q³⁵⁴⁶ i³⁵⁴⁷ m³⁵⁴⁸ g³⁵⁴⁹ v³⁵⁵⁰ v³⁵⁵¹ m³⁵⁵² q³⁵⁵³
s³⁵⁵⁴ g³⁵⁵⁵ v³⁵⁵⁶ r³⁵⁵⁷ k³⁵⁵⁸ v³⁵⁵⁹ t³⁵⁶⁰ y³⁵⁶¹ g³⁵⁶² t³⁵⁶³ a³⁵⁶⁴ h³⁵⁶⁵ w³⁵⁶⁶ l³⁵⁶⁷ f³⁵⁶⁸ a³⁵⁶⁹ t³⁵⁷⁰ l³⁵⁷¹ v³⁵⁷²
s³⁵⁷³ t³⁵⁷⁴ y³⁵⁷⁵ v³⁵⁷⁶ i³⁵⁷⁷ i³⁵⁷⁸ l³⁵⁷⁹ q³⁵⁸⁰ a³⁵⁸¹ t³⁵⁸² k³⁵⁸³ f³⁵⁸⁴ t³⁵⁸⁵ l³⁵⁸⁶ w³⁵⁸⁷ n³⁵⁸⁸ y³⁵⁸⁹ l³⁵⁹⁰ f³⁵⁹¹
e³⁵⁹² t³⁵⁹³ i³⁵⁹⁴ p³⁵⁹⁵ t³⁵⁹⁶ q³⁵⁹⁷ l³⁵⁹⁸ f³⁵⁹⁹ p³⁶⁰⁰ l³⁶⁰¹ l³⁶⁰² f³⁶⁰³ v³⁶⁰⁴ t³⁶⁰⁵ m³⁶⁰⁶ a³⁶⁰⁷ f³⁶⁰⁸ v³⁶⁰⁹ m³⁶¹⁰
l³⁶¹¹ l³⁶¹² v³⁶¹³ k³⁶¹⁴ h³⁶¹⁵ k³⁶¹⁶ h³⁶¹⁷ t³⁶¹⁸ f³⁶¹⁹ l³⁶²⁰ t³⁶²¹ l³⁶²² f³⁶²³ l³⁶²⁴ l³⁶²⁵ p³⁶²⁶ v³⁶²⁷ a³⁶²⁸ i³⁶²⁹
c³⁶³⁰ l³⁶³¹ t³⁶³² y³⁶³³ a³⁶³⁴ n³⁶³⁵ i³⁶³⁶ v³⁶³⁷ y³⁶³⁸ e³⁶³⁹ p³⁶⁴⁰ t³⁶⁴¹ t³⁶⁴² p³⁶⁴³ i³⁶⁴⁴ s³⁶⁴⁵ s³⁶⁴⁶ a³⁶⁴⁷ l³⁶⁴⁸
i³⁶⁴⁹ a³⁶⁵⁰ v³⁶⁵¹ a³⁶⁵² n³⁶⁵³ w³⁶⁵⁴ l³⁶⁵⁵ a³⁶⁵⁶ p³⁶⁵⁷ t³⁶⁵⁸ a³⁶⁵⁹ a³⁶⁶⁰ y³⁶⁶¹ m³⁶⁶² r³⁶⁶³ t³⁶⁶⁴ t³⁶⁶⁵ h³⁶⁶⁶ t³⁶⁶⁷
d³⁶⁶⁸ i³⁶⁶⁹ g³⁶⁷⁰ v³⁶⁷¹ y³⁶⁷² i³⁶⁷³ s³⁶⁷⁴ m³⁶⁷⁵ s³⁶⁷⁶ l³⁶⁷⁷ v³⁶⁷⁸ l³⁶⁷⁹ v³⁶⁸⁰ i³⁶⁸¹ v³⁶⁸² v³⁶⁸³ k³⁶⁸⁴ r³⁶⁸⁵ l³⁶⁸⁶
y³⁶⁸⁷ n³⁶⁸⁸ p³⁶⁸⁹ s³⁶⁹⁰ l³⁶⁹¹ s³⁶⁹² n³⁶⁹³ f³⁶⁹⁴ a³⁶⁹⁵ l³⁶⁹⁶ a³⁶⁹⁷ l³⁶⁹⁸ c³⁶⁹⁹ s³⁷⁰⁰ g³⁷⁰¹ v³⁷⁰² m³⁷⁰³ w³⁷⁰⁴ l³⁷⁰⁵
y³⁷⁰⁶ t³⁷⁰⁷ y³⁷⁰⁸ s³⁷⁰⁹ i³⁷¹⁰ g³⁷¹¹ e³⁷¹² a³⁷¹³ s³⁷¹⁴ s³⁷¹⁵ p³⁷¹⁶ i³⁷¹⁷ a³⁷¹⁸ y³⁷¹⁹ l³⁷²⁰ v³⁷²¹ f³⁷²² v³⁷²³ t³⁷²⁴
t³⁷²⁵ l³⁷²⁶ t³⁷²⁷ s³⁷²⁸ d³⁷²⁹ y³⁷³⁰ t³⁷³¹ i³⁷³² t³⁷³³ v³⁷³⁴ f³⁷³⁵ v³⁷³⁶ t³⁷³⁷ v³⁷³⁸ n³⁷³⁹ l³⁷⁴⁰ a³⁷⁴¹ k³⁷⁴² v³⁷⁴³
c³⁷⁴⁴ t³⁷⁴⁵ y³⁷⁴⁶ a³⁷⁴⁷ i³⁷⁴⁸ f³⁷⁴⁹ a³⁷⁵⁰ y³⁷⁵¹ s³⁷⁵² p³⁷⁵³ q³⁷⁵⁴ l³⁷⁵⁵ t³⁷⁵⁶ l³⁷⁵⁷ v³⁷⁵⁸ f³⁷⁵⁹ p³⁷⁶⁰ e³⁷⁶¹ v³⁷⁶²
k³⁷⁶³ m³⁷⁶⁴ i³⁷⁶⁵ l³⁷⁶⁶ l³⁷⁶⁷ l³⁷⁶⁸ y³⁷⁶⁹ t³⁷⁷⁰ c³⁷⁷¹ l³⁷⁷² g³⁷⁷³ f³⁷⁷⁴ m³⁷⁷⁵ c³⁷⁷⁶ t³⁷⁷⁷ c³⁷⁷⁸ y³⁷⁷⁹ f³⁷⁸⁰ g³⁷⁸¹
v³⁷⁸² f³⁷⁸³ s³⁷⁸⁴ l³⁷⁸⁵ l³⁷⁸⁶ n³⁷⁸⁷ l³⁷⁸⁸ k³⁷⁸⁹ l³⁷⁹⁰ r³⁷⁹¹ a³⁷⁹² p³⁷⁹³ m³⁷⁹⁴ g³⁷⁹⁵ v³⁷⁹⁶ y³⁷⁹⁷ d³⁷⁹⁸ f³⁷⁹⁹ k³⁸⁰⁰
v³⁸⁰¹ s³⁸⁰² t³⁸⁰³ q³⁸⁰⁴ e³⁸⁰⁵ f³⁸⁰⁶ r³⁸⁰⁷ f³⁸⁰⁸ m³⁸⁰⁹ t³⁸¹⁰ a³⁸¹¹ n³⁸¹² n³⁸¹³ l³⁸¹⁴ t³⁸¹⁵ a³⁸¹⁶ p³⁸¹⁷ r³⁸¹⁸ n³⁸¹⁹
s³⁸²⁰ w³⁸²¹ e³⁸²² a³⁸²³ m³⁸²⁴ a³⁸²⁵ l³⁸²⁶ n³⁸²⁷ f³⁸²⁸ k³⁸²⁹ l³⁸³⁰ i³⁸³¹ g3⁸³² i³⁸³³ g³⁸³⁴ g³⁸³⁵ t³⁸³⁶ p³⁸³⁷ c³⁸³⁸
i³⁸³⁹ k³⁸⁴⁰ v³⁸⁴¹ a³⁸⁴² a³⁸⁴³ m³⁸⁴⁴ q³⁸⁴⁵ s³⁸⁴⁶ k³⁸⁴⁷ l³⁸⁴⁸ t³⁸⁴⁹ d³⁸⁵⁰ l³⁸⁵¹ k³⁸⁵² c³⁸⁵³ t³⁸⁵⁴ s³⁸⁵⁵ v³⁸⁵⁶ v³⁸⁵⁷
l³⁸⁵⁸ l³⁸⁵⁹ s³⁸⁶⁰ v³⁸⁶¹ l³⁸⁶² q³⁸⁶³ q³⁸⁶⁴ l³⁸⁶⁵ h³⁸⁶⁶ l³⁸⁶⁷ e³⁸⁶⁸ a³⁸⁶⁹ n³⁸⁷⁰ s³⁸⁷¹ r³⁸⁷² a³⁸⁷³ w³⁸⁷⁴ a³⁸⁷⁵ f³⁸⁷⁶
c³⁸⁷⁷ v³⁸⁷⁸ k³⁸⁷⁹ c³⁸⁸⁰ h³⁸⁸¹ n³⁸⁸² d³⁸⁸³ i³⁸⁸⁴ l³⁸⁸⁵ a³⁸⁸⁶ a³⁸⁸⁷ t³⁸⁸⁸ d³⁸⁸⁹ p³⁸⁹⁰ s³⁸⁹¹ e³⁸⁹² a³⁸⁹³ f³⁸⁹⁴ e³⁸⁹⁵
k³⁸⁹⁶ f³⁸⁹⁷ v³⁸⁹⁸ s³⁸⁹⁹ l³⁹⁰⁰ f³⁹⁰¹ a³⁹⁰² t³⁹⁰³ l³⁹⁰⁴ m³⁹⁰⁵ t³⁹⁰⁶ f³⁹⁰⁷ s³⁹⁰⁸ g³⁹⁰⁹ a³⁹¹⁰ v³⁹¹¹ d³⁹¹² l³⁹¹³ d³⁹¹⁴
a³⁹¹⁵ l³⁹¹⁶ a³⁹¹⁷ s³⁹¹⁸ d³⁹¹⁹ i³⁹²⁰ f³⁹²¹ d³⁹²² t³⁹²³ p³⁹²⁴ s³⁹²⁵ v³⁹²⁶ l³⁹²⁷ q³⁹²⁸ a³⁹²⁹ t³⁹³⁰ l³⁹³¹ s³⁹³² e³⁹³³
f³⁹³⁴ s³⁹³⁵ h³⁹³⁶ l³⁹³⁷ a³⁹³⁸ t³⁹³⁹ f³⁹⁴⁰ a³⁹⁴¹ e³⁹⁴² l³⁹⁴³ e³⁹⁴⁴ a³⁹⁴⁵ a³⁹⁴⁶ q³⁹⁴⁷ k³⁹⁴⁸ a³⁹⁴⁹ y³⁹⁵⁰ q³⁹⁵¹ e³⁹⁵²
a³⁹⁵³ m³⁹⁵⁴ d³⁹⁵⁵ s³⁹⁵⁶ g³⁹⁵⁷ d³⁹⁵⁸ t³⁹⁵⁹ s³⁹⁶⁰ p³⁹⁶¹ q³⁹⁶² v³⁹⁶³ l³⁹⁶⁴ k³⁹⁶⁵ a³⁹⁶⁶ l³⁹⁶⁷ q³⁹⁶⁸ k³⁹⁶⁹ a³⁹⁷⁰ v³⁹⁷¹
n³⁹⁷² i³⁹⁷³ a³⁹⁷⁴ k³⁹⁷⁵ n³⁹⁷⁶ a³⁹⁷⁷ y³⁹⁷⁸ e³⁹⁷⁹ k³⁹⁸⁰ d³⁹⁸¹ k³⁹⁸² a³⁹⁸³ v³⁹⁸⁴ a³⁹⁸⁵ r³⁹⁸⁶ k³⁹⁸⁷ l³⁹⁸⁸ e³⁹⁸⁹ r³⁹⁹⁰
m³⁹⁹¹ a³⁹⁹² d³⁹⁹³ q³⁹⁹⁴ a³⁹⁹⁵ m³⁹⁹⁶ t³⁹⁹⁷ s³⁹⁹⁸ m³⁹⁹⁹ y⁴⁰⁰⁰ k⁴⁰⁰¹ q⁴⁰⁰² a⁴⁰⁰³ r⁴⁰⁰⁴ a⁴⁰⁰⁵ e⁴⁰⁰⁶ d⁴⁰⁰⁷ k⁴⁰⁰⁸ k⁴⁰⁰⁹
a⁴⁰¹⁰ k⁴⁰¹¹ i⁴⁰¹² v⁴⁰¹³ s⁴⁰¹⁴ a⁴⁰¹⁵ m⁴⁰¹⁶ q⁴⁰¹⁷ t⁴⁰¹⁸ m⁴⁰¹⁹ l⁴⁰²⁰ f⁴⁰²¹ g⁴⁰²² m⁴⁰²³ i⁴⁰²⁴ k⁴⁰²⁵ k⁴⁰²⁶ l⁴⁰²⁷ d⁴⁰²⁸
n⁴⁰²⁹ d⁴⁰³⁰ v⁴⁰³¹ l⁴⁰³² n⁴⁰³³ g⁴⁰³⁴ i⁴⁰³⁵ i⁴⁰³⁶ s⁴⁰³⁷ n⁴⁰³⁸ a⁴⁰³⁹ r⁴⁰⁴⁰ n⁴⁰⁴¹ g⁴⁰⁴² c⁴⁰⁴³ i⁴⁰⁴⁴ p⁴⁰⁴⁵ l⁴⁰⁴⁶ s⁴⁰⁴⁷
v⁴⁰⁴⁸ i⁴⁰⁴⁹ p⁴⁰⁵⁰ l⁴⁰⁵¹ c⁴⁰⁵² a⁴⁰⁵³ s⁴⁰⁵⁴ n⁴⁰⁵⁵ k⁴⁰⁵⁶ l⁴⁰⁵⁷ r⁴⁰⁵⁸ v⁴⁰⁵⁹ v⁴⁰⁶⁰ i⁴⁰⁶¹ p⁴⁰⁶² d⁴⁰⁶³ f⁴⁰⁶⁴ t⁴⁰⁶⁵ v⁴⁰⁶⁶
w⁴⁰⁶⁷ n⁴⁰⁶⁸ q⁴⁰⁶⁹ v⁴⁰⁷⁰ v⁴⁰⁷¹ t⁴⁰⁷² y⁴⁰⁷³ p⁴⁰⁷⁴ s⁴⁰⁷⁵ l⁴⁰⁷⁶ n⁴⁰⁷⁷ y⁴⁰⁷⁸ a⁴⁰⁷⁹ g⁴⁰⁸⁰ a⁴⁰⁸¹ l⁴⁰⁸² w⁴⁰⁸³ d⁴⁰⁸⁴ i⁴⁰⁸⁵
t⁴⁰⁸⁶ v⁴⁰⁸⁷ i⁴⁰⁸⁸ n⁴⁰⁸⁹ n⁴⁰⁹⁰ v⁴⁰⁹¹ d⁴⁰⁹² n⁴⁰⁹³ e⁴⁰⁹⁴ i⁴⁰⁹⁵ v⁴⁰⁹⁶ k⁴⁰⁹⁷ s⁴⁰⁹⁸ s⁴⁰⁹⁹ d⁴¹⁰⁰ v⁴¹⁰¹ v⁴¹⁰² d⁴¹⁰³ s⁴¹⁰⁴
n⁴¹⁰⁵ e⁴¹⁰⁶ n⁴¹⁰⁷ l⁴¹⁰⁸ t⁴¹⁰⁹ w⁴¹¹⁰ p⁴¹¹¹ l⁴¹¹² v⁴¹¹³ l⁴¹¹⁴ e⁴¹¹⁵ c⁴¹¹⁶ t⁴¹¹⁷ r⁴¹¹⁸ a⁴¹¹⁹ d⁴¹²⁰ t⁴¹²¹ s⁴¹²² a⁴¹²³
v⁴¹²⁴ k⁴¹²⁵ l⁴¹²⁶ q⁴¹²⁷ n⁴¹²⁸ n⁴¹²⁹ e⁴¹³⁰ i⁴¹³¹ k⁴¹³² p⁴¹³³ s⁴¹³⁴ g⁴¹³⁵ l⁴¹³⁶ k⁴¹³⁷ t⁴¹³⁸ m⁴¹³⁹ v⁴¹⁴⁰ v⁴¹⁴¹ s⁴¹⁴²
a⁴¹⁴³ g⁴¹⁴⁴ q⁴¹⁴⁵ e⁴¹⁴⁶ q⁴¹⁴⁷ t⁴¹⁴⁸ n⁴¹⁴⁹ c⁴¹⁵⁰ n⁴¹⁵¹ t⁴¹⁵² s⁴¹⁵³ s⁴¹⁵⁴ l⁴¹⁵⁵ a⁴¹⁵⁶ y⁴¹⁵⁷ y⁴¹⁵⁸ e⁴¹⁵⁹ p⁴¹⁶⁰ v⁴¹⁶¹
q⁴¹⁶² g⁴¹⁶³ r⁴¹⁶⁴ k⁴¹⁶⁵ m⁴¹⁶⁶ l⁴¹⁶⁷ m⁴¹⁶⁸ a⁴¹⁶⁹ l⁴¹⁷⁰ l⁴¹⁷¹ s⁴¹⁷² d⁴¹⁷³ n⁴¹⁷⁴ a⁴¹⁷⁵ y⁴¹⁷⁶ l⁴¹⁷⁷ k⁴¹⁷⁸ w⁴¹⁷⁹ a⁴¹⁸⁰
r⁴¹⁸¹ v⁴¹⁸² e⁴¹⁸³ g⁴¹⁸⁴ k⁴¹⁸⁵ d⁴¹⁸⁶ g⁴¹⁸⁷ f⁴¹⁸⁸ v⁴¹⁸⁹ s⁴¹⁹⁰ v⁴¹⁹¹ e⁴¹⁹² l⁴¹⁹³ q⁴¹⁹⁴ p⁴¹⁹⁵ p⁴¹⁹⁶ c⁴¹⁹⁷ k⁴¹⁹⁸ f⁴¹⁹⁹
l⁴²⁰⁰ i⁴²⁰¹ a⁴²⁰² g⁴²⁰³ p⁴²⁰⁴ k⁴²⁰⁵ g⁴²⁰⁶ p⁴²⁰⁷ e⁴²⁰⁸ i⁴²⁰⁹ r⁴²¹⁰ y⁴²¹¹ l⁴²¹² y⁴²¹³ f⁴²¹⁴ v⁴²¹⁵ k⁴²¹⁶ n⁴²¹⁷ l⁴²¹⁸
n⁴²¹⁹ n⁴²²⁰ l⁴²²¹ h⁴²²² r⁴²²³ g⁴²²⁴ q⁴²²⁵ v⁴²²⁶ l⁴²²⁷ g⁴²²⁸ h⁴²²⁹ i⁴²³⁰ a⁴²³¹ a⁴²³² t⁴²³³ v⁴²³⁴ r⁴²³⁵ l⁴²³⁶ q⁴²³⁷
a⁴²³⁸ g⁴²³⁹ s⁴²⁴⁰ n⁴²⁴¹ t⁴²⁴² e⁴²⁴³ f⁴²⁴⁴a⁴²⁴⁵ s⁴²⁴⁶ n⁴²⁴⁷ s⁴²⁴⁸ s⁴²⁴⁹ v⁴²⁵⁰ l⁴²⁵¹ s⁴²⁵² l⁴²⁵³ v⁴²⁵⁴ n⁴²⁵⁵ f⁴²⁵⁶
t⁴²⁵⁷ v⁴²⁵⁸ d⁴²⁵⁹ p⁴²⁶⁰ q⁴²⁶¹ k⁴²⁶² a⁴²⁶³ y⁴²⁶⁴ l⁴²⁶⁵ d⁴²⁶⁶ f⁴²⁶⁷ v⁴²⁶⁸ n⁴²⁶⁹ a⁴²⁷⁰ g⁴²⁷¹ g⁴²⁷² a⁴²⁷³ p⁴²⁷⁴ l⁴²⁷⁵
t⁴²⁷⁶ n⁴²⁷⁷ c⁴²⁷⁸ v⁴²⁷⁹ k⁴²⁸⁰ m⁴²⁸¹ l⁴²⁸² t⁴²⁸³ p⁴²⁸⁴ k⁴²⁸⁵ t⁴²⁸⁶ g⁴²⁸⁷ t⁴²⁸⁸ g⁴²⁸⁹ i⁴²⁹⁰ a⁴²⁹¹ i⁴²⁹² s⁴²⁹³ v⁴²⁹⁴
k⁴²⁹⁵ p⁴²⁹⁶ e⁴²⁹⁷ s⁴²⁹⁸ t⁴²⁹⁹ a⁴³⁰⁰ d⁴³⁰¹ q⁴³⁰² e⁴³⁰³ t⁴³⁰⁴ y⁴³⁰⁵ g⁴³⁰⁶ g⁴³⁰⁷ a⁴³⁰⁸ s⁴³⁰⁹ v⁴³¹⁰ c⁴³¹¹ l⁴³¹² y⁴³¹³
c⁴³¹⁴ r⁴³¹⁵ a⁴³¹⁶ h⁴³¹⁷ i⁴³¹⁸ e⁴³¹⁹ h⁴³²⁰ p⁴³²¹ d⁴³²² v⁴³²³ s⁴³²⁴ g⁴³²⁵ v⁴³²⁶ c⁴³²⁷ k⁴³²⁸ y⁴³²⁹ k⁴³³⁰ g⁴³³¹ k⁴³³²
f⁴³³³ v⁴³³⁴ q⁴³³⁵ i⁴³³⁶ p⁴³³⁷ a⁴³³⁸ q⁴³³⁹ c⁴³⁴⁰ v⁴³⁴¹ r⁴³⁴² d⁴³⁴³ p⁴³⁴⁴ v⁴³⁴⁵ g⁴³⁴⁶ f⁴³⁴⁷ c⁴³⁴⁸ l⁴³⁴⁹ s⁴³⁵⁰ n⁴³⁵¹
t⁴³⁵² p⁴³⁵³ c⁴³⁵⁴ n⁴³⁵⁵ v⁴³⁵⁶ c⁴³⁵⁷ q⁴³⁵⁸ y⁴³⁵⁹ w⁴³⁶⁰ i⁴³⁶¹ g⁴³⁶² y⁴³⁶³ g⁴³⁶⁴ c⁴³⁶⁵ n⁴³⁶⁶ c⁴³⁶⁷ d⁴³⁶⁸ s⁴³⁶⁹ l⁴³⁷⁰
r⁴³⁷¹ q⁴³⁷² a⁴³⁷³ a⁴³⁷⁴ l⁴³⁷⁵ p⁴³⁷⁶ q⁴³⁷⁷ s⁴³⁷⁸ k⁴³⁷⁹ d⁴³⁸⁰ s⁴³⁸¹ n⁴³⁸² f⁴³⁸³ l⁴³⁸⁴ n⁴³⁸⁵ r⁴³⁸⁶ v⁴³⁸⁷ r⁴³⁸⁸ g⁴³⁸⁹
s⁴³⁹⁰ i⁴³⁹¹ v⁴³⁹² n⁴³⁹³ a⁴³⁹⁴ r⁴³⁹⁵ i⁴³⁹⁶ e⁴³⁹⁷ p⁴³⁹⁸ c⁴³⁹⁹ s⁴⁴⁰⁰ s⁴⁴⁰¹ g⁴⁴⁰² l⁴⁴⁰³ s⁴⁴⁰⁴ t⁴⁴⁰⁵ d⁴⁴⁰⁶ v⁴⁴⁰⁷ v⁴⁴⁰⁸
f⁴⁴⁰⁹ r⁴⁴¹⁰ a⁴⁴¹¹ f⁴⁴¹² d⁴⁴¹³ i⁴⁴¹⁴ c⁴⁴¹⁵ n⁴⁴¹⁶ y⁴⁴¹⁷ k⁴⁴¹⁸ a⁴⁴¹⁹ k⁴⁴²⁰ v⁴⁴²¹ a⁴⁴²² g⁴⁴²³ i⁴⁴²⁴ g⁴⁴²⁵ k⁴⁴²⁶ y⁴⁴²⁷
y⁴⁴²⁸ k⁴⁴²⁹ t⁴⁴³⁰ n⁴⁴³¹ t⁴⁴³² c⁴⁴³³ r⁴⁴³⁴ f¹⁴³⁵ v⁴⁴³⁶ e⁴⁴³⁷ l⁴⁴³⁸ d⁴⁴³⁹ d⁴⁴⁴⁰ q⁴⁴⁴¹ g⁴⁴⁴² h⁴⁴⁴³ h⁴⁴⁴⁴ l⁴⁴⁴⁵ d⁴⁴⁴⁶
s⁴⁴⁴⁷ y⁴⁴⁴⁸ f⁴⁴⁴⁹ v⁴⁴⁵⁰ v⁴⁴⁵¹ k⁴⁴⁵²r⁴⁴⁵³ h⁴⁴⁵⁴ t⁴⁴⁵⁵ m⁴⁴⁵⁶ e⁴⁴⁵⁷ n⁴⁴⁵⁸ y⁴⁴⁵⁹ e⁴⁴⁶⁰ l⁴⁴⁶¹ e⁴⁴⁶² k⁴⁴⁶³ h⁴⁴⁶⁴ c⁴⁴⁶⁵
y⁴⁴⁶⁶ d⁴⁴⁶⁷ l⁴⁴⁶⁸ l⁴⁴⁶⁹ r⁴⁴⁷⁰ d⁴⁴⁷¹ c⁴⁴⁷² d⁴⁴⁷³ a⁴⁴⁷⁴ v⁴⁴⁷⁵ a⁴⁴⁷⁶ p⁴⁴⁷⁷ h⁴⁴⁷⁸ d⁴⁴⁷⁹ f⁴⁴⁸⁰ f⁴⁴⁸¹ i⁴⁴⁸² f⁴⁴⁸³ d⁴⁴⁸⁴
v⁴⁴⁸⁵ d⁴⁴⁸⁶ k⁴⁴⁸⁷ v⁴⁴⁸⁸ k⁴⁴⁸⁹ t⁴⁴⁹⁰ p⁴⁴⁹¹ h⁴⁴⁹² i⁴⁴⁹³ v⁴⁴⁹⁴ r⁴⁴⁹⁵ q⁴⁴⁹⁶ r⁴⁴⁹⁷ l⁴⁴⁹⁸ t⁴⁴⁹⁹ e⁴⁵⁰⁰ y⁴⁵⁰¹ t⁴⁵⁰² m⁴⁵⁰³
m⁴⁵⁰⁴ d⁴⁵⁰⁵ l⁴⁵⁰⁶ v⁴⁵⁰⁷ y⁴⁵⁰⁸ a⁴⁵⁰⁹ l⁴⁵¹⁰ r⁴⁵¹¹ h⁴⁵¹² f⁴⁵¹³ d⁴⁵¹⁴ q⁴⁵¹⁵ n⁴⁵¹⁶ s⁴⁵¹⁷ e⁴⁵¹⁸ v⁴⁵¹⁹ l⁴⁵²⁰ k⁴⁵²¹ a⁴⁵²²
i⁴⁵²³ l⁴⁵²⁴ v⁴⁵²⁵ k⁴⁵²⁶ y⁴⁵²⁷ g⁴⁵²⁸ c⁴⁵²⁹ c⁴⁵³⁰ d⁴⁵³¹ v⁴⁵³² t⁴⁵³³ y⁴⁵³⁴ f⁴⁵³⁵ e⁴⁵³⁶ n⁴⁵³⁷ k⁴⁵³⁸ l⁴⁵³⁹ w⁴⁵⁴⁰ f⁴⁵⁴¹
d⁴⁵⁴² f⁴⁵⁴³ v⁴⁵⁴⁴ e⁴⁵⁴⁵ n⁴⁵⁴⁶ p⁴⁵⁴⁷ s⁴⁵⁴⁸ v⁴⁵⁴⁹ i⁴⁵⁵⁰ g⁴⁵⁵¹ v⁴⁵⁵² y⁴⁵⁵³ j⁴⁵⁵⁴ k⁴⁵⁵⁵ l⁴⁵⁵⁶ g⁴⁵⁵⁷ e⁴⁵⁵⁸ r⁴⁵⁵⁹ v⁴⁵⁶⁰
r⁴⁵⁶¹ q⁴⁵⁶² a⁴⁵⁶³ i⁴⁵⁶⁴ l⁴⁵⁶⁵ n⁴⁵⁶⁶ t⁴⁵⁶⁷ v⁴⁵⁶⁸ k⁴⁵⁶⁹ f⁴⁵⁷⁰ c⁴⁵⁷¹ d⁴⁵⁷² h⁴⁵⁷³ m⁴⁵⁷⁴ v⁴⁵⁷⁵ k⁴⁵⁷⁶ a⁴⁵⁷⁷ g⁴⁵⁷⁸ l⁴⁵⁷⁹
v⁴⁵⁸⁰ g⁴⁵⁸¹ v⁴⁵⁸² l⁴⁵⁸³ t⁴⁵⁸⁴ l⁴⁵⁸⁵ d⁴⁵⁸⁶ n⁴⁵⁸⁷ g⁴⁵⁸⁸ d⁴⁵⁸⁹ l⁴⁵⁹⁰ n⁴⁵⁹¹ g⁴⁵⁹² k⁴⁵⁹³ w⁴⁵⁹⁴ y⁴⁵⁹⁵ d⁴⁵⁹⁶ f⁴⁵⁹⁷ g⁴⁵⁹⁸
d⁴⁵⁹⁹ f⁴⁶⁰⁰ v⁴⁶⁰¹ i⁴⁶⁰² t⁴⁶⁰³ q⁴⁶⁰⁴ p⁴⁶⁰⁵ g⁴⁶⁰⁶ s⁴⁶⁰⁷ g⁴⁶⁰⁸ v⁴⁶⁰⁹ a⁴⁶¹⁰ i⁴⁶¹¹ v⁴⁶¹² d⁴⁶¹³ s⁴⁶¹⁴ y⁴⁶¹⁵ y⁴⁶¹⁶ s⁴⁶¹⁷
y⁴⁶¹⁸ l⁴⁶¹⁹ m⁴⁶²⁰ p⁴⁶²¹ v⁴⁶²² l⁴⁶²³ s⁴⁶²⁴ m⁴⁶²⁵ t⁴⁶²⁶ d⁴⁶²⁷ c⁴⁶²⁸ l⁴⁶²⁹ a⁴⁶³⁰ a⁴⁶³¹ e⁴⁶³² t⁴⁶³³ h⁴⁶³⁴ r⁴⁶³⁵ d⁴⁶³⁶
c⁴⁶³⁷ d⁴⁶³⁸ f⁴⁶³⁹ n⁴⁶⁴⁰ k⁴⁶⁴¹ p⁴⁶⁴² l⁴⁶⁴³ i⁴⁶¹⁴ e⁴⁶⁴⁵ w⁴⁶⁴⁶ p⁴⁶⁴⁷ l⁴⁶⁴⁸ t⁴⁶⁴⁹ e⁴⁶⁵⁰ y⁴⁶⁵¹ d⁴⁶⁵² f⁴⁶⁵³ t⁴⁶⁵⁴ d⁴⁶⁵⁵
y⁴⁶⁵⁶ k⁴⁶⁵⁷ v⁴⁶⁵⁸ q⁴⁶⁵⁹ l⁴⁶⁶⁰ f⁴⁶⁶¹ e⁴⁶⁶² k⁴⁶⁶³ y⁴⁶⁶⁴ f⁴⁶⁶⁵ k⁴⁶⁶⁶ y⁴⁶⁶⁷ w⁴⁶⁶⁸ d⁴⁶⁶⁹ q⁴⁶⁷⁰ t⁴⁶⁷¹ y⁴⁶⁷² h⁴⁶⁷³ a⁴⁶⁷⁴
n⁴⁶⁷⁵ c⁴⁶⁷⁶ v⁴⁶⁷⁷ n⁴⁶⁷⁸ c⁴⁶⁷⁹ t⁴⁶⁸⁰ d⁴⁶⁸¹ d⁴⁶⁸² r⁴⁶⁸³ c⁴⁶⁸⁴ v⁴⁶⁸⁵ l⁴⁶⁸⁶ h⁴⁶⁸⁷ c⁴⁶⁸⁸ a⁴⁶⁸⁹ n⁴⁶⁹⁰ f⁴⁶⁹¹ n⁴⁶⁹² v⁴⁶⁹³
l⁴⁶⁹⁴ f⁴⁶⁹⁵ a⁴⁶⁹⁶ m⁴⁶⁹⁷ t⁴⁶⁹⁸ m⁴⁶⁹⁹ p⁴⁷⁰⁰ k⁴⁷⁰¹ t⁴⁷⁰² c⁴⁷⁰³ f⁴⁷⁰⁴ g⁴⁷⁰⁵ p⁴⁷⁰⁶ i⁴⁷⁰⁷ v⁴⁷⁰⁸ r⁴⁷⁰⁹ k⁴⁷¹⁰ i⁴⁷¹¹ f⁴⁷¹²
v⁴⁷¹³ d⁴⁷¹⁴ g⁴⁷¹⁵ v⁴⁷¹⁶ p⁴⁷¹⁷ f⁴⁷¹⁸ v⁴⁷¹⁹ v⁴⁷²⁰ s⁴⁷²¹ c⁴⁷²² g⁴⁷²³ y⁴⁷²⁴ h⁴⁷²⁵ y⁴⁷²⁶ k⁴⁷²⁷ e⁴⁷²⁸ l⁴⁷²⁹ g⁴⁷³⁰ l⁴⁷³¹
v⁴⁷³² m⁴⁷³³ n⁴⁷³⁴ m⁴⁷³⁵ d⁴⁷³⁶ v⁴⁷³⁷ s⁴⁷³⁸ l⁴⁷³⁹ h⁴⁷⁴⁰ r⁴⁷⁴¹ h⁴⁷⁴² r⁴⁷⁴³ l⁴⁷⁴⁴ s⁴⁷⁴⁵ l⁴⁷⁴⁶ k⁴⁷⁴⁷ e⁴⁷⁴⁸ l⁴⁷⁴⁹ m⁴⁷⁵⁰
m⁴⁷⁵¹ y⁴⁷⁵² a⁴⁷⁵³ a⁴⁷⁵⁴ d⁴⁷⁵⁵ p⁴⁷⁵⁶ a⁴⁷⁵⁷ m⁴⁷⁵⁸ h⁴⁷⁵⁹ i⁴⁷⁶⁰ a⁴⁷⁶¹ s⁴⁷⁶² s⁴⁷⁶³ n⁴⁷⁶⁴ a⁴⁷⁶⁵ f⁴⁷⁶⁶ l⁴⁷⁶⁷ d⁴⁷⁶⁸ l⁴⁷⁶⁹
r⁴⁷⁷⁰ t⁴⁷⁷¹ s⁴⁷⁷² c⁴⁷⁷³ f⁴⁷⁷⁴ s⁴⁷⁷⁵ v⁴⁷⁷⁶ a⁴⁷⁷⁷ a⁴⁷⁷⁸ l⁴⁷⁷⁹ t⁴⁷⁸⁰ t⁴⁷⁸¹ g⁴⁷⁸² l⁴⁷⁸³ t⁴⁷⁸⁴ f⁴⁷⁸⁵ q⁴⁷⁸⁶ t⁴⁷⁸⁷ v⁴⁷⁸⁸
r⁴⁷⁸⁹ p⁴⁷⁹⁰ g⁴⁷⁹¹ n⁴⁷⁹² f⁴⁷⁹³ n⁴⁷⁹⁴ q⁴⁷⁹⁵ d⁴⁷⁹⁶ f⁴⁷⁹⁷ y⁴⁷⁹⁸ d⁴⁷⁹⁹ f⁴⁸⁰⁰ v⁴⁸⁰¹ v⁴⁸⁰² s⁴⁸⁰³ k⁴⁸⁰⁴ g⁴⁸⁰⁵ f⁴⁸⁰⁶ f⁴⁸⁰⁷
k⁴⁸⁰⁸ e⁴⁸⁰⁹ g⁴⁸¹⁰ s⁴⁸¹¹ s⁴⁸¹² v⁴⁸¹³ t⁴⁸¹⁴ l⁴⁸¹⁵ k⁴⁸¹⁶ h⁴⁸¹⁷ f⁴⁸¹⁸ f⁴⁸¹⁹ f⁴⁸²⁰ a⁴⁸²¹ q⁴⁸²² d⁴⁸²³ g⁴⁸²⁴ n⁴⁸²⁵ a⁴⁸²⁶
a⁴⁸²⁷ i⁴⁸²⁸ t⁴⁸²⁹ d⁴⁸³⁰ y⁴⁸³¹ n⁴⁸³² y⁴⁸³³ y⁴⁸³⁴ s⁴⁸³⁵ y⁴⁸³⁶ n⁴⁸³⁷ l⁴⁸³⁸ p⁴⁸³⁹ t⁴⁸⁴⁰ m⁴⁹⁴¹ c⁴⁸⁴² d⁴⁹⁴³ i⁴⁸⁴⁴ k⁴⁸⁴⁵
q⁴⁸⁴⁶ m⁴⁸⁴⁷ l⁴⁸⁴⁸ f⁴⁸⁴⁹ c⁴⁸⁵⁰ m⁴⁸⁵¹ e⁴⁸⁵² v⁴⁸⁵³ v⁴⁸⁵⁴ n⁴⁸⁵⁵ k⁴⁸⁵⁶ y⁴⁸⁵⁷ f⁴⁸⁵⁸ e⁴⁸⁵⁹ i⁴⁸⁶⁰ y⁴⁸⁶¹ d⁴⁸⁶² g⁴⁸⁶³ g⁴⁸⁶⁴
c⁴⁸⁶⁵ l⁴⁸⁶⁶ n⁴⁸⁶⁷ a⁴⁸⁶⁸ s⁴⁸⁶⁹ e⁴⁸⁷⁰ v⁴⁸⁷¹ v⁴⁸⁷² v⁴⁸⁷³ n⁴⁸⁷⁴ n⁴⁸⁷⁵ l⁴⁸⁷⁶ d⁴⁸⁷⁷ k⁴⁸⁷⁸ s⁴⁸⁷⁹ a⁴⁸⁸⁰ g⁴⁸⁸¹ h⁴⁸⁸² p⁴⁸⁸³
f⁴⁸⁸⁴ n⁴⁸⁸⁵ k⁴⁸⁸⁶ f⁴⁸⁸⁷ g⁴⁸⁸⁸ k⁴⁸⁸⁹ a⁴⁸⁹⁰ r⁴⁸⁹¹ v⁴⁸⁹² y⁴⁸⁹³ y⁴⁸⁹⁴ e⁴⁸⁹⁵ s⁴⁸⁹⁶ m⁴⁸⁹⁷ s⁴⁸⁹⁸ y⁴⁸⁹⁹ q⁴⁹⁰⁰ e⁴⁹⁰¹ q⁴⁹⁰²
d⁴⁹⁰³ e⁴⁹⁰⁴ l⁴⁹⁰⁵ f⁴⁹⁰⁶ a⁴⁹⁰⁷ m⁴⁹⁰⁸ t⁴⁹⁰⁹ k⁴⁹¹⁰ r⁴⁹¹¹ n⁴⁹¹² v⁴⁹¹³ i⁴⁹¹⁴ p⁴⁹¹⁵ t⁴⁹¹⁶ m⁴⁹¹⁷ t⁴⁹¹⁸ q⁴⁹¹⁹ m⁴⁹²⁰ n⁴⁹²¹
l⁴⁹²² k⁴⁹²³ y⁴⁹²⁴ a⁴⁹²⁵ i⁴⁹²⁶ s⁴⁹²⁷ a⁴⁹²⁸ k⁴⁹²⁹ n⁴⁹³⁰ r⁴⁹³¹ a⁴⁹³² r⁴⁹³³ t⁴⁹³⁴ v⁴⁹³⁵ a⁴⁹³⁶ g⁴⁹³⁷ v⁴⁹³⁸ s⁴⁹³⁹ i⁴⁹⁴⁰
l⁴⁹⁴¹ s⁴⁹⁴² t⁴⁹⁴³ m⁴⁹⁴⁴ t⁴⁹⁴⁵ n⁴⁹⁴⁶ r⁴⁹⁴⁷ q⁴⁹⁴⁸ y⁴⁹⁴⁹ h⁴⁹⁵⁰ q⁴⁹⁵¹ k⁴⁹⁵² m⁴⁹⁵³ l⁴⁹⁵⁴ k⁴⁹⁵⁵ s⁴⁹⁵⁶ m⁴⁹⁵⁷ a⁴⁹⁵⁸ a⁴⁹⁵⁹
t⁴⁹⁶⁰ r⁴⁹⁶¹ g⁴⁹⁶² a⁴⁹⁶³ t⁴⁹⁶⁴ c⁴⁹⁶⁵ v⁴⁹⁶⁶ i⁴⁹⁶⁷ g⁴⁹⁶⁸ t⁴⁹⁶⁹ t⁴⁹⁷⁰ k⁴⁹⁷¹ f⁴⁹⁷² y⁴⁹⁷³ g⁴⁹⁷⁴ g⁴⁹⁷⁵ w⁴⁹⁷⁶ d⁴⁹⁷⁷ f⁴⁹⁷⁸
m⁴⁹⁷⁹ l⁴⁹⁸⁰ k⁴⁹⁸¹ t⁴⁹⁸² l⁴⁹⁸³ y⁴⁹⁸⁴ k⁴⁹⁸⁵ d⁴⁹⁸⁶ v⁴⁹⁸⁷ d⁴⁹⁸⁸ n⁴⁹⁸⁹ p⁴⁹⁹⁰ h⁴⁹⁹¹ l⁴⁹⁹² m⁴⁹⁹³ g⁴⁹⁹⁴ w⁴⁹⁹⁵ d⁴⁹⁹⁶ y⁴⁹⁹⁷
p⁴⁹⁹⁸ k⁴⁹⁹⁹ c⁵⁰⁰⁰ d⁵⁰⁰¹ r⁵⁰⁰² a⁵⁰⁰³ m⁵⁰⁰⁴ p⁵⁰⁰⁵ n⁵⁰⁰⁶ m⁵⁰⁰⁷ c⁵⁰⁰⁸ r⁵⁰⁰⁹ i⁵⁰¹⁰ f⁵⁰¹¹ a⁵⁰¹² s⁵⁰¹³ l⁵⁰¹⁴ i⁵⁰¹⁵ l⁵⁰¹⁶
a⁵⁰¹⁷ r⁵⁰¹⁸ k⁵⁰¹⁹ h⁵⁰²⁰ g⁵⁰²¹ t⁵⁰²² c⁵⁰²³ c⁵⁰²⁴ t⁵⁰²⁵ t⁵⁰²⁶ r⁵⁰²⁷ d⁵⁰²⁸ r⁵⁰²⁹ f⁵⁰³⁰ y⁵⁰³¹ r⁵⁰³² l⁵⁰³³ a⁵⁰³⁴ n⁵⁰³⁵
e⁵⁰³⁶ c⁵⁰³⁷ a⁵⁰³⁸ q⁵⁰³⁹ v⁵⁰⁴⁰ l⁵⁰⁴¹ s⁵⁰⁴² e⁵⁰⁴³ y⁵⁰⁴⁴ v⁵⁰⁴⁵ l⁵⁰⁴⁶ c⁵⁰⁴⁷ g⁵⁰⁴⁸ g⁵⁰⁴⁹ g⁵⁰⁵⁰ y⁵⁰⁵¹ y⁵⁰⁵² v⁵⁰⁵³ k⁵⁰⁵⁴
p⁵⁰⁵⁵ g⁵⁰⁵⁶ g⁵⁰⁵⁷ t⁵⁰⁵⁸ s⁵⁰⁵⁹ s⁵⁰⁶⁰ g⁵⁰⁶¹ d⁵⁰⁶² a⁵⁰⁶³ t⁵⁰⁶⁴ t⁵⁰⁶⁵ a⁵⁰⁶⁶ y⁵⁰⁶⁷ a⁵⁰⁶⁸ n⁵⁰⁶⁹ s⁵⁰⁷⁰ v⁵⁰⁷¹ f⁵⁰⁷² n⁵⁰⁷³
i⁵⁰⁷⁴ l⁵⁰⁷⁵ q⁵⁰⁷⁶ a⁵⁰⁷⁷ t⁵⁰⁷⁸ t⁵⁰⁷⁹ a⁵⁰⁸⁰ n⁵⁰⁸¹ v⁵⁰⁸² s⁵⁰⁸³ a⁵⁰⁸⁴ l⁵⁰⁸⁵ m⁵⁰⁸⁶ g⁵⁰⁸⁷ a⁵⁰⁸⁸ n⁵⁰⁸⁹ g⁵⁰⁹⁰ n⁵⁰⁹¹ k⁵⁰⁹²
i⁵⁰⁹³ v⁵⁰⁹⁴ d⁵⁰⁹⁵ k⁵⁰⁹⁶ e⁵⁰⁹⁷ v⁵⁰⁹⁸ k⁵⁰⁹⁹ d⁵¹⁰⁰ m⁵¹⁰¹ q⁵¹⁰² f⁵¹⁰³ d⁵¹⁰⁴ l⁵¹⁰⁵ y⁵¹⁰⁶ v⁵¹⁰⁷ n⁵¹⁰⁸ v⁵¹⁰⁹ y⁵¹¹⁰ r⁵¹¹¹
s⁵¹¹² t⁵¹¹³ s⁵¹¹⁴ p⁵¹¹⁵ d⁵¹¹⁶ p⁵¹¹⁷ k⁵¹¹⁸ f⁵¹¹⁹ v⁵¹²⁰ d⁵¹²¹ k⁵¹²² y⁵¹²³ y⁵¹²⁴ a⁵¹²⁵ f⁵¹²⁶ l⁵¹²⁷ n⁵¹²⁸ k⁵¹²⁹ h⁵¹³⁰
f⁵¹³¹ s⁵¹³² m⁵¹³³ m⁵¹³⁴ i⁵¹³⁵ l⁵¹³⁶ s⁵¹³⁷ d⁵¹³⁸ d⁵¹³⁹ g⁵¹⁴⁰ v⁵¹⁴¹ v⁵¹⁴² c⁵¹⁴³ y⁵¹⁴⁴ n⁵¹⁴⁵ s⁵¹⁴⁶ d⁵¹⁴⁷ y⁵¹⁴⁸ a⁵¹⁴⁹
a⁵¹⁵⁰ k⁵¹⁵¹ g⁵¹⁵² y⁵¹⁵³ i⁵¹⁵⁴ f⁵¹⁵⁵ g⁵¹⁵⁶ i⁵¹⁵⁷ q⁵¹⁵⁸ n⁵¹⁵⁹ f⁵¹⁶⁰ k⁵¹⁶¹ e⁵¹⁶² t⁵¹⁶³ l⁵¹⁶⁴ y⁵¹⁶⁵ y⁵¹⁶⁶ q⁵¹⁶⁷ n⁵¹⁶⁸
n⁵¹⁶⁹ v⁵¹⁷⁰ f⁵¹⁷¹ m⁵¹⁷² s⁵¹⁷³ e⁵¹⁷⁴ a⁵¹⁷⁵ k⁵¹⁷⁶ c⁵¹⁷⁷ w⁵¹⁷⁸ v⁵¹⁷⁹ e⁵¹⁸⁰ t⁵¹⁸¹ d⁵¹⁸² l⁵¹⁸³ k⁵¹⁸⁴ k⁵¹⁸⁵ g⁵¹⁸⁶ p⁵¹⁸⁷
h⁵¹⁸⁸ e⁵¹⁸⁹ f⁵¹⁹⁰ c⁵¹⁹¹ s⁵¹⁹² q⁵¹⁹³ h⁵¹⁹⁴ t⁵¹⁹⁵ l⁵¹⁹⁶ y⁵¹⁹⁷ i⁵¹⁹⁸ k⁵¹⁹⁹ d⁵²⁰⁰ g⁵²⁰¹ d⁵²⁰² d⁵²⁰³ g⁵²⁰⁴ y⁵²⁰⁵ f⁵²⁰⁶
l⁵²⁰⁷ p⁵²⁰⁸ y⁵²⁰⁹ p⁵²¹⁰ d⁵²¹¹ p⁵²¹² s⁵²¹³ r⁵²¹⁴ i⁵²¹⁵ l⁵²¹⁶ s⁵²¹⁷ a⁵²¹⁸ g⁵²¹⁹ c⁵²²⁰ f⁵²²¹ v⁵²²² d⁵²²³ d⁵²²⁴ i⁵²²⁵
v⁵²²⁶ k⁵²²⁷ t⁵²²⁸ d⁵²²⁹ g⁵²³⁰ t⁵²³¹ l⁵²³² m⁵²³³ v⁵²³⁴ e⁵²³⁵ r⁵²³⁶ f⁵²³⁷ v⁵²³⁸ s⁵²³⁹ l⁵²⁴⁰ a⁵²⁴¹ i⁵²⁴² d⁵²⁴³ a⁵²⁴⁴
y⁵²⁴⁵ p⁵²⁴⁶ l⁵²⁴⁷ t⁵²⁴⁸ k⁵²⁴⁹ h⁵²⁵⁰ e⁵²⁵¹ d⁵²⁵² i⁵²⁵³ e⁵²⁵⁴ y⁵²⁵⁵ q⁵²⁵⁶ n⁵²⁵⁷ v⁵²⁵⁸ f⁵²⁵⁹ w⁵²⁶⁰ v⁵²⁶¹ y⁵²⁶² l⁵²⁶³
q⁵²⁶⁴ y⁵²⁶⁵ i⁵²⁶⁶ e⁵²⁶⁷ k⁵²⁶⁸ l⁵²⁶⁹ y⁵²⁷⁰ k⁵²⁷¹ d⁵²⁷² l⁵²⁷³ t⁵²⁷⁴ g⁵²⁷⁵ h⁵²⁷⁶ m⁵²⁷⁷ l⁵²⁷⁸ d⁵²⁷⁹ s⁵²⁸⁰ y⁵²⁸¹ s⁵²⁸²
v⁵²⁸³ m⁵²⁸⁴ l⁵²⁸⁵ c⁵²⁸⁶ g⁵²⁸⁷ d⁵²⁸⁸ n⁵²⁸⁹ s⁵²⁹⁰ a⁵²⁹¹ k⁵²⁹² f⁵²⁹³ w⁵²⁹⁴ e⁵²⁹⁵ e⁵²⁹⁶ a⁵²⁹⁷ f⁵²⁹⁸ y⁵²⁹⁹ r⁵³⁰⁰ d⁵³⁰¹
l⁵³⁰² y⁵³⁰³ s⁵³⁰⁴ s⁵³⁰⁵ p⁵³⁰⁶ t⁵³⁰⁷ t⁵³⁰⁸ l⁵³⁰⁹ q⁵³¹⁰ a⁵³¹¹ v⁵³¹² g⁵³¹³ s⁵³¹⁴ c⁵³¹⁵ v⁵³¹⁶ v⁵³¹⁷ c⁵³¹⁸ h⁵³¹⁹ s⁵³²⁰
q⁵³²¹ t⁵³²² s⁵³²³ l⁵³²⁴ r⁵³²⁵ c⁵³²⁶ g⁵³²⁷ t⁵³²⁸ c⁵³²⁹ i⁵³³⁰ r⁵³³¹ r⁵³³² p⁵³³³ f⁵³³⁴ l⁵³³⁵ c⁵³³⁶ c⁵³³⁷ k⁵³³⁸ c⁵³³⁹
c⁵³⁴⁰ y⁵³⁴¹ d⁵³⁴² h⁵³⁴³ v⁵³⁴⁴ i⁵³⁴⁵ a⁵³⁴⁶ t⁵³⁴⁷ p⁵³⁴⁸ h⁵³⁴⁹ k⁵³⁵⁰ m⁵³⁵¹ v⁵³⁵² l⁵³⁵³ s⁵³⁵⁴ v⁵³⁵⁵ s⁵³⁵⁶ p⁵³⁵⁷ y⁵³⁵⁸
v⁵³⁵⁹ c⁵³⁶⁰ n⁵³⁶¹ a⁵³⁶² p⁵³⁶³ g⁵³⁶⁴ c⁵³⁶⁵ g⁵³⁶⁶ v⁵³⁶⁷ s⁵³⁶⁸ d⁵³⁶⁹ v⁵³⁷⁰ t⁵³⁷¹ k⁵³⁷² l⁵³⁷³ y⁵³⁷⁴ l⁵³⁷⁵ g⁵³⁷⁶ g⁵³⁷⁷
m⁵³⁷⁸ s⁵³⁷⁹ y⁵³⁸⁰ f⁵³⁸¹ c⁵³⁸² v⁵³⁸³ d⁵³⁸⁴ h⁵³⁸⁵ r⁵³⁸⁶ p⁵³⁸⁷ v⁵³⁸⁸ c⁵³⁸⁹ s⁵³⁹⁰ f⁵³⁹¹ p⁵³⁹² l⁵³⁹³ c⁵³⁹⁴ a⁵³⁹⁵ n⁵³⁹⁶
g⁵³⁹⁷ l⁵³⁹⁸ v⁵³⁹⁹ f⁵⁴⁰⁰ g⁵⁴⁰¹ l⁵⁴⁰² y⁵⁴⁰³ k⁵⁴⁰⁴ n⁵⁴⁰⁵ m⁵⁴⁰⁶ c⁵⁴⁰⁷ t⁵⁴⁰⁸ g⁵⁴⁰⁹ s⁵⁴¹⁰ p⁵⁴¹¹ s⁵⁴¹² i⁵⁴¹³ v⁵⁴¹⁴ e⁵⁴¹⁵
f⁵⁴¹⁶ n⁵⁴¹⁷ r⁵⁴¹⁸ l⁵⁴¹⁹ a⁵⁴²⁰ t⁵⁴²¹ c⁵⁴²² d⁵⁴²³ w⁵⁴²⁴ t⁵⁴²⁵ e⁵⁴²⁶ s⁵⁴²⁷ g⁵⁴²⁸ d⁵⁴²⁹ y⁵⁴³⁰ t⁵⁴³¹ l⁵⁴³² a⁵⁴³³ n⁵⁴³⁴
t⁵⁴³⁵ t⁵⁴³⁶ t⁵⁴³⁷ e⁵⁴³⁸ p⁵⁴³⁹ l⁵⁴⁴⁰ k⁵⁴⁴¹ l⁵⁴⁴² f⁵⁴⁴³ a⁵⁴⁴⁴ a⁵⁴⁴⁵ e⁵⁴⁴⁶ t⁵⁴⁴⁷ l⁵⁴⁴⁸ r⁵⁴⁴⁹ a⁵⁴⁵⁰ t⁵⁴⁵¹ e⁵⁴⁵² e⁵⁴⁵³
a⁵⁴⁵⁴ s⁵⁴⁵⁵ k⁵⁴⁵⁶ q⁵⁴⁵⁷ s⁵⁴⁵⁸ y⁵⁴⁵⁹ a⁵⁴⁶⁰ i⁵⁴⁶¹ a⁵⁴⁶² t⁵⁴⁶³ i⁵⁴⁶⁴ k⁵⁴⁶⁵ e⁵⁴⁶⁶ i⁵⁴⁶⁷ v⁵⁴⁶⁸ g⁵⁴⁶⁹ e⁵⁴⁷⁰ r⁵⁴⁷¹ q⁵⁴⁷²
l⁵⁴⁷³ l⁵⁴⁷⁴ l⁵⁴⁷⁵ v⁵⁴⁷⁶ w⁵⁴⁷⁷ e⁵⁴⁷⁸ a⁵⁴⁷⁹ g⁵⁴⁸⁰ k⁵⁴⁸¹ s⁵⁴⁸² k⁵⁴⁸³ p⁵⁴⁸⁴ p⁵⁴⁸⁵ l⁵⁴⁸⁶ n⁵⁴⁸⁷ r⁵⁴⁸⁸ n⁵⁴⁸⁹ y⁵⁴⁹⁰ v⁵⁴⁹¹
f⁵⁴⁹² t⁵⁴⁹³ g⁵⁴⁹⁴ y⁵⁴⁹⁵ h⁵⁴⁹⁶ i⁵⁴⁹⁷ t⁵⁴⁹⁸ k⁵⁴⁹⁹ n⁵⁵⁰⁰ s⁵⁵⁰¹ k⁵⁵⁰² v⁵⁵⁰³ q⁵⁵⁰⁴ l⁵⁵⁰⁵ g⁵⁵⁰⁶ e⁵⁵⁰⁷ y⁵⁵⁰⁸ i⁵⁵⁰⁹ f⁵⁵¹⁰
e⁵⁵¹¹ r⁵⁵¹² i⁵⁵¹³ d⁵⁵¹⁴ y⁵⁵¹⁵ s⁵⁵¹⁶ d⁵⁵¹⁷ a⁵⁵¹⁸ v⁵⁵¹⁹ s⁵⁵²⁰ y⁵⁵²¹ k⁵⁵²² s⁵⁵²³ s⁵⁵²⁴ t⁵⁵²⁵ t⁵⁵²⁶ y⁵⁵²⁷ k⁵⁵²⁸ l⁵⁵²⁹
t⁵⁵³⁰ v⁵⁵³¹ g⁵⁵³² d⁵⁵³³ i⁵⁵³⁴ f⁵⁵³⁵ v⁵⁵³⁶ l⁵⁵³⁷ t⁵⁵³⁸ s⁵⁵³⁹ h⁵⁵⁴⁰ s⁵⁵⁴¹ v⁵⁵⁴² a⁵⁵⁴³ t⁵⁵⁴⁴ l⁵⁵⁴⁵ t⁵⁵⁴⁶ a⁵⁵⁴⁷ p⁵⁵⁴⁸
t⁵⁵⁴⁹ i⁵⁵⁵⁰ v⁵⁵⁵¹ n⁵⁵⁵² q⁵⁵⁵³ e⁵⁵⁵⁴ r⁵⁵⁵⁵ y⁵⁵⁵⁶ v⁵⁵⁵⁷ k⁵⁵⁵⁸ i⁵⁵⁵⁹ t⁵⁵⁶⁰ g⁵⁵⁶¹ l⁵⁵⁶² y⁵⁵⁶³ p⁵⁵⁶⁴ t⁵⁵⁶⁵ i⁵⁵⁶⁶ t⁵⁵⁶⁷
v⁵⁵⁶⁸ p⁵⁵⁶⁹ e⁵⁵⁷⁰ e⁵⁵⁷¹ f⁵⁵⁷² a⁵⁵⁷³ s⁵⁵⁷⁴ h⁵⁵⁷⁵ v⁵⁵⁷⁶ a⁵⁵⁷⁷ n⁵⁵⁷⁸ f⁵⁵⁷⁹ q⁵⁵⁸⁰ k⁵⁵⁸¹ s⁵⁵⁸² g⁵⁵⁸³ y⁵⁵⁸⁴ s⁵⁵⁸⁵ k⁵⁵⁸⁶
y⁵⁵⁸⁷ v⁵⁵⁸⁸ t⁵⁵⁸⁹ v⁵⁵⁹⁰ q⁵⁵⁹¹ g⁵⁵⁹² p⁵⁵⁹³ p⁵⁵⁹⁴ g⁵⁵⁹⁵ t⁵⁵⁹⁶ g⁵⁵⁹⁷ k⁵⁵⁹⁸ s⁵⁵⁹⁹ h⁵⁶⁰⁰ f⁵⁶⁰¹ a⁵⁶⁰² i⁵⁶⁰³ g⁵⁶⁰⁴ l⁵⁶⁰⁵
a⁵⁶⁰⁶ i⁵⁶⁰⁷ y⁵⁶⁰⁸ y⁵⁶⁰⁹ p⁵⁶¹⁰ t⁵⁶¹¹ a⁵⁶¹² r⁵⁶¹³ v⁵⁶¹⁴ v⁵⁶¹⁵ y⁵⁶¹⁶ t⁵⁶¹⁷ a⁵⁶¹⁸ c⁵⁶¹⁹ s⁵⁶²⁰ h⁵⁶²¹ a⁵⁶²² a⁵⁶²³ v⁵⁶²⁴
d⁵⁶²⁵ a⁵⁶²⁶ l⁵⁶²⁷ c⁵⁶²⁸ e⁵⁶²⁹ k⁵⁶³⁰ a⁵⁶³¹ f⁵⁶³² k⁵⁶³³ y⁵⁶³⁴ l⁵⁶³⁵ n⁵⁶³⁶ i⁵⁶³⁷ a⁵⁶³⁸ k⁵⁶³⁹ c⁵⁶⁴⁰ s⁵⁶⁴¹ r⁵⁶⁴² i⁵⁶⁴³
i⁵⁶⁴⁴ p⁵⁶⁴⁵ a⁵⁶⁴⁶ k⁵⁶⁴⁷ a⁵⁶⁴⁸ r⁵⁶⁴⁹ v⁵⁶⁵⁰ e⁵⁶⁵¹ c⁵⁶⁵² y⁵⁶⁵³ d⁵⁶⁵⁴ r⁵⁶⁵⁵ f⁵⁶⁵⁶ k⁵⁶⁵⁷ v⁵⁶⁵⁸ n⁵⁶⁵⁹ e⁵⁶⁶⁰ t⁵⁶⁶¹ n⁵⁶⁶²
s⁵⁶⁶³ q⁵⁶⁶⁴ y⁵⁶⁶⁵ l⁵⁶⁶⁶ f⁵⁶⁶⁷ s⁵⁶⁶⁸ t⁵⁶⁶⁹ i⁵⁶⁷⁰ n⁵⁶⁷¹ a⁵⁶⁷² l⁵⁶⁷³ p⁵⁶⁷⁴ e⁵⁶⁷⁵ t⁵⁶⁷⁶ s⁵⁶⁷⁷ a⁵⁶⁷⁸ d⁵⁶⁷⁹ i⁵⁶⁸⁰ l⁵⁶⁸¹
v⁵⁶⁸² v⁵⁶⁸³ d⁵⁶⁸⁴ e⁵⁶⁸⁵ v⁵⁶⁸⁶ s⁵⁶⁸⁷ m⁵⁶⁸⁸ c⁵⁶⁸⁹ t⁵⁶⁹⁰ n⁵⁶⁹¹ y⁵⁶⁹² d⁵⁶⁹³ l⁵⁶⁹⁴ s⁵⁶⁹⁵ i⁵⁶⁹⁶ i⁵⁶⁹⁷ n⁵⁶⁹⁸ a⁵⁶⁹⁹ r⁵⁷⁰⁰
i⁵⁷⁰¹ k⁵⁷⁰² a⁵⁷⁰³ k⁵⁷⁰⁴ h⁵⁷⁰⁵ i⁵⁷⁰⁶ v⁵⁷⁰⁷ y⁵⁷⁰⁸ v⁵⁷⁰⁹ g⁵⁷¹⁰ d⁵⁷¹¹ p⁵⁷¹² a⁵⁷¹³ q⁵⁷¹⁴ l⁵⁷¹⁵ p⁵⁷¹⁶ a⁵⁷¹⁷ p⁵⁷¹⁸ r⁵⁷¹⁹
t⁵⁷²⁰ l⁵⁷²¹ l⁵⁷²² t⁵⁷²³ r⁵⁷²⁴ g⁵⁷²⁵ t⁵⁷²⁶ l⁵⁷²⁷ e⁵⁷²⁸ p⁵⁷²⁹ e⁵⁷³⁰ n⁵⁷³¹ f⁵⁷³² n⁵⁷³³ s⁵⁷³⁴ v⁵⁷³⁵ t⁵⁷³⁶ r⁵⁷³⁷ l⁵⁷³⁸
m⁵⁷³⁹ c⁵⁷⁴⁰ n⁵⁷⁴¹ l⁵⁷⁴² g⁵⁷⁴³ p⁵⁷⁴⁴ d⁵⁷⁴⁵ i⁵⁷⁴⁶ f⁵⁷⁴⁷ l⁵⁷⁴⁸ s⁵⁷⁴⁹ m⁵⁷⁵⁰ c⁵⁷⁵¹ y⁵⁷⁵² r⁵⁷⁵³ c⁵⁷⁵⁴ p⁵⁷⁵⁵ k⁵⁷⁵⁶ e⁵⁷⁵⁷
i⁵⁷⁵⁸ v⁵⁷⁵⁹ s⁵⁷⁶⁰ t⁵⁷⁶¹ v⁵⁷⁶² s⁵⁷⁶³ a⁵⁷⁶⁴ l⁵⁷⁶⁵ v⁵⁷⁶⁶ y⁵⁷⁶⁷ n⁵⁷⁶⁸ n⁵⁷⁶⁹ k⁵⁷⁷⁰ l⁵⁷⁷¹ l⁵⁷⁷² a⁵⁷⁷³ k⁵⁷⁷⁴ k⁵⁷⁷⁵ e⁵⁷⁷⁶
l⁵⁷⁷⁷ s⁵⁷⁷⁸ g⁵⁷⁷⁹ q⁵⁷⁸⁰ c⁵⁷⁸¹ f⁵⁷⁸² k⁵⁷⁸³ i⁵⁷⁸⁴ l⁵⁷⁸⁵ y⁵⁷⁸⁶ k⁵⁷⁸⁷ g⁵⁷⁸⁸ n⁵⁷⁸⁹ v⁵⁷⁹⁰ t⁵⁷⁹¹ h⁵⁷⁹² d⁵⁷⁹³ a⁵⁷⁹⁴ s⁵⁷⁹⁵
s⁵⁷⁹⁶ a⁵⁷⁹⁷ i⁵⁷⁹⁸ n⁵⁷⁹⁹ r⁵⁸⁰⁰ p⁵⁸⁰¹ q⁵⁸⁰² l⁵⁸⁰³ t⁵⁸⁰⁴ f⁵⁸⁰⁵ v⁵⁸⁰⁶ k⁵⁸⁰⁷ n⁵⁸⁰⁸ f⁵⁸⁰⁹ i⁵⁸¹⁰ t⁵⁸¹¹ a⁵⁸¹² n⁵⁸¹³ p⁵⁸¹⁴
a⁵⁸¹⁵ w⁵⁸¹⁶ s⁵⁸¹⁷ k⁵⁸¹⁸ a⁵⁸¹⁹ v⁵⁸²⁰ f⁵⁸²¹ i⁵⁸²² s⁵⁸²³ p⁵⁸²⁴ y⁵⁸²⁵ n⁵⁸²⁶ s⁵⁸²⁷ q⁵⁸²⁸ n⁵⁸²⁹ a⁵⁸³⁰ v⁵⁸³¹ a⁵⁸³² r⁵⁸³³
s⁵⁸³⁴ m⁵⁸³⁵ l⁵⁸³⁶ g⁵⁸³⁷ l⁵⁸³⁸ t⁵⁸³⁹ t⁵⁸⁴⁰ q⁵⁸⁴¹ t⁵⁸⁴² v⁵⁸⁴³ d⁵⁸⁴⁴ s⁵⁸⁴⁵ s⁵⁸⁴⁶ q⁵⁸⁴⁷ g⁵⁸⁴⁸ s⁵⁸⁴⁹ e⁵⁸⁵⁰ y⁵⁸⁵¹ q⁵⁸⁵²
y⁵⁸⁵³ v⁵⁸⁵⁴ i⁵⁸⁵⁵ f⁵⁸⁵⁶ c⁵⁸⁵⁷ q⁵⁸⁵⁸ t⁵⁸⁵⁹ a⁵⁸⁶⁰ d⁵⁸⁶¹ t⁵⁸⁶² a⁵⁸⁶³ h⁵⁸⁶⁴ a⁵⁸⁶⁵ n⁵⁸⁶⁶ n⁵⁸⁶⁷ i⁵⁸⁶⁸ n⁵⁸⁶⁹ r⁵⁸⁷⁰ f⁵⁸⁷¹
n⁵⁸⁷² v⁵⁸⁷³ a⁵⁸⁷⁴ i⁵⁸⁷⁵ t⁵⁸⁷⁶ r⁵⁸⁷⁷ a⁵⁸⁷⁸ q⁵⁸⁷⁹ k⁵⁸⁸⁰ g⁵⁸⁸¹ i⁵⁸⁸² l⁵⁸⁸³ e⁵⁸⁸⁴ v⁵⁸⁸⁵ m⁵⁸⁸⁶ t⁵⁸⁸⁷ s⁵⁸⁸⁸ q⁵⁸⁸⁹ a⁵⁸⁹⁰
l⁵⁸⁹¹ f⁵⁸⁹² e⁵⁸⁹³ s⁵⁸⁹⁴ l⁵⁸⁹⁵ e⁵⁸⁹⁶ f⁵⁸⁹⁷ t⁵⁸⁹⁸ e⁵⁸⁹⁹ l⁵⁹⁰⁰ s⁵⁹⁰¹ f⁵⁹⁰² t⁵⁹⁰³ n⁵⁹⁰⁴ y⁵⁹⁰⁵ k⁵⁹⁰⁶ l⁵⁹⁰⁷ q⁵⁹⁰⁸ s⁵⁹⁰⁹
q⁵⁹¹⁰ i⁵⁹¹¹ v⁵⁹¹² t⁵⁹¹³ g⁵⁹¹⁴ l⁵⁹¹⁵ f⁵⁹¹⁶ k⁵⁹¹⁷ d⁵⁹¹⁸ c⁵⁹¹⁹ s⁵⁹²⁰ r⁵⁹²¹ e⁵⁹²² t⁵⁹²³ s⁵⁹²⁴ g⁵⁹²⁵ l⁵⁹²⁶ s⁵⁹²⁷ p⁵⁹²⁸
a⁵⁹²⁹ y⁵⁹³⁰ a⁵⁹³¹ p⁵⁹³² t⁵⁹³³ y⁵⁹³⁴ v⁵⁹³⁵ s⁵⁹³⁶ v⁵⁹³⁷ d⁵⁹³⁸ d⁵⁹³⁹ k⁵⁹⁴⁰ y⁵⁹⁴¹ k⁵⁹⁴² t⁵⁹⁴³ s⁵⁹⁴⁴ d⁵⁹⁴⁵ e⁵⁹⁴⁶ l⁵⁹⁴⁷
c⁵⁹⁴⁸ v⁵⁹⁴⁹ n⁵⁹⁵⁰ l⁵⁹⁵¹ n⁵⁹⁵² l⁵⁹⁵³ p⁵⁹⁵⁴ a⁵⁹⁵⁵ n⁵⁹⁵⁶ v⁵⁹⁵⁷ p⁵⁹⁵⁸ y⁵⁹⁵⁹ s⁵⁹⁶⁰ r⁵⁹⁶¹ v⁵⁹⁶² i⁵⁹⁶³ s⁵⁹⁶⁴ r⁵⁹⁶⁵ m⁵⁹⁶⁶
g⁵⁹⁶⁷ f⁵⁹⁶⁸ k⁵⁹⁶⁹ l⁵⁹⁷⁰ d⁵⁹⁷¹ a⁵⁹⁷² t⁵⁹⁷³ v⁵⁹⁷⁴ p⁵⁹⁷⁵ g⁵⁹⁷⁶ y⁵⁹⁷⁷ p⁵⁹⁷⁸ k⁵⁹⁷⁹ l⁵⁹⁸⁰ f⁵⁹⁸¹ i⁵⁹⁸² t⁵⁹⁸³ r⁵⁹⁸⁴ e⁵⁹⁸⁵
e⁵⁹⁸⁶ a⁵⁹⁸⁷ v⁵⁹⁸⁸ r⁵⁹⁸⁹ q⁵⁹⁹⁰ v⁵⁹⁹¹ r⁵⁹⁹² s⁵⁹⁹³ w⁵⁹⁹⁴ i⁵⁹⁹⁵ g⁵⁹⁹⁶ f⁵⁹⁹⁷ d⁵⁹⁹⁸ v⁵⁹⁹⁹ e⁶⁰⁰⁰ g⁶⁰⁰¹ a⁶⁰⁰² h⁶⁰⁰³ a⁶⁰⁰⁴
s⁶⁰⁰⁵ r⁶⁰⁰⁶ n⁶⁰⁰⁷ a⁶⁰⁰⁸ c⁶⁰⁰⁹ g⁶⁰¹⁰ t⁶⁰¹¹ n⁶⁰¹² v⁶⁰¹³ p⁶⁰¹⁴ l⁶⁰¹⁵ q⁶⁰¹⁶ l⁶⁰¹⁷ g⁶⁰¹⁸ f⁶⁰¹⁹ s⁶⁰²⁰ t⁶⁰²¹ g⁶⁰²² v⁶⁰²³
n⁶⁰²⁴ f⁶⁰²⁵ v⁶⁰²⁶ v⁶⁰²⁷ q⁶⁰²⁸ p⁶⁰²⁹ v⁶⁰³⁰ g⁶⁰³¹ v⁶⁰³² v⁶⁰³³ d⁶⁰³⁴ t⁶⁰³⁵ e⁶⁰³⁶ w⁶⁰³⁷ g⁶⁰³⁸ n⁶⁰³⁹ m⁶⁰⁴⁰ l⁶⁰⁴¹ t⁶⁰⁴²
g⁶⁰⁴³ i⁶⁰⁴⁴ a⁶⁰⁴⁵ a⁶⁰⁴⁶ r⁶⁰⁴⁷ p⁶⁰⁴⁸ p⁶⁰⁴⁹ p⁶⁰⁵⁰ g⁶⁰⁵¹ e⁶⁰⁵² q⁶⁰⁵³ f⁶⁰⁵⁴ k⁶⁰⁵⁵ h⁶⁰⁵⁶ l⁶⁰⁵⁷ v⁶⁰⁵⁸ p⁶⁰⁵⁹ l⁶⁰⁶⁰ m⁶⁰⁶¹
h⁶⁰⁶² k⁶⁰⁶³ g⁶⁰⁶⁴ a⁶⁰⁶⁵ a⁶⁰⁶⁶ w⁶⁰⁶⁷ p⁶⁰⁶⁸ i⁶⁰⁶⁹ v⁶⁰⁷⁰ r⁶⁰⁷¹ r⁶⁰⁷² r⁶⁰⁷³ i⁶⁰⁷⁴ v⁶⁰⁷⁵ q⁶⁰⁷⁶ m⁶⁰⁷⁷ l⁶⁰⁷⁸ s⁶⁰⁷⁹ d⁶⁰⁸⁰
t⁶⁰⁸¹ l⁶⁰⁸² d⁶⁰⁸³ k⁶⁰⁸⁴ l⁶⁰⁸⁵ s⁶⁰⁸⁶ d⁶⁰⁸⁷ y⁶⁰⁸⁸ c⁶⁰⁸⁹ t⁶⁰⁹⁰ f⁶⁰⁹¹ v⁶⁰⁹² c⁶⁰⁹³ w⁶⁰⁹⁴ a⁶⁰⁹⁵ h⁶⁰⁹⁶ g⁶⁰⁹⁷ f⁶⁰⁹⁸ e⁶⁰⁹⁹
l⁶¹⁰⁰ t⁶¹⁰¹ s⁶¹⁰² a⁶¹⁰³ s⁶¹⁰⁴ y⁶¹⁰⁵ f⁶¹⁰⁶ c⁶¹⁰⁷ k⁶¹⁰⁸ i⁶¹⁰⁹ g⁶¹¹⁰ k⁶¹¹¹ e⁶¹¹² q⁶¹¹³ k⁶¹¹⁴ c⁶¹¹⁵ c⁶¹¹⁶ m⁶¹¹⁷ c⁶¹¹⁸
n⁶¹¹⁹ r⁶¹²⁰ r⁶¹²¹ a⁶¹²² a⁶¹²³ a⁶¹²⁴ y⁶¹²⁵ s⁶¹²⁶ s⁶¹²⁷ p⁶¹²⁸ l⁶¹²⁹ q⁶¹³⁰ s⁶¹³¹ y⁶¹³² a⁶¹³³ c⁶¹³⁴ w⁶¹³⁵ t⁶¹³⁶ h⁶¹³⁷
s⁶¹³⁸ c⁶¹³⁹ g⁶¹⁴⁰ y⁶¹⁴¹ d⁶¹⁴² y⁶¹⁴³ v⁶¹⁴⁴ y⁶¹⁴⁵ n⁶¹⁴⁶ p⁶¹⁴⁷ f⁶¹⁴⁸ f⁶¹⁴⁹ v⁶¹⁵⁰ d⁶¹⁵¹ v⁶¹⁵² q⁶¹⁵³ q⁶¹⁵⁴ w⁶¹⁵⁵ g⁶¹⁵⁶
y⁶¹⁵⁷ v⁶¹⁵⁸ g⁶¹⁵⁹ n⁶¹⁶⁰ l⁶¹⁶¹ a⁶¹⁶² t⁶¹⁶³ n⁶¹⁶⁴ h⁶¹⁶⁵ d⁶¹⁶⁶ r⁶¹⁶⁷ y⁶¹⁶⁸ c⁶¹⁶⁹ s⁶¹⁷⁰ v⁶¹⁷¹ h⁶¹⁷² q⁶¹⁷³ g⁶¹⁷⁴ a⁶¹⁷⁵
h⁶¹⁷⁶ v⁶¹⁷⁷ a⁶¹⁷⁸ s⁶¹⁷⁹ n⁶¹⁸⁰ d⁶¹⁸¹ a⁶¹⁸² i⁶¹⁸³ m⁶¹⁸⁴ t⁶¹⁸⁵ r⁶¹⁸⁶ c⁶¹⁸⁷ l⁶¹⁸⁸ a⁶¹⁸⁹ i⁶¹⁹⁰ h⁶¹⁹¹ s⁶¹⁹² c⁶¹⁹³ f⁶¹⁹⁴
i⁶¹⁹⁵ e⁶¹⁹⁶ r⁶¹⁹⁷ v⁶¹⁹⁸ d⁶¹⁹⁹ w⁶²⁰⁰ d⁶²⁰¹ i⁶²⁰² e⁶²⁰³ y⁶²⁰⁴ p⁶²⁰⁵ y⁶²⁰⁶ i⁶²⁰⁷ s⁶²⁰⁸ h⁶²⁰⁹ e⁶²¹⁰ k⁶²¹¹ k⁶²¹² l⁶²¹³
n⁶²¹⁴ s⁶²¹⁵ c⁶²¹⁶ c⁶²¹⁷ r⁶²¹⁸ i⁶²¹⁹ v⁶²²⁰ e⁶²²¹ r⁶²²² n⁶²²³ v⁶²²⁴ v⁶²²⁵ r⁶²²⁶ a⁶²²⁷ a⁶²²⁸ l⁶²²⁹ l⁶²³⁰ a⁶²³¹ g⁶²³²
s⁶²³³ f⁶²³⁴ d⁶²³⁵ k⁶²³⁶ v⁶²³⁷ y⁶²³⁸ d⁶²³⁹ i⁶²⁴⁰ g⁶²⁴¹ n⁶²⁴² p⁶²⁴³ k⁶²⁴⁴ g⁶²⁴⁵ i⁶²⁴⁶ p⁶²⁴⁷ i⁶²⁴⁸ v⁶²⁴⁹ d⁶²⁵⁰ d⁶²⁵¹
p⁶²⁵² v⁶²⁵³ v⁶²⁵⁴ d⁶²⁵⁵ w⁶²⁵⁶ h⁶²⁵⁷ y⁶²⁵⁸ f⁶²⁵⁹ d⁶²⁶⁰ a⁶²⁶¹ q⁶²⁶² p⁶²⁶³ l⁶²⁶⁴ t⁶²⁶⁵ r⁶²⁶⁶ k⁶²⁶⁷ v⁶²⁶⁸ q⁶²⁶⁹ q⁶²⁷⁰
l⁶²⁷¹ f⁶²⁷² y⁶²⁷³ t⁶²⁷⁴ e⁶²⁷⁵ d⁶²⁷⁶ m⁶²⁷⁷ a⁶²⁷⁸ s⁶²⁷⁹ r⁶²⁸⁰ f⁶²⁸¹ a⁶²⁸² d⁶²⁸³ g⁶²⁸⁴ l⁶²⁸⁵ c⁶²⁸⁶ l⁶²⁸⁷ f⁶²⁸⁸ w⁶²⁸⁹
n⁶²⁹⁰ c⁶²⁹¹ n⁶²⁹² v⁶²⁹³ p⁶²⁹⁴ k⁶²⁹⁵ y⁶²⁹⁶ p⁶²⁹⁷ n⁶²⁹⁸ n⁶²⁹⁹ a⁶³⁰⁰ i⁶³⁰¹ v⁶³⁰² c⁶³⁰³ r⁶³⁰⁴ f⁶³⁰⁵ d⁶³⁰⁶ t⁶³⁰⁷ r⁶³⁰⁸
v⁶³⁰⁹ h⁶³¹⁰ s⁶³¹¹ e⁶³¹² f⁶³¹³ n⁶³¹⁴ l⁶³¹⁵ p⁶³¹⁶ g⁶³¹⁷ c⁶³¹⁸ d⁶³¹⁹ g⁶³²⁰ g⁶³²¹ s⁶³²² l⁶³²³ y⁶³²⁴ v⁶³²⁵ n⁶³²⁶ k⁶³²⁷
h⁶³²⁸ a⁶³²⁹ f⁶³³⁰ h⁶³³¹ t⁶³³² p⁶³³³ a⁶³³⁴ y⁶³³⁵ d⁶³³⁶ v⁶³³⁷ s⁶³³⁸ a⁶³³⁹ f⁶³⁴⁰ r⁶³⁴¹ d⁶³⁴² l⁶³⁴³ k⁶³⁴⁴ p⁶³⁴⁵ l⁶³⁴⁶
p⁶³⁴⁷ f⁶³⁴⁸ f⁶³⁴⁹ y⁶³⁵⁰ y⁶³⁵¹ s⁶³⁵² t⁶³⁵³ t⁶³⁵⁴ p⁶³⁵⁵ c⁶³⁵⁶ e⁶³⁵⁷ v⁶³⁵⁸ h⁶³⁵⁹ g⁶³⁶⁰ n⁶³⁶¹ g⁶³⁶² s⁶³⁶³ m⁶³⁶⁴ i⁶³⁶⁵
e⁶³⁶⁶ d⁶³⁶⁷ i⁶³⁶⁸ d⁶³⁶⁹ y⁶³⁷⁰ v⁶³⁷¹ p⁶³⁷² l⁶³⁷³ k⁶³⁷⁴ s⁶³⁷⁵ a⁶³⁷⁶ v⁶³⁷⁷ c⁶³⁷⁸ i⁶³⁷⁹ t⁶³⁸⁰ a⁶³⁸¹ c⁶³⁸² n⁶³⁸³ l⁶³⁸⁴
g⁶³⁸⁵ g⁶³⁸⁶ a⁶³⁸⁷ v⁶³⁸⁸ c⁶³⁸⁹ r⁶³⁹⁰ k⁶³⁹¹ h⁶³⁹² a⁶³⁹³ t⁶³⁹⁴ e⁶³⁹⁵ y⁶³⁹⁶ r⁶³⁹⁷ e⁶³⁹⁸ y⁶³⁹⁹ m⁶⁴⁰⁰ e⁶⁴⁰¹ a⁶⁴⁰² y⁶⁴⁰³
n⁶⁴⁰⁴ l⁶⁴⁰⁵ v⁶⁴⁰⁶ s⁶⁴⁰⁷ a⁶⁴⁰⁸ s⁶⁴⁰⁹ g⁶⁴¹⁰ f⁶⁴¹¹ r⁶⁴¹² l⁶⁴¹³ w⁶⁴¹⁴ c⁶⁴¹⁵ y⁶⁴¹⁶ k⁶⁴¹⁷ t⁶⁴¹⁸ f⁶⁴¹⁹ d⁶⁴²⁰ i⁶⁴²¹ y⁶⁴²²
n⁶⁴²³ l⁶⁴²⁴ w⁶⁴²⁵ s⁶⁴²⁶ t⁶⁴²⁷ f⁶⁴²⁸ t⁶⁴²⁹ k⁶⁴³⁰ v⁶⁴³¹ q⁶⁴³² g⁶⁴³³ l⁶⁴³⁴ e⁶⁴³⁵ n⁶⁴³⁶ i⁶⁴³⁷ a⁶⁴³⁸ f⁶⁴³⁹ n⁶⁴⁴⁰ v⁶⁴⁴¹
v⁶⁴⁴² k⁶⁴⁴³ q⁶⁴⁴⁴ g⁶⁴⁴⁵ h⁶⁴⁴⁶ f⁶⁴⁴⁷ i⁶⁴⁴⁸ g⁶⁴⁴⁹ v⁶⁴⁵⁰ e⁶⁴⁵¹ g⁶⁴⁵² e⁶⁴⁵³ l⁶⁴⁵⁴ p⁶⁴⁵⁵ v⁶⁴⁵⁶ a⁶⁴⁵⁷ v⁶⁴⁵⁸ v⁶⁴⁵⁹ n⁶⁴⁶⁰
d⁶⁴⁶¹ k⁶⁴⁶² i⁶⁴⁶³ f⁶⁴⁶⁴ t⁶⁴⁶⁵ k⁶⁴⁶⁶ s⁶⁴⁶⁷ g⁶⁴⁶⁸ v⁶⁴⁶⁹ n⁶⁴⁷⁰ d⁶⁴⁷¹ i⁶⁴⁷² c⁶⁴⁷³ m⁶⁴⁷⁴ f⁶⁴⁷⁵ e⁶⁴⁷⁶ n⁶⁴⁷⁷ k⁶⁴⁷⁸ t⁶⁴⁷⁹
t⁶⁴⁸⁰ l⁶⁴⁸¹ p⁶⁴⁸² t⁶⁴⁸³ n⁶⁴⁸⁴ i⁶⁴⁸⁵ a⁶⁴⁸⁶ f⁶⁴⁸⁷ e⁶⁴⁸⁸ l⁶⁴⁸⁹ y⁶⁴⁹⁰ a⁶⁴⁹¹ k⁶⁴⁹² r⁶⁴⁹³ a⁶⁴⁹⁴ v⁶⁴⁹⁵ r⁶⁴⁹⁶ s⁶⁴⁹⁷ h⁶⁴⁹⁸
p⁶⁴⁹⁹ d⁶⁵⁰⁰ f⁶⁵⁰¹ k⁶⁵⁰² l⁶⁵⁰³ l⁶⁵⁰⁴ h⁶⁵⁰⁵ n⁶⁵⁰⁶ l⁶⁵⁰⁷ q⁶⁵⁰⁸ a⁶⁵⁰⁹ d⁶⁵¹⁰ i⁶⁵¹¹ c⁶⁵¹² y⁶⁵¹³ k⁶⁵¹⁴ f⁶⁵¹⁵ v⁶⁵¹⁶ l⁶⁵¹⁷
w⁶⁵¹⁸ d⁶⁵¹⁹ y⁶⁵²⁰ e⁶⁵²¹ r⁶⁵²² s⁶⁵²³ n⁶⁵²⁴ i⁶⁵²⁵ y⁶⁵²⁶ g⁶⁵²⁷ t⁶⁵²⁸ a⁶⁵²⁹ t⁶⁵³⁰ i⁶⁵³¹ g⁶⁵³² v⁶⁵³³ c⁶⁵³⁴ k⁶⁵³⁵ y⁶⁵³⁶
t⁶⁵³⁷ d⁶⁵³⁸ i⁶⁵³⁹ d⁶⁵⁴⁰ v⁶⁵⁴¹ n⁶⁵⁴² s⁶⁵⁴³ a⁶⁵⁴⁴ l⁶⁵⁴⁵ n⁶⁵⁴⁶ i⁶⁵⁴⁷ c⁶⁵⁴⁸ f⁶⁵⁴⁹ d⁶⁵⁵⁰ i⁶⁵⁵¹ r⁶⁵⁵² d⁶⁵⁵³ n⁶⁵⁵⁴ g⁶⁵⁵⁵
s⁶⁵⁵⁶ l⁶⁵⁵⁷ e⁶⁵⁵⁸ k⁶⁵⁵⁹ f⁶⁵⁶⁰ m⁶⁵⁶¹ s⁶⁵⁶² t⁶⁵⁶³ p⁶⁵⁶⁴ n⁶⁵⁶⁵ a⁶⁵⁶⁶ i⁶⁵⁶⁷ f⁶⁵⁶⁸ i⁶⁵⁶⁹ s⁶⁵⁷⁰ d⁶⁵⁷¹ r⁶⁵⁷² k⁶⁵⁷³ i⁶⁵⁷⁴
k⁶⁵⁷⁵ k⁶⁵⁷⁶ y⁶⁵⁷⁷ p⁶⁵⁷⁸ c⁶⁵⁷⁹ m⁶⁵⁸⁰ v⁶⁵⁸¹ g⁶⁵⁸² p⁶⁵⁸³ d⁶⁵⁸⁴ y⁶⁵⁸⁵ a⁶⁵⁸⁶ y⁶⁵⁸⁷ f⁶⁵⁸⁸ n⁶⁵⁸⁹ g⁶⁵⁹⁰ a⁶⁵⁹¹ i⁶⁵⁹² i⁶⁵⁹³
r⁶⁵⁹⁴ d⁶⁵⁹⁵ s⁶⁵⁹⁶ d⁶⁵⁹⁷ v⁶⁵⁹⁸ v⁶⁵⁹⁹ k⁶⁶⁰⁰ q⁶⁶⁰¹ p⁶⁶⁰² v⁶⁶⁰³ k⁶⁶⁰⁴ f⁶⁶⁰⁵ y⁶⁶⁰⁶ l⁶⁶⁰⁷ y⁶⁶⁰⁸ k⁶⁶⁰⁹ k⁶⁶¹⁰ v⁶⁶¹¹ n⁶⁶¹²
n⁶⁶¹³ e⁶⁶¹⁴ f⁶⁶¹⁵ i⁶⁶¹⁶ d⁶⁶¹⁷ p⁶⁶¹⁸ t⁶⁶¹⁹ e⁶⁶²⁰ c⁶⁶²¹ i⁶⁶²² y⁶⁶²³ t⁶⁶²⁴ q⁶⁶²⁵ s⁶⁶²⁶ r⁶⁶²⁷ s⁶⁶²⁸ c⁶⁶²⁹ s⁶⁶³⁰ d⁶⁶³¹
f⁶⁶³² l⁶⁶³³ p⁶⁶³⁴ l⁶⁶³⁵ s⁶⁶³⁶ d⁶⁶³⁷ m⁶⁶³⁸ e⁶⁶³⁹ k⁶⁶⁴⁰ d⁶⁶⁴¹ f⁶⁶⁴² l⁶⁶⁴³ s⁶⁶⁴⁴ f⁶⁶⁴⁵ d⁶⁶⁴⁶ s⁶⁶⁴⁷ d⁶⁶⁴⁸ v⁶⁶⁴⁹ f⁶⁶⁵⁰
i⁶⁶⁵¹ k⁶⁶⁵² k⁶⁶⁵³ y⁶⁶⁵⁴ g⁶⁶⁵⁵ l⁶⁶⁵⁶ e⁶⁶⁵⁷ n⁶⁶⁵⁸ y⁶⁶⁵⁹ a⁶⁶⁶⁰ f⁶⁶⁶¹ e⁶⁶⁶² h⁶⁶⁶³ v⁶⁶⁶⁴ v⁶⁶⁶⁵ y⁶⁶⁶⁶ g⁶⁶⁶⁷ d⁶⁶⁶⁸ f⁶⁶⁶⁹
s⁶⁶⁷⁰ h⁶⁶⁷¹ t⁶⁶⁷² t⁶⁶⁷³ l⁶⁶⁷⁴ g⁶⁶⁷⁵ g⁶⁶⁷⁶ l⁶⁶⁷⁷ h⁶⁶⁷⁸ l⁶⁶⁷⁹ l⁶⁶⁸⁰ i⁶⁶⁸¹ g⁶⁶⁸² l⁶⁶⁸³ y⁶⁶⁸⁴ k⁶⁶⁸⁵ k⁶⁶⁸⁶ q⁶⁶⁸⁷ q⁶⁶⁸⁸
e⁶⁶⁸⁹ g⁶⁶⁹⁰ h⁶⁶⁹¹ i⁶⁶⁹² i⁶⁶⁹³ m⁶⁶⁹⁴ e⁶⁶⁹⁵ e⁶⁶⁹⁶ m⁶⁶⁹⁷ l⁶⁶⁹⁸ k⁶⁶⁹⁹ g⁶⁷⁰⁰ s⁶⁷⁰¹ s⁶⁷⁰² t⁶⁷⁰³ i⁶⁷⁰⁴ h⁶⁷⁰⁵ n⁶⁷⁰⁶ y⁶⁷⁰⁷
f⁶⁷⁰⁸ i⁶⁷⁰⁹ t⁶⁷¹⁰ e⁶⁷¹¹ t⁶⁷¹² n⁶⁷¹³ t⁶⁷¹⁴ a⁶⁷¹⁵ a⁶⁷¹⁶ f⁶⁷¹⁷ k⁶⁷¹⁸ a⁶⁷¹⁹ e⁶⁷²⁰ c⁶⁷²¹ s⁶⁷²² v⁶⁷²³ i⁶⁷²⁴ d⁶⁷²⁵ l⁶⁷²⁶
k⁶⁷²⁷ l⁶⁷²⁸ d⁶⁷²⁹ d⁶⁷³⁰ f⁶⁷³¹ v⁶⁷³² m⁶⁷³³ i⁶⁷³⁴ l⁶⁷³⁵ k⁶⁷³⁶ s⁶⁷³⁷ q⁶⁷³⁸ d⁶⁷³⁹ l⁶⁷⁴⁰ g⁶⁷⁴¹ v⁶⁷⁴² v⁶⁷⁴³ s⁶⁷⁴⁴ k⁶⁷⁴⁵
v⁶⁷⁴⁶ v⁶⁷⁴⁷ k⁶⁷⁴⁸ v⁶⁷⁴⁹ p⁶⁷⁵⁰ i⁶⁷⁵¹ d⁶⁷⁵² l⁶⁷⁵³ t⁶⁷⁵⁴ m⁶⁷⁵⁵ i⁶⁷⁵⁶ e⁶⁷⁵⁷ f⁶⁷⁵⁸ m⁶⁷⁵⁹ l⁶⁷⁶⁰ w⁶⁷⁶¹ c⁶⁷⁶² k⁶⁷⁶³ d⁶⁷⁶⁴
g⁶⁷⁶⁵ q⁶⁷⁶⁶ v⁶⁷⁶⁷ q⁶⁷⁶⁸ t⁶⁷⁶⁹ f⁶⁷⁷⁰ y⁶⁷⁷¹ p⁶⁷⁷² r⁶⁷⁷³ l⁶⁷⁷⁴ q⁶⁷⁷⁵ a⁶⁷⁷⁶ s⁶⁷⁷⁷ a⁶⁷⁷⁸ d⁶⁷⁷⁹ w⁶⁷⁸⁰ k⁶⁷⁸¹ p⁶⁷⁸² g⁶⁷⁸³
h⁶⁷⁸⁴ a⁶⁷⁸⁵ m⁶⁷⁸⁶ p⁶⁷⁸⁷ s⁶⁷⁸⁸ l⁶⁷⁸⁹ f⁶⁷⁹⁰ k⁶⁷⁹¹ v⁶⁷⁹² q⁶⁷⁹³ n⁶⁷⁹⁴ v⁶⁷⁹⁵ n⁶⁷⁹⁶ l⁶⁷⁹⁷ e⁶⁷⁹⁸ r⁶⁷⁹⁹ c⁶⁸⁰⁰ e⁶⁸⁰¹ l⁶⁸⁰²
a⁶⁸⁰³ n⁶⁸⁰⁴ y⁶⁸⁰⁵ k⁶⁸⁰⁶ q⁶⁸⁰⁷ s⁶⁸⁰⁸ i⁶⁸⁰⁹ p⁶⁸¹⁰ m⁶⁸¹¹ p⁶⁸¹² r⁶⁸¹³ g⁶⁸¹⁴ v⁶⁸¹⁵ h⁶⁸¹⁶ m⁶⁸¹⁷ n⁶⁸¹⁸ i⁶⁸¹⁹ a⁶⁸²⁰ k⁶⁸²¹
y⁶⁸²² m⁶⁸²³ q⁶⁸²⁴ l⁶⁸²⁵ c⁶⁸²⁶ q⁶⁸²⁷ y⁶⁸²⁸ l⁶⁸²⁹ n⁶⁸³⁰ t⁶⁸³¹ c⁶⁸³² t⁶⁸³³ l⁶⁸³⁴ a⁶⁸³⁵ v⁶⁸³⁶ p⁶⁸³⁷ a⁶⁸³⁸ n⁶⁸³⁹ m⁶⁸⁴⁰
r⁶⁸⁴¹ v⁶⁸⁴² i⁶⁸⁴³ h⁶⁸⁴⁴ f⁶⁸⁴⁵ g⁶⁸⁴⁶ a⁶⁸⁴⁷ g⁶⁸⁴⁸ s⁶⁸⁴⁹ d⁶⁸⁵⁰ k⁶⁸⁵¹ g⁶⁸⁵² i⁶⁸⁵³ a⁶⁸⁵⁴ p⁶⁸⁵⁵ g⁶⁸⁵⁶ t⁶⁸⁵⁷ s⁶⁸⁵⁸ v⁶⁸⁵⁹
l⁶⁸⁶⁰ r⁶⁸⁶¹ q⁶⁸⁶² w⁶⁸⁶³ l⁶⁸⁶⁴ p⁶⁸⁶⁵ t⁶⁸⁶⁶ d⁶⁸⁶⁷ a⁶⁸⁶⁸ i⁶⁸⁶⁹ i⁶⁸⁷⁰ i⁶⁸⁷¹ d⁶⁸⁷² n⁶⁸⁷³ d⁶⁸⁷⁴ l⁶⁸⁷⁵ n⁶⁸⁷⁶ e⁶⁸⁷⁷ f⁶⁸⁷⁸
v⁶⁸⁷⁹ s⁶⁸⁸⁰ d⁶⁸⁸¹ a⁶⁸⁸² d⁶⁸⁸³ i⁶⁸⁸⁴ t⁶⁸⁸⁵ l⁶⁸⁸⁶ f⁶⁸⁸⁷ g⁶⁸⁸⁸ d⁶⁸⁸⁹ c⁶⁸⁹⁰ v⁶⁸⁹¹ t⁶⁸⁹² v⁶⁸⁹³ r⁶⁸⁹⁴ v⁶⁸⁹⁵ g⁶⁸⁹⁶ q⁶⁸⁹⁷
q⁶⁸⁹⁸ v⁶⁸⁹⁹ d⁶⁹⁰⁰ l⁶⁹⁰¹ v⁶⁹⁰² i⁶⁹⁰³ s⁶⁹⁰⁴ d⁶⁹⁰⁵ m⁶⁹⁰⁶ y⁶⁹⁰⁷ d⁶⁹⁰⁸ p⁶⁹⁰⁹ t⁶⁹¹⁰ t⁶⁹¹¹ k⁶⁹¹² n⁶⁹¹³ v⁶⁹¹⁴ t⁶⁹¹⁵ g⁶⁹¹⁶
s⁶⁹¹⁷ n⁶⁹¹⁸ e⁶⁹¹⁹ s⁶⁹²⁰ k⁶⁹²¹ a⁶⁹²² l⁶⁹²³ f⁶⁹²⁴ f⁶⁹²⁵ t⁶⁹²⁶ y⁶⁹²⁷ l⁶⁹²⁸ c⁶⁹²⁹ n⁶⁹³⁰ l⁶⁹³¹ i⁶⁹³² n⁶⁹³³ n⁶⁹³⁴ n⁶⁹³⁵
l⁶⁹³⁶ a⁶⁹³⁷ l⁶⁹³⁸ g⁶⁹³⁹ g⁶⁹⁴⁰ s⁶⁹⁴¹ v⁶⁹⁴² a⁶⁹⁴³ i⁶⁹⁴⁴ k⁶⁹⁴⁵ i⁶⁹⁴⁶ t⁶⁹⁴⁷ e⁶⁹⁴⁸ h⁶⁹⁴⁹ s⁶⁹⁵⁰ w⁶⁹⁵¹ s⁶⁹⁵² v⁶⁹⁵³ e⁶⁹⁵⁴
l⁶⁹⁵⁵y⁶⁹⁵⁶ e⁶⁹⁵⁷ l⁶⁹⁵⁸ m⁶⁹⁵⁹ g⁶⁹⁶⁰ k⁶⁹⁶¹ f⁶⁹⁶² a⁶⁹⁶³ w⁶⁹⁶⁴ w⁶⁹⁶⁵ t⁶⁹⁶⁶ v⁶⁹⁶⁷ f⁶⁹⁶⁸ c⁶⁹⁶⁹ t⁶⁹⁷⁰ n⁶⁹⁷¹ a⁶⁹⁷² n⁶⁹⁷³
a⁶⁹⁷⁴ s⁶⁹⁷⁵ s⁶⁹⁷⁶ s⁶⁹⁷⁷ e⁶⁹⁷⁸ g⁶⁹⁷⁹ f⁶⁹⁸⁰ l⁶⁹⁸¹ l⁶⁹⁸² g⁶⁹⁸³ i⁶⁹⁸⁴ n⁶⁹⁸⁵ y⁶⁹⁸⁶ l⁶⁹⁸⁷ g⁶⁹⁸⁸ t⁶⁹⁸⁹ i⁶⁹⁹⁰ k⁶⁹⁹¹ e⁶⁹⁹²
n⁶⁹⁹³ i⁶⁹⁹⁴ d⁶⁹⁹⁵ g⁶⁹⁹⁶ g⁶⁹⁹⁷ a⁶⁹⁹⁸ m⁶⁹⁹⁹ h⁷⁰⁰⁰ a⁷⁰⁰¹ n⁷⁰⁰² y⁷⁰⁰³ i⁷⁰⁰⁴ f⁷⁰⁰⁵ w⁷⁰⁰⁶ r⁷⁰⁰⁷ n⁷⁰⁰⁸ s⁷⁰⁰⁹ t⁷⁰¹⁰ p⁷⁰¹¹
m⁷⁰¹² n⁷⁰¹³ l⁷⁰¹⁴ s⁷⁰¹⁵ t⁷⁰¹⁶ y⁷⁰¹⁷ s⁷⁰¹⁸ l⁷⁰¹⁹ f⁷⁰²⁰ d⁷⁰²¹ l⁷⁰²² s⁷⁰²³ k⁷⁰²⁴ f⁷⁰²⁵ q⁷⁰²⁶ l⁷⁰²⁷ k⁷⁰²⁸ l⁷⁰²⁹ k⁷⁰³⁰
g⁷⁰³¹ t⁷⁰³² p⁷⁰³³ v⁷⁰³⁴ l⁷⁰³⁵ q⁷⁰³⁶ l⁷⁰³⁷ k⁷⁰³⁸ e⁷⁰³⁹ s⁷⁰⁴⁰ q⁷⁰⁴¹ i⁷⁰⁴² n⁷⁰⁴³ e⁷⁰⁴⁴ l⁷⁰⁴⁵ v⁷⁰⁴⁶ i⁷⁰⁴⁷ s⁷⁰⁴⁸ l⁷⁰⁴⁹
l⁷⁰⁵⁰ s⁷⁰⁵¹ q⁷⁰⁵² g⁷⁰⁵³ k⁷⁰⁵⁴ l⁷⁰⁵⁵ l⁷⁰⁵⁶ i⁷⁰⁵⁷ r⁷⁰⁵⁸ d⁷⁰⁵⁹ n⁷⁰⁶⁰ d⁷⁰⁶¹ t⁷⁰⁶² l⁷⁰⁶³ s⁷⁰⁶⁴ v⁷⁰⁶⁵ s⁷⁰⁶⁶ t⁷⁰⁶⁷ d⁷⁰⁶⁸
v⁷⁰⁶⁹ l⁷⁰⁷⁰ v⁷⁰⁷¹ n⁷⁰⁷² t⁷⁰⁷³ y⁷⁰⁷⁴ r⁷⁰⁷⁵ k⁷⁰⁷⁶ l⁷⁰⁷⁷ r⁷⁰⁷⁸
Amino-terminal
(SEQ ID NO: 30)
h²⁵_q²⁶_d²⁷_h²⁸_v²⁹_s³⁰_l³¹_t³²_v³³_p³⁴_l³⁵_c³⁶_g³⁷_s³⁸_g³⁹_n⁴⁰_l⁴¹_v⁴²_e⁴³_k⁴⁴_l⁴⁵_s⁴⁶_p⁴⁷_w⁴⁸_f⁴⁹_m⁵⁰_
d⁵¹_g⁵² e⁵³_n⁵⁴_a⁵⁵_y⁵⁶_e⁵⁷_v⁵⁸_v⁵⁹_k⁶⁰_a⁶¹_m⁶²_l⁶³_l⁶⁴_k⁶⁵_k⁶⁶
(SEQ ID NO: 31)
h²⁸_v²⁹_s³⁰_l³¹_t³²_v³³_p³⁴_l³⁵_c³⁶_g³⁷_s³⁸_g³⁹_n⁴⁰_l⁴¹_v⁴²_e⁴³_k⁴⁴_l⁴⁵_s⁴⁶_p⁴⁷_w⁴⁸_f⁴⁹_m⁵⁰_d⁵¹_g⁵²_e⁵³_
n⁵⁴_a⁵⁵ y⁵⁶_e⁵⁷_v⁵⁸_v⁵⁹_k⁶⁰_a⁶¹_m⁶²_l⁶³_l⁶⁴_l⁶⁵_k⁶⁶
(SEQ ID NO: 15)
k⁶⁰_a⁶¹_m⁶²_l⁶³_l⁶⁴_k⁶⁵_k⁶⁶_e⁶⁷_p⁶⁸_l⁶⁹_l⁷⁰_y⁷¹_v⁷²_p⁷³_i⁷⁴_r⁷⁵_l⁷⁶_a⁷⁷_g⁷⁸_h⁷⁹
(SEQ ID NO: 32)
k⁶⁰_a⁶¹_m⁶²_l⁶³_l⁶⁴_k⁶⁵_k⁶⁶_e⁶⁷_p⁶⁸_l⁶⁹_l⁷⁰_y⁷¹_v⁷²_p⁷³_i⁷⁴_r⁷⁵_l⁷⁶_a⁷⁷_g⁷⁸_h⁷⁹_t⁸⁰_r⁸¹_h⁸²
(SEQ ID NO: 33)
k¹⁴⁰_q¹⁴¹_n¹⁴²_i¹⁴³_l¹⁴⁴_l¹⁴⁵_r¹⁴⁶_k¹⁴⁷_y¹⁴⁸_g¹⁴⁹_r¹⁵⁰_g¹⁵¹_g¹⁵²_y¹⁵³_h¹⁵⁴
(SEQ ID NO: 34)
k¹⁴⁰_q¹⁴¹_n¹⁴²_i¹⁴³_l¹⁴⁴_l¹⁴⁵_r¹⁴⁶_k¹⁴⁷_y¹⁴⁸_g¹⁴⁹_r¹⁵⁰_g¹⁵¹_g¹⁵²_y153_h¹⁵⁴_y¹⁵⁵_t¹⁵⁶_p¹⁵⁷_f¹⁵⁸_h¹⁵⁹
(SEQ ID NO: 35)
h¹⁵⁴_y¹⁵⁵_t¹⁵⁶_p¹⁵⁷_f¹⁵⁸_h¹⁵⁹_y¹⁶⁰_e¹⁶¹_r¹⁶²_d¹⁶³_n¹⁶⁴_t¹⁶⁵_s¹⁶⁶_c¹⁶⁷_p¹⁶⁸_e¹⁶⁹_w¹⁷⁰_m¹⁷¹_d¹⁷²_d¹⁷³_f¹⁷⁴_e¹⁷⁵_
a¹⁷⁶ d¹⁷⁷_p¹⁷⁸_k¹⁷⁹_g¹⁸⁰_k¹⁸¹_y¹⁸²_a¹⁸³_q¹⁸⁴_n¹⁸⁵_l¹⁸⁶_l¹⁸⁷_k¹⁸⁸
(SEQ ID NO: 36)
h¹⁵⁴_y¹⁵⁵_t¹⁵⁶_p¹⁵⁷_f¹⁵⁸_h¹⁵⁹_y¹⁶⁰_e¹⁶¹_r¹⁶²_d¹⁶³_n¹⁶⁴_t¹⁶⁵_s¹⁶⁶_c¹⁶⁷_p¹⁶⁸_e¹⁶⁹_w¹⁷⁰_m¹⁷¹_d¹⁷²_d¹⁷³_f¹⁷⁴_e¹⁷⁵_
a¹⁷⁶ d¹⁷⁷_p¹⁷⁸_k¹⁷⁹_g¹⁸⁰_k¹⁸¹_y¹⁸²_a¹⁸³_q¹⁸⁴_n¹⁸⁵_l¹⁸⁶_l¹⁸⁷_k¹⁸⁸_k¹⁸⁹
(SEQ ID NO: 37)
h¹⁵⁹_y¹⁶⁰_e¹⁶¹_r¹⁶²_d¹⁶³_n¹⁶⁴_t¹⁶⁵_s¹⁶⁶_c¹⁶⁷_p¹⁶⁸_e¹⁶⁹_w¹⁷⁰_m¹⁷¹_d¹⁷²_d¹⁷³_f¹⁷⁴_e¹⁷⁵_a¹⁷⁶_d¹⁷⁷_p¹⁷⁸_k¹⁷⁹_g¹⁸⁰_
k¹⁸¹ y¹⁸²_a¹⁸³_q¹⁸⁴_n¹⁸⁵_l¹⁸⁶_l¹⁸⁷_k¹⁸⁸
(SEQ ID NO: 38)
h¹⁵⁹_y¹⁶⁰_e¹⁶¹_r¹⁶²_d¹⁶³_n¹⁶⁴_t¹⁶⁵_s¹⁶⁶_c¹⁶⁷_p¹⁶⁸_e¹⁶⁹_w¹⁷⁰_m¹⁷¹_d¹⁷²_d¹⁷³_f¹⁷⁴_e¹⁷⁵_a¹⁷⁶_d¹⁷⁷_p¹⁷⁸_k¹⁷⁹_g¹⁸⁰_
k¹⁸¹ y¹⁸²_a¹⁸³_q¹⁸⁴_n¹⁸⁵_l¹⁸⁶_l¹⁸⁷_k¹⁸⁸_k¹⁸⁹
(SEQ ID NO: 39)
h²⁵⁴_v²⁵⁵_e²⁵⁶_r²⁵⁷_k²⁵⁸_d²⁵⁹_v²⁶⁰_p²⁶¹_y²⁶²_p²⁶³_k²⁶⁴_q²⁶⁵_s²⁶⁶_i²⁶⁷_f²⁶⁸_t²⁶⁹_i²⁷⁰_n²⁷¹_s²⁷²_v²⁷³_v²⁷⁴_q²⁷⁵_
k²⁷⁶ d²⁷⁷_g²⁷⁸_v²⁷⁹_e²⁸⁰_n²⁸¹_t²⁸²_p²⁸³_p²⁸⁴_h²⁸⁵_y²⁸⁶_f²⁸⁷_t²⁸⁸_l²⁸⁹_g²⁹⁰_c²⁹¹_k²⁹²_i²⁹³_l²⁹⁴_t²⁹⁵_l²⁹⁶_t²⁹⁷_
p²⁹⁸_r²⁹⁹_n³⁰⁰ k³⁰¹
(SEQ ID NO: 26)
h²⁵⁴_v²⁵⁵_e²⁵⁶_r²⁵⁷_k²⁵⁸_d²⁵⁹_v²⁶⁰_p²⁶¹_y²⁶²_p²⁶³_k²⁶⁴
(SEQ ID NO: 40)
k²⁵⁸_d²⁵⁹_v²⁶⁰_p²⁶¹_y²⁶²_p²⁶³_k²⁶⁴_q²⁶⁵_s²⁶⁶_i²⁶⁷_f²⁶⁸_t²⁶⁹_i²⁷⁰_n²⁷¹_s²⁷²_v²⁷³_v²⁷⁴_q²⁷⁵_k²⁷⁶_d²⁷⁷_g²⁷⁸_v²⁷⁹_
e²⁸⁰ n²⁸¹_t²⁸²_p²⁸³_p²⁸⁴_h²⁸⁵
(SEQ ID NO: 41)
h²⁸⁵_y²⁸⁶_f²⁸⁷_t²⁸⁸_l²⁸⁹_g²⁹⁰_c²⁹¹_k²⁹²_i²⁹³_l²⁹⁴_t²⁹⁵_l²⁹⁶_t²⁹⁷_p²⁹⁸_r²⁹⁹_n³⁰⁰_k³⁰¹
(SEQ ID NO: 42)
h²⁸⁵_y²⁸⁶_f²⁸⁷_t²⁸⁸_l²⁸⁹_g²⁹⁰_c²⁹¹_k²⁹²_i²⁹³_l²⁹⁴_t²⁹⁵_l²⁹⁶_t²⁹⁷_p²⁹⁸_r²⁹⁹_n³⁰⁰_k³⁰¹_w³⁰²_s³⁰³_g³⁰⁴_v³⁰⁵_s³⁰⁶_
d³⁰⁷_l³⁰⁸ s³⁰⁹_l³¹⁰_k³¹¹
(SEQ ID NO: 43)
h²⁸⁵_y²⁸⁶_f²⁸⁷_t²⁸⁸_l²⁸⁹_g²⁹⁰_c²⁹¹_k²⁹²_i²⁹³_l²⁹⁴_t²⁹⁵_l²⁹⁶_t²⁹⁷_p²⁹⁸_r²⁹⁹_n³⁰⁰_k³⁰¹_w³⁰²_s³⁰³_g³⁰⁴_v³⁰⁵_s³⁰⁶_
d³⁰⁷_l³⁰⁸ s³⁰⁹_l³¹⁰_k³¹¹_q³¹²_k³¹³_l³¹⁴_l³¹⁵_y³¹⁶_t³¹⁷_f³¹⁸_y³¹⁹_g³²⁰_k³²¹
(SEQ ID NO: 44)
k²⁹²_i²⁹³_l²⁹⁴_t²⁹⁵_l²⁹⁶_t²⁹⁷_p²⁹⁸_r²⁹⁹_n³⁰⁰_k³⁰¹_w³⁰²_s³⁰³_g³⁰⁴_v³⁰⁵_s³⁰⁶_d³⁰⁷_l³⁰⁸_s³⁰⁹_l³¹⁰_k³¹¹_q³¹²_k³¹³_
l³¹⁴_l³¹⁵ y³¹⁶_t³¹⁷_f³¹⁸_y³¹⁹_g³²⁰_k³²¹_e³²²_s³²³_l³²⁴_e³²⁵_n³²⁶_p³²⁷_t³²⁸_y³²⁹_i³³⁰_y³³¹_h³³²
(SEQ ID NO: 45)
k³⁰¹_w³⁰²_s³⁰³_g³⁰⁴_v³⁰⁵_s³⁰⁶_d³⁰⁷_l³⁰⁸_s³⁰⁹_l³¹⁰_k³¹¹_q³¹²_k³¹³_l³¹⁴_l³¹⁵_y³¹⁶_t³¹⁷_f³¹⁸_y³¹⁹_g³²⁰_k³²¹_e³²²_
s³²³_l³²⁴ e³²⁵_n³²⁶_p³²⁷_t³²⁸_y³²⁹_i³³⁰_y³³¹_h³³²
(SEQ ID NO: 46)
k³¹¹_q³¹²_k³¹³_l³¹⁴_l³¹⁵_y³¹⁶_t³¹⁷_f³¹⁸_y³¹⁹_g³²⁰_k³²¹_e³²²_s³²³_l³²⁴_e³²⁵_n³²⁶_p³²⁷_t³²⁸_y³²⁹_i³³⁰_y³³¹_h³³²
(SEQ ID NO: 47)
k³¹³_l³¹⁴_l³¹⁵_y³¹⁶_t³¹⁷_f³¹⁸_y³¹⁹_g³²⁰_k³²¹_e³²²_s³²³_l³²⁴_e³²⁵_n³²⁶_p³²⁷_t³²⁸_y³²⁹_i³³⁰_y³³¹_h³³²
(SEQ ID NO: 13)
k³⁹⁰_g³⁹¹_d³⁹²_s³⁹³_c³⁹⁴_s³⁹⁵_s³⁹⁶_n³⁹⁷_c³⁹⁸_k³⁹⁹_h⁴⁰⁰
(SEQ ID NO: 48)
h⁴⁸²_s⁴⁸³_l⁴⁸⁴_n⁴⁸⁵_f⁴⁸⁶_v⁴⁸⁷_g⁴⁸⁸_e⁴⁸⁹_f⁴⁹⁰_v⁴⁹¹_v⁴⁹²_n⁴⁹³_d⁴⁹⁴_v⁴⁹⁵_v⁴⁹⁶_l⁴⁹⁷_a⁴⁹⁸_i⁴⁹⁹_l⁵⁰⁰_s⁵⁰¹_g⁵⁰²_t⁵⁰³_
t⁵⁰⁴_t⁵⁰⁵ n⁵⁰⁶_v⁵⁰⁷_d⁵⁰⁸_k⁵⁰⁹_i⁵¹⁰_r⁵¹¹_q⁵¹²_l⁵¹³_l⁵¹⁴_k⁵¹⁵_g⁵¹⁶_v⁵¹⁷_t⁵¹⁸_l⁵¹⁹_d⁵²⁰_k⁵²¹
(SEQ ID NO: 49)
k⁵⁷⁰_k⁵⁷¹_v⁵⁷²_a⁵⁷³_t⁵⁷⁴_i⁵⁷⁵_p⁵⁷⁶_y⁵⁷⁷_k⁵⁷⁸_v⁵⁷⁹_c⁵⁸⁰_n⁵⁸¹_s⁵⁸²_v⁵⁸³_k⁵⁸⁴_d⁵⁸⁵_t⁵⁸⁶_l⁵⁸⁷_t⁵⁸⁸_y⁵⁸⁹_y⁵⁹⁰_a⁵⁹¹_
h⁵⁹²
(SEQ ID NO: 50)
k⁵⁷¹_v⁵⁷²_a⁵⁷³_t⁵⁷⁴_i⁵⁷⁵_p⁵⁷⁶_y⁵⁷⁷_k⁵⁷⁸_v⁵⁷⁹_c⁵⁸⁰_n⁵⁸¹_s⁵⁸²_v⁵⁸³_k⁵⁸⁴_d⁵⁸⁵_t⁵⁸⁶_l⁵⁸⁷_t⁵⁸⁸_y⁵⁸⁹_y⁵⁹⁰_a⁵⁹¹_h⁵⁹²
(SEQ ID NO: 51)
k⁵⁷⁸_v⁵⁷⁹9_c⁵⁸⁰_n⁵⁸¹_s⁵⁸²_v⁵⁸³_k⁵⁸⁴_d⁵⁸⁵_t⁵⁸⁶_l⁵⁸⁷_t⁵⁸⁸_y⁵⁸⁹_y⁵⁹⁰_a⁵⁹¹_h⁵⁹²
(SEQ ID NO: 52)
k⁶⁹⁷_v⁶⁹⁸_s⁶⁹⁹_t⁷⁰⁰_l⁷⁰¹_v⁷⁰²_k⁷⁰³_q⁷⁰⁴_v⁷⁰⁵_l⁷⁰⁶_d⁷⁰⁷_l⁷⁰⁸_l⁷⁰⁹_n⁷¹⁰_k⁷¹¹_g⁷¹²_m⁷¹³_q⁷¹⁴_l⁷¹⁵_l⁷¹⁶_h⁷¹⁷
(SEQ ID NO: 53)
k⁷⁰³_q⁷⁰⁴_v⁷⁰⁵_l⁷⁰⁶_d⁷⁰⁷_l⁷⁰⁸_l⁷⁰⁹_n⁷¹⁰_k⁷¹¹_g⁷¹²_m⁷¹³_q⁷¹⁴_l⁷¹⁵_l⁷¹⁶_h⁷¹⁷
(SEQ ID NO: 27)
k⁷¹¹_g⁷¹²_m⁷¹³_q⁷¹⁴_l⁷¹⁵_l⁷¹⁶_h⁷¹⁷_t⁷¹⁸_k⁷¹⁹
(SEQ ID NO: 54)
h⁸⁰⁶_s⁸⁰⁷_p⁸⁰⁸_d⁸⁰⁹_v⁸¹⁰_i⁸¹¹_v⁸¹²_g⁸¹³_d⁸¹⁴_y⁸¹⁵_v⁸¹⁶_i⁸¹⁷_i⁸¹⁸_s⁸¹⁹_e⁸²⁰_k⁸²¹_l⁸²²_f⁸²³_v⁸²⁴_r⁸²⁵_s⁸²⁶_k⁸²⁷
(SEQ ID NO: 55)
k⁸²¹_l⁸²²_f⁸²³_v⁸²⁴_r⁸²⁵_s⁸²⁶_k⁸²⁷_e⁸²⁸_e⁸²⁹_d⁸³⁰_g⁸³¹_f⁸³²_a⁸³³_f⁸³⁴_y⁸³⁵_p⁸³⁶_a⁸³⁷_c⁸³⁸_t⁸³⁹_n⁸⁴⁰_g⁸⁴¹_h⁸⁴²
(SEQ ID NO: 56)
k⁸²¹_l⁸²²_f⁸²³_v⁸²⁴_r⁸²⁵_s⁸²⁶_k⁸²⁷_e⁸²⁸_e⁸²⁹_d⁸³⁰_g⁸³¹_f⁸³²_a⁸³³_f⁸³⁴_y⁸³⁵_p⁸³⁶_a⁸³⁷_c⁸³⁸_t⁸³⁹_n⁸⁴⁰_g⁸⁴¹_h⁸⁴²_
a⁸⁴³ v⁸⁴⁴_p⁸⁴⁵_t⁸⁴⁶_l⁸⁴⁷_f⁸⁴⁸_r⁸⁴⁹_l⁸⁵⁰_k⁸⁵¹_g⁸⁵²_g⁸⁵³_a⁸⁵⁴_p⁸⁵⁵_v⁸⁵⁶_k⁸⁵⁷_k⁸⁵⁸_v⁸⁵⁹_a⁸⁶⁰_f⁸⁶¹_g⁸⁶²_g⁸⁶³_d⁸⁶⁴_
q⁸⁶⁵_v⁸⁶⁶ h⁸⁶⁷
(SEQ ID NO: 57)
h⁸⁴²_a⁸⁴³_v⁸⁴⁴_p⁸⁴⁵_t⁸⁴⁶_l⁸⁴⁷_f⁸⁴⁸_r⁸⁴⁹_l⁸⁵⁰_k⁸⁵¹_g⁸⁵²_g⁸⁵³_a⁸⁵⁴_p⁸⁵⁵_v⁸⁵⁶_k⁸⁵⁷
(SEQ ID NO: 58)
h⁸⁴²_a⁸⁴³_v⁸⁴⁴_p⁸⁴⁵_t⁸⁴⁶_l⁸⁴⁷_f⁸⁴⁸_r⁸⁴⁹_l⁸⁵⁰_k⁸⁵¹_g⁸⁵²_g⁸⁵³_a⁸⁵⁴_p⁸⁵⁵_v⁸⁵⁶_k⁸⁵⁷_k⁸⁵⁸
(SEQ ID NO: 59)
k⁸⁵¹_g⁸⁵²_g⁸⁵³_a⁸⁵⁴_p⁸⁵⁵_v⁸⁵⁶_k⁸⁵⁷_k⁸⁵⁸_v⁸⁵⁹_a⁸⁶⁰_f⁸⁶¹_g⁸⁶²_g⁸⁶³_d⁸⁶⁴_q⁸⁶⁵_v⁸⁶⁶_h⁸⁶⁷
(SEQ ID NO: 60)
k⁸⁵¹_g⁸⁵²_g⁸⁵³_a⁸⁵⁴_p⁸⁵⁵_v⁸⁵⁶_k⁸⁵⁷_k⁸⁵⁸_v⁸⁵⁹_a⁸⁶⁰_f⁸⁶¹_g⁸⁶²_g⁸⁶³_d⁸⁶⁴_q⁸⁶⁵_v⁸⁶⁶_h⁸⁶⁷_e⁸⁶⁸_v⁸⁶⁹_a⁸⁷⁰_a⁸⁷¹_v⁸⁷²_
r⁸⁷³ s⁸⁷⁴_v⁸⁷⁵_t⁸⁷⁶_v⁸⁷⁷_e⁸⁷⁸_y⁸⁷⁹_n⁸⁸⁰_i⁸⁸¹_h⁸⁸²
(SEQ ID NO: 61)
h⁸⁸²_a⁸⁸³_v⁸⁸⁴_l⁸⁸⁵_d⁸⁸⁶_t⁸⁸⁷_l⁸⁸⁸_l⁸⁸⁹_a⁸⁹⁰_s⁸⁹¹_s⁸⁹²_s⁸⁹³_l⁸⁹⁴_r⁸⁹⁵_t⁸⁹⁶_f⁸⁹⁷_v⁸⁹⁸_v⁸⁹⁹_d⁹⁰⁰_k⁹⁰¹_s⁹⁰²_l⁹⁰³_
s⁹⁰⁴_i⁹⁰⁵ e⁹⁰⁶_e⁹⁰⁷_f⁹⁰⁸_a⁹⁰⁹_d⁹¹⁰_v⁹¹¹_v⁹¹²_k⁹¹³_e⁹¹⁴_q⁹¹⁵_v⁹¹⁶_s⁹¹⁷_d⁹¹⁸_l⁹¹⁹_l⁹²⁰_v⁹²¹_k⁹²²
(SEQ ID NO: 62)
k¹⁰⁹⁸_p¹⁰⁹⁹_k¹¹⁰⁰_r¹¹⁰¹_l¹¹⁰²_r¹¹⁰³_k¹¹⁰⁴_k¹¹⁰⁵_r¹¹⁰⁶_n¹¹⁰⁷_v¹¹⁰⁸_d¹¹⁰⁹_p¹¹¹⁰_l¹¹¹¹_s¹¹¹²_n¹¹¹³_f¹¹¹⁴_e¹¹¹⁵_h¹¹¹⁶
(SEQ ID NO: 63)
h¹¹⁴⁹_l¹¹⁵⁰_k¹¹⁵¹_h¹¹⁵²_g¹¹⁵³_g¹¹⁵⁴_g¹¹⁵⁵_i¹¹⁵⁶_a¹¹⁵⁷_g¹¹⁵⁸_a¹¹⁵⁹_i¹¹⁶⁰_n¹¹⁶¹_a¹¹⁶²_a¹¹⁶³_s¹¹⁶⁴_k¹¹⁶⁵_g¹¹⁶⁶_a¹¹⁶⁷
v¹¹⁶⁸_q¹¹⁶⁹_k¹¹⁷⁰_e¹¹⁷¹_s¹¹⁷²_d¹¹⁷³_e¹¹⁷⁴_y¹¹⁷⁵_i¹¹⁷⁶_l¹¹⁷⁷_a¹¹⁷⁸_k¹¹⁷⁹
(SEQ ID NO: 64)
h¹¹⁵²_g¹¹⁵³_g¹¹⁵⁴_g¹¹⁵⁵_i¹¹⁵⁶_a¹¹⁵⁷_g¹¹⁵⁸_a¹¹⁵⁹_i¹¹⁶⁰_n¹¹⁶¹_a¹¹⁶²_a¹¹⁶³_s¹¹⁶⁴_k¹¹⁶⁵_g¹¹⁶⁶_a¹¹⁶⁷_v¹¹⁶⁸_q¹¹⁶⁹_k¹¹⁷⁰
e¹¹⁷¹_s¹¹⁷²_d¹¹⁷³_e¹¹⁷⁴_y¹¹⁷⁵_i¹¹⁷⁶_l¹¹⁷⁷_a¹¹⁷⁸_k¹¹⁷⁹
(SEQ ID NO: 65)
k¹¹⁷⁰_e¹¹⁷¹_s¹¹⁷²_d¹¹⁷³_e¹¹⁷⁴_y¹¹⁷⁵_i¹¹⁷⁶_l¹¹⁷⁷_a¹¹⁷⁸_k¹¹⁷⁹_g¹¹⁸⁰_p¹¹⁸¹_l¹¹⁸²_q¹¹⁸³_v¹¹⁸⁴_g¹¹⁸⁵_d¹¹⁸⁶_s¹¹⁸⁷_v¹¹⁸⁸_
l¹¹⁸⁹ l¹¹⁹⁰_q¹¹⁹¹_g¹¹⁹²_h¹¹⁹³_s¹¹⁹⁴_l¹¹⁹⁵_a¹¹⁹⁶_k¹¹⁹⁷_n¹¹⁹⁸_i¹¹⁹⁹_l¹²⁰⁰_h¹²⁰¹
(SEQ ID NO: 66)
k¹¹⁷⁰_e¹¹⁷¹_s¹¹⁷²_d¹¹⁷³_e¹¹⁷⁴_y¹¹⁷⁵_i¹¹⁷⁶_l¹¹⁷⁷_a¹¹⁷⁸_k¹¹⁷⁹_g¹¹⁸⁰_p¹¹⁸¹_l¹¹⁸²_q¹¹⁸³_v¹¹⁸⁴_g¹¹⁸⁵_d¹¹⁸⁶_s¹¹⁸⁷_v¹¹⁸⁸_
l¹¹⁸⁹ l¹¹⁹⁰_q¹¹⁹¹_g¹¹⁹²_h¹¹⁹³
(SEQ ID NO: 67)
h¹¹⁹³_s¹¹⁹⁴_l¹¹⁹⁵_a¹¹⁹⁶_k¹¹⁹⁷_n¹¹⁹⁸_i¹¹⁹⁹_l¹²⁰⁰_h¹²⁰¹_v¹²⁰²_v¹²⁰³_g¹²⁰⁴_p¹²⁰⁵_d¹²⁰⁶_a¹²⁰⁷_r¹²⁰⁸_a¹²⁰⁹_k¹²¹⁰_q¹²¹¹
d¹²¹²_v¹²¹³_s¹²¹⁴_l¹²¹⁵_l¹²¹⁶_s¹²¹⁷_k¹²¹⁸
(SEQ ID NO: 68)
h¹²⁰¹_v¹²⁰²_v¹²⁰³_g¹²⁰⁴_p¹²⁰⁵_d¹²⁰⁶_a¹²⁰⁷_r¹²⁰⁸_a¹²⁰⁹_k¹²¹⁰_q¹²¹¹_d¹²¹²_v¹²¹³_s¹²¹⁴_l¹²¹⁵_l¹²¹⁶_s¹²¹⁷_k¹²¹⁸
(SEQ ID NO: 69)
k¹³¹⁷_g¹³¹⁸_v¹³¹⁹_d¹³²⁰_y¹³²¹_t¹³²²_k¹³²³_k¹³²⁴_f¹³²⁵_l¹³²⁶_t¹³²⁷_v¹³²⁸_d¹³²⁹_g¹³³⁰_v¹³³¹_q¹³³²_y¹³³³_y¹³³⁴_c¹³³⁵
y¹³³⁶_t¹³³⁷_s¹³³⁸_k¹³³⁹_d¹³⁴⁰_t¹³⁴¹_l¹³⁴²_d¹³⁴³_d¹³⁴⁴_i¹³⁴⁵_l¹³⁴⁶_q¹³⁴⁷_q¹³⁴⁸_a¹³⁴⁹_n¹³⁵⁰_k¹³⁵¹_s¹³⁵²_v¹³⁵³_g¹³⁵⁴_
i¹³⁵⁵ i¹³⁵⁶_s¹³⁵⁷_m¹³⁵⁸_p¹³⁵⁹_l¹³⁶⁰_g¹³⁶¹_y¹³⁶²_v¹³⁶³_s¹³⁶⁴_h¹³⁶⁵
(SEQ ID NO: 70)
h¹³⁶⁵_g¹³⁶⁶_l¹³⁶⁷_d¹³⁶⁸_l¹³⁶⁹_i¹³⁷⁰_q¹³⁷¹_a¹³⁷²_g¹³⁷³_s¹³⁷⁴_v¹³⁷⁵_v¹³⁷⁶_r¹³⁷⁷_r¹³⁷⁸_v¹³⁷⁹_n¹³⁸⁰_v¹³⁸¹_p¹³⁸²_y¹³⁸³_
v¹³⁸⁴ c¹³⁸⁵_l¹³⁸⁶_l¹³⁸⁷_a¹³⁸⁸_n¹³⁸⁹_k¹³⁹⁰_e¹³⁹¹_q¹³⁹²_e¹³⁹³_a¹³⁹⁴_i¹³⁹⁵_l¹³⁹⁶_m¹³⁹⁷_s¹³⁹⁸_e¹³⁹⁹_d¹⁴⁰⁰_v¹⁴⁰¹_k¹⁴⁰²_
l¹⁴⁰³_n¹⁴⁰⁴ p¹⁴⁰⁵_s¹⁴⁰⁶_e¹⁴⁰⁷_d¹⁴⁰⁸_f¹⁴⁰⁹_i¹⁴¹⁰_k¹⁴¹¹
(SEQ ID NO: 13)
k¹⁴⁰²_l¹⁴⁰³_n¹⁴⁰⁴_p¹⁴⁰⁵_s¹⁴⁰⁶_e¹⁴⁰⁷_d¹⁴⁰⁸_f¹⁴⁰⁹_i¹⁴¹⁰_k¹⁴¹¹_h¹⁴¹²
(SEQ ID NO: 71)
k¹⁴⁰²_l¹⁴⁰³_n¹⁴⁰⁴_p¹⁴⁰⁵_s¹⁴⁰⁶_e¹⁴⁰⁷_d¹⁴⁰⁸_f¹⁴⁰⁹_i¹⁴¹⁰_k¹⁴¹¹_h¹⁴¹²_v¹⁴¹³_r¹⁴¹⁴_t¹⁴¹⁵_n¹⁴¹⁶_g¹⁴¹⁷_g¹⁴¹⁸_y¹⁴¹⁹_n¹⁴²⁰_
s¹⁴²¹ w¹⁴²²_h¹⁴²³
(SEQ ID NO: 72)
k¹⁴⁰²_l¹⁴⁰³_n¹⁴⁰⁴_p¹⁴⁰⁵_s¹⁴⁰⁶_e¹⁴⁰⁷_d¹⁴⁰⁸_f¹⁴⁰⁹_i¹⁴¹⁰_k¹⁴¹¹_h¹⁴¹²_v¹⁴¹³_r¹⁴¹⁴_t¹⁴¹⁵_n¹⁴¹⁶_g¹⁴¹⁷_g¹⁴¹⁸_y¹⁴¹⁹_n¹⁴²⁰_
s¹⁴²¹ w¹⁴²²_h¹⁴²³_l¹⁴²⁴_v¹⁴²⁵_e¹⁴²⁶_g¹⁴²⁷_e¹⁴²⁸_l¹⁴²⁹_l¹⁴³⁰_v¹⁴³¹_q¹⁴³²_d¹⁴³³_l¹⁴³⁴_r¹⁴³⁵_l¹⁴³⁶_n¹⁴³⁷_k¹⁴³⁸_l¹⁴³⁹_
l¹⁴⁴⁰_h¹⁴⁴¹
(SEQ ID NO: 73)
h¹⁴¹²_v¹⁴¹³_r¹⁴¹⁴_t¹⁴¹⁵_n¹⁴¹⁶_g¹⁴¹⁷_g¹⁴¹⁸_y¹⁴¹⁹_n¹⁴²⁰_s¹⁴²¹_w¹⁴²²_h¹⁴²³_l¹⁴²⁴_v¹⁴²⁵_e¹⁴²⁶_g¹⁴²⁷_e¹⁴²⁸_l¹⁴²⁹_l¹⁴³⁰_
v¹⁴³¹ q¹⁴³²_d¹⁴³³_l¹⁴³⁴_r¹⁴³⁵_l¹⁴³⁶_n¹⁴³⁷_k¹⁴³⁸_l¹⁴³⁹_l¹⁴⁴⁰_h¹⁴⁴¹_w¹⁴⁴²_s¹⁴⁴³_d¹⁴⁴⁴_q¹⁴⁴⁵_t¹⁴⁴⁶_i¹⁴⁴⁷_c¹⁴⁴⁸_y¹⁴⁴⁹_
k¹⁴⁵⁰_d¹⁴⁵¹ s¹⁴⁵²_v¹⁴⁵³_f¹⁴⁵⁴_y¹⁴⁵⁵_v¹⁴⁵⁶_v¹⁴⁵⁷_k¹⁴⁵⁸
(SEQ ID NO: 74)
h¹⁴²³_l¹⁴²⁴_v¹⁴²⁵_e¹⁴²⁶_g¹⁴²⁷_e¹⁴²⁸_l¹⁴²⁹_l¹⁴³⁰_v¹⁴³¹_q¹⁴³²_d¹⁴³³_l¹⁴³⁴_r¹⁴³⁵_l¹⁴³⁶_n¹⁴³⁷_k¹⁴³⁸_l¹⁴³⁹_l¹⁴⁴⁰_h¹⁴⁴¹_
w¹⁴⁴² s¹⁴⁴³_d¹⁴⁴⁴_q¹⁴⁴⁵_t¹⁴⁴⁶_i¹⁴⁴⁷_c¹⁴⁴⁸_y¹⁴⁴⁹_k¹⁴⁵⁰_d¹⁴⁵¹_s¹⁴⁵²_v¹⁴⁵³_f¹⁴⁵⁴_y¹⁴⁵⁵_v¹⁴⁵⁶_v¹⁴⁵⁷_k¹⁴⁵⁸
(SEQ ID NO: 75)
h¹⁴⁴¹_w¹⁴⁴²_s¹⁴⁴³_d¹⁴⁴⁴_q¹⁴⁴⁵_t¹⁴⁴⁶_i¹⁴⁴⁷_c¹⁴⁴⁸_y¹⁴⁴⁹_k¹⁴⁵⁰_d¹⁴⁵¹_s¹⁴⁵²_v¹⁴⁵³_f¹⁴⁵⁴_y¹⁴⁵⁵_v¹⁴⁵⁶_v¹⁴⁵⁷_k¹⁴⁵⁸
(SEQ ID NO: 76)
h¹⁵³³_s¹⁵³⁴_l¹⁵³⁵_y¹⁵³⁶_l¹⁵³⁷_a¹⁵³⁸_d¹⁵³⁹_n¹⁵⁴⁰_l¹⁵⁴¹_t¹⁵⁴²_a¹⁵⁴³_d¹⁵⁴⁴_e¹⁵⁴⁵_t¹⁵⁴⁶_k¹⁵⁴⁷_a¹⁵⁴⁸_l¹⁵⁴⁹_k¹⁵⁵⁰_e¹⁵⁵¹_
l¹⁵⁵² y¹⁵⁵³_g¹⁵⁵⁴_p¹⁵⁵⁵_v¹⁵⁵⁶_d¹⁵⁵⁷_p¹⁵⁵⁸_t¹⁵⁵⁹_f¹⁵⁶⁰_l¹⁵⁶¹_h¹⁵⁶²_r¹⁵⁶³_f¹⁵⁶⁴_y¹⁵⁶⁵_s¹⁵⁶⁶_l¹⁵⁶⁷_k¹⁵⁶⁸_a¹⁵⁶⁹_a¹⁵⁷⁰_
v¹⁵⁷¹_h¹⁵⁷² k¹⁵⁷³_w¹⁵⁷⁴_k¹⁵⁷⁵
(SEQ ID NO: 77)
h¹⁵³³_s¹⁵³⁴_l¹⁵³⁵_y¹⁵³⁶_l¹⁵³⁷_a¹⁵³⁸_d¹⁵³⁹_n¹⁵⁴⁰_l¹⁵⁴¹_t¹⁵⁴²_a¹⁵⁴³_d¹⁵⁴⁴_e¹⁵⁴⁵_t¹⁵⁴⁶_k¹⁵⁴⁷_a¹⁵⁴⁸_l¹⁵⁴⁹_k¹⁵⁵⁰_e¹⁵⁵¹_
l¹⁵⁵² y¹⁵⁵³_g¹⁵⁵⁴_p¹⁵⁵⁵_v¹⁵⁵⁶_d¹⁵⁵⁷_p¹⁵⁵⁸_t¹⁵⁵⁹_f¹⁵⁶⁰_l¹⁵⁶¹_h¹⁵⁶²_r¹⁵⁶³_f¹⁵⁶⁴_y¹⁵⁶⁵_s¹⁵⁶⁶_l¹⁵⁶⁷_k¹⁵⁶⁸_a¹⁵⁶⁹_a¹⁵⁷⁰_
v¹⁵⁷¹_h¹⁵⁷² k¹⁵⁷³_w¹⁵⁷⁴_k¹⁵⁷⁵_m¹⁵⁷⁶_v¹⁵⁷⁷_v¹⁵⁷⁸_c¹⁵⁷⁹_d¹⁵⁸⁰_k¹⁵⁸¹
(SEQ ID NO: 78)
h¹⁵⁶²_r¹⁵⁶³_f¹⁵⁶⁴_y¹⁵⁶⁵_s¹⁵⁶⁶_l¹⁵⁶⁷_k¹⁵⁶⁸_a¹⁵⁶⁹_a¹⁵⁷⁰_v¹⁵⁷¹_h¹⁵⁷²_k¹⁵⁷³_w¹⁵⁷⁴_k¹⁵⁷⁵_m¹⁵⁷⁶_v¹⁵⁷⁷_v¹⁵⁷⁸_c¹⁵⁷⁹_d¹⁵⁸⁰
k1581
(SEQ ID NO: 79)
h¹⁵⁶²_r¹⁵⁶³_f¹⁵⁶⁴_y¹⁵⁶⁵_s¹⁵⁶⁶_l¹⁵⁶⁷_k¹⁵⁶⁸_a¹⁵⁶⁹_a¹⁵⁷⁰_v¹⁵⁷¹_h¹⁵⁷²_k¹⁵⁷³_w¹⁵⁷⁴_k¹⁵⁷⁵
(SEQ ID NO: 80)
k¹⁵⁶⁸_a¹⁵⁶⁹_a¹⁵⁷⁰_v¹⁵⁷¹_h¹⁵⁷²_k¹⁵⁷³_w¹⁵⁷⁴_k¹⁵⁷⁵_m¹⁵⁷⁶_v¹⁵⁷⁷_v¹⁵⁷⁸_c¹⁵⁷⁹_d¹⁵⁸⁰_k¹⁵⁸¹_v¹⁵⁸²_r¹⁵⁸³_s¹⁵⁸⁴_l¹⁵⁸⁵_k¹⁵⁸⁶
l¹⁵⁸⁷_s¹⁵⁸⁸_d¹⁵⁸⁹_n¹⁵⁹⁰_n¹⁵⁹¹_c¹⁵⁹²_y¹⁵⁹³_l¹⁵⁹⁴_n¹⁵⁹⁵_a¹⁵⁹⁶_v¹⁵⁹⁷_i¹⁵⁹⁸_m¹⁵⁹⁹_t¹⁶⁰⁰_l¹⁶⁰¹_d¹⁶⁰²_l¹⁶⁰³_l¹⁶⁰⁴_k¹⁶⁰⁵_
d¹⁶⁰⁶ i¹⁶⁰⁷_k¹⁶⁰⁸_f¹⁶⁰⁹_v¹⁶¹⁰_i¹⁶¹¹_p¹⁶¹²_a¹⁶¹³_l¹⁶¹⁴_q¹⁶¹⁵_h¹⁶¹⁶
(SEQ ID NO: 18)
k¹⁵⁶⁸_a¹⁵⁶⁹_a¹⁵⁷⁰_v¹⁵⁷¹_h¹⁵⁷²_k¹⁵⁷³_w¹⁵⁷⁴_k¹⁵⁷⁵
(SEQ ID NO: 7)
h¹⁵⁷²_k¹⁵⁷³_w¹⁵⁷⁴_k¹⁵⁷⁵_m¹⁵⁷⁶_v¹⁵⁷⁷_v¹⁵⁷⁸_c¹⁵⁷⁹_d¹⁵⁸⁰_k¹⁵⁸¹
(SEQ ID NO: 81)
k¹⁵⁷³_w¹⁵⁷⁴_k¹⁵⁷⁵_m¹⁵⁷⁶_v¹⁵⁷⁷_v¹⁵⁷⁸_c¹⁵⁷⁹_d¹⁵⁸⁰_k¹⁵⁸¹_v¹⁵⁸²_r¹⁵⁸³_s¹⁵⁸⁴_l¹⁵⁸⁵_k¹⁵⁸⁶_l¹⁵⁸⁷_s¹⁵⁸⁸_d¹⁵⁸⁹_n¹⁵⁹⁰_n¹⁵⁹¹
c¹⁵⁹²_y¹⁵⁹³_l¹⁵⁹⁴_n¹⁵⁹⁵_a¹⁵⁹⁶_v¹⁵⁹⁷_i¹⁵⁹⁸_m¹⁵⁹⁹_t¹⁶⁰⁰_l¹⁶⁰¹_d¹⁶⁰²_l¹⁶⁰³_l¹⁶⁰⁴_k¹⁶⁰⁵_d¹⁶⁰⁶_i¹⁶⁰⁷_k¹⁶⁰⁸_f¹⁶⁰⁹_v¹⁶¹⁰_
i¹⁶¹¹ p¹⁶¹²_a¹⁶¹³_l¹⁶¹⁴_q¹⁶¹⁵_h¹⁶¹⁶
(SEQ ID NO: 82)
k¹⁵⁷³_w¹⁵⁷⁴_k¹⁵⁷⁵_m¹⁵⁷⁶_v¹⁵⁷⁷_v¹⁵⁷⁸_c¹⁵⁷⁹_d¹⁵⁸⁰_k¹⁵⁸¹_v¹⁵⁸²_r¹⁵⁸³_s¹⁵⁸⁴_l¹⁵⁸⁵_k¹⁵⁸⁶_l¹⁵⁸⁷_s¹⁵⁸⁸_d¹⁵⁸⁹_n¹⁵⁹⁰_n¹⁵⁹¹
c¹⁵⁹²_y¹⁵⁹³_l¹⁵⁹⁴_n¹⁵⁹⁵_a¹⁵⁹⁶_v¹⁵⁹⁷_i¹⁵⁹⁸_m¹⁵⁹⁹_t¹⁶⁰⁰_l¹⁶⁰¹_d¹⁶⁰²_l¹⁶⁰³_l¹⁶⁰⁴_k¹⁶⁰⁵_d¹⁶⁰⁶_i¹⁶⁰⁷_k¹⁶⁰⁸_f¹⁶⁰⁹_v¹⁶¹⁰_
i¹⁶¹¹ p¹⁶¹²_a¹⁶¹³_l¹⁶¹⁴_q¹⁶¹⁵_h¹⁶¹⁶_a¹⁶¹⁷_f¹⁶¹⁸_m¹⁶¹⁹_k¹⁶²⁰_h¹⁶²¹
(SEQ ID NO: 83)
k¹⁵⁷⁵_m¹⁵⁷⁶_v¹⁵⁷⁷_v¹⁵⁷⁸_c¹⁵⁷⁹_d¹⁵⁸⁰_k¹⁵⁸¹_v¹⁵⁸²_r¹⁵⁸³_s¹⁵⁸⁴_l¹⁵⁸⁵_k¹⁵⁸⁶_l¹⁵⁸⁷_s¹⁵⁸⁸_d¹⁵⁸⁹_n¹⁵⁹⁰_n¹⁵⁹¹_c¹⁵⁹²_y¹⁵⁹³
l¹⁵⁹⁴_n¹⁵⁹⁵_a¹⁵⁹⁶_v¹⁵⁹⁷_i¹⁵⁹⁸_m¹⁵⁹⁹_t¹⁶⁰⁰_l¹⁶⁰¹_d¹⁶⁰²_l¹⁶⁰³_l¹⁶⁰⁴_k¹⁶⁰⁵_d¹⁶⁰⁶_i¹⁶⁰⁷_k¹⁶⁰⁸_f¹⁶⁰⁹_v¹⁶¹⁰_i¹⁶¹¹_p¹⁶¹²_
a¹⁶¹³ l¹⁶¹⁴_q¹⁶¹⁵_h¹⁶¹⁶
(SEQ ID NO: 84)
k¹⁵⁷⁵_m¹⁵⁷⁶_v¹⁵⁷⁷_v¹⁵⁷⁸_c¹⁵⁷⁹_d¹⁵⁸⁰_k¹⁵⁸¹_v¹⁵⁸²_r¹⁵⁸³_s¹⁵⁸⁴_l¹⁵⁸⁵_k¹⁵⁸⁶_l¹⁵⁸⁷_s¹⁵⁸⁸_d¹⁵⁸⁹_n¹⁵⁹⁰_n¹⁵⁹¹_c¹⁵⁹²_y¹⁵⁹³
l¹⁵⁹⁴_n¹⁵⁹⁵_a¹⁵⁹⁶_v¹⁵⁹⁷_i¹⁵⁹⁸_m¹⁵⁹⁹_t¹⁶⁰⁰_l¹⁶⁰¹_d¹⁶⁰²_l¹⁶⁰³_l¹⁶⁰⁴_k¹⁶⁰⁵_d¹⁶⁰⁶_i¹⁶⁰⁷_k¹⁶⁰⁸_f¹⁶⁰⁹_v¹⁶¹⁰_i¹⁶¹¹_p¹⁶¹²_
a¹⁶¹³ l¹⁶¹⁴_q¹⁶¹⁵_h¹⁶¹⁶_a¹⁶¹⁷_f¹⁶¹⁸_m¹⁶¹⁹_k¹⁶²⁰_h¹⁶²¹
(SEQ ID NO: 85)
h¹⁶⁵²_t¹⁶⁵³_v¹⁶⁵⁴_l¹⁶⁵⁵_a¹⁶⁵⁶_k¹⁶⁵⁷_a¹⁶⁵⁸_e¹⁶⁵⁹_l¹⁶⁶⁰_c¹⁶⁶¹_c¹⁶⁶²_s¹⁶⁶³_a¹⁶⁶⁴_r¹⁶⁶⁵_m¹⁶⁶⁶_v¹⁶⁶⁷_w¹⁶⁶⁸_r¹⁶⁶⁹_e¹⁶⁷⁰
w¹⁶⁷¹_c¹⁶⁷²_n¹⁶⁷³_v¹⁶⁷⁴_c¹⁶⁷⁵_g¹⁶⁷⁶_i¹⁶⁷⁷_k¹⁶⁷⁸_d¹⁶⁷⁹_v¹⁶⁸⁰_v¹⁶⁸¹_l¹⁶⁸²_q¹⁶⁸³_g¹⁶⁸⁴_l¹⁶⁸⁵_k¹⁶⁸⁶
(SEQ ID NO: 86)
h¹⁷⁵⁹_y¹⁷⁶⁰_v¹⁷⁶¹_h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²_f¹⁷⁷³_d¹⁷⁷⁴_s¹⁷⁷⁵_g¹⁷⁷⁶_t¹⁷⁷⁷_
v¹⁷⁷⁸ s¹⁷⁷⁹_k¹⁷⁸⁰
(SEQ ID NO: 87)
h¹⁷⁵⁹_y¹⁷⁶⁰_v¹⁷⁶¹_h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²_f¹⁷⁷³_d¹⁷⁷⁴_s¹⁷⁷⁵_g¹⁷⁷⁶_t¹⁷⁷⁷_
v¹⁷⁷⁸ s¹⁷⁷⁹_k¹⁷⁸⁰_t¹⁷⁸¹_s¹⁷⁸²_d¹⁷⁸³_w¹⁷⁸⁴_k¹⁷⁸⁵_c¹⁷⁸⁶_k¹⁷⁸⁷
(SEQ ID NO: 88)
h¹⁷⁵⁹_y¹⁷⁶⁰_v¹⁷⁶¹_h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²_f¹⁷⁷³_d¹⁷⁷⁴_s¹⁷⁷⁵_g¹⁷⁷⁶_t¹⁷⁷⁷_
v¹⁷⁷⁸ s¹⁷⁷⁹_k¹⁷⁸⁰_t¹⁷⁸¹_s¹⁷⁸²_d¹⁷⁸³_w¹⁷⁸⁴_k¹⁷⁸⁵_c¹⁷⁸⁶_k¹⁷⁸⁷_v¹⁷⁸⁸_d¹⁷⁹⁰_v¹⁷⁹¹_l¹⁷⁹²_f¹⁷⁹³_s¹⁷⁹⁴_g¹⁷⁹⁵_q¹⁷⁹⁶_k¹⁷⁹⁷
(SEQ ID NO: 89)
h¹⁷⁵⁹_y¹⁷⁶⁰_v¹⁷⁶¹_h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²
(SEQ ID NO: 14)
h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²
(SEQ ID NO: 90)
h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²_f¹⁷⁷³_d¹⁷⁷⁴_s¹⁷⁷⁵_g¹⁷⁷⁶_t¹⁷⁷⁷_v¹⁷⁷⁸_s¹⁷⁷⁹_k¹⁷⁸⁰
(SEQ ID NO: 91)
h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²_f¹⁷⁷³_d¹⁷⁷⁴_s¹⁷⁷⁵_g¹⁷⁷⁶_t¹⁷⁷⁷_v¹⁷⁷⁸_s¹⁷⁷⁹_k¹⁷⁸⁰_
t¹⁷⁸¹ s¹⁷⁸²_d¹⁷⁸³_w¹⁷⁸⁴_k¹⁷⁸⁵_c¹⁷⁸⁶_k¹⁷⁸⁷
(SEQ ID NO: 92)
h¹⁷⁶²_a¹⁷⁶³_r¹⁷⁶⁴_l¹⁷⁶⁵_k¹⁷⁶⁶_g¹⁷⁶⁷_g¹⁷⁶⁸_l¹⁷⁶⁹_i¹⁷⁷⁰_l¹⁷⁷¹_k¹⁷⁷²_f¹⁷⁷³_d¹⁷⁷⁴_s¹⁷⁷⁵_g¹⁷⁷⁶_t¹⁷⁷⁷_v¹⁷⁷⁸_s¹⁷⁷⁹_k¹⁷⁸⁰_
t¹⁷⁸¹ s¹⁷⁸²_d¹⁷⁸³_w¹⁷⁸⁴_k¹⁷⁸⁵_c¹⁷⁸⁶_k¹⁷⁸⁷_v¹⁷⁸⁸_t¹⁷⁸⁹_d¹⁷⁹⁰_v¹⁷⁹¹_l¹⁷⁹²_f¹⁷⁹³_s¹⁷⁹⁴_g¹⁷⁹⁵_q¹⁷⁹⁶_k¹⁷⁹⁷
(SEQ ID NO: 93)
k¹⁹⁸⁶_c¹⁹⁸⁷_k¹⁹⁸⁸_g¹⁹⁸⁹_l¹⁹⁹⁰_n¹⁹⁹¹_k¹⁹⁹²_p¹⁹⁹³_f¹⁹⁹⁴_v¹⁹⁹⁵_k¹⁹⁹⁶_d¹⁹⁹⁷_n¹⁹⁹⁸_v¹⁹⁹⁹_s²⁰⁰⁰_f²⁰⁰¹_v²⁰⁰²_a²⁰⁰³_d²⁰⁰⁴
d²⁰⁰⁵_s²⁰⁰⁶_g²⁰⁰⁷_t²⁰⁰⁸_p²⁰⁰⁹_v²⁰¹⁰_v²⁰¹¹_e²⁰¹²_y²⁰¹³_l²⁰¹⁴_s²⁰¹⁵_k²⁰¹⁶_e²⁰¹⁷_d²⁰¹⁸_l²⁰¹⁹_h²⁰²⁰
(SEQ ID NO: 94)
k¹⁹⁸⁸_g¹⁹⁸⁹_l¹⁹⁹⁰_n¹⁹⁹¹_k¹⁹⁹²_p¹⁹⁹³_f¹⁹⁹⁴_v¹⁹⁹⁵_k¹⁹⁹⁶_d¹⁹⁹⁷_n¹⁹⁹⁸_v¹⁹⁹⁹_s²⁰⁰⁰_f²⁰⁰¹_v²⁰⁰²_a²⁰⁰³_d²⁰⁰⁴_d²⁰⁰⁵_s²⁰⁰⁶
g²⁰⁰⁷_t²⁰⁰⁸_p²⁰⁰⁹_v²⁰¹⁰_v²⁰¹¹_e²⁰¹²_y²⁰¹³_l²⁰¹⁴_s²⁰¹⁵_k²⁰¹⁶_e²⁰¹⁷_d²⁰¹⁸_l²⁰¹⁹_h²⁰²⁰
(SEQ ID NO: 95)
h²⁰²⁰_t²⁰²¹_l²⁰²²_y²⁰²³_v²⁰²⁴_d²⁰²⁵_p²⁰²⁶_k²⁰²⁷_y²⁰²⁸_q²⁰²⁹_v²⁰³⁰_i²⁰³¹_v²⁰³²_l²⁰³³_k²⁰³⁴
(SEQ ID NO: 96)
k²⁰²⁷_y²⁰²⁸_q²⁰²⁹_v²⁰³⁰_i²⁰³¹_v²⁰³²_l²⁰³³_k²⁰³⁴_d²⁰³⁵_n²⁰³⁶_v²⁰³⁷_l²⁰³⁸_s²⁰³⁹_s²⁰⁴⁰_m²⁰⁴¹_l²⁰⁴²_r²⁰⁴³_l²⁰⁴⁴_h²⁰⁴⁵
(SEQ ID NO: 97)
h²⁰⁴⁵_t²⁰⁴⁶_v²⁰⁴⁷_e²⁰⁴⁸_s²⁰⁴⁹_g²⁰⁵⁰_d²⁰⁵¹_i²⁰⁵²_n²⁰⁵³_v²⁰⁵⁴_v²⁰⁵⁵_a²⁰⁵⁶_a²⁰⁵⁷_s²⁰⁵⁸_g²⁰⁵⁹_s²⁰⁶⁰_l²⁰⁶¹_t²⁰⁶²_r²⁰⁶³_
k²⁰⁶⁴ v²⁰⁶⁵_k²⁰⁶⁶_l²⁰⁶⁷_l²⁰⁶⁸_f²⁰⁶⁹_r²⁰⁷⁰_a²⁰⁷¹_s²⁰⁷²_f²⁰⁷³_y²⁰⁷⁴_f²⁰⁷⁵_k²⁰⁷⁶
(SEQ ID NO: 98)
k²⁰⁶⁶_l²⁰⁶⁷_l²⁰⁶⁸_f²⁰⁶⁹_r²⁰⁷⁰_a²⁰⁷¹_s²⁰⁷²_f²⁰⁷³_y²⁰⁷⁴_f²⁰⁷⁵_k²⁰⁷⁶_e²⁰⁷⁷_f²⁰⁷⁸_a²⁰⁷⁹_t²⁰⁸⁰_r²⁰⁸¹_t²⁰⁸²_f²⁰⁸³_t²⁰⁸⁴_
a²⁰⁸⁵ t²⁰⁸⁶_t²⁰⁸⁷_a²⁰⁸⁸_v²⁰⁸⁹_g²⁰⁹⁰_s²⁰⁹¹_c²⁰⁹²_i²⁰⁹³_k²⁰⁹⁴_s²⁰⁹⁵_v²⁰⁹⁶_v²⁰⁹⁷_r²⁰⁹⁸_h²⁰⁹⁹
(SEQ ID NO: 10)
k²⁰⁹⁴_s²⁰⁹⁵_v²⁰⁹⁶_v²⁰⁹⁷_r²⁰⁹⁸_h²⁰⁹⁹_l²¹⁰⁰_g²¹⁰¹_v²¹⁰²_t²¹⁰³_k²¹⁰⁴
(SEQ ID NO: 99)
h²⁰⁹⁹_l²¹⁰⁰_g²¹⁰¹_v²¹⁰²_t²¹⁰³_k²¹⁰⁴_g²¹⁰⁵_i²¹⁰⁶_l²¹⁰⁷_t²¹⁰⁸_g²¹⁰⁹_c²¹¹⁰_f²¹¹¹_s²¹¹²_f²¹¹³_v²¹¹⁴_k²¹¹⁵_m²¹¹⁶_l²¹¹⁷_
f²¹¹⁸ m²¹¹⁹_l²¹²⁰_p²¹²¹_l²¹²²_a²¹²³_y²¹²⁴_f²¹²⁵_s²¹²⁶_d²¹²⁷_s²¹²⁸_k²¹²⁹_l²¹³⁰_g²¹³¹_t²¹³²_t²¹³³_e²¹³⁴_v²¹³⁵_k²¹³⁶
(SEQ ID NO: 100)
k²¹⁶⁵_l²¹⁶⁶_r²¹⁶⁷_r²¹⁶⁸_v²¹⁶⁹_d²¹⁷⁰_w²¹⁷¹_k²¹⁷²_s²¹⁷³_t²¹⁷⁴_l²¹⁷⁵_r²¹⁷⁶_l²¹⁷⁷_l²¹⁷⁸_l²¹⁷⁹_m²¹⁸⁰_l²¹⁸¹_c²¹⁸²_t²¹⁸³_
t²¹⁸⁴ m²¹⁸⁵_v²¹⁸⁶_l²¹⁸⁷_l²¹⁸⁸_s²¹⁸⁹_s²¹⁹⁰_v²¹⁹¹_y²¹⁹²_h²¹⁹³
(SEQ ID NO: 101)
h²³⁴³_i²³⁴⁴_p²³⁴⁵_m²³⁴⁶_a²³⁴⁷_g²³⁴⁸_l²³⁴⁹_v²³⁵⁰_r²³⁵¹_m²³⁵²_y²³⁵³_n²³⁵⁴_l²³⁵⁵_l²³⁵⁶_a²³⁵⁷_c²³⁵⁸_l²³⁵⁹_w²³⁶⁰_l²³⁶¹_
t²³⁶² r²³⁶³_k²³⁶⁴_f²³⁶⁵_y²³⁶⁶_q²³⁶⁷_h²³⁶⁸_v²³⁶⁹_i²³⁷⁰_n²³⁷¹_g²³⁷²_c²³⁷³_k²³⁷⁴
(SEQ ID NO: 102)
h²³⁴³_i²³⁴⁴_p²³⁴⁵_m²³⁴⁶_a²³⁴⁷_g²³⁴⁸_l²³⁴⁹_v²³⁵⁰_r²³⁵¹_m²³⁵²_y²³⁵³_n²³⁵⁴_l²³⁵⁵_l²³⁵⁶_a²³⁵⁷_c²³⁵⁸_l²³⁵⁹_w²³⁶⁰_l²³⁶¹_
t²³⁶² r²³⁶³_k²³⁶⁴_f²³⁶⁵_y²³⁶⁶_q²³⁶⁷_h²³⁶⁸_v²³⁶⁹_i²³⁷⁰_n²³⁷¹_g²³⁷²_c²³⁷³_k²³⁷⁴_d²³⁷⁵_t²³⁷⁶_a²³⁷⁷_c²³⁷⁸_l²³⁷⁹_l²³⁸⁰_
c²³⁸¹_y²³⁸² k²³⁸³
Mid-molecule
(SEQ ID NO: 11)
k²³⁶⁴_f²³⁶⁵_y²³⁶⁶_q²³⁶⁷_h²³⁶⁸_v²³⁶⁹_i²³⁷⁰_n²³⁷¹_g²³⁷²_c²³⁷³_k²³⁷⁴
(SEQ ID NO: 103)
h²³⁶⁸_v²³⁶⁹_i²³⁷⁰_n²³⁷¹_g²³⁷²_c²³⁷³_k²³⁷⁴_d²³⁷⁵_t²³⁷⁶_a²³⁷⁷_c²³⁷⁸_l²³⁷⁹_l²³⁸⁰_c²³⁸¹_y²³⁸²_k²³⁸³
(SEQ ID NO: 104)
k²³⁷⁴_d²³⁷⁵_t²³⁷⁶_a²³⁷⁷_c²³⁷⁸_l²³⁷⁹_l²³⁸⁰_c²³⁸¹_y²³⁸²_k²³⁸³_r²³⁸⁴_n²³⁸⁵_r²³⁸⁶_l²³⁸⁷_t²³⁸⁸_r²³⁸⁹_v²³⁹⁰_e²³⁹¹_a²³⁹²_
s²³⁹³ t²³⁹⁴_v²³⁹⁵_v²³⁹⁶_c²³⁹⁷_g²³⁹⁸_g²³⁹⁹_k²⁴⁰⁰_r²⁴⁰¹_t²⁴⁰²_f²⁴⁰³_y²⁴⁰⁴_i²⁴⁰⁵_t²⁴⁰⁶_a²⁴⁰⁷_n²⁴⁰⁸_g²⁴⁰⁹_g²⁴¹⁰_i²⁴¹¹_
s²⁴¹²_f²⁴¹³ c²⁴¹⁴_r²⁴¹⁵_r²⁴¹⁶_h²⁴¹⁷
(SEQ ID NO: 105)
h²⁴⁵⁷_y²⁴⁵⁸_y²⁴⁵⁹_v²⁴⁶⁰_d²⁴⁶¹_s²⁴⁶²_v²⁴⁶³_t²⁴⁶⁴_v²⁴⁶⁵_k²⁴⁶⁶_e²⁴⁶⁷_t²⁴⁶⁸_v²⁴⁶⁹_v²⁴⁷⁰_q²⁴⁷¹_f²⁴⁷²_n²⁴⁷³_y²⁴⁷⁴_r²⁴⁷⁵
r²⁴⁷⁶_d²⁴⁷⁷_g²⁴⁷⁸_q²⁴⁷⁹_p²⁴⁸⁰_f²⁴⁸¹_y²⁴⁸²_e²⁴⁸³_r²⁴⁸⁴_f²⁴⁸⁵_p²⁴⁸⁶_l²⁴⁸⁷_c²⁴⁸⁸_a²⁴⁸⁹_f²⁴⁹⁰_t²⁴⁹¹_n²⁴⁹²_l²⁴⁹³_d²⁴⁹⁴_
k²⁴⁹⁵ l²⁴⁹⁶_k²⁴⁹⁷_f²⁴⁹⁸_k²⁴⁹⁹_e²⁵⁰⁰_v²⁵⁰¹_c²⁵⁰²_k²⁵⁰³
(SEQ ID NO: 106)
h³¹⁰⁹_v³¹¹⁰_s³¹¹¹_w³¹¹²_y³¹¹³_i³¹¹⁴_m³¹¹⁵_f³¹¹⁶_g³¹¹⁷_p³¹¹⁸_i³¹¹⁹_v³¹²⁰_p³¹²¹_i³¹²²_w³¹²³_m³¹²⁴_t³¹²⁵_c³¹²⁶_v³¹²⁷
y³¹²⁸_t³¹²⁹_v³¹³⁰_a³¹³¹_m³¹³²_c³¹³³_f³¹³⁴_r³¹³⁵_h³¹³⁶_f³¹³⁷_f³¹³⁸_w³¹³⁹_v³¹⁴⁰_l³¹⁴¹_a³¹⁴²_y³¹⁴³_f³¹⁴⁴_s³¹⁴⁵_k³¹⁴⁶_
k³¹⁴⁷ h³¹⁴⁸_v³¹⁴⁹_e³¹⁵⁰_v³¹⁵¹_f³¹⁵²_t³¹⁵³_d³¹⁵⁴_g³¹⁵⁵_k³¹⁵⁶
(SEQ ID NO: 107)
h³¹³⁶_f³¹³⁷_f³¹³⁸_w³¹³⁹_v³¹⁴⁰_l³¹⁴¹_a³¹⁴²_y³¹⁴³_f³¹⁴⁴_s³¹⁴⁵_k³¹⁴⁶_k³¹⁴⁷_h³¹⁴⁸_v³¹⁴⁹_e³¹⁵⁰_v³¹⁵¹_f³¹⁵²_t³¹⁵³_d³¹⁵⁴_
g³¹⁵⁵ k³¹⁵⁶
(SEQ ID NO: 108)
h³¹³⁶_f³¹³⁷_f³¹³⁸_w³¹³⁹_v³¹⁴⁰_l³¹⁴¹_a³¹⁴²_y³¹⁴³_f³¹⁴⁴_s³¹⁴⁵_k³¹⁴⁶_k³¹⁴⁷h³¹⁴⁸_v³¹⁴⁹_e³¹⁵⁰_v³¹⁵¹_f³¹⁵²_t³¹⁵³_d³¹⁵⁴_
g³¹⁵⁵_k³¹⁵⁶
(SEQ ID NO: 6)
k³¹⁴⁷h³¹⁴⁸_v³¹⁴⁹_e³¹⁵⁰_v³¹⁵¹_f³¹⁵²_t³¹⁵³_d³¹⁵⁴_g³¹⁵⁵_k³¹⁵⁶
(SEQ ID NO: 108)
h³⁴²²_t³⁴²³_g³⁴²⁴_s³⁴²⁵_a³⁴²⁶_f³⁴²⁷_d³⁴²⁸_g³⁴²⁹_t³⁴³⁰_m³⁴³¹_y³⁴³²_g³⁴³³_a³⁴³⁴_f³⁴³⁵_m³⁴³⁶_d³⁴³⁷_k³⁴³⁸_q³⁴³⁹_v³⁴⁴⁰
h³⁴⁴¹_q³⁴⁴²_v³⁴⁴³_q³⁴⁴⁴_l³⁴⁴⁵_t³⁴⁴⁶_d³⁴⁴⁷_k³⁴⁴⁸
(SEQ ID NO: 12)
d³⁴³⁷_k³⁴³⁸_q³⁴³⁹_v³⁴⁴⁰_h³⁴⁴¹_q³⁴⁴²_v³⁴⁴³_q³⁴⁴⁴_l³⁴⁴⁵_t³⁴⁴⁶_d³⁴⁴⁷_k³⁴⁴⁸
(SEQ ID NO: 109)
k³⁸⁴⁰_v³⁸⁴¹_a³⁸⁴²_a³⁸⁴³_m³⁸⁴⁴_q³⁸⁴⁵_s³⁸⁴⁶_k³⁸⁴⁷_l³⁸⁴⁸_t³⁸⁴⁹_d³⁸⁵⁰_l³⁸⁵¹_k³⁸⁵²_c³⁸⁵³_t³⁸⁵⁴_s³⁸⁵⁵_v³⁸⁵⁶_v³⁸⁵⁷_l³⁸⁵⁸_
l³⁸⁵⁹ s³⁸⁶⁰_v³⁸⁶¹_l³⁸⁶²_q³⁸⁶³_q³⁸⁶⁴_l³⁸⁶⁵_h³⁸⁶⁶_l³⁸⁶⁷_e³⁸⁶⁸_a³⁸⁶⁹_n³⁸⁷⁰_s³⁸⁷¹_r³⁸⁷²_a³⁸⁷³_w³⁸⁷⁴_a³⁸⁷⁵_f³⁸⁷⁶_c³⁸⁷⁷_
v³⁸⁷⁸_k³⁸⁷⁹ c³⁸⁸⁰_h³⁸⁸¹
(SEQ ID NO: 110)
k³⁸⁴⁰_v³⁸⁴¹_a³⁸⁴²_a³⁸⁴³_m³⁸⁴⁴_q³⁸⁴⁵_s³⁸⁴⁶_k³⁸⁴⁷_l³⁸⁴⁸_t³⁸⁴⁹_d³⁸⁵⁰_l³⁸⁵¹_k³⁸⁵²_c³⁸⁵³_t³⁸⁵⁴_s³⁸⁵⁵_v³⁸⁵⁶_v³⁸⁵⁷_l³⁸⁵⁸_
l³⁸⁵⁹ s³⁸⁶⁰_v³⁸⁶¹_l³⁸⁶²_q³⁸⁶³_q³⁸⁶⁴_l³⁸⁶⁵_h³⁸⁶⁶
(SEQ ID NO: 111)
h³⁹³⁶_l³⁹³⁷_a³⁹³⁸_t³⁹³⁹_f³⁹⁴⁰_a³⁹⁴¹_e³⁹⁴²_l³⁹⁴³_e³⁹⁴⁴_a³⁹⁴⁵_a³⁹⁴⁶_q³⁹⁴⁷_k³⁹⁴⁸_a³⁹⁴⁹_y³⁹⁵⁰_q³⁹⁵¹_e³⁹⁵²_a³⁹⁵³_m³⁹⁵⁴
d³⁹⁵⁵_s³⁹⁵⁶_g³⁹⁵⁷_d³⁹⁵⁸_t³⁹⁵⁹_s³⁹⁶⁰_p³⁹⁶¹_q³⁹⁶²_v³⁹⁶³_l³⁹⁶⁴_k³⁹⁶⁵_a³⁹⁶⁶_l³⁹⁶⁷_q³⁹⁶⁸_k³⁹⁶⁹_a³⁹⁷⁰_v³⁹⁷¹_n³⁹⁷²_i³⁹⁷³_
a³⁹⁷⁴ k³⁹⁷⁵
(SEQ ID NO: 112)
h³⁹³⁶_l³⁹³⁷_a³⁹³⁸_t³⁹³⁹_f³⁹⁴⁰_a³⁹⁴¹_e³⁹⁴²_l³⁹⁴³_e³⁹⁴⁴_a³⁹⁴⁵_a³⁹⁴⁶_q³⁹⁴⁷_k³⁹⁴⁸_a³⁹⁴⁹_y³⁹⁵⁰_q³⁹⁵¹_e³⁹⁵²_a³⁹⁵³_m³⁹⁵⁴
d³⁹⁵⁵_s³⁹⁵⁶_g³⁹⁵⁷_d³⁹⁵⁸_t³⁹⁵⁹_s³⁹⁶⁰_p³⁹⁶¹_q³⁹⁶²_v³⁹⁶³_l³⁹⁶⁴_k³⁹⁶⁵_a³⁹⁶⁶_l³⁹⁶⁷_q³⁹⁶⁸_k³⁹⁶⁹_a³⁹⁷⁰_v³⁹⁷¹_n³⁹⁷²_i³⁹⁷³_
a³⁹⁷⁴ k³⁹⁷⁵_n³⁹⁷⁶_a³⁹⁷⁷_y³⁹⁷⁸_e³⁹⁷⁹_k³⁹⁸⁰_d³⁹⁸¹_k³⁹⁸²
(SEQ ID NO: 113)
k⁴¹⁷⁸_w⁴¹⁷⁹_a⁴¹⁸⁰_r⁴¹⁸¹_v⁴¹⁸²_e⁴¹⁸³_g⁴¹⁸⁴_k⁴¹⁸⁵_d⁴¹⁸⁶_g⁴¹⁸⁷_f⁴¹⁸⁸_v⁴¹⁸⁹_s⁴¹⁹⁰_v⁴¹⁹¹_e⁴¹⁹²_l⁴¹⁹³_q⁴¹⁹⁴_p⁴¹⁹⁵_p⁴¹⁹⁶
c⁴¹⁹⁷_k⁴¹⁹⁸_f⁴¹⁹⁹_l⁴²⁰⁰_i⁴²⁰¹_a⁴²⁰²_g⁴²⁰³_p⁴²⁰⁴_k⁴²⁰⁵_g⁴²⁰⁶_p⁴²⁰⁷_e⁴²⁰⁸_i⁴²⁰⁹_r⁴²¹⁰_y⁴²¹¹_l⁴²¹²_y⁴²¹³_f⁴²¹⁴_v⁴²¹⁵_
k⁴²¹⁶ n⁴²¹⁷_l⁴²¹⁸_n⁴²¹⁹_n⁴²²⁰_l⁴²²¹_h⁴²²²
(SEQ ID NO: 114)
k⁴¹⁹⁸_f⁴¹⁹⁹_l⁴²⁰⁰_i⁴²⁰¹_a⁴²⁰²_g⁴²⁰³_p⁴²⁰⁴_k⁴²⁰⁵_g⁴²⁰⁶_p⁴²⁰⁷_e⁴²⁰⁸_i⁴²⁰⁹_r⁴²¹⁰_y⁴²¹¹_l⁴²¹²_y⁴²¹³_f⁴²¹⁴_v⁴²¹⁵_k⁴²¹⁶_
n⁴²¹⁷ l⁴²¹⁸_n⁴²¹⁹_n⁴²²⁰_l⁴²²¹_h⁴²²²
(SEQ ID NO: 115)
k⁴¹⁹⁸_f⁴¹⁹⁹_l⁴²⁰⁰_i⁴²⁰¹_a⁴²⁰²_g⁴²⁰³_p⁴²⁰⁴_k⁴²⁰⁵_g⁴²⁰⁶_p⁴²⁰⁷_e⁴²⁰⁸_i⁴²⁰⁹_r⁴²¹⁰_y⁴²¹¹_l⁴²¹²_y⁴²¹³_f⁴²¹⁴_v⁴²¹⁵_k⁴²¹⁶_
n⁴²¹⁷ l⁴²¹⁸_n⁴²¹⁹_n⁴²²⁰_l⁴²²¹_h⁴²²²_r⁴²²³_g⁴²²⁴_q⁴²²⁵_v⁴²²⁶_l⁴²²⁷_g⁴²²⁸_h⁴²²⁹
(SEQ ID NO: 116)
k⁴²⁸⁵_t⁴²⁸⁶_g⁴²⁸⁷_t⁴²⁸⁸_g⁴²⁸⁹_i⁴²⁹⁰_a⁴²⁹¹_i⁴²⁹²_s⁴²⁹³_v⁴²⁹⁴_k⁴²⁹⁵_p⁴²⁹⁶_e⁴²⁹⁷_s⁴²⁹⁸_t⁴²⁹⁹_a⁴³⁰⁰_d⁴³⁰¹_q⁴³⁰²_e⁴³⁰³_
t⁴³⁰⁴ y⁴³⁰⁵_g⁴³⁰⁶_g⁴³⁰⁷_a⁴³⁰⁸_s⁴³⁰⁹_v⁴³¹⁰_c⁴³¹¹_l⁴³¹²_y⁴³¹³_c⁴³¹⁴_r⁴³¹⁵_a⁴³¹⁶_h⁴³¹⁷
(SEQ ID NO: 117)
k⁴⁴¹⁸_a⁴⁴¹⁹_k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_
e⁴⁴³⁷ l⁴⁴³⁸_d⁴⁴³⁹_d⁴⁴⁴⁰_q⁴⁴⁴¹_g⁴⁴⁴²_h⁴⁴⁴³
(SEQ ID NO: 118)
k⁴⁴¹⁸_a⁴⁴¹⁹_k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_
e⁴⁴³⁷ l⁴⁴³⁸_d⁴⁴³⁹_d⁴⁴⁴⁰_q⁴⁴⁴¹_g⁴⁴⁴²_h⁴⁴⁴³_h⁴⁴⁴⁴
(SEQ ID NO: 119)
k⁴⁴¹⁸_a⁴⁴¹⁹_k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_
e⁴⁴³⁷ l⁴⁴³⁸_d⁴⁴³⁹_d⁴⁴⁴⁰_q⁴⁴⁴¹_g⁴⁴⁴²_h⁴⁴⁴³_h⁴⁴⁴⁴_l⁴⁴⁴⁵_d⁴⁴⁴⁶_s⁴⁴⁴⁷_y⁴⁴⁴⁸_f⁴⁴⁴⁹_v⁴⁴⁵⁰_v⁴⁴⁵¹_k⁴⁴⁵²_r⁴⁴⁵³_h⁴⁴⁵⁴
(SEQ ID NO: 120)
k⁴⁴¹⁸_a⁴⁴¹⁹_k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_
e⁴⁴³⁷ l⁴⁴³⁸_d⁴⁴³⁹_d⁴⁴⁴⁰_q⁴⁴⁴¹_g⁴⁴⁴²_h⁴⁴⁴³_h⁴⁴⁴⁴_l⁴⁴⁴⁵_d⁴⁴⁴⁶_s⁴⁴⁴⁷_y⁴⁴⁴⁸_f⁴⁴⁴⁹_v⁴⁴⁵⁰_v⁴⁴⁵¹_k⁴⁴⁵²_r⁴⁴⁵³_h⁴⁴⁵⁴_
t⁴⁴⁵⁵_m⁴⁴⁵⁶ e⁴⁴⁵⁷_n⁴⁴⁵⁸_y⁴⁴⁵⁹_e⁴⁴⁶⁰_l⁴⁴⁶¹_e⁴⁴⁶²_k⁴⁴⁶³_h⁴⁴⁶⁴
(SEQ ID NO: 121)
k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_e⁴⁴³⁷_l⁴⁴³⁸_
d⁴⁴³⁹ d⁴⁴⁴⁰_q⁴⁴⁴¹_g⁴⁴⁴²_h⁴⁴⁴³_h⁴⁴⁴⁴_l⁴⁴⁴⁵_d⁴⁴⁴⁶_s⁴⁴⁴⁷_y⁴⁴⁴⁸_f⁴⁴⁴⁹_v⁴⁴⁵⁰_v⁴⁴⁵¹_k⁴⁴⁵²_r⁴⁴⁵³_h⁴⁴⁵⁴
(SEQ ID NO: 122)
k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_e⁴⁴³⁷_l⁴⁴³⁸_
d⁴⁴³⁹ d⁴⁴⁴⁰_q⁴⁴⁴¹_g⁴⁴⁴²_h⁴⁴⁴³_h⁴⁴⁴⁴_l⁴⁴⁴⁵_d⁴⁴⁴⁶_s⁴⁴⁴⁷_y⁴⁴⁴⁸_f⁴⁴⁴⁹_v⁴⁴⁵⁰_v⁴⁴⁵¹_k⁴⁴⁵²_r⁴⁴⁵³_h⁴⁴⁵⁴_t⁴⁴⁵⁵_m⁴⁴⁵⁶_e⁴⁴⁵⁷_
n⁴⁴⁵⁸ y⁴⁴⁵⁹_e⁴⁴⁶⁰_l⁴⁴⁶¹_e⁴⁴⁶²_k⁴⁴⁶³_h⁴⁴⁶⁴
(SEQ ID NO: 123)
k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_e⁴⁴³⁷_l⁴⁴³⁸_
d⁴⁴³⁹ d⁴⁴⁴⁰_q⁴⁴⁴¹_g⁴⁴⁴²_h⁴⁴⁴³
(SEQ ID NO: 124)
k⁴⁴²⁰_v⁴⁴²¹_a⁴⁴²²_g⁴⁴²³_i⁴⁴²⁴_g⁴⁴²⁵_k⁴⁴²⁶_y⁴⁴²⁷_y⁴⁴²⁸_k⁴⁴²⁹_t⁴⁴³⁰_n⁴⁴³¹_t⁴⁴³²_c⁴⁴³³_r⁴⁴³⁴_f⁴⁴³⁵_v⁴⁴³⁶_e⁴⁴³⁷_l⁴⁴³⁸_
d⁴⁴³⁹ d⁴⁴⁴⁰_q⁴⁴⁴¹_g⁴⁴⁴²_h⁴⁴⁴³_h⁴⁴⁴⁴
(SEQ ID NO: 125)
h⁴⁵⁵⁴_k⁴⁵⁵⁵_l⁴⁵⁵⁶_g⁴⁵⁵⁷_e⁴⁵⁵⁸_r⁴⁵⁵⁹_v⁴⁵⁶⁰_r⁴⁵⁶¹_q⁴⁵⁶²_a⁴⁵⁶³_i⁴⁵⁶⁴_l⁴⁵⁶⁵_n⁴⁵⁶⁶_l⁴⁵⁶⁷_t⁴⁵⁶⁸_k⁴⁵⁶⁹_f⁴⁵⁷⁰_c⁴⁵⁷¹_d⁴⁵⁷²_
h⁴⁵⁷³ m⁴⁵⁷⁴_v⁴⁵⁷⁵_k⁴⁵⁷⁶
(SEQ ID NO: 20)
k⁴⁵⁶⁹_f⁴⁵⁷⁰_c⁴⁵⁷¹_d⁴⁵⁷²_h⁴⁵⁷³_m⁴⁵⁷⁴_v⁴⁵⁷⁵_k⁴⁵⁷⁶
(SEQ ID NO: 126)
h⁴⁶³⁴_r⁴⁶³⁵_d⁴⁶³⁶_c⁴⁶³⁷_d⁴⁶³⁸_f⁴⁶³⁹_n⁴⁶⁴⁰_k⁴⁶⁴¹_p⁴⁶⁴²_l⁴⁶⁴³_i⁴⁶⁴⁴_e⁴⁶⁴⁵_w⁴⁶⁴⁶_p⁴⁶⁴⁷_l⁴⁶⁴⁸_t⁴⁶⁴⁹_e⁴⁶⁵⁰_y⁴⁶⁵¹_d⁴⁶⁵²_
f⁴⁶⁵³ t⁴⁶⁵⁴_d⁴⁶⁵⁵_y⁴⁶⁵⁶_k⁴⁶⁵⁷_v⁴⁶⁵⁸_q⁴⁶⁵⁹_l⁴⁶⁶⁰_f⁴⁶⁶¹_e⁴⁶⁶²_k⁴⁶⁶³
(SEQ ID NO: 127)
h⁴⁶³⁴_r⁴⁶³⁵_d⁴⁶³⁶_c⁴⁶³⁷_d⁴⁶³⁸_f⁴⁶³⁹_n⁴⁶⁴⁰_k⁴⁶⁴¹_p⁴⁶⁴²_l⁴⁶⁴³_i⁴⁶⁴⁴_e⁴⁶⁴⁵_w⁴⁶⁴⁶_p⁴⁶⁴⁷_l⁴⁶⁴⁸_t⁴⁶⁴⁹_e⁴⁶⁵⁰_y⁴⁶⁵¹_d⁴⁶⁵²_
f⁴⁶⁵³ t⁴⁶⁵⁴_d⁴⁶⁵⁵_y⁴⁶⁵⁶_k⁴⁶⁵⁷_v⁴⁶⁵⁸_q⁴⁶⁵⁹_l⁴⁶⁶⁰_f⁴⁶⁶¹_e⁴⁶⁶²_k⁴⁶⁶³_y⁴⁶⁶⁴_f⁴⁶⁶⁵_k⁴⁶⁶⁶
(SEQ ID NO: 128)
k⁴⁶⁵⁷_v⁴⁶⁵⁸_q⁴⁶⁵⁹_l⁴⁶⁶⁰_f⁴⁶⁶¹_e⁴⁶⁶²_k⁴⁶⁶³_y⁴⁶⁶⁴_f⁴⁶⁶⁵_k⁴⁶⁶⁶_y⁴⁶⁶⁷_w⁴⁶⁶⁸_d⁴⁶⁶⁹_q⁴⁶⁷⁰_t⁴⁶⁷¹_y⁴⁶⁷²_h⁴⁶⁷³
(SEQ ID NO: 129)
k⁴⁶⁵⁷_v⁴⁶⁵⁸_q⁴⁶⁵⁹_l⁴⁶⁶⁰_f⁴⁶⁶¹_e⁴⁶⁶²_k⁴⁶⁶³_y⁴⁶⁶⁴_f⁴⁶⁶⁵_k⁴⁶⁶⁶_y⁴⁶⁶⁷_w⁴⁶⁶⁸_d⁴⁶⁶⁹_q⁴⁶⁷⁰_t⁴⁶⁷¹_y⁴⁶⁷²_h⁴⁶⁷³_a⁴⁶⁷⁴_n⁴⁶⁷⁵
c⁴⁶⁷⁶_v⁴⁶⁷⁷_n⁴⁶⁷⁸_c⁴⁶⁷⁹_t⁴⁶⁸⁰_d⁴⁶⁸¹_d⁴⁶⁸²_r⁴⁶⁸³_c⁴⁶⁸⁴_v⁴⁶⁸⁵_l⁴⁶⁸⁶_h⁴⁶⁸⁷
(SEQ ID NO: 130)
h⁴⁶⁸⁷_c⁴⁶⁸⁸_a⁴⁶⁸⁹_n⁴⁶⁹⁰_f⁴⁶⁹¹_n⁴⁶⁹²_v⁴⁶⁹³_l⁴⁶⁹⁴_f⁴⁶⁹⁵_a⁴⁶⁹⁶_m⁴⁶⁹⁷_t⁴⁶⁹⁸_m⁴⁶⁹⁹_p⁴⁷⁰⁰_k⁴⁷⁰¹_t⁴⁷⁰²_c⁴⁷⁰³_f⁴⁷⁰⁴_g⁴⁷⁰⁵
p⁴⁷⁰⁶_i⁴⁷⁰⁷_v⁴⁷⁰⁸_r⁴⁷⁰⁹_k⁴⁷¹⁰
(SEQ ID NO: 131)
k⁴⁷⁰¹_t⁴⁷⁰²_c⁴⁷⁰³_f⁴⁷⁰⁴_g⁴⁷⁰⁵_p⁴⁷⁰⁶_i⁴⁷⁰⁷_v⁴⁷⁰⁸_r⁴⁷⁰⁹_k⁴⁷¹⁰_i⁴⁷¹¹_f⁴⁷¹²_v⁴⁷¹³_d⁴⁷¹⁴_g⁴⁷¹⁵_v⁴⁷¹⁶_p⁴⁷¹⁷_f⁴⁷¹⁸_v⁴⁷¹⁹_
v⁴⁷²⁰ s⁴⁷²¹_c⁴⁷²²_g⁴⁷²³_y⁴⁷²⁴_h⁴⁷²⁵
(SEQ ID NO: 132)
k⁴⁷⁰¹_t⁴⁷⁰²_c⁴⁷⁰³_f⁴⁷⁰⁴_g⁴⁷⁰⁵_p⁴⁷⁰⁶_i⁴⁷⁰⁷_v⁴⁷⁰⁸_r⁴⁷⁰⁹_k⁴⁷¹⁰_i⁴⁷¹¹_f⁴⁷¹²_v⁴⁷¹³_d⁴⁷¹⁴_g⁴⁷¹⁵_v⁴⁷¹⁶_p⁴⁷¹⁷_f⁴⁷¹⁸_v⁴⁷¹⁹_
v⁴⁷²⁰ s⁴⁷²¹_c⁴⁷²²_g⁴⁷²³_y⁴⁷²⁴_h⁴⁷²⁵_y⁴⁷²⁶_k⁴⁷²⁷_e⁴⁷²⁸_l⁴⁷²⁹_g⁴⁷³⁰_l⁴⁷³¹_v⁴⁷³²_m⁴⁷³³_n⁴⁷³⁴_m⁴⁷³⁵_d⁴⁷³⁶_v⁴⁷³⁷_s⁴⁷³⁸_
l⁴⁷³⁹_h⁴⁷⁴⁰
(SEQ ID NO: 133)
k⁴⁷⁰¹_t⁴⁷⁰²_c⁴⁷⁰³_f⁴⁷⁰⁴_g⁴⁷⁰⁵_p⁴⁷⁰⁶_i⁴⁷⁰⁷_v⁴⁷⁰⁸_r⁴⁷⁰⁹_k⁴⁷¹⁰_i⁴⁷¹¹_f⁴⁷¹²_v⁴⁷¹³_d⁴⁷¹⁴_g⁴⁷¹⁵_v⁴⁷¹⁶_p⁴⁷¹⁷_f⁴⁷¹⁸_v⁴⁷¹⁹_
v⁴⁷²⁰ s⁴⁷²¹_c⁴⁷²²_g⁴⁷²³_y⁴⁷²⁴_h⁴⁷²⁵_y⁴⁷²⁶_k⁴⁷²⁷_e⁴⁷²⁸_l⁴⁷²⁹_g⁴⁷³⁰_l⁴⁷³¹_v⁴⁷³²_m⁴⁷³³_n⁴⁷³⁴_m⁴⁷³⁵_d⁴⁷³⁶_v⁴⁷³⁷_s⁴⁷³⁸_
l⁴⁷³⁹ h⁴⁷⁴⁰_r⁴⁷⁴¹_h⁴⁷⁴²
Carboxy-terminal
(SEQ ID NO: 28)
k⁴⁸⁰⁸_e⁴⁸⁰⁹_g⁴⁸¹⁰_s⁴⁸¹¹_s⁴⁸¹²_v⁴⁸¹³_t⁴⁸¹⁴_l⁴⁸¹⁵_k⁴⁸¹⁶_h⁴⁸¹⁷
(SEQ ID NO: 2)
k⁴⁸⁷⁸_s⁴⁸⁷⁹_a⁴⁸⁸⁰_g⁴⁸⁸¹_h⁴⁸⁸²_p⁴⁸⁸³_f⁴⁸⁸⁴_n⁴⁸⁸⁵_k⁴⁸⁸⁶
(SEQ ID NO: 134)
h⁴⁸⁸²_p⁴⁸⁸³_f⁴⁸⁸⁴_n⁴⁸⁸⁵_k⁴⁸⁸⁶_f⁴⁸⁸⁷_g⁴⁸⁸⁸_k⁴⁸⁸⁹_a⁴⁸⁹⁰_r⁴⁸⁹¹_v⁴⁸⁹²_v⁴⁸⁹³_y⁴⁸⁹⁴_e⁴⁸⁹⁵_s⁴⁸⁹⁶_m⁴⁸⁹⁷_s⁴⁸⁹⁸_y⁴⁸⁹⁹_q⁴⁹⁰⁰
e⁴⁹⁰¹_q⁴⁹⁰²_d⁴⁹⁰³_e⁴⁹⁰⁴_l⁴⁹⁰⁵_f⁴⁹⁰⁶_a⁴⁹⁰⁷_m⁴⁹⁰⁸_t⁴⁹⁰⁹_k⁴⁹¹⁰_r⁴⁹¹¹_n⁴⁹¹²_v⁴⁹¹³_i⁴⁹¹⁴_p⁴⁹¹⁵_t⁴⁹¹⁶_m⁴⁹¹⁷_t⁴⁹¹⁸_q⁴⁹¹⁹
m⁴⁹²⁰_n⁴⁹²¹_l⁴⁹²²_k⁴⁹²³_y⁴⁹²⁴_a⁴⁹²⁵_i⁴⁹²⁶_s⁴⁹²⁷_a⁴⁹²⁸_k⁴⁹²⁹
(SEQ ID NO: 135)
k⁴⁹²³_y⁴⁹²⁴_a⁴⁹²⁵_i⁴⁹²⁶_s⁴⁹²⁷_a⁴⁹²⁸_k⁴⁹²⁹_n⁴⁹³⁰_r⁴⁹³¹_a⁴⁹³²_r⁴⁹³³_t⁴⁹³⁴_v⁴⁹³⁵_a⁴⁹³⁶_g⁴⁹³⁷_v⁴⁹³⁸_s⁴⁹³⁹_i⁴⁹⁴⁰_l⁴⁹⁴¹_
s⁴⁹⁴² t⁴⁹⁴³_m⁴⁹⁴⁴_t⁴⁹⁴⁵_n⁴⁹⁴⁶_r⁴⁹⁴⁷_q⁴⁹⁴⁸_y⁴⁹⁴⁹_h⁴⁹⁵⁰
(SEQ ID NO: 136)
h⁴⁹⁵⁰_q⁴⁹⁵¹_k⁴⁹⁵²_m⁴⁹⁵³_l⁴⁹⁵⁴_k⁴⁹⁵⁵_s⁴⁹⁵⁶_m⁴⁹⁵⁷_a⁴⁹⁵⁸_a⁴⁹⁵⁹_t⁴⁹⁶⁰_r⁴⁹⁶¹_g⁴⁹⁶²_a⁴⁹⁶³_t⁴⁹⁶⁴_c⁴⁹⁶⁵_v⁴⁹⁶⁶_i⁴⁹⁶⁷_g⁴⁹⁶⁸
t⁴⁹⁶⁹_t⁴⁹⁷⁰_k⁴⁹⁷¹_f⁴⁹⁷²_y⁴⁹⁷³_g⁴⁹⁷⁴_g⁴⁹⁷⁵_w⁴⁹⁷⁶_d⁴⁹⁷⁷_f⁴⁹⁷⁸_m⁴⁹⁷⁹_l⁴⁹⁸⁰_k⁴⁹⁸¹
(SEQ ID NO: 137)
k⁴⁹⁷¹_f⁴⁹⁷²_y⁴⁹⁷³_g⁴⁹⁷⁴_g⁴⁹⁷⁵_w⁴⁹⁷⁶_d⁴⁹⁷⁷_f⁴⁹⁷⁸_m⁴⁹⁷⁹_l⁴⁹⁸⁰_k⁴⁹⁸¹_t⁴⁹⁸²_l⁴⁹⁸³_y⁴⁹⁸⁴_k⁴⁹⁸⁵_d⁴⁹⁸⁶_v⁴⁹⁸⁷_d⁴⁹⁸⁸_n⁴⁹⁸⁹
p⁴⁹⁹⁰_h⁴⁹⁹¹
(SEQ ID NO: 138)
k⁴⁹⁷¹_f⁴⁹⁷²_y⁴⁹⁷³_g⁴⁹⁷⁴_g⁴⁹⁷⁵_w⁴⁹⁷⁶_d⁴⁹⁷⁷_f⁴⁹⁷⁸_m⁴⁹⁷⁹_l⁴⁹⁸⁰_k⁴⁹⁸¹_t⁴⁹⁸²_l⁴⁹⁸³_y⁴⁹⁸⁴_k⁴⁹⁸⁵_d⁴⁹⁸⁶_v⁴⁹⁸⁷_d⁴⁹⁸⁸_n⁴⁹⁸⁹
p⁴⁹⁹⁰_h⁴⁹⁹¹_l⁴⁹⁹²_m⁴⁹⁹³_g⁴⁹⁹⁴_w⁴⁹⁹⁵_d⁴⁹⁹⁶_y⁴⁹⁹⁷_p⁴⁹⁹⁸_k⁴⁹⁹⁹_c⁵⁰⁰⁰_d⁵⁰⁰¹_r⁵⁰⁰²_a⁵⁰⁰³_m⁵⁰⁰⁴_p⁵⁰⁰⁵_n⁵⁰⁰⁶_m⁵⁰⁰⁷_c⁵⁰⁰⁸
r⁵⁰⁰⁹_i⁵⁰¹⁰_f⁵⁰¹¹_a⁵⁰¹²_s⁵⁰¹³_l⁵⁰¹⁴_i⁵⁰¹⁵_l⁵⁰¹⁶_a⁵⁰¹⁷_r⁵⁰¹⁸_k⁵⁰¹⁹_h⁵⁰²⁰
(SEQ ID NO: 139)
k⁵⁰⁹²_i⁵⁰⁹³_v⁵⁰⁹⁴_d⁵⁰⁹⁵_k⁵⁰⁹⁶_e⁵⁰⁹⁷_v⁵⁰⁹⁸_k⁵⁰⁹⁹_d⁵¹⁰⁰_m⁵¹⁰¹_q⁵¹⁰²_f⁵¹⁰³_d⁵¹⁰⁴_l⁵¹⁰⁵_y⁵¹⁰⁶_v⁵¹⁰⁷_n⁵¹⁰⁸_v⁵¹⁰⁹_y⁵¹¹⁰
r⁵¹¹¹_s⁵¹¹²_t⁵¹¹³_s⁵¹¹⁴_p⁵¹¹⁵_d⁵¹¹⁶_p⁵¹¹⁷_k⁵¹¹⁸_f⁵¹¹⁹_v⁵¹²⁰_d⁵¹²¹_k⁵¹²²_y⁵¹²³_y⁵¹²⁴_a⁵¹²⁵_f⁵¹²⁶_l⁵¹²⁷_n⁵¹²⁸_k⁵¹²⁹
h⁵¹³⁰
(SEQ ID NO: 17)
k⁵¹²²_y⁵¹²³_y⁵¹²⁴_a⁵¹²⁵_f⁵¹²⁶_l⁵¹²⁷_n⁵¹²⁸_k⁵¹²⁹_h⁵¹³⁰
(SEQ ID NO: 140)
h⁵¹³⁰_f⁵¹³¹_s⁵¹³²_m⁵¹³³_m⁵¹³⁴_i⁵¹³⁵_l⁵¹³⁶_s⁵¹³⁷_d⁵¹³⁸_d⁵¹³⁹_g⁵¹⁴⁰_v⁵¹⁴¹_v⁵¹⁴²_c⁵¹⁴³_y⁵¹⁴⁴_n⁵¹⁴⁵_s⁵¹⁴⁶_d⁵¹⁴⁷_y⁵¹⁴⁸
a⁵¹⁴⁹_a⁵¹⁵⁰_k⁵¹⁵¹_g⁵¹⁵²_y⁵¹⁵³_i⁵¹⁵⁴_a⁵¹⁵⁵_g⁵¹⁵⁶_i⁵¹⁵⁷_q⁵¹⁵⁸_n⁵¹⁵⁹_f⁵¹⁶⁰_k⁵¹⁶¹
(SEQ ID NO: 141)
g⁵¹⁵²_y⁵¹⁵³_i⁵¹⁵⁴_a⁵¹⁵⁵_g⁵¹⁵⁶_i⁵¹⁵⁷_q⁵¹⁵⁸_n⁵¹⁵⁹_f⁵¹⁶⁰_k⁵¹⁶¹_e⁵¹⁶²_t⁵¹⁶³_l⁵¹⁶⁴_y⁵¹⁶⁵_y⁵¹⁶⁶_q⁵¹⁶⁷_n⁵¹⁶⁸_n⁵¹⁶⁹_v⁵¹⁷⁰
f⁵¹⁷¹_m⁵¹⁷²_s⁵¹⁷³_e⁵¹⁷⁴_a⁵¹⁷⁵_k⁵¹⁷⁶_c⁵¹⁷⁷_w⁵¹⁷⁸_v⁵¹⁷⁹_e⁵¹⁸⁰_t⁵¹⁸¹_d⁵¹⁸²_l⁵¹⁸³_k⁵¹⁸⁴_k⁵¹⁸⁵_g⁵¹⁸⁶_p⁵¹⁸⁷_h⁵¹⁸⁸
(SEQ ID NO: 142)
g⁵¹⁵²_y⁵¹⁵³_i⁵¹⁵⁴_a⁵¹⁵⁵_g⁵¹⁵⁶_i⁵¹⁵⁷_q⁵¹⁵⁸_n⁵¹⁵⁹_f⁵¹⁶⁰_k⁵¹⁶¹_e⁵¹⁶²_t⁵¹⁶³_l⁵¹⁶⁴_y⁵¹⁶⁵_y⁵¹⁶⁶_q⁵¹⁶⁷_n⁵¹⁶⁸_n⁵¹⁶⁹_v⁵¹⁷⁰
f⁵¹⁷¹_m⁵¹⁷²_s⁵¹⁷³_e⁵¹⁷⁴_a⁵¹⁷⁵_k⁵¹⁷⁶_c⁵¹⁷⁷_w⁵¹⁷⁸_v⁵¹⁷⁹_e⁵¹⁸⁰_t⁵¹⁸¹_d⁵¹⁸²_l⁵¹⁸³_k⁵¹⁸⁴_k⁵¹⁸⁵_g⁵¹⁸⁶_p⁵¹⁸⁷_h⁵¹⁸⁸_e⁵¹⁸⁹
f⁵¹⁹⁰_c⁵¹⁹¹_s⁵¹⁹²_q⁵¹⁹³_h⁵⁹¹⁴
(SEQ ID NO: 143)
k⁵¹⁷⁶_c⁵¹⁷⁷_w⁵¹⁷⁸_v⁵¹⁷⁹_e⁵¹⁸⁰_t⁵¹⁸¹_d⁵¹⁸²_l⁵¹⁸³_k⁵¹⁸⁴_k⁵¹⁸⁵_g⁵¹⁸⁶_p⁵¹⁸⁷_h⁵¹⁸⁸
(SEQ ID NO: 144)
k⁵¹⁷⁶_c⁵¹⁷⁷_w⁵¹⁷⁸_v⁵¹⁷⁹_e⁵¹⁸⁰_t⁵¹⁸¹_d⁵¹⁸²_l⁵¹⁸³_k⁵¹⁸⁴_k⁵¹⁸⁵_g⁵¹⁸⁶_p⁵¹⁸⁷_h⁵¹⁸⁸_e⁵¹⁸⁹_f⁵¹⁹⁰_c⁵¹⁹¹_s⁵¹⁹²_q⁵¹⁹³_h⁵⁹¹⁴
(SEQ ID NO: 145)
k⁵⁴⁵⁶_q⁵⁴⁵⁷_s⁵⁴⁵⁸_y⁵⁴⁵⁹_a⁵⁴⁶⁰_i⁵⁴⁶¹_a⁵⁴⁶²_t⁵⁴⁶³_i⁵⁴⁶⁴_k⁵⁴⁶⁵_e⁵⁴⁶⁶_i⁵⁴⁶⁷_v⁵⁴⁶⁸_g⁵⁴⁶⁹_e⁵⁴⁷⁰_r⁵⁴⁷¹_q⁵⁴⁷²_l⁵⁴⁷³_l⁵⁴⁷⁴_
l⁵⁴⁷⁵ v⁵⁴⁷⁶_w⁵⁴⁷⁷_e⁵⁴⁷⁸_a⁵⁴⁷⁹_g⁵⁴⁸⁰_k⁵⁴⁸¹_s⁵⁴⁸²_k⁵⁴⁸³_p⁵⁴⁸⁴_p⁵⁴⁸⁵_l⁵⁴⁸⁶_n⁵⁴⁸⁷_r⁵⁴⁸⁸_n⁵⁴⁸⁹_y⁵⁴⁹⁰_v⁵⁴⁹¹_f⁵⁴⁹²_t⁵⁴⁹³_
g⁵⁴⁹⁴ y⁵⁴⁹⁵_h⁵⁴⁹⁶
(SEQ ID NO: 146)
h⁵⁴⁹⁶_i⁵⁴⁹⁷_t⁵⁴⁹⁸_k⁵⁴⁹⁹_n⁵⁵⁰⁰_s⁵⁵⁰¹_k⁵⁵⁰²_v⁵⁵⁰³_q⁵⁵⁰⁴_l⁵⁵⁰⁵_g⁵⁵⁰⁶_e⁵⁵⁰⁷_y⁵⁵⁰⁸_i⁵⁵⁰⁹_f⁵⁵¹⁰_e⁵⁵¹¹_r⁵⁵¹²_i⁵⁵¹³_d⁵⁵¹⁴_
y⁵⁵¹⁵ s⁵⁵¹⁶_d⁵⁵¹⁷_a⁵⁵¹⁸_v⁵⁵¹⁹_s⁵⁵²⁰_y⁵⁵²¹_k⁵⁵²²_s⁵⁵²³_s⁵⁵²⁴_t⁵⁵²⁵_t⁵⁵²⁶_y⁵⁵²⁷_k⁵⁵²⁸
(SEQ ID NO: 147)
k⁵⁵²²_s⁵⁵²³_s⁵⁵²⁴_t⁵⁵²⁵_t⁵⁵²⁶_y⁵⁵²⁷_k⁵⁵²⁸_l⁵⁵²⁹_t⁵⁵³⁰_v⁵⁵³¹_g⁵⁵³²_d⁵⁵³³_i⁵⁵³⁴_f⁵⁵³⁵_v⁵⁵³⁶_l⁵⁵³⁷_t⁵⁵³⁸_s⁵⁵³⁹_h⁵⁵⁴⁰
(SEQ ID NO: 148)
h⁵⁶⁰⁰_f⁵⁶⁰¹_a⁵⁶⁰²_i⁵⁶⁰³_g⁵⁶⁰⁴_l⁵⁶⁰⁵_a⁵⁶⁰⁶_i⁵⁶⁰⁷_y⁵⁶⁰⁸_y⁵⁶⁰⁹_p⁵⁶¹⁰_t⁵⁶¹¹_a⁵⁶¹²_r⁵⁶¹³_v⁵⁶¹⁴_v⁵⁶¹⁵_y⁵⁶¹⁶_t⁵⁶¹⁷_a⁵⁶¹⁸_
c⁵⁶¹⁹ s⁵⁶²⁰_h⁵⁶²¹_a⁵⁶²²_a⁵⁶²³_v⁵⁶²⁴_d⁵⁶²⁵_a⁵⁶²⁶_l⁵⁶²⁷_c⁵⁶²⁸_e⁵⁶²⁹_k⁵⁶³⁰_a⁵⁶³¹_f⁵⁶³²_k⁵⁶³³_y⁵⁶³⁴_l⁵⁶³⁵_n⁵⁶³⁶_i⁵⁶³⁷_
a⁵⁶³⁸_k⁵⁶³⁹
(SEQ ID NO: 149)
h⁵⁶⁰⁰_f⁵⁶⁰¹_a⁵⁶⁰²_i⁵⁶⁰³_g⁵⁶⁰⁴_l⁵⁶⁰⁵_a⁵⁶⁰⁶_i⁵⁶⁰⁷_y⁵⁶⁰⁸_y⁵⁶⁰⁹_p⁵⁶¹⁰_t⁵⁶¹¹_a⁵⁶¹²_r⁵⁶¹³_v⁵⁶¹⁴_v⁵⁶¹⁵_y⁵⁶¹⁶_t⁵⁶¹⁷_a⁵⁶¹⁸_
c⁵⁶¹⁹ s⁵⁶²⁰_h⁵⁶²¹_a⁵⁶²²_a⁵⁶²³_v⁵⁶²⁴_d⁵⁶²⁵_a⁵⁶²⁶_l⁵⁶²⁷_c⁵⁶²⁸_e⁵⁶²⁹_k⁵⁶³⁰_a⁵⁶³¹_f⁵⁶³²_k⁵⁶³³_y⁵⁶³⁴_l⁵⁶³⁵_n⁵⁶³⁶_i⁵⁶³⁷_
a⁵⁶³⁸_k⁵⁶³⁹c⁵⁶⁴⁰_s⁵⁶⁴¹_r⁵⁶⁴²_i⁵⁶⁴³_i⁵⁶⁴⁴_p⁵⁶⁴⁵_a⁵⁶⁴⁶_k⁵⁶⁴⁷
(SEQ ID NO: 150)
h⁵⁶²¹_a⁵⁶²²_a⁵⁶²³_v⁵⁶²⁴_d⁵⁶²⁵_a⁵⁶²⁶_l⁵⁶²⁷_c⁵⁶²⁸_e⁵⁶²⁹_k⁵⁶³⁰_a⁵⁶³¹_f⁵⁶³²_k⁵⁶³³_y⁵⁶³⁴_l⁵⁶³⁵_n⁵⁶³⁶_i⁵⁶³⁷_a⁵⁶³⁸_k⁵⁶³⁹
(SEQ ID NO: 151)
h⁵⁶²¹_a⁵⁶²²_a⁵⁶²³_v⁵⁶²⁴_d⁵⁶²⁵_a⁵⁶²⁶_l⁵⁶²⁷_c⁵⁶²⁸_e⁵⁶²⁹_k⁵⁶³⁰_a⁵⁶³¹_f⁵⁶³²_k⁵⁶³³_y⁵⁶³⁴_l⁵⁶³⁵_n⁵⁶³⁶_i⁵⁶³⁷_a⁵⁶³⁸_k⁵⁶³⁹_
c⁵⁶⁴⁰ s⁵⁶⁴¹_r⁵⁶⁴²_i⁵⁶⁴³_i⁵⁶⁴⁴_p⁵⁶⁴⁵_a⁵⁶⁴⁶_k⁵⁶⁴⁷
(SEQ ID NO: 152)
h⁵⁶²¹_a⁵⁶²²_a⁵⁶²³_v⁵⁶²⁴_d⁵⁶²⁵_a⁵⁶²⁶_l⁵⁶²⁷_c⁵⁶²⁸_e⁵⁶²⁹_k⁵⁶³⁰_a⁵⁶³¹_f⁵⁶³²_k⁵⁶³³_y⁵⁶³⁴_l⁵⁶³⁵_n⁵⁶³⁶_i⁵⁶³⁷_a⁵⁶³⁸_k⁵⁶³⁹_
c⁵⁶⁴⁰ s⁵⁶⁴¹_r⁵⁶⁴²_i⁵⁶⁴³_i⁵⁶⁴⁴_p⁵⁶⁴⁵_a⁵⁶⁴⁶_k⁵⁶⁴⁷_a⁵⁶⁴⁸_r⁵⁶⁴⁹_v⁵⁶⁵⁰_e⁵⁶⁵¹_c⁵⁶⁵²_y⁵⁶⁵³_d⁵⁶⁵⁴_r⁵⁶⁵⁵_f⁵⁶⁵⁶_k⁵⁶⁵⁷
(SEQ ID NO: 153)
k⁵⁷⁷⁴_k⁵⁷⁷⁵_e⁵⁷⁷⁶_l⁵⁷⁷⁷_s⁵⁷⁷⁸_g⁵⁷⁷⁹_q⁵⁷⁸⁰_c⁵⁷⁸¹_f⁵⁷⁸²_k⁵⁷⁸³_i⁵⁷⁸⁴_l⁵⁷⁸⁵_y⁵⁷⁸⁶_k⁵⁷⁸⁷_g⁵⁷⁸⁸_n⁵⁷⁸⁹_v⁵⁷⁹⁰_t⁵⁷⁹¹_h⁵⁷⁹²
(SEQ ID NO: 154)
k⁵⁷⁷⁵_e⁵⁷⁷⁶_l⁵⁷⁷⁷_s⁵⁷⁷⁸_g⁵⁷⁷⁹_q⁵⁷⁸⁰_c⁵⁷⁸¹_f⁵⁷⁸²_k⁵⁷⁸³_i⁵⁷⁸⁴_l⁵⁷⁸⁵_y⁵⁷⁸⁶_k⁵⁷⁸⁷_g⁵⁷⁸⁸_n⁵⁷⁸⁹_v⁵⁷⁹⁰_t⁵⁷⁹¹_h⁵⁷⁹²
(SEQ ID NO: 16)
k⁶⁰⁵⁵_h⁶⁰⁵⁶_l⁶⁰⁵⁷_v⁶⁰⁵⁸_p⁶⁰⁵⁹_l⁶⁰⁶⁰_m⁶⁰⁶¹_h⁶⁰⁶²_k⁶⁰⁶³
(SEQ ID NO: 155)
k⁶⁰⁵⁵_h⁶⁰⁵⁶_l⁶⁰⁵⁷_v⁶⁰⁵⁸_p⁶⁰⁵⁹_l⁶⁰⁶⁰_m⁶⁰⁶¹_h⁶⁰⁶²_k⁶⁰⁶³_g⁶⁰⁶⁴_a⁶⁰⁶⁵_a⁶⁰⁶⁶_w⁶⁰⁶⁷_p⁶⁰⁶⁸_i⁶⁰⁶⁹_v⁶⁰⁷⁰_r⁶⁰⁷¹_r⁶⁰⁷²_r⁶⁰⁷³
i⁶⁰⁷⁴_v⁶⁰⁷⁵_q⁶⁰⁷⁶_m⁶⁰⁷⁷_l⁶⁰⁷⁸_s⁶⁰⁷⁹_d⁶⁰⁸⁰_t⁶⁰⁸¹_l⁶⁰⁸²_d⁶⁰⁸³_k⁶⁰⁸⁴_l⁶⁰⁸⁵_s⁶⁰⁸⁶_d⁶⁰⁸⁷_y⁶⁰⁸⁸_c⁶⁰⁸⁹_t⁶⁰⁹⁰_f⁶⁰⁹¹_v⁶⁰⁹²_
c⁶⁰⁹³ w⁶⁰⁹⁴_a⁶⁰⁹⁵_h⁶⁰⁹⁶
(SEQ ID NO: 156)
h⁶⁰⁹⁶_g⁶⁰⁹⁷_f⁶⁰⁹⁸_e⁶⁰⁹⁹_l⁶¹⁰⁰_t⁶¹⁰¹_s⁶¹⁰²_a⁶¹⁰³_s⁶¹⁰⁴_y⁶¹⁰⁵_f⁶¹⁰⁶_c⁶¹⁰⁷_k⁶¹⁰⁸_i⁶¹⁰⁹_g⁶¹¹⁰_k⁶¹¹¹_e⁶¹¹²_q⁶¹¹³_k⁶¹¹⁴
(SEQ ID NO: 157)
k⁶¹⁰⁸_i⁶¹⁰⁹_g⁶¹¹⁰_k⁶¹¹¹_e⁶¹¹²_q⁶¹¹³_k⁶¹¹⁴_c⁶¹¹⁵_c⁶¹¹⁶_m⁶¹¹⁷_c⁶¹¹⁸_n⁶¹¹⁹_r⁶¹²⁰_r⁶¹²¹_a⁶¹²²_a⁶¹²³_a⁶¹²⁴_y⁶¹²⁵_s⁶¹²⁶
s⁶¹²⁷_p⁶¹²⁸_l⁶¹²⁹_q⁶¹³⁰_s⁶¹³¹_y⁶¹³²_a⁶¹³³_c⁶¹³⁴_w⁶¹³⁵_t⁶¹³⁶_h⁶¹³⁷
(SEQ ID NO: 158)
h⁶²⁰⁹_e⁶²¹⁰_k⁶²¹¹_k⁶²¹²_l⁶²¹³_n⁶²¹⁴_s⁶²¹⁵_c⁶²¹⁶_c⁶²¹⁷_r⁶²¹⁸_i⁶²¹⁹_v⁶²²⁰_e⁶²²¹_r⁶²²²_n⁶²²³_v⁶²²⁴_v⁶²²⁵_r⁶²²⁶_a⁶²²⁷_
a⁶²²⁸ l⁶²²⁹_l⁶²³⁰_a⁶²³¹_g⁶²³²_s⁶²³³_f⁶²³⁴_d⁶²³⁵_k⁶²³⁶_v⁶²³⁷_y⁶²³⁸_d⁶²³⁹_i⁶²⁴⁰_g⁶²⁴¹_n⁶²⁴²_p⁶²⁴³_k⁶²⁴⁴
(SEQ ID NO: 159)
k⁶²³⁶_v⁶²³⁷_y⁶²³⁸_d⁶²³⁹_i⁶²⁴⁰_g⁶²⁴¹_n⁶²⁴²_p⁶²⁴³_k⁶²⁴⁴_g⁶²⁴⁵_i⁶²⁴⁶_p⁶²⁴⁷_i⁶²⁴⁸_v⁶²⁴⁹_d⁶²⁵⁰_d⁶²⁵¹_p⁶²⁵²_v⁶²⁵³_v⁶²⁵⁴
d⁶²⁵⁵_w⁶²⁵⁶_h⁶²⁵⁷
(SEQ ID NO: 160)
k⁶⁴⁹²_r⁶⁴⁹³_a⁶⁴⁹⁴_v⁶⁴⁹⁵_r⁶⁴⁹⁶_s⁶⁴⁹⁷_h⁶⁴⁹⁸_p⁶⁴⁹⁹_d⁶⁵⁰⁰_f⁶⁵⁰¹_k⁶⁵⁰²
(SEQ ID NO: 161)
k⁶⁴⁹²_r⁶⁴⁹³_a⁶⁴⁹⁴_v⁶⁴⁹⁵_r⁶⁴⁹⁶_s⁶⁴⁹⁷_h⁶⁴⁹⁸_p⁶⁴⁹⁹_d⁶⁵⁰⁰_f⁶⁵⁰¹_k⁶⁵⁰²_l⁶⁵⁰³_l⁶⁵⁰⁴_h⁶⁵⁰⁵
(SEQ ID NO: 162)
h⁶⁶⁷⁸_l⁶⁶⁷⁹_l⁶⁶⁸⁰_i⁶⁶⁸¹_g⁶⁶⁸²_l⁶⁶⁸³_y⁶⁶⁸⁴_k⁶⁶⁸⁵_k⁶⁶⁸⁶_q⁶⁶⁸⁷_q⁶⁶⁸⁸_e⁶⁶⁸⁹_g⁶⁶⁹⁰_h⁶⁶⁹¹_i⁶⁶⁹²_i⁶⁶⁹³_m⁶⁶⁹⁴_e⁶⁶⁹⁵_e⁶⁶⁹⁶
m⁶⁶⁹⁷_l⁶⁶⁹⁸_k⁶⁶⁹⁹_g⁶⁷⁰⁰_s⁶⁷⁰¹_s⁶⁷⁰²_t⁶⁷⁰³_i⁶⁷⁰⁴_h⁶⁷⁰⁵_n⁶⁷⁰⁶_y⁶⁷⁰⁷_f⁶⁷⁰⁸_i⁶⁷⁰⁹_t⁶⁷¹⁰_e⁶⁷¹¹_t⁶⁷¹²_n⁶⁷¹³_t⁶⁷¹⁴_a⁶⁷¹⁵_
a⁶⁷¹⁶ f⁶⁷¹⁷_k⁶⁷¹⁸_a⁶⁷¹⁹_v⁶⁷²⁰_c⁶⁷²¹_s⁶⁷²²_v⁶⁷²³_i⁶⁷²⁴_d⁶⁷²⁵_l⁶⁷²⁶_k⁶⁷²⁷
(SEQ ID NO: 163)
h⁶⁶⁹¹_i⁶⁶⁹²_i⁶⁶⁹³_m⁶⁶⁹⁴_e⁶⁶⁹⁵_e⁶⁶⁹⁶_m⁶⁶⁹⁷_l⁶⁶⁹⁸_k⁶⁶⁹⁹_g⁶⁷⁰⁰_s⁶⁷⁰¹_s⁶⁷⁰²_t⁶⁷⁰³_i⁶⁷⁰⁴_h⁶⁷⁰⁵_n⁶⁷⁰⁶_y⁶⁷⁰⁷_f⁶⁷⁰⁸_i⁶⁷⁰⁹_
t⁶⁷¹⁰ e⁶⁷¹¹_t⁶⁷¹²_n⁶⁷¹³_t⁶⁷¹⁴_a⁶⁷¹⁵_a⁶⁷¹⁶_f⁶⁷¹⁷_k⁶⁷¹⁸_a⁶⁷¹⁹_v⁶⁷²⁰_c⁶⁷²¹_s⁶⁷²²_v⁶⁷²³_i⁶⁷²⁴_d⁶⁷²⁵_l⁶⁷²⁶_k⁶⁷²⁷
(SEQ ID NO: 164)
h⁶⁶⁹¹_i⁶⁶⁹²_i⁶⁶⁹³_m⁶⁶⁹⁴_e⁶⁶⁹⁵_e⁶⁶⁹⁶_m⁶⁶⁹⁷_l⁶⁶⁹⁸_k⁶⁶⁹⁹_g⁶⁷⁰⁰_s⁶⁷⁰¹_s⁶⁷⁰²_t⁶⁷⁰³_i⁶⁷⁰⁴_h⁶⁷⁰⁵_n⁶⁷⁰⁶_y⁶⁷⁰⁷_f⁶⁷⁰⁸_i⁶⁷⁰⁹_
t⁶⁷¹⁰ e⁶⁷¹¹_t⁶⁷¹²_n⁶⁷¹³_t⁶⁷¹⁴_a⁶⁷¹⁵_a⁶⁷¹⁶_f⁶⁷¹⁷_k⁶⁷¹⁸_a⁶⁷¹⁹_v⁶⁷²⁰_c⁶⁷²¹_s⁶⁷²²_v⁶⁷²³_i⁶⁷²⁴_d⁶⁷²⁵_l⁶⁷²⁶_k⁶⁷²⁷_l⁶⁷²⁸_
d⁶⁷²⁹_d⁶⁷³⁰ f⁶⁷³¹_v⁶⁷³²_m⁶⁷³³_i⁶⁷³⁴_l⁶⁷³⁵_k⁶⁷³⁶
(SEQ ID NO: 165)
h⁶⁷⁰⁵_n⁶⁷⁰⁶_y⁶⁷⁰⁷_f⁶⁷⁰⁸_i⁶⁷⁰⁹_t⁶⁷¹⁰_e⁶⁷¹¹_t⁶⁷¹²_n⁶⁷¹³_t⁶⁷¹⁴_a⁶⁷¹⁵_a⁶⁷¹⁶_f⁶⁷¹⁷_k⁶⁷¹⁸_a⁶⁷¹⁹_v⁶⁷²⁰_c⁶⁷²¹_s⁶⁷²²_v⁶⁷²³_
i⁶⁷²⁴ d⁶⁷²⁵_l⁶⁷²⁶_k⁶⁷²⁷_l⁶⁷²⁸_d⁶⁷²⁹_d⁶⁷³⁰_f⁶⁷³¹_v⁶⁷³²_m⁶⁷³³_i⁶⁷³⁴_l⁶⁷³⁵_k⁶⁷³⁶
(SEQ ID NO: 166)
h⁶⁷⁰⁵_n⁶⁷⁰⁶_y⁶⁷⁰⁷_f⁶⁷⁰⁸_i⁶⁷⁰⁹_t⁶⁷¹⁰_e⁶⁷¹¹_t⁶⁷¹²_n⁶⁷¹³_t⁶⁷¹⁴_a⁶⁷¹⁵_a⁶⁷¹⁶_f⁶⁷¹⁷_k⁶⁷¹⁸_a⁶⁷¹⁹_v⁶⁷²⁰_c⁶⁷²¹_s⁶⁷²²_v⁶⁷²³_
i⁶⁷²⁴ d⁶⁷²⁵_l⁶⁷²⁶_k⁶⁷²⁷_l⁶⁷²⁸_d⁶⁷²⁹_d⁶⁷³⁰_f⁶⁷³¹_v⁶⁷³²_m⁶⁷³³_i⁶⁷³⁴_l⁶⁷³⁵_k⁶⁷³⁶_s⁶⁷³⁷_q⁶⁷³⁸_d⁶⁷³⁹_l⁶⁷⁴⁰_g⁶⁷⁴¹_v⁶⁷⁴²_
v⁶⁷⁴³_s⁶⁷⁴⁴ k⁶⁷⁴⁵
(SEQ ID NO: 167)
h⁶⁷⁰⁵_n⁶⁷⁰⁶_y⁶⁷⁰⁷_f⁶⁷⁰⁸_i⁶⁷⁰⁹_t⁶⁷¹⁰_e⁶⁷¹¹_t⁶⁷¹²_n⁶⁷¹³_t⁶⁷¹⁴_a⁶⁷¹⁵_a⁶⁷¹⁶_f⁶⁷¹⁷_k⁶⁷¹⁸_a⁶⁷¹⁹_v⁶⁷²⁰_c⁶⁷²¹_s⁶⁷²²_v⁶⁷²³_
i⁶⁷²⁴ d⁶⁷²⁵_l⁶⁷²⁶_k⁶⁷²⁷
(SEQ ID NO: 168)
s⁶⁷³⁷_q⁶⁷³⁸_d⁶⁷³⁹_l⁶⁷⁴⁰_g⁶⁷⁴¹_v⁶⁷⁴²_v⁶⁷⁴³_s⁶⁷⁴⁴_k⁶⁷⁴⁵_v⁶⁷⁴⁶_v⁶⁷⁴⁷_k⁶⁷⁴⁸_v⁶⁷⁴⁹_p⁶⁷⁵⁰_i⁶⁷⁵¹_d⁶⁷⁵²_l⁶⁷⁵³_t⁶⁷⁵⁴ m⁶⁷⁵⁵_
i⁶⁷⁵⁶_e⁶⁷⁵⁷_f⁶⁷⁵⁸_m⁶⁷⁵⁹_l⁶⁷⁶⁰_w⁶⁷⁶¹_c⁶⁷⁶²_k⁶⁷⁶³_d⁶⁷⁶⁴_g⁶⁷⁶⁵_q⁶⁷⁶⁶_v⁶⁷⁶⁷_q⁶⁷⁶⁸_t⁶⁷⁶⁹_f⁶⁷⁷⁰_y⁶⁷⁷¹_p⁶⁷⁷²_r⁶⁷⁷³ l⁶⁷⁷⁴_
q⁶⁷⁷⁵_a⁶⁷⁷⁶_s⁶⁷⁷⁷_a⁶⁷⁷⁸_d⁶⁷⁷⁹_w⁶⁷⁸⁰_k⁶⁷⁸¹_p⁶⁷⁸²_g⁶⁷⁸³_h⁶⁷⁸⁴
(SEQ ID NO: 21)
k⁶⁷⁸¹_p⁶⁷⁸²_g⁶⁷⁸³_h⁶⁷⁸⁴_a⁶⁷⁸⁵_m⁶⁷⁸⁶_p⁶⁷⁸⁷_s⁶⁷⁸⁸_l⁶⁷⁸⁹_f⁶⁷⁹⁰_k⁶⁷⁹¹
(SEQ ID NO: 169)
k⁶⁷⁸¹_p⁶⁷⁸²_g⁶⁷⁸³_h⁶⁷⁸⁴_a⁶⁷⁸⁵_m⁶⁷⁸⁶_p⁶⁷⁸⁷_s⁶⁷⁸⁸_l⁶⁷⁸⁹_f⁶⁷⁹⁰_k⁶⁷⁹¹_v⁶⁷⁹²_q⁶⁷⁹³_n⁶⁷⁹⁴_v⁶⁷⁹⁵_n⁶⁷⁹⁶_l⁶⁷⁹⁷_e⁶⁷⁹⁸_r⁶⁷⁹⁹
c⁶⁸⁰⁰_e⁶⁸⁰¹_l⁶⁸⁰²_a⁶⁸⁰³_n⁶⁸⁰⁴_y⁶⁸⁰⁵_k⁶⁸⁰⁶_q⁶⁸⁰⁷_s⁶⁸⁰⁸_i⁶⁸⁰⁹_p⁶⁸¹⁰_m⁶⁸¹¹_p⁶⁸¹²_r⁶⁸¹³_g⁶⁸¹⁴_v⁶⁸¹⁵_h⁶⁸¹⁶
(SEQ ID NO: 170)
k⁶⁹¹²_n⁶⁹¹³_v⁶⁹¹⁴_t⁶⁹¹⁵_g⁶⁹¹⁶_s⁶⁹¹⁷_n⁶⁹¹⁸_e⁶⁹¹⁹_s⁶⁹²⁰_k⁶⁹²¹_a⁶⁹²²_l⁶⁹²³_f⁶⁹²⁴_f⁶⁹²⁵_t⁶⁹²⁶_y⁶⁹²⁷_l⁶⁹²⁸_c⁶⁹²⁹_n⁶⁹³⁰_
l⁶⁹³¹ i⁶⁹³²_n⁶⁹³³_n⁶⁹³⁴_n⁶⁹³⁵_l⁶⁹³⁶_a⁶⁹³⁷_l⁶⁹³⁸_g⁶⁹³⁹_g⁶⁹⁴⁰_s⁶⁹⁴¹_v⁶⁹⁴²_a⁶⁹⁴³_i⁶⁹⁴⁴_k⁶⁹⁴⁵_i⁶⁹⁴⁶_t⁶⁹⁴⁷_e⁶⁹⁴⁸_h⁶⁹⁴⁹
(SEQ ID NO: 171)
h⁷⁰⁰⁰_a⁷⁰⁰¹_n⁷⁰⁰²_y⁷⁰⁰³_i⁷⁰⁰⁴_f⁷⁰⁰⁵_w⁷⁰⁰⁶_r⁷⁰⁰⁷_n⁷⁰⁰⁸_s⁷⁰⁰⁹_t⁷⁰¹⁰_p⁷⁰¹¹_m⁷⁰¹²_n⁷⁰¹³_l⁷⁰¹⁴_s⁷⁰¹⁵_t⁷⁰¹⁶_y⁷⁰¹⁷_s⁷⁰¹⁸_
l⁷⁰¹⁹ f⁷⁰²⁰_d⁷⁰²¹_l⁷⁰²²_s⁷⁰²³_k⁷⁰²⁴_f⁷⁰²⁵_q⁷⁰²⁶_l⁷⁰²⁷_k⁷⁰²⁸_l⁷⁰²⁹_k⁷⁰³⁰
(SEQ ID NO: 172)
h⁷⁰⁰⁰_a⁷⁰⁰¹_n⁷⁰⁰²_y⁷⁰⁰³_i⁷⁰⁰⁴_f⁷⁰⁰⁵_w⁷⁰⁰⁶_r⁷⁰⁰⁷_n⁷⁰⁰⁸_s⁷⁰⁰⁹_t⁷⁰¹⁰_p⁷⁰¹¹_m⁷⁰¹²_n⁷⁰¹³_l⁷⁰¹⁴_s⁷⁰¹⁵_t⁷⁰¹⁶_y⁷⁰¹⁷_s⁷⁰¹⁸_
l⁷⁰¹⁹ f⁷⁰²⁰_d⁷⁰²¹_l⁷⁰²²_s⁷⁰²³_k⁷⁰²⁴_f⁷⁰²⁵_q⁷⁰²⁶_l⁷⁰²⁷_k⁷⁰²⁸_l⁷⁰²⁹_k⁷⁰³⁰_g⁷⁰³¹_t⁷⁰³²_p⁷⁰³³_v⁷⁰³⁴_l⁷⁰³⁵_q⁷⁰³⁶_l⁷⁰³⁷_
k⁷⁰³⁸
Replikin Count in AHX71944 is the number of Replikin sequences per 100 amino acid
residues, which is 162/7078 and equals 2.3.
Sequence History by Year for Replikin Sequences Identified in Accession No.
AHX71944
(SEQ ID NO: 10)
KSVVRHLGVTK
All occurrences of the sequence by year:
2012
YP_007188577 position 2094, YP 007188578 position 2094, AGV08378 position 2094,
AGV08377 position 2094, AGV08583 position 2094, AGV08582 position 2094,
AGV08407 position 2094, AGV08406 position 2094, AFY13306 position 2094,
AFY13305 position 2094, AGG22541 position 2094, AGG22540 position 2094,
AGH58716 position 2094, AGH58715 position 2094, AFV09327 position 2094,
AFS88944 position 2094.
2013
AHY22564 position 2094, AHY22563 position 2094, AHY22554 position 2094,
AHY22553 position 2094, AHY22544 position 2094, AHY22543 position 2094,
AHY22534 position 2094, AHY22533 position 2094, AHY22524 position 2094,
AHY22523 position 2094, AHE78107 position 2094, AHE78106 position 2094,
AHE78096 position 2094, AHE78095 position 2094, AHX00730 position 2094,
AHX00729 position 2094, AHX00720 position 2094, AHX00719 position 2094,
AHX00710 position 2094, AHX00709 position 2094, K9N638 position 2094,
K9N7C7 position 2094, AHN10811 position 2094, AHN10810 position 2094,
AHI48798 position 1460, AHI48796 position 799, AHI48795 position 785,
AHI48794 position 1460, AHI48792 position 1460, AHI48789 position 1415,
AHI48787 position 764, AHI48786 position 133, AHI48784 position 1460,
AHI48777 position 1451, AHI48776 position 1460, AHI48769 position 2094,
AHI48768 position 2094, AHI48767 position 2094, AHI48766 position 2094,
AHI48765 position 2094, AHI48764 position 2094, AHI48763 position 2094,
AHI48762 position 2094, AHI48761 position 2094, AHI48760 position 2094,
AHI48759 position 2094, AHI48758 position 2094, AHI48757 position 2094,
AHI48756 position 2094, AHI48755 position 2094, AHI48754 position 2094,
AHI48625 position 1361, AHI48624 position 1361, AHI48615 position 1475,
AHI48614 position 1475, AHI48604 position 2094, AHI48603 position 2094,
AHI48593 position 2094, AHI48592 position 2094, AHI48582 position 2094,
AHI48581 position 2094, AHI48571 position 2094, AHI48570 position 2094,
AHI48560 position 2094, AHI48559 position 2094, AHI48549 position 2094,
AHI48548 position 2094, AHI48538 position 2094, AHI48537 position 2094,
AHI48527 position 2094, AHI48526 position 2094, AHI48516 position 2094,
AHI48515 position 2094, AHL18091 position 2094, AHL18089 position 2094,
AHC74096 position 2094, AHC74086 position 2094, AGV08596 position 2094,
AGV08595 position 2094, AGV08572 position 2094, AGV08571 position 2094,
AGV08557 position 2094, AGV08556 position 2094, AGV08555 position 681,
AGV08545 position 2094, AGV08544 position 2094, AGV08534 position 2094,
AGV08533 position 2094, AGV08522 position 767, AGV08515 position 136,
AGV08491 position 2094, AGV08490 position 2094, AGV08489 position 339,
AGV08479 position 2094, AGV08478 position 2094, AGV08466 position 2094,
AGV08465 position 2094, AGV08454 position 2094, AGV08453 position 2094,
AGV08443 position 2094, AGV08442 position 2094, AGV08402 position 682,
AGV08401 position 138, AGV08389 position 2094, AGV08388 position 2094,
AGN72639 position 2094, AGN72638 position 2094, AGN70972 position 2094,
AGN70971 position 2094, AGN70961 position 2094, AGN70960 position 2094,
AGN70950 position 2094, AGN70949 position 2094, AGN70928 position 2094,
AGN70927 position 2094.
2014
AHY21468 position 2094, AHY21467 position 2094, AHX71945 position 2094,
AHX71944 position 2094.
(SEQ ID NO: 11)
KFYQHVINGCK
All occurrences of the sequence by year:
2012
YP_007188577 position 2364, YP_007188578 position 2364, AGV08378 position 2364,
AGV08377 position 2364, AGV08583 position 2364, AGV08582 position 2364,
AGV08407 position 2364, AGV08406 position 2364, AFY13306 position 2364,
AFY13305 position 2364, AGG22541 position 2364, AGG22540 position 2364,
AGH58716 position 2364, AGH58715 position 2364, AFV09327 position 2364,
AFS88944 position 2364.
2013
AHY22564 position 2364, AHY22563 position 2364, AHY22554 position 2364,
AHY22553 position 2364, AHY22544 position 2364, AHY22543 position 2364,
AHY22534 position 2364, AHY22533 position 2364, AHY22524 position 2364,
AHY22523 position 2364, AHE78107 position 2364, AHE78106 position 2364,
AHE78096 position 2364, AHE78095 position 2364, AHX00730 position 2364,
AHX00729 position 2364, AHX00720 position 2364, AHX00719 position 2364,
AHX00710 position 2364, AHX00709 position 2364, K9N638 position 2364,
K9N7C7 position 2364, AHN10811 position 2364, AHN10810 position 2364,
AHI48798 position 1730, AHI48796 position 1069, AHI48795 position 1055,
AHI48794 position 1730, AHI48792 position 1730, AHI48789 position 1685,
AHI48786 position 403, AHI48784 position 1730, AHI48776 position 1730,
AHI48769 position 2364, AHI48768 position 2364, AHI48767 position 2364,
AHI48766 position 2364, AHI48765 position 2364, AHI48764 position 2364,
AHI48763 position 2364, AHI48762 position 2364, AHI48761 position 2364,
AHI48760 position 2364, AHI48759 position 2364, AHI48758 position 2364,
AHI48757 position 2364, AHI48756 position 2364, AHI48755 position 2364,
AHI48754 position 2364, AHI48625 position 1631, AHI48624 position 1631,
AHI48615 position 1745, AHI48614 position 1745, AHI48604 position 2364,
AHI48603 position 2364, AHI48593 position 2364, AHI48592 position 2364,
AHI48582 position 2364, AHI48581 position 2364, AHI48571 position 2364,
AHI48570 position 2364, AHI48560 position 2364, AHI48559 position 2364,
AHI48549 position 2364, AHI48548 position 2364, AHI48538 position 2364,
AHI48537 position 2364, AHI48527 position 2364, AHI48526 position 2364,
AHI48516 position 2364, AHI48515 position 2364, AHL18091 position 2364,
AHL18089 position 2364, AHC74096 position 2364, AHC74086 position 2364,
AHC74080 position 221, AHB33325 position 2364, AHB33324 position 2364,
AGV08596 position 2364, AGV08595 position 2364, AGV08572 position 2364,
AGV08571 position 2364, AGV08557 position 2364, AGV08556 position 2364,
AGV08555 position 951, AGV08545 position 2364, AGV08544 position 2364,
AGV08534 position 2364, AGV08533 position 2364, AGV08522 position 1037,
AGV08515 position 406, AGV08491 position 2364, AGV08490 position 2364,
AGV08489 position 609, AGV08479 position 2364, AGV08478 position 2364,
AGV08466 position 2364, AGV08465 position 2364, AGV08454 position 2364,
AGV08453 position 2364, AGV08443 position 2364, AGV08442 position 2364,
AGV08402 position 952, AGV08401 position 408, AGV08389 position 2364,
AGV08388 position 2364, AGN72639 position 2364, AGN72638 position 2364,
AGN70972 position 2364, AGN70971 position 2364, AGN70961 position 2364,
AGN70960 position 2364, AGN70950 position 2364, AGN70949 position 2364,
AGN70928 position 2364, AGN70927 position 2364.
2014
AHY21468 position 2364, AHY21467 position 2364, AHX71945 position 2364,
AHX71944 position 2364.
(SEQ ID NO: 6)
KHVEVFTDGK
All occurrences of the sequence by year:
2012
YP 007188577 position 3147, YP 007188578 position 3147, AGV08378 position 3147,
AGV08377 position 3147, AGV08583 position 3147, AGV08582 position 3147,
AGV08407 position 3147, AGV08406 position 3147, AFY13306 position 3147,
AFY13305 position 3147, AGG22541 position 3147, AGG22540 position 3147,
AGH58716 position 3147, AGH58715 position 3147, AFV09327 position 3147,
AFS88944 position 3147.
2013
AHY22564 position 3147, AHY22563 position 3147, AHY22554 position 3147,
AHY22553 position 3147, AHY22544 position 3147, AHY22543 position 3147,
AHY22534 position 3147, AHY22533 position 3147, AHY22524 position 3147,
AHY22523 position 3147, AHE78107 position 3147, AHE78106 position 3147,
AHE78096 position 3147, AHE78095 position 3147, AHX00730 position 3147,
AHX00729 position 3147, AHX00720 position 3147, AHX00719 position 3147,
AHX00710 position 3147, AHX00709 position 3147, K9N638 position 3147,
K9N7C7 position 3147, AHN10811 position 3147, AHN10810 position 3147,
AHI48798 position 2513, AHI48796 position 1852, AHI48795 position 1838,
AHI48794 position 2513, AHI48790 position 480, AHI48788 position 700,
AHI48780 position 449, AHI48776 position 2513, AHI48769 position 3147,
AHI48768 position 3147, AHI48767 position 3147, AHI48766 position 3147,
AHI48765 position 3147, AHI48764 position 3147, AHI48763 position 3147,
AHI48762 position 3147, AHI48761 position 3147, AHI48760 position 3147,
AHI48759 position 3147, AHI48758 position 3147, AHI48757 position 3147,
AHI48756 position 3147, AHI48755 position 3147, AHI48754 position 3147,
AHI48747 position 337, AHI48746 position 337, AHI48625 position 2414,
AHI48624 position 2414, AHI48615 position 2528, AHI48614 position 2528,
AHI48604 position 3147, AHI48603 position 3147, AHI48593 position 3147,
AHI48592 position 3147, AHI48582 position 3147, AHI48581 position 3147,
AHI48571 position 3147, AHI48570 position 3147, AHI48560 position 3147,
AHI48559 position 3147, AHI48549 position 3147, AHI48548 position 3147,
AHI48538 position 3147, AHI48537 position 3147, AHI48527 position 3147,
AHI48526 position 3147, AHI48516 position 3147, AHI48515 position 3147,
AHL18091 position 3147, AHL18089 position 3147, AHC74096 position 3147,
AHC74086 position 3147, AHB33325 position 3147, AHB33324 position 3147,
AGV08596 position 3147, AGV08595 position 3147, AGV08572 position 3147,
AGV08571 position 3147, AGV08557 position 3147, AGV08556 position 3147,
AGV08555 position 1734, AGV08545 position 3147, AGV08544 position 3147,
AGV08534 position 3147, AGV08533 position 3147, AGV08515 position 1189,
AGV08491 position 3147, AGV08490 position 3147, AGV08479 position 3147,
AGV08478 position 3147, AGV08466 position 3147, AGV08465 position 3147,
AGV08454 position 3147, AGV08453 position 3147, AGV08443 position 3147,
AGV08442 position 3147, AGV08401 position 1191, AGV08389 position 3147,
AGV08388 position 3147, AGN72639 position 3147, AGN72638 position 3147,
AGN70972 position 3147, AGN70971 position 3147, AGN70961 position 3147,
AGN70960 position 3147, AGN70950 position 3147, AGN70949 position 3147,
AGN70928 position 3147, AGN70927 position 3147.
2014
AHY21468 position 3147, AHY21467 position 3147, AHX71945 position 3147,
AHX71944 position 3147.

### Example 5

### Analysis of Accession No. AFY13314 for Replikin Sequences Conserved in Human Host and Shared with Isolates in Camel Hosts

PubMed Code:
PubMed Code:
AFY13314
Description:
Direct Submission Direct Submission
Isolated:
2012 in United Kingdom
Source:
Betacoronavirus England 1
Strain:
England 1
(SEQ ID NO: 173)
m¹ a² s³ p⁴ a⁵ a⁶ p⁷ r⁸ a⁹ v¹⁰ s¹¹ l⁴² a¹³ d¹⁴ n¹⁵ n¹⁶ d¹⁷ i¹⁸ t¹⁹ n²⁰ t²¹ n²² l²³ s²⁴ r²⁵ g²⁶ r²⁷ g²⁸ r²⁹
n³⁰ p³¹ k³² p³³ r³⁴ a³⁵ a³⁶ p³⁷ n³⁸ n³⁹ t⁴⁰ v⁴¹ s⁴² w⁴³ y⁴⁴ t⁴⁵ g⁴⁶ l⁴⁷ t⁴⁸ q⁴⁹ h⁵⁰ g⁵¹ k⁵² v⁵³ p⁵⁴ l⁵⁵ t⁵⁶
f⁵⁷ p⁵⁸ p⁵⁹ g⁶⁰ q⁶¹ g⁶² v⁶³ p⁶⁴ l⁶⁵ n⁶⁶ a⁶⁷ n⁶⁸ s⁶⁹ t⁷⁰ p⁷¹ a⁷² q⁷³ n⁷⁴ a⁷⁵ g⁷⁶ y⁷⁷ w⁷⁸ r⁷⁹ r⁸⁰ q⁸¹ d⁸² r⁸³
k⁸⁴ i⁸⁵ n⁸⁶ t⁸⁷ g⁸⁸ n⁸⁹ g⁹⁰ i⁹¹ k⁹² q⁹³ l⁹⁴ a⁹⁵ p⁹⁶ r⁹⁷ w⁹⁸ y⁹⁹ f¹⁰⁰ y¹⁰¹ y¹⁰² t¹⁰³ g¹⁰⁴ t¹⁰⁵ g¹⁰⁶ p¹⁰⁷ e¹⁰⁸
a¹⁰⁹ a¹¹⁰ l¹¹¹ p¹¹² f¹¹³ r¹¹⁴ a¹¹⁵ v¹¹⁶ k¹¹⁷ d¹¹⁸ g¹¹⁹ l¹²⁰ v¹²¹ w¹²² v¹²³ h¹²⁴ e¹²⁵ d¹²⁶ g¹²⁷ a¹²⁸ t¹²⁹
d¹³⁰ a¹³¹ p¹³² s¹³³ t¹³⁴ l¹³⁵ g¹³⁶ t¹³⁷ r¹³⁸ a¹³⁹ p¹⁴⁰ a¹⁴¹ a¹⁴² d¹⁴³ s¹⁴⁴ a¹⁴⁵ i¹⁴⁶ v¹⁴⁷ t¹⁴⁸ q¹⁴⁹ l⁴⁵⁰ a¹⁵¹ p¹⁵²
g¹⁵³ t¹⁵⁴ k¹⁵⁵ l¹⁵⁶ p¹⁵⁷ k¹⁵⁸ p¹⁵⁹ l⁴⁶⁰ h¹⁶¹ i¹⁶² e¹⁶³ g¹⁶⁴ t¹⁶⁵ g¹⁶⁶ g¹⁶⁷ a¹⁶⁸ s¹⁶⁹ q¹⁷⁰ s¹⁷¹ s¹⁷² s¹⁷³ r¹⁷⁴ a¹⁷⁵
s¹⁷⁶ s¹⁷⁷ v¹⁷⁸ s¹⁷⁹ r¹⁸⁰ a¹⁸¹ s¹⁸² s¹⁸³ r¹⁸⁴ s¹⁸⁵ s¹⁸⁶ s¹⁸⁷ q¹⁸⁸ g¹⁸⁹ s¹⁹⁰ r¹⁹¹ s¹⁹² g¹⁹³ a¹⁹⁴ s¹⁹⁵ t¹⁹⁶ r¹⁹⁷ g¹⁹⁸
t¹⁹⁹ s²⁰⁰ p²⁰¹ g²⁰² p²⁰³ s²⁰⁴ g²⁰⁵ i²⁰⁶ g²⁰⁷ a²⁰⁸ v²⁰⁹ g²¹⁰ g²¹¹ d²¹² l²¹³ l²¹⁴ y²¹⁵ l²¹⁶ d²¹⁷ l²¹⁸ l²¹⁹ a²²⁰ r²²¹
l²²² q²²³ a²²⁴ l²²⁵ e²²⁶ s²²⁷ g²²⁸ k²²⁹ e²³⁰ k²³¹ q²³² s²³³ q²³⁴ p²³⁵ k²³⁶ v²³⁷ i²³⁸ t²³⁹ k²⁴⁰ k²⁴¹ d²⁴² a²⁴³ a²⁴⁴
a²⁴⁵ a²⁴⁶ k²⁴⁷ a²⁴⁸ k²⁴⁹ m²⁵⁰ r²⁵¹ h²⁵² k²⁵³ r²⁵⁴ t²⁵⁵ s²⁵⁶ t²⁵⁷ k²⁵⁸ s²⁵⁹ f²⁶⁰ a²⁶¹ m²⁶² v²⁶³ q²⁶⁴ a²⁶⁵ f²⁶⁶ g²⁶⁷
l²⁶⁸ r²⁶⁹ g²⁷⁰ p²⁷¹ g²⁷² d²⁷³ l²⁷⁴ q²⁷⁵ g²⁷⁶ a²⁷⁷ f²⁷⁸ g²⁷⁹ d²⁸⁰ l²⁸¹ q²⁸² l²⁸³ a²⁸⁴ k²⁸⁵ l²⁸⁶ g²⁸⁷ t²⁸⁸ e²⁸⁹ d²⁹⁰
p²⁹¹ r²⁹² w²⁹³ p²⁹⁴ q²⁹⁵ i²⁹⁶ a²⁹⁷ e²⁹⁸ l²⁹⁹ a³⁰⁰ p³⁰¹ t³⁰² a³⁰³ s³⁰⁴ a³⁰⁵ f³⁰⁶ m³⁰⁷ g³⁰⁸ m³⁰⁹ s³¹⁰ q³¹¹ f³¹² k³¹³
l³¹⁴ t³¹⁵ h³¹⁶ q³¹⁷ a³¹⁸ a³¹⁹ d³²⁰ d³²¹ h³²² g³²³ a³²⁴ p³²⁵ v³²⁶ y³²⁷ f³²⁸ l³²⁹ r³³⁰ y³³¹ s³³² g³³³ a³³⁴ i³³⁵ k³³⁶
l³³⁷ d³³⁸ p³³⁹ k³⁴⁰ p³⁴¹ p³⁴² a³⁴³ y³⁴⁴ p³⁴⁵ k³⁴⁶ w³⁴⁷ l³⁴⁸ e³⁴⁹ l³⁵⁰ l³⁵¹ e³⁵² q³⁵³ a³⁵⁴ i³⁵⁵ d³⁵⁶ a³⁵⁷ y³⁵⁸ k³⁵⁹
t³⁶⁰ f³⁶¹ p³⁶² k³⁶³ k³⁶⁴ e³⁶⁵ k³⁶⁶ k³⁶⁷ q³⁶⁸ k³⁶⁹ a³⁷⁰ p³⁷¹ k³⁷² e³⁷³ e³⁷⁴ s³⁷⁵ t³⁷⁶ d³⁷⁷ q³⁷⁸ m³⁷⁹ s³⁸⁰ e³⁸¹ p³⁸²
p³⁸³ k³⁸⁴ e³⁸⁵ h³⁸⁶ r³⁸⁷ v³⁸⁸ q³⁸⁹ g³⁹⁰ t³⁹¹ q³⁹² r³⁹³ t³⁹⁴ r³⁹⁵ t³⁹⁶ r³⁹⁷ p³⁹⁸ s³⁹⁹ v⁴⁰⁰ q⁴⁰¹ p⁴⁰² g⁴⁰³ p⁴⁰⁴ m⁴⁰⁵
i⁴⁰⁶ d⁴⁰⁷ v⁴⁰⁸ a⁴⁰⁹ t⁴¹⁰ d⁴¹¹
Amino-terminal
(SEQ ID NO: 174)
h⁵⁰_g⁵¹_k⁵²_v⁵³_p⁵⁴_l⁵⁵_t⁵⁶_f⁵⁷_p⁵⁸_p⁵⁹_g⁶⁰_q⁶¹_g⁶²_v⁶³_p⁶⁴_l⁶⁵_n⁶⁶_a⁶⁷_n⁶⁸_s⁶⁹_t⁷⁰_p⁷¹_a⁷²_q⁷³_n⁷⁴_a⁷⁵_g⁷⁶_y⁷⁷
w⁷⁸_r⁷⁹_r⁸⁰_q⁸¹_d⁸²_r⁸³_k⁸⁴_i⁸⁵_n⁸⁶_t⁸⁷_g⁸⁸_n⁸⁹_g⁹⁰_i⁹¹_k⁹²
(SEQ ID NO: 175)
k⁸⁴_i⁸⁵_n⁸⁶_t⁸⁷_g⁸⁸_n⁸⁹_g⁹⁰_i⁹¹_k⁹² q⁹³ l⁹⁴_a⁹⁵_p⁹⁶_r⁹⁷_w⁹⁸_y⁹⁹_f¹⁰⁰_y¹⁰¹_y¹⁰²_t¹⁰³_g¹⁰⁴_t¹⁰⁵_g¹⁰⁶_p¹⁰⁷_e¹⁰⁸_a¹⁰⁹
a¹¹⁰_l¹¹¹_p¹¹²_f¹¹³_r¹¹⁴_a¹¹⁵_v¹¹⁶ k¹¹⁷_d¹¹⁸_g¹¹⁹_i¹²⁰_v¹²¹_w¹²²_v¹²³_h¹²⁴
Mid-molecule
(SEQ ID NO: 176)
k²²⁹_v²³⁰_k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹
h²⁵²
(SEQ ID NO: 177)
k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 196)
k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 24)
k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 22)
k²⁴⁷_n²⁴⁸_k²⁴⁹_m²⁵⁰_r²⁵¹_h²⁵²_k²⁵³
(SEQ ID NO: 25)
k²⁴⁹_m²⁵⁰_r²⁵¹_h²⁵²_k²⁵³_r²⁵⁴_t²⁵⁵_s²⁵⁶_t²⁵⁷_k²⁵⁸
Carboxy-terminal
(SEQ ID NO: 178)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p³⁴²_n³⁴³_y³⁴⁴_n³⁴⁵_k³⁴⁶
(SEQ ID NO: 179)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹
p³⁶²_k³⁶³_k³⁶⁴_e³⁶⁵_k³⁶⁶
(SEQ ID NO: 180)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶
(SEQ ID NO: 181)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰ l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶
(SEQ ID NO: 182)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰ l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
(SEQ ID NO: 183)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰ l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
g³⁶⁸_k³⁶⁹
(SEQ ID NO: 184)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰ l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
g³⁶⁸_k³⁶⁹ a³⁷⁰_p³⁷¹_k³⁷²
(SEQ ID NO: 185)
k³³⁶_l³³⁷_d³³⁸_p³³⁹_k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸
k³⁵⁹_t³⁶⁰_f³⁶¹_p³⁶²_k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹
p³⁸²_p³⁸³_k³⁸⁴_e³⁸⁵_h³⁸⁶
(SEQ ID NO: 186)
k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²
k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵
h³⁸⁶
(SEQ ID NO: 187)
k³⁵⁹_t³⁶⁰_f³⁶¹_p³⁶²_k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹
p³⁸²_p³⁸³_k³⁸⁴_e³⁸⁵_h³⁸⁶
(SEQ ID NO: 188)
k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵
h³⁸⁶
(SEQ ID NO: 189)
k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵_h³⁸⁶
(SEQ ID NO: 190)
k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵_h³⁸⁶
Replikin Count in AFY13314 is the number of Replikin sequences per 100 amino acid
residues, which is 21/411 and equals 5.1.
Sequence History by Year for Replikin Sequences Identified in Accession No.
AFY13314
(SEQ ID NO: 12)
KQVHQVQLTDK
All occurrences of the sequence by year:
2012
YP_007188577 position 3438, YP_007188578 position 3438, AGV08378 position 3438,
AGV08377 position 3438, AGV08583 position 3438, AGV08582 position 3438,
AGV08407 position 3438, AGV08406 position 3438, AFY13306 position 3438,
AFY13305 position 3438, AGG22541 position 3438, AGG22540 position 3438,
AGH58716 position 3438, AGH58715 position 3438, AFV09327 position 3438,
AFS 88944 position 3438.
2013
AHY22564 position 3438, AHY22563 position 3438, AHY22554 position 3438,
AHY22553 position 3438, AHY22544 position 3438, AHY22543 position 3438,
AHY22534 position 3438, AHY22533 position 3438, AHY22524 position 3438,
AHY22523 position 3438, AHE78107 position 3438, AHE78106 position 3438,
AHE78096 position 3438, AHE78095 position 3438, AHX00730 position 3438,
AHX00729 position 3438, AHX00720 position 3438, AHX00719 position 3438,
AHX00710 position 3438, AHX00709 position 3438, K9N638 position 3438, K9N7C7
position 3438, AHN10811 position 3438, AHN10810 position 3438, AHI48798 position
2804, AHI48795 position 2129, AHI48794 position 2804, AHI48776 position 2804,
AHI48769 position 3438, AHI48768 position 3438, AHI48767 position 3438, AHI48766
position 3438, AHI48765 position 3438, AHI48764 position 3438, AHI48763 position
3438, AHI48762 position 3438, AHI48761 position 3438, AHI48760 position 3438,
AHI48759 position 3438, AHI48758 position 3438, AHI48757 position 3438, AHI48756
position 3438, AHI48755 position 3438, AHI48754 position 3438, AHI48749 position 117,
AHI48748 position 117, AHI48747 position 628, AHI48746 position 628, AHI48745
position 219, AHI48744 position 219, AHI48625 position 2705, AHI48624 position 2705,
AHI48615 position 2819, AHI48614 position 2819, AHI48604 position 3438, AHI48603
position 3438, AHI48593 position 3438, AHI48592 position 3438, AHI48582 position
3438, AHI48581 position 3438, AHI48571 position 3438, AHI48570 position 3438,
AHI48560 position 3438, AHI48559 position 3438, AHI48549 position 3438, AHI48548
position 3438, AHI48538 position 3438, AHI48537 position 3438, AHI48527 position
3438, AHI48526 position 3438, AHI48516 position 3438, AHI48515 position 3438,
AHL18091 position 3438, AHL18089 position 3438, AHC74096 position 3438,
AHC74086 position 3438, AHC74081 position 173, AHB33325 position 3438, AHB33324
position 3438, AGV08596 position 3438, AGV08595 position 3438, AGV08594 position
116, AGV08572 position 3438, AGV08571 position 3438, AGV08557 position 3438,
AGV08556 position 3438, AGV08555 position 2025, AGV08545 position 3438,
AGV08544 position 3438, AGV08534 position 3438, AGV08533 position 3438,
AGV08515 position 1480, AGV08491 position 3438, AGV08490 position 3438,
AGV08479 position 3438, AGV08478 position 3438, AGV08466 position 3438,
AGV08465 position 3438, AGV08454 position 3438, AGV08453 position 3438,
AGV08443 position 3438, AGV08442 position 3438, AGV08401 position 1482,
AGV08389 position 3438, AGV08388 position 3438, AGN72639 position 3438,
AGN72638 position 3438, AGN70972 position 3438, AGN70971 position 3438,
AGN70961 position 3438, AGN70960 position 3438, AGN70950 position 3438,
AGN70949 position 3438, AGN70928 position 3438, AGN70927 position 3438.
2014
AHY21468 position 3438, AHY21467 position 3438, AHX71945 position 3438,
AHX71944 position 3438.
(SEQ ID NO: 13)
KGDSCSSNCKH
All occurrences of the sequence by year:
2012
YP_007188577 position 390, YP_007188578 position 390, AGV08378 position 390,
AGV08377 position 390, AGV08583 position 390, AGV08582 position 390, AGV08407
position 390, AGV08406 position 390, AFY13306 position 390, AFY13305 position 390,
AGG22541 position 390, AGG22540 position 390, AGH58716 position 390, AGH58715
position 390, AFV09327 position 390, AFS88944 position 390.
2013
AHY22564 position 390, AHY22563 position 390, AHY22554 position 390, AHY22553
position 390, AHY22544 position 390, AHY22543 position 390, AHY22534 position 390,
AHY22533 position 390, AHY22524 position 390, AHY22523 position 390, AHE78107
position 390, AHE78106 position 390, AHE78096 position 390, AHE78095 position 390,
AHX00730 position 390, AHX00729 position 390, AHX00720 position 390, AHX00719
position 390, AHX00710 position 390, AHX00709 position 390, K9N638 position 390,
K9N7C7 position 390, AHN10811 position 390, AHN10810 position 390, AHI48797
position 390, AHI48793 position 390, AHI48791 position 390, AHI48785 position 105,
AHI48783 position 390, AHI48782 position 390, AHI48778 position 390, AHI48769
position 390, AHI48768 position 390, AHI48767 position 390, AHI48766 position 390,
AHI48765 position 390, AHI48764 position 390, AHI48763 position 390, AHI48762
position 390, AHI48761 position 390, AHI48760 position 390, AHI48759 position 390,
AHI48758 position 390, AHI48757 position 390, AHI48756 position 390, AHI48755
position 390, AHI48754 position 390, AHI48604 position 390, AHI48603 position 390,
AHI48593 position 390, AHI48592 position 390, AHI48582 position 390, AHI48581
position 390, AHI48571 position 390, AHI48570 position 390, AHI48560 position 390,
AHI48559 position 390, AHI48549 position 390, AHI48548 position 390, AHI48538
position 390, AHI48537 position 390, AHI48527 position 390, AHI48526 position 390,
AHI48516 position 390, AHI48515 position 390, AHL18091 position 390, AHL18089
position 390, AHC74096 position 390, AHC74086 position 390, AHB33325 position 390,
AHB33324 position 390, AGV08596 position 390, AGV08595 position 390, AGV08593
position 161, AGV08572 position 390, AGV08571 position 390, AGV08567 position 390,
AGV08557 position 390, AGV08556 position 390, AGV08545 position 390, AGV08544
position 390, AGV08534 position 390, AGV08533 position 390, AGV08491 position 390,
AGV08490 position 390, AGV08479 position 390, AGV08478 position 390, AGV08466
position 390, AGV08465 position 390, AGV08464 position 390, AGV08454 position 390,
AGV08453 position 390, AGV08443 position 390, AGV08442 position 390, AGV08403
position 390, AGV08389 position 390, AGV08388 position 390, AGN72639 position 390,
AGN72638 position 390, AGN70972 position 390, AGN70971 position 390, AGN70961
position 390, AGN70960 position 390, AGN70950 position 390, AGN70949 position 390,
AGN70928 position 390, AGN70927 position 390.
2014
AHY21468 position 390, AHY21467 position 390, AHX71945 position 390, AHX71944
position 390.

### Example 6

### Analysis of Accession No. YP_007188586 for Replikin Sequences Conserved in Human Host and Shared with Isolates in Camel Hosts

PubMed Code:
YP_007188586
Description:
Direct Submission Direct Submission Direct Submission
Isolated:
2012 in United Kingdom
Source:
Middle East respiratory syndrome coronavirus (MERS-CoV)
Strain:
England 1
(SEQ ID NO: 191)
m¹ a² s³ p⁴ a⁵ a⁶ p⁷ r⁸ a⁹ v¹⁰ s¹¹ f¹² a¹³ d¹⁴ n¹⁵ n¹⁶ d¹⁷ i¹⁸ t¹⁹ n²⁰ t²¹ n²² l²³ s²⁴ r²⁵ g²⁶ r²⁷ g²⁸ r²⁹ n³⁰ p³¹
k³² p³³ r³⁴ a³⁵ a³⁶ p³⁷ n³⁸ n³⁹ t⁴⁰ v⁴¹ s⁴² w⁴³ y⁴⁴ t⁴⁵ g⁴⁶ l⁴⁷ t⁴⁸ q⁴⁹ h⁵⁰ g⁵¹ k⁵² v⁵³ p⁵⁴ l⁵⁵ t⁵⁶ f⁵⁷ p⁵⁸ p⁵⁹ g⁶⁰
q⁶¹ g⁶² v⁶³ p⁶⁴ l⁶⁵ n⁶⁶ a⁶⁷ n⁶⁸ s⁶⁹ t⁷⁰ p⁷¹ a⁷² q⁷³ n⁷⁴ a⁷⁵ g⁷⁶ y⁷⁷ w⁷⁸ r⁷⁹ r⁸⁰ q⁸¹ d⁸² r⁸³ k⁸⁴ i⁸⁵ n⁸⁶ t⁸⁷ g⁸⁸ n⁸⁹
g⁹⁰ i⁹¹ k⁹² q⁹³ l⁹⁴ a⁹⁵ p⁹⁶ r⁹⁷ w⁹⁸ y⁹⁹ f¹⁰⁰ y¹⁰¹ y¹⁰² t¹⁰³ g¹⁰⁴ t¹⁰⁵ g¹⁰⁶ p¹⁰⁷ e¹⁰⁸ a¹⁰⁹ a¹¹⁰ l¹¹¹ p¹¹² f¹¹³ r¹¹⁴ a¹¹⁵
v¹¹⁶ k¹¹⁷ d¹¹⁸ g¹¹⁹ i¹²⁰ v¹²¹ w¹²² v¹²³ h¹²⁴ e¹²⁵ d¹²⁶ g¹²⁷ a¹²⁸ t¹²⁹ d¹³⁰ a¹³¹ p¹³² s¹³³ t¹³⁴ f¹³⁵ g¹³⁶ t¹³⁷ r¹³⁸ n¹³⁹
p¹⁴⁰ n¹⁴¹ n¹⁴² d¹⁴³ s¹⁴⁴ a¹⁴⁵ i¹⁴⁶ v¹⁴⁷ t¹⁴⁸ q¹⁴⁹ f¹⁵⁰ a¹⁵¹ p¹⁵² g¹⁵³ t¹⁵⁴ k¹⁵⁵ l¹⁵⁶ p¹⁵⁷ k¹⁵⁸ n¹⁵⁹ f¹⁶⁰ h¹⁶¹ i¹⁶² e¹⁶³
g¹⁶⁴ t¹⁶⁵ g¹⁶⁶ g¹⁶⁷ n¹⁶⁸ s¹⁶⁹ q¹⁷⁰ s¹⁷¹ s¹⁷² s¹⁷³ r¹⁷⁴ a¹⁷⁵ s¹⁷⁶ s¹⁷⁷ v¹⁷⁸ s¹⁷⁹ r¹⁸⁰ n¹⁸¹ s¹⁸² s¹⁸³ r¹⁸⁴ s¹⁸⁵ s¹⁸⁶ s¹⁸⁷
q¹⁸⁸ g¹⁸⁹ s¹⁹⁰ r¹⁹¹ s¹⁹² g¹⁹³ n¹⁹⁴ s¹⁹⁵ t¹⁹⁶ r¹⁹⁷ g¹⁹⁸ t¹⁹⁹ s²⁰⁰ p²⁰¹ g²⁰² p²⁰³ s²⁰⁴ g²⁰⁵ i²⁰⁶ g²⁰⁷ a²⁰⁸ v²⁰⁹ g²¹⁰ g²¹¹
d²¹² l²¹³ l²¹⁴ y²¹⁵ l²¹⁶ d²¹⁷ l²¹⁸ l²¹⁹ n²²⁰ r²²¹ l²²² q²²³ a²²⁴ l²²⁵ e²²⁶ s²²⁷ g²²⁸ k²²⁹ v²³⁰ k²³¹ q²³² s²³³ q²³⁴ p²³⁵
k²³⁶ v²³⁷ i²³⁸ t²³⁹ k²⁴⁰ k²⁴¹ d²⁴² a²⁴³ a²⁴⁴ a²⁴⁵ a²⁴⁶ k²⁴⁷ n²⁴⁸ k²⁴⁹ m²⁵⁰ r²⁵¹ h²⁵² k²⁵³ r²⁵⁴ t²⁵⁵ s²⁵⁶ t²⁵⁷ k²⁵⁸ s²⁵⁹
f²⁶⁰ n²⁶¹ m²⁶² v²⁶³ q²⁶⁴ a²⁶⁵ f²⁶⁶ g²⁶⁷ l²⁶⁸ r²⁶⁹ g²⁷⁰ p²⁷¹ g²⁷² d²⁷³ l²⁷⁴ q²⁷⁵ g²⁷⁶ n²⁷⁷ f²⁷⁸ g²⁷⁹ d²⁸⁰ l²⁸¹ q²⁸² l²⁸³
n²⁸⁴ k²⁸⁵ l²⁸⁶ g²⁸⁷ t²⁸⁸ e²⁸⁹ d²⁹⁰ p²⁹¹ r²⁹² w²⁹³ p²⁹⁴ q²⁹⁵ i²⁹⁶ a²⁹⁷ e²⁹⁸ l²⁹⁹ a³⁰⁰ p³⁰¹ t³⁰² a³⁰³ s³⁰⁴ a³⁰⁵ f³⁰⁶ m³⁰⁷
g³⁰⁸ m³⁰⁹ s³¹⁰ q³¹¹ f³¹² k³¹³ l³¹⁴ t³¹⁵ h³¹⁶ q³¹⁷ n³¹⁸ n³¹⁹ d³²⁰ d³²¹ h³²² g³²³ n³²⁴ p³²⁵ v³²⁶ y³²⁷ f³²⁸ l³²⁹ r³³⁰ y³³¹
s³³² g³³³ a³³⁴ i³³⁵ k³³⁶ l³³⁷ d³³⁸ p³³⁹ k³⁴⁰ p³⁴¹ p³⁴² n³⁴³ y³⁴⁴ p³⁴⁵ k³⁴⁶ w³⁴⁷ l³⁴⁸ e³⁴⁹ l³⁵⁰ l³⁵¹ e³⁵² q³⁵³ n³⁵⁴ i³⁵⁵
d³⁵⁶ a³⁵⁷ y³⁵⁸ k³⁵⁹ t³⁶⁰ f³⁶¹ p³⁶² k³⁶³ k³⁶⁴ e³⁶⁵ k³⁶⁶ k³⁶⁷ q³⁶⁸ k³⁶⁹ a³⁷⁰ p³⁷¹ k³⁷² e³⁷³ e³⁷⁴ s³⁷⁵ t³⁷⁶ d³⁷⁷ q³⁷⁸ m³⁷⁹
s³⁸⁰ e³⁸¹ p³⁸² p³⁸³ k³⁸⁴ e³⁸⁵ h³⁸⁶ r³⁸⁷ v³⁸⁸ q³⁸⁹ g³⁹⁰ t³⁹¹ q³⁹² r³⁹³ t³⁹⁴ r³⁹⁵ t³⁹⁶ r³⁹⁷ p³⁹⁸ s³⁹⁹ V⁴⁰⁰ q⁴⁰¹ p⁴⁰² g⁴⁰
p⁴⁰⁴ m⁴⁰⁵ i⁴⁰⁶ d⁴⁰⁷ v⁴⁰⁸ n⁴⁰⁹ t⁴¹⁰ d⁴¹¹
Amino-terminal
(SEQ ID NO: 191)
h⁵⁰_g⁵¹_k⁵²_v⁵³_p⁵⁴_l⁵⁵_t⁵⁶_f⁵⁷_p⁵⁸_p⁵⁹_g⁶⁰_g⁶¹_g⁶²_v⁶³_p⁶⁴_l⁶⁵_n⁶⁶_a⁶⁷_n⁶⁸_s⁶⁹_t⁷⁰_p⁷¹_a⁷²_q⁷³_n⁷⁴_a⁷⁵_g⁷⁶_y⁷⁷
w⁷⁸_r⁷⁹_r⁸⁰_q⁸¹_d⁸²_r⁸³_k⁸⁴_i⁸⁵_n⁸⁶_t⁸⁷_g⁸⁸_n⁸⁹_g⁹⁰_i⁹¹_k⁹²
(SEQ ID NO: 192)
k⁸⁴_i⁸⁵_n⁸⁶_t⁸⁷_g⁸⁸_n⁸⁹_g⁹⁰_i⁹¹_k⁹²_q⁹³_l⁹⁴_a⁹⁵_p⁹⁶_r⁹⁷_w⁹⁸_y⁹⁹_f¹⁰⁰_y¹⁰¹_y¹⁰²_t¹⁰³_g¹⁰⁴_t¹⁰⁵_g¹⁰⁶_p¹⁰⁷_e¹⁰⁸_a¹⁰⁹
a¹¹⁰_l¹¹¹_p¹¹²_f¹¹³_r¹¹⁴_a¹¹⁵_v¹¹⁶_k¹¹⁷_d¹¹⁸_g¹¹⁹_i¹²⁰_v¹²¹_w¹²²_v¹²³_h¹²⁴
Mid-molecule
(SEQ ID NO: 194)
k²²⁹_v²³⁰_k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹
h²⁵²
(SEQ ID NO: 195)
k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 196)
k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 24)
k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 22)
k²⁴⁷_n²⁴⁸_k²⁴⁹_m²⁵⁰_r²⁵¹_h²⁵²_k²⁵³
(SEQ ID NO: 25)
k²⁴⁹_m²⁵⁰_r²⁵¹_h²⁵²_k²⁵³_r²⁵⁴_t²⁵⁵_s²⁵⁶_t²⁵⁷_k²⁵⁸
Carboxy-terminal
(SEQ ID NO: 197)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p³⁴²_n³⁴³_y³⁴⁴_n³⁴⁵_k³⁴⁶
(SEQ ID NO: 198)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹
p³⁶²_k³⁶³_k³⁶⁴_e³⁶⁵_k³⁶⁶
(SEQ ID NO: 199)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶
(SEQ ID NO: 200)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
a³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶
(SEQ ID NO: 201)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
a³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
(SEQ ID NO: 202)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
a³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
q³⁶⁸_k³⁶⁹
(SEQ ID NO: 203)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
q³⁶⁸_k³⁶⁹_a³⁷⁰_p³⁷¹_k³⁷²
(SEQ ID NO: 204)
k³³⁶_l³³⁷_d³³⁸_p³³⁹_k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸
k³⁵⁹_t³⁶⁰_f³⁶¹_p³⁶²_k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹
p³⁸²_p³⁸³_k³⁸⁴_e³⁸⁵_h³⁸⁶
(SEQ ID NO: 205)
k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²
k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵
h³⁸⁶
(SEQ ID NO: 206)
k³⁵⁹_t³⁶⁰_f³⁶¹_p³⁶²_k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹
p³⁸²_p³⁸³_k³⁸⁴_e³⁸⁵_h³⁸⁶
(SEQ ID NO: 207)
k³⁶³_k³⁶⁴_e³⁶⁵_k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵
h³⁸⁶
(SEQ ID NO: 208)
k³⁶⁴_e³⁶⁵_k³⁶⁶_k³⁶⁷_q³⁶⁸_k³⁶⁹_a³⁷⁰_p³⁷¹_k³⁷²_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵_h³⁸⁶
(SEQ ID NO: 209)
k³⁶⁶_k³⁶⁷_q³⁶⁸_k³⁶⁹_a³⁷⁰_p³⁷¹_k³⁷²_e³⁷³_e³⁷⁴_s³⁷⁵_t³⁷⁶_d³⁷⁷_q³⁷⁸_m³⁷⁹_s³⁸⁰_e³⁸¹_p³⁸²_p³⁸³_k³⁸⁴_e³⁸⁵_h³⁸⁶
Replikin Count in AFY13314 is the number of Replikin sequences per 100 amino acid
residues, which is 21/411 and equals 5.1.
Sequence History by Year for Replikin Sequences Identified in Accession No.
YP_007188586
(SEQ ID NO: 14)
HARLKGGLILK
All occurrences of the sequence by year:
2012
YP_007188577 position 1762, YP_007188578 position 1762, AGV08378 position 1762,
AGV08377 position 1762, AGV08583 position 1762, AGV08582 position 1762,
AGV08407 position 1762, AGV08406 position 1762, AFY13306 position 1762,
AFY13305 position 1762, AGG22541 position 1762, AGG22540 position 1762,
AGH58716 position 1762, AGH58715 position 1762, AFV09327 position 1762,
AFS88944 position 1762.
2013
AHY22564 position 1762, AHY22563 position 1762, AHY22554 position 1762,
AHY22553 position 1762, AHY22544 position 1762, AHY22543 position 1762,
AHY22534 position 1762, AHY22533 position 1762, AHY22524 position 1762,
AHY22523 position 1762, AHE78107 position 1762, AHE78106 position 1762,
AHE78096 position 1762, AHE78095 position 1762, AHX00730 position 1762,
AHX00729 position 1762, AHX00720 position 1762, AHX00719 position 1762,
AHX00710 position 1762, AHX00709 position 1762, K9N638 position 1762, K9N7C7
position 1762, AHN10811 position 1762, AHN10810 position 1762, AHI48796 position
467, AHI48795 position 453, AHI48794 position 1128, AHI48792 position 1128,
AHI48789 position 1083, AHI48787 position 432, AHI48784 position 1128, AHI48779
position 433, AHI48777 position 1119, AHI48776 position 1128, AHI48769 position 1762,
AHI48768 position 1762, AHI48767 position 1762, AHI48766 position 1762, AHI48765
position 1762, AHI48764 position 1762, AHI48761 position 1762, AHI48760 position
1762, AHI48759 position 1762, AHI48758 position 1762, AHI48757 position 1762,
AHI48756 position 1762, AHI48755 position 1762, AHI48754 position 1762, AHI48625
position 1029, AHI48624 position 1029, AHI48615 position 1143, AHI48614 position
1143, AHI48604 position 1762, AHI48603 position 1762, AHI48593 position 1762,
AHI48592 position 1762, AHI48582 position 1762, AHI48581 position 1762, AHI48571
position 1762, AHI48570 position 1762, AHI48549 position 1762, AHI48548 position
1762, AHI48527 position 1762, AHI48526 position 1762, AHI48516 position 1762,
AHI48515 position 1762, AHL18091 position 1762, AHL18089 position 1762, AHC74096
position 1762, AHC74086 position 1762, AHB33325 position 1762, AHB33324 position
1762, AGV08596 position 1762, AGV08595 position 1762, AGV08572 position 1762,
AGV08571 position 1762, AGV08557 position 1762, AGV08556 position 1762,
AGV08555 position 349, AGV08545 position 1762, AGV08544 position 1762,
AGV08534 position 1762, AGV08533 position 1762, AGV08522 position 435,
AGV08491 position 1762, AGV08490 position 1762, AGV08489 position 7, AGV08479
position 1762, AGV08478 position 1762, AGV08466 position 1762, AGV08465 position
1762, AGV08454 position 1762, AGV08453 position 1762, AGV08443 position 1762,
AGV08442 position 1762, AGV08402 position 350, AGN72639 position 1762,
AGN72638 position 1762, AGN70972 position 1762, AGN70971 position 1762,
AGN70961 position 1762, AGN70960 position 1762, AGN70950 position 1762,
AGN70949 position 1762, AGN70928 position 1762, AGN70927 position 1762.
2014
AHY21468 position 1762, AHY21467 position 1762, AHX71945 position 1762,
AHX71944 position 1762.
(SEQ ID NO: 15)
KAMLLKKEPLLYVPIRLAGH
All occurrences of the sequence by year:
2012
YP_007188577 position 60, YP_007188578 position 60, AGV08378 position 60,
AGV08377 position 60, AGV08583 position 60, AGV08582 position 60, AGV08407
position 60, AGV08406 position 60, AFY13306 position 60, AFY13305 position 60,
AGG22541 position 60, AGG22540 position 60, AGH58716 position 60, AGH58715
position 60, AFV09327 position 60, AFS88944 position 60.
2013
AHY22564 position 60, AHY22563 position 60, AHY22554 position 60, AHY22553
position 60, AHY22544 position 60, AHY22543 position 60, AHY22534 position 60,
AHY22533 position 60, AHY22524 position 60, AHY22523 position 60, AHE78107
position 60, AHE78106 position 60, AHE78096 position 60, AHE78095 position 60,
AHX00730 position 60, AHX00729 position 60, AHX00720 position 60, AHX00719
position 60, AHX00710 position 60, AHX00709 position 60, K9N638 position 60,
K9N7C7 position 60, AHN10811 position 60, AHN10810 position 60, AHI48797 position
60, AHI48793 position 60, AHI48791 position 60, AHI48783 position 60, AHI48782
position 60, AHI48778 position 60, AHI48769 position 60, AHI48768 position 60,
AHI48767 position 60, AHI48766 position 60, AHI48765 position 60, AHI48764 position
60, AHI48763 position 60, AHI48762 position 60, AHI48761 position 60, AHI48760
position 60, AHI48759 position 60, AHI48758 position 60, AHI48757 position 60,
AHI48756 position 60, AHI48755 position 60, AHI48754 position 60, AHI48604 position
60, AHI48603 position 60, AHI48593 position 60, AHI48592 position 60, AHI48582
position 60, AHI48581 position 60, AHI48571 position 60, AHI48570 position 60,
AHI48560 position 60, AHI48559 position 60, AHI48549 position 60, AHI48548 position
60, AHI48538 position 60, AHI48537 position 60, AHI48527 position 60, AHI48526
position 60, AHI48516 position 60, AHI48515 position 60, AHL18091 position 60,
AHL18089 position 60, AHC74096 position 60, AHC74086 position 60, AHB33325
position 60, AHB33324 position 60, AGV08596 position 60, AGV08595 position 60,
AGV08572 position 60, AGV08571 position 60, AGV08567 position 60, AGV08557
position 60, AGV08556 position 60, AGV08545 position 60, AGV08544 position 60,
AGV08534 position 60, AGV08533 position 60, AGV08491 position 60, AGV08490
position 60, AGV08479 position 60, AGV08478 position 60, AGV08466 position 60,
AGV08465 position 60, AGV08464 position 60, AGV08454 position 60, AGV08453
position 60, AGV08443 position 60, AGV08442 position 60, AGV08403 position 60,
AGV08389 position 60, AGV08388 position 60, AGN72639 position 60, AGN72638
position 60, AGN70972 position 60, AGN70971 position 60, AGN70961 position 60,
AGN70960 position 60, AGN70950 position 60, AGN70949 position 60, AGN70928
position 60, AGN70927 position 60 .
2014
AHY21468 position 60, AHY21467 position 60, AHX71945 position 60, AHX71944
position 60 .
(SEQ ID NO: 2)
KSAGHPFNK
All occurrences of the sequence by year:
2012
YP_007188577 position 4878, AGV08377 position 4878, AGV08582 position 4878,
AGV08406 position 4878, AFY13306 position 4878, AGG22540 position 4878,
AGH58716 position 4878, AFS88944 position 4878.
2013
AHY22563 position 4878, AHY22553 position 4878, AHY22543 position 4878,
AHY22533 position 4878, AHY22523 position 4878, AHE78106 position 4878,
AHE78095 position 4878, AHX00730 position 4878, AHX00720 position 4878,
AHX00710 position 4878, K9N7C7 position 4878, AHN10810 position 4878, AHI48776
position 4244, AHI48774 position 200, AHI48772 position 200, AHI48768 position 4878,
AHI48766 position 4878, AHI48764 position 4878, AHI48762 position 4878, AHI48760
position 4878, AHI48758 position 4878, AHI48756 position 4878, AHI48754 position
4878, AHI48748 position 1557, AHI48744 position 1659, AHI48624 position 4145,
AHI48614 position 4259, AHI48603 position 4878, AHI48592 position 4878, AHI48581
position 4878, AHI48570 position 4878, AHI48559 position 4878, AHI48548 position
4878, AHI48537 position 4878, AHI48526 position 4878, AHI48515 position 4878,
AHL18091 position 4878, AHC74097 position 485, AHC74087 position 485, AHB33324
position 4878, AGV08595 position 4878, AGV08571 position 4878, AGV08556 position
4878, AGV08544 position 4878, AGV08533 position 4878, AGV08523 position 257,
AGV08504 position 89, AGV08490 position 4878, AGV08478 position 4878, AGV08465
position 4878, AGV08453 position 4878, AGV08442 position 4878, AGV08437 position
83, AGV08435 position 182, AGV08388 position 4878, AGN72639 position 4878,
AGN70971 position 4878, AGN70960 position 4878, AGN70949 position 4878,
AGN70927 position 4878 .
2014
AHY21468 position 4878 , AHX71944 position 4878 .
(SEQ ID NO: 16)
KHLVPLMHK
All occurrences of the sequence by year:
2012
YP_007188577 position 6055, AGV08377 position 6055, AGV08582 position 6055,
AGV08406 position 6055, AFY13306 position 6055, AGG22540 position 6055,
AGH58716 position 6055, AFS88944 position 6055.
2013
AHY22563 position 6055, AHY22553 position 6055, AHY22543 position 6055,
AHY22533 position 6055, AHY22523 position 6055, AHE78106 position 6055,
AHE78095 position 6055, AHX00730 position 6055, AHX00720 position 6055,
AHX00710 position 6055, K9N7C7 position 6055, AHN10810 position 6055, AHI48776
position 5421, AHI48770 position 703, AHI48768 position 6055, AHI48766 position 6055,
AHI48764 position 6055, AHI48762 position 6055, AHI48760 position 6055, AHI48756
position 6055, AHI48754 position 6055, AHI48624 position 5322, AHI48614 position
5436, AHI48603 position 6055, AHI48592 position 6055, AHI48581 position 6055,
AHI48570 position 6055, AHI48559 position 6055, AHI48548 position 6055, AHI48537
position 6055, AHI48526 position 6055, AHI48515 position 6055, AHC74097 position
1662, AHC74087 position 1662, AHB33324 position 6055, AGV08597 position 521,
AGV08595 position 6055, AGV08571 position 6055, AGV08556 position 6055,
AGV08544 position 6055, AGV08533 position 6055, AGV08523 position 1434,
AGV08504 position 1266, AGV08490 position 6055, AGV08478 position 6055,
AGV08465 position 6055, AGV08453 position 6055, AGV08442 position 6055,
AGV08437 position 1260, AGV08388 position 6055, AGN72639 position 6055,
AGN70971 position 6055, AGN70960 position 6055, AGN70949 position 6055,
AGN70927 position 6055.
2014
AHY21468 position 6055 , AHX71944 position 6055 .
(SEQ ID NO: 8)
KKHVEVFTDGK
All occurrences of the sequence by year:
2012
YP_007188577 position 3146, YP_007188578 position 3146, AGV08378 position 3146,
AGV08377 position 3146, AGV08583 position 3146, AGV08582 position 3146,
AGV08407 position 3146, AGV08406 position 3146, AFY13306 position 3146,
AFY13305 position 3146, AGG22541 position 3146, AGG22540 position 3146,
AGH58716 position 3146, AGH58715 position 3146, AFV09327 position 3146,
AFS88944 position 3146.
2013
AHY22564 position 3146, AHY22563 position 3146, AHY22554 position 3146,
AHY22553 position 3146, AHY22544 position 3146, AHY22543 position 3146,
AHY22534 position 3146, AHY22533 position 3146, AHY22524 position 3146,
AHY22523 position 3146, AHE78107 position 3146, AHE78106 position 3146,
AHE78096 position 3146, AHE78095 position 3146, AHX00730 position 3146,
AHX00729 position 3146, AHX00720 position 3146, AHX00719 position 3146,
AHX00710 position 3146, AHX00709 position 3146, K9N638 position 3146 , K9N7C7
position 3146, AHN10811 position 3146, AHN10810 position 3146, AHI48798 position
2512, AHI48796 position 1851, AHI48795 position 1837, AHI48794 position 2512,
AHI48790 position 479, AHI48788 position 699, AHI48780 position 448, AHI48776
position 2512, AHI48769 position 3146, AHI48768 position 3146, AHI48767 position
3146, AHI48766 position 3146, AHI48765 position 3146, AHI48764 position 3146,
AHI48763 position 3146, AHI48762 position 3146, AHI48761 position 3146, AHI48760
position 3146, AHI48759 position 3146, AHI48758 position 3146, AHI48757 position
3146, AHI48756 position 3146, AHI48755 position 3146, AHI48754 position 3146,
AHI48747 position 336, AHI48746 position 336, AHI48625 position 2413, AHI48624
position 2413, AHI48615 position 2527, AHI48614 position 2527, AHI48604 position
3146, AHI48603 position 3146, AHI48593 position 3146, AHI48592 position 3146,
AHI48582 position 3146, AHI48581 position 3146, AHI48571 position 3146, AHI48570
position 3146, AHI48560 position 3146, AHI48559 position 3146, AHI48549 position
3146, AHI48548 position 3146, AHI48538 position 3146, AHI48537 position 3146,
AHI48527 position 3146, AHI48526 position 3146, AHI48516 position 3146, AHI48515
position 3146, AHL18091 position 3146, AHL18089 position 3146, AHC74096 position
3146, AHC74086 position 3146, AHB33325 position 3146, AHB33324 position 3146,
AGV08596 position 3146, AGV08595 position 3146, AGV08572 position 3146,
AGV08571 position 3146, AGV08557 position 3146, AGV08556 position 3146,
AGV08555 position 1733, AGV08545 position 3146, AGV08544 position 3146,
AGV08534 position 3146, AGV08533 position 3146, AGV08515 position 1188,
AGV08491 position 3146, AGV08490 position 3146, AGV08479 position 3146,
AGV08478 position 3146, AGV08466 position 3146, AGV08465 position 3146,
AGV08454 position 3146, AGV08453 position 3146, AGV08443 position 3146,
AGV08442 position 3146, AGV08401 position 1190, AGV08389 position 3146,
AGV08388 position 3146, AGN72639 position 3146, AGN72638 position 3146,
AGN70972 position 3146, AGN70971 position 3146, AGN70961 position 3146,
AGN70960 position 3146, AGN70950 position 3146, AGN70949 position 3146,
AGN70928 position 3146, AGN70927 position 3146.
2014
AHY21468 position 3146, AHY21467 position 3146, AHX71945 position 3146,
AHX71944 position 3146.
(SEQ ID NO: 17)
KYYAFLNKH
All occurrences of the sequence by year:
2012
YP_007188577 position 5122, AGV08377 position 5122, AGV08582 position 5122,
AGV08406 position 5122, AFY13306 position 5122, AGG22540 position 5122,
AGH58716 position 5122, AFS88944 position 5122.
2013
AHY22563 position 5122, AHY22553 position 5122, AHY22543 position 5122,
AHY22533 position 5122, AHY22523 position 5122, AHE78106 position 5122,
AHE78095 position 5122, AHX00730 position 5122, AHX00720 position 5122,
AHX00710 position 5122, K9N7C7 position 5122, AHN10810 position 5122, AHI48776
position 4488, AHI48774 position 444, AHI48773 position 143, AHI48772 position 444,
AHI48768 position 5122, AHI48766 position 5122, AHI48764 position 5122, AHI48762
position 5122, AHI48760 position 5122, AHI48758 position 5122, AHI48756 position
5122, AHI48754 position 5122, AHI48748 position 1801, AHI48744 position 1903,
AHI48624 position 4389, AHI48614 position 4503, AHI48603 position 5122, AHI48592
position 5122, AHI48581 position 5122, AHI48570 position 5122, AHI48559 position
5122, AHI48548 position 5122, AHI48537 position 5122, AHI48526 position 5122,
AHI48515 position 5122, AHL18091 position 5122, AHC74097 position 729, AHC74087
position 729 , AHB33324 position 5122, AGV08595 position 5122, AGV08571 position
5122, AGV08556 position 5122, AGV08544 position 5122, AGV08533 position 5122,
AGV08523 position 501, AGV08504 position 333, AGV08490 position 5122, AGV08478
position 5122, AGV08465 position 5122, AGV08453 position 5122, AGV08442 position
5122, AGV08437 position 327, AGV08435 position 426, AGV08388 position 5122,
AGN72639 position 5122, AGN70971 position 5122, AGN70960 position 5122,
AGN70949 position 5122, AGN70927 position 5122.
2014
AHY21468 position 5122, AHX71944 position 5122.
(SEQ ID NO: 18)
KAAVHKWK
All occurrences of the sequence by year:
2012
YP_007188577 position 1568, YP 007188578 position 1568, AGV08378 position 1568,
AGV08377 position 1568, AGV08583 position 1568, AGV08582 position 1568,
AGV08407 position 1568, AGV08406 position 1568, AFY13306 position 1568,
AFY13305 position 1568, AGG22541 position 1568, AGG22540 position 1568.
2013
AHY22564 position 1568, AHY22563 position 1568, AHY22554 position 1568,
AHY22553 position 1568, AHY22544 position 1568, AHY22543 position 1568,
AHY22534 position 1568, AHY22533 position 1568, AHY22524 position 1568,
AHY22523 position 1568, AHX00730 position 1568, AHX00729 position 1568,
AHX00720 position 1568, AHX00719 position 1568, AHX00710 position 1568,
AHX00709 position 1568, K9N638 position 1568, K9N7C7 position 1568, AHI48798
position 934, AHI48796 position 273, AHI48795 position 259, AHI48794 position 934,
AHI48792 position 934, AHI48791 position 1568, AHI48789 position 889, AHI48787
position 238, AHI48779 position 239, AHI48777 position 925, AHI48776 position 934,
AHI48769 position 1568, AHI48768 position 1568, AHI48767 position 1568, AHI48766
position 1568, AHI48765 position 1568, AHI48764 position 1568, AHI48763 position
1568, AHI48762 position 1568, AHI48761 position 1568, AHI48760 position 1568
AHI48759 position 1568, AHI48758 position 1568, AHI48757 position 1568, AHI48756
position 1568, AHI48755 position 1568, AHI48754 position 1568, AHI48625 position 835,
AHI48624 position 835, AHI48615 position 949, AHI48614 position 949, AHI48604
position 1568, AHI48603 position 1568, AHI48571 position 1568, AHI48570 position
1568, AHI48560 position 1568, AHI48559 position 1568, AHI48538 position 1568,
AHI48537 position 1568, AHI48527 position 1568, AHI48526 position 1568, AHI48516
position 1568, AHI48515 position 1568, AHC74096 position 1568, AHC74086 position
1568, AHB33325 position 1568, AHB33324 position 1568, AGV08596 position 1568,
AGV08595 position 1568, AGV08572 position 1568, AGV08571 position 1568,
AGV08557 position 1568, AGV08556 position 1568, AGV08555 position 155,
AGV08545 position 1568, AGV08544 position 1568, AGV08534 position 1568,
AGV08533 position 1568, AGV08522 position 241, AGV08491 position 1568,
AGV08490 position 1568, AGV08479 position 1568, AGV08478 position 1568,
AGV08466 position 1568, AGV08465 position 1568, AGV08454 position 1568,
AGV08453 position 1568, AGV08443 position 1568, AGV08442 position 1568,
AGV08436 position 525, AGV08402 position 156, AGV08389 position 1568, AGV08388
position 1568, AGN72639 position 1568, AGN72638 position 1568, AGN70972 position
1568, AGN70971 position 1568, AGN70961 position 1568, AGN70960 position 1568,
AGN70950 position 1568, AGN70949 position 1568, AGN70928 position 1568,
AGN70927 position 1568.
2014
AHX71945 position 1568, AHX71944 position 1568.
(SEQ ID NO: 19)
KLNPSEDFIKH
All occurrences of the sequence by year:
2012
YP_007188577 position 1402, YP_007188578 position 1402, AGV08378 position 1402,
AGV08377 position 1402, AGV08583 position 1402, AGV08582 position 1402,
AGV08407 position 1402, AGV08406 position 1402, AFY13306 position 1402,
AFY13305 position 1402, AGG22541 position 1402, AGG22540 position 1402,
AGH58716 position 1402, AGH58715 position 1402, AFV09327 position 1402,
AFS88944 position 1402.
2013
AHY22564 position 1402, AHY22563 position 1402, AHY22554 position 1402,
AHY22553 position 1402, AHY22544 position 1402, AHY22543 position 1402,
AHY22534 position 1402, AHY22533 position 1402, AHY22524 position 1402,
AHY22523 position 1402, AHE78107 position 1402, AHE78106 position 1402,
AHE78096 position 1402, AHE78095 position 1402, AHX00730 position 1402,
AHX00729 position 1402, AHX00720 position 1402, AHX00719 position 1402,
AHX00710 position 1402, AHX00709 position 1402, K9N638 position 1402, K9N7C7
position 1402, AHN10811 position 1402, AHN10810 position 1402, AHI48798 position
768, AHI48796 position 107, AHI48795 position 93, AHI48794 position 768, AHI48792
position 768, AHI48791 position 1402, AHI48789 position 723, AHI48787 position 72,
AHI48784 position 768, AHI48779 position 73, AHI48777 position 759, AHI48776
position 768, AHI48769 position 1402, AHI48768 position 1402, AHI48767 position 1402,
AHI48766 position 1402, AHI48765 position 1402, AHI48764 position 1402, AHI48763
position 1402, AHI48762 position 1402, AHI48761 position 1402, AHI48760 position
1402, AHI48759 position 1402, AHI48758 position 1402, AHI48757 position 1402,
AHI48756 position 1402, AHI48755 position 1402, AHI48754 position 1402, AHI48625
position 669, AHI48624 position 669, AHI48615 position 783, AHI48614 position 783,
AHI48604 position 1402, AHI48603 position 1402, AHI48593 position 1402, AHI48592
position 1402, AHI48582 position 1402, AHI48581 position 1402, AHI48571 position
1402, AHI48570 position 1402, AHI48560 position 1402, AHI48559 position 1402,
AHI48549 position 1402, AHI48548 position 1402, AHI48538 position 1402, AHI48537
position 1402 , AHI48527 position 1402, AHI48526 position 1402, AHI48516 position
1402, AHI48515 position 1402, AHL18091 position 1402, AHL18089 position 1402,
AHC74096 position 1402, AHC74086 position 1402, AHB33325 position 1402,
AHB33324 position 1402, AGV08596 position 1402, AGV08595 position 1402,
AGV08572 position 1402, AGV08571 position 1402, AGV08557 position 1402,
AGV08556 position 1402, AGV08545 position 1402, AGV08544 position 1402,
AGV08534 position 1402, AGV08533 position 1402, AGV08522 position 75, AGV08491
position 1402, AGV08490 position 1402, AGV08479 position 1402, AGV08478 position
1402, AGV08466 position 1402, AGV08465 position 1402, AGV08454 position 1402,
AGV08453 position 1402, AGV08443 position 1402, AGV08442 position 1402,
AGV08436 position 359, AGV08389 position 1402, AGV08388 position 1402,
AGN72639 position 1402, AGN72638 position 1402, AGN70972 position 1402,
AGN70971 position 1402, AGN70961 position 1402, AGN70960 position 1402,
AGN70950 position 1402, AGN70949 position 1402, AGN70928 position 1402,
AGN70927 position 1402.
2014
AHY21468 position 1402, AHY21467 position 1402, AHX71945 position 1402,
AHX71944 position 1402.
(SEQ ID NO: 20)
KFCDHMVK
All occurrences of the sequence by year:
2012
YP_007188577 position 4569, AGV08377 position 4569, AGV08582 position 4569,
AGV08406 position 4569, AFY13306 position 4569, AGG22540 position 4569,
AGH58716 position 4569, AFS88944 position 4569.
2013
AHY22563 position 4569, AHY22553 position 4569, AHY22543 position 4569,
AHY22533 position 4569, AHY22523 position 4569, AHE78106 position 4569,
AHE78095 position 4569, AHX00730 position 4569, AHX00720 position 4569,
AHX00710 position 4569, K9N7C7 position 4569, AHN10810 position 4569, AHI48776
position 3935, AHI48775 position 518, AHI48768 position 4569, AHI48766 position 4569,
AHI48764 position 4569, AHI48762 position 4569, AHI48760 position 4569, AHI48758
position 4569, AHI48756 position 4569, AHI48754 position 4569, AHI48752 position 510,
AHI48750 position 606, AHI48748 position 1248, AHI48744 position 1350, AHI48740
position 520, AHI48624 position 3836, AHI48614 position 3950, AHI48603 position 4569,
AHI48592 position 4569, AHI48581 position 4569, AHI48570 position 4569, AHI48559
position 4569, AHI48548 position 4569, AHI48537 position 4569, AHI48526 position
4569, AHI48515 position 4569, AHL18091 position 4569, AHC74097 position 176,
AHC74087 position 176, AHB33324 position 4569, AGV08595 position 4569, AGV08571
position 4569, AGV08556 position 4569, AGV08544 position 4569, AGV08533 position
4569, AGV08490 position 4569, AGV08478 position 4569, AGV08465 position 4569,
AGV08453 position 4569, AGV08442 position 4569, AGV08388 position 4569,
AGN72639 position 4569, AGN70971 position 4569, AGN70960 position 4569,
AGN70949 position 4569, AGN70927 position 4569.
2014
AHY21468 position 4569 , AHX71944 position 4569 .
(SEQ ID NO: 21)
KPGHAMPSLFK
All occurrences of the sequence by year:
2012
YP_007188577 position 6781, AGV08377 position 6781, AGV08582 position 6781,
AGV08406 position 6781, AFY13306 position 6781, AGG22540 position 6781,
AGH58716 position 6781, AFS88944 position 6781.
2013
AHY22563 position 6781, AHY22553 position 6781, AHY22543 position 6781,
AHY22533 position 6781, AHY22523 position 6781, AHE78106 position 6781,
AHE78095 position 6781, AHX00730 position 6781, AHX00720 position 6781,
AHX00710 position 6781, K9N7C7 position 6781, AHI48776 position 6147, AHI48738
position 240, AHI48736 position 657, AHI48734 position 125, AHI48732 position 125,
AHI48730 position 125, AHI48728 position 180, AHI48726 position 154, AHI48724
position 119, AHI48722 position 125, AHI48720 position 124, AHI48710 position 125,
AHI48701 position 131, AHI48691 position 415, AHI48681 position 125, AHI48671
position 125, AHI48661 position 520, AHI48624 position 6048, AHI48614 position 6162,
AHI48603 position 6781, AHI48592 position 6781, AHI48581 position 6781, AHI48570
position 6781, AHI48559 position 6781, AHI48548 position 6781, AHI48537 position
6781, AHI48526 position 6781, AHI48515 position 6781, AHL31379 position 83,
AHL31377 position 83, AHL31375 position 83, AHL31373 position 83, AHL31371
position 83, AHL31369 position 83, AHL18091 position 6781, AHC74097 position 2388,
AHC74087 position 2388, AHB33324 position 6781, AGV08571 position 6781,
AGV08568 position 125, AGV08556 position 6781, AGV08544 position 6781,
AGV08533 position 6781, AGV08523 position 2160, AGV08519 position 67, AGV08504
position 1992, AGV08490 position 6781, AGV08478 position 6781, AGV08476 position
105, AGV08465 position 6781, AGV08453 position 6781, AGV08442 position 6781,
AGV08437 position 1986, AGV08425 position 125, AGV08399 position 125, AGV08388
position 6781, AGN72639 position 6781, AGN70971 position 6781, AGN70960 position
6781, AGN70949 position 6781, AGN70927 position 6781..
2014
AHY21468 position 6781, AHX71944 position 6781.

### Example 7

### Analysis of Accession No. AHY21476 for Replikin Sequences Conserved in Human Host and Shared with Isolates in Camel Hosts

PubMed Code:
AHY21476
Description:
Full Genome Sequence of Human Betacoronavirus 2c Jordan-N3/2012 after Serial
Passage in Mammalian Cells Direct Submission
Isolated:
2014
Source:
Human betacoronavirus 2c Jordan-N3/2012
Strain:
Jordan-N3/2012
(SEQ ID NO: 210)
m¹ a² s³ p⁴ a⁵ a⁶ p⁷ r⁸ a⁹ v¹⁰ s¹¹ f¹² a¹³ y¹⁴ n¹⁵ n¹⁶ d¹⁷ i¹⁸ t¹⁹ n²⁰ t²¹ n²² l²³ s²⁴ r²⁵ g²⁶ r²⁷ g²⁸ r²⁹ n³⁰ p³¹
k³² p³³ r³⁴ a³⁵ a³⁶ p³⁷ n³⁸ n³⁹ t⁴⁰ v⁴¹ s⁴² w⁴³ y⁴⁴ t⁴⁵ g⁴⁶ l⁴⁷ t⁴⁸ q⁴⁹ h⁵⁰ g⁵¹ k⁵² v⁵³ p⁵⁴ l⁵⁵ t⁵⁶ f⁵⁷ p⁵⁸ p⁵⁹ g⁶⁰
q⁶¹ g⁶² v⁶³ p⁶⁴ l⁶⁵ n⁶⁶ a⁶⁷ n⁶⁸ s⁶⁹ t⁷⁰ p⁷¹ a⁷² q⁷³ n⁷⁴ a⁷⁵ g⁷⁶ y⁷⁷ w⁷⁸ r⁷⁹ r⁸⁰ q⁸¹ d⁸² r⁸³ k⁸⁴ i⁸⁵ n⁸⁶ t⁸⁷ g⁸⁸ n⁸⁹
g⁹⁰ i⁹¹ k⁹² q⁹³ l⁹⁴ a⁹⁵ p⁹⁶ r⁹⁷ w⁹⁸ y⁹⁹ f¹⁰⁰ y¹⁰¹ y¹⁰² t¹⁰³ g¹⁰⁴ t¹⁰⁵ g¹⁰⁶ p¹⁰⁷ e¹⁰⁸ a¹⁰⁹ a¹¹⁰ l¹¹¹ p¹¹² f¹¹³ r¹¹⁴ a¹¹⁵
v¹¹⁶ k¹¹⁷ d¹¹⁸ g¹¹⁹ i¹²⁰ v¹²¹ w¹²² v¹²³ h¹²⁴ e¹²⁵ d¹²⁶ g¹²⁷ a¹²⁸ t¹²⁹ d¹³⁰ a¹³¹ p¹³² s¹³³ t¹³⁴ f¹³⁵ g¹³⁶ t¹³⁷ r¹³⁸ n¹³⁹
p¹⁴⁰ n¹⁴¹ n¹⁴² d¹⁴³ s¹⁴⁴ a¹⁴⁵ i¹⁴⁶ v¹⁴⁷ t¹⁴⁸ q¹⁴⁹ f¹⁵⁰ a¹⁵¹ p¹⁵² g¹⁵³ t¹⁵⁴ k¹⁵⁵ l¹⁵⁶ p¹⁵⁷ k¹⁵⁸ n¹⁵⁹ f¹⁶⁰ h¹⁶¹ i¹⁶² e¹⁶³
g¹⁶⁴ t¹⁶⁵ g¹⁶⁶ g¹⁶⁷ n¹⁶⁸ s¹⁶⁹ q¹⁷⁰ s¹⁷¹ s¹⁷² s¹⁷³ r¹⁷⁴ a¹⁷⁵ s¹⁷⁶ s¹⁷⁷ v¹⁷⁸ s¹⁷⁹ r¹⁸⁰ n¹⁸¹ s¹⁸² s¹⁸³ r¹⁸⁴ s¹⁸⁵
s¹⁸⁶ s¹⁸⁷ q¹⁸⁸ g¹⁸⁹ s¹⁹⁰ r¹⁹¹ s¹⁹² g¹⁹³ n¹⁹⁴ s¹⁹⁵ t¹⁹⁶ r¹⁹⁷ g¹⁹⁸ t¹⁹⁹ s²⁰⁰ p²⁰¹ g²⁰² p²⁰³ s²⁰⁴ g²⁰⁵ i²⁰⁶ g²⁰⁷ a²⁰⁸ v²⁰⁹
g²¹⁰ g²¹¹ d²¹² l²¹³ l²¹⁴ y²¹⁵ l²¹⁶ d²¹⁷ l²¹⁸ l²¹⁹ n²²⁰ r²²¹ l²²² q²²³ a²²⁴ l²²⁵ e²²⁶ s²²⁷ g²²⁸ k²²⁹ v²³⁰ k²³¹ q²³² s²³³
q²³⁴ p²³⁵ k²³⁶ v²³⁷ i²³⁸ t²³⁹ k²⁴⁰ k²⁴¹ d²⁴² a²⁴³ a²⁴⁴ a²⁴⁵ a²⁴⁶ k²⁴⁷ n²⁴⁸ k²⁴⁹ m²⁵⁰ r²⁵¹ h²⁵² k²⁵³ r²⁵⁴ t²⁵⁵ s²⁵⁶ t²⁵⁷
k²⁵⁸ s²⁵⁹ f²⁶⁰ n²⁶¹ m²⁶² v²⁶³ q²⁶⁴ a²⁶⁵ f²⁶⁶ g²⁶⁷ l²⁶⁸ r²⁶⁹ g²⁷⁰ p²⁷¹ g²⁷² d²⁷³ l²⁷⁴ q²⁷⁵ g²⁷⁶ n²⁷⁷ f²⁷⁸ g²⁷⁹ d²⁸⁰ l²⁸¹
q²⁸² l²⁸³ n²⁸⁴ k²⁸⁵ l²⁸⁶ g²⁸⁷ t²⁸⁸ e²⁸⁹ d²⁹⁰ p²⁹¹ r²⁹² w²⁹³ p²⁹⁴ q²⁹⁵ i²⁹⁶ a²⁹⁷ e²⁹⁸ l²⁹⁹ a³⁰⁰ p³⁰¹ t³⁰² a³⁰³ s³⁰⁴ a³⁰⁵
f³⁰⁶ m³⁰⁷ g³⁰⁸ m³⁰⁹ s³¹⁰ q³¹¹ f³¹² k³¹³ l³¹⁴ t³¹⁵ h³¹⁶ q³¹⁷ n³¹⁸ n³¹⁹ d³²⁰ d³²¹ h³²² g³²³ n³²⁴ p³²⁵ v³²⁶ y³²⁷ f³²⁸ l³²⁹
r³³⁰ y³³¹ s³³² g³³³ a³³⁴ i³³⁵ k³³⁶ l³³⁷ d³³⁸ p³³⁹ k³⁴⁰ p³⁴¹ p³⁴² n³⁴³ y³⁴⁴ p³⁴⁵ k³⁴⁶ w³⁴⁷ l³⁴⁸ e³⁴⁹ l³⁵⁰ l³⁵¹ e³⁵² q³⁵³
n³⁵⁴ i³⁵⁵ d³⁵⁶ a³⁵⁷ y³⁵⁸ k³⁵⁹ t³⁶⁰ f³⁶¹ p³⁶² k³⁶³ k³⁶⁴ e³⁶⁵ k³⁶⁶ k³⁶⁷ q³⁶⁸ k³⁶⁹ a³⁷⁰ p³⁷¹ k³⁷² e³⁷³ e³⁷⁴ s³⁷⁵ t³⁷⁶ d³⁷⁷
q³⁷⁸ m³⁷⁹ s³⁸⁰ e³⁸¹ p³⁸² p³⁸³ k³⁸⁴ e³⁸⁵ q³⁸⁶ r³⁸⁷ v³⁸⁸ q³⁸⁹ g³⁹⁰ s³⁹¹ i³⁹² t³⁹³ q³⁹⁴ r³⁹⁵ t³⁹⁶ r³⁹⁷ p³⁹⁸ s³⁹⁹ V⁴⁰⁰ q⁴⁰¹
p⁴⁰² g⁴⁰³ p⁴⁰⁴ m⁴⁰⁵ p⁴⁰⁶ m⁴⁰⁷ i⁴⁰⁸ d⁴⁰⁹ v⁴¹⁰ n⁴¹¹ t⁴¹² d⁴¹³
Amino-terminal
(SEQ ID NO: 211)
h⁵⁰_g⁵¹_k⁵²_v⁵³_p⁵⁴_l⁵⁵_t⁵⁶_f⁵⁷_p⁵⁸_p⁵⁹_g⁶⁰_q⁶¹_g⁶²_v⁶³_p⁶⁴_l⁶⁵_n⁶⁶_a⁶⁷_n⁶⁸_s⁶⁹_t⁷⁰_p⁷¹_a⁷²_q⁷³_n⁷⁴_a⁷⁵_g⁷⁶_y⁷⁷
w⁷⁸_r⁷⁹_r⁸⁰_q⁸¹_d⁸²_r⁸³_k⁸⁴_i⁸⁵_n⁸⁶_t⁸⁷_g⁸⁸_n⁸⁹_g⁹⁰_i⁹¹_k⁹²
(SEQ ID NO: 212)
k⁸⁴_i⁸⁵_n⁸⁶_t⁸⁷_g⁸⁸_n⁸⁹_g⁹⁰_i⁹¹_k⁹²__l⁹⁴_a⁹⁵_p⁹⁶_r⁹⁷_w⁹⁸_y⁹⁹_f¹⁰⁰_y¹⁰¹_y¹⁰²_t¹⁰³_g¹⁰⁴_t¹⁰⁵_g¹⁰⁶_p¹⁰⁷_e¹⁰⁸_a¹⁰⁹
a¹¹⁰_l¹¹¹_p¹¹²_f¹¹³_r¹¹⁴_a¹¹⁵_v¹¹⁶_k¹¹⁷_d¹¹⁸_g¹¹⁹_i¹²⁰_v¹²¹_w¹²²_v¹²³_h¹²⁴
Mid-molecule
(SEQ ID NO: 213)
k²²⁹_v²³⁰_k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹
h²⁵²
(SEQ ID NO: 214)
k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 215)
k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 24)
k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 22)
k²⁴⁷_a²⁴⁸_k²⁴⁹_m²⁵⁰_r²⁵¹_h²⁵²_k²⁵³
(SEQ ID NO: 25)
k²⁴⁹_m²⁵⁰_r²⁵¹_h²⁵²_k²⁵³_r²⁵⁴_t²⁵⁵_s²⁵⁶_t²⁵⁷_k²⁵⁸
Carboxy-terminal
(SEQ ID NO: 216)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p3⁴²_n³⁴³_y³⁴⁴_n³⁴⁵_k³⁴⁶
(SEQ ID NO: 217)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p3⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹
p³⁶²_k³⁶³_k³⁶⁴_e³⁶⁵_k³⁶⁶
(SEQ ID NO: 218)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶
(SEQ ID NO: 219)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶
(SEQ ID NO: 220)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
(SEQ ID NO: 221)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
q³⁶⁸_k³⁶⁹
(SEQ ID NO: 222)
h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
q³⁶⁸_k³⁶⁹_a³⁷⁰_p³⁷¹_k³⁷²
Replikin Count in AFY13314 is the number of Replikin sequences per 100 amino acid
residues, which is 15/413 and equals 3.6.
(SEQ ID NO: 22)
KNKMRHK
All occurrences of the sequence by year:
2012
YP_007188586 position 247, AGV08386 position 247, AGV08591 position 247,
AGV08415 position 247, AFY13314 position 247, AGG22549 position 247, AGH58724
position 247, AFS88943 position 247.
2013
AHY22571 position 247, AHY22561 position 247, AHY22551 position 247, AHY22541
position 247, AHY22531 position 247, AHE78112 position 247, AHE78101 position 247,
AHX00738 position 247, AHX00728 position 247, AHX00718 position 247, AHN10819
position 247, AHI48838 position 247, AHI48819 position 247, AHI48812 position 247,
AHI48810 position 247, AHI48808 position 247, AHI48806 position 247, AHI48804
position 247, AHI48718 position 247, AHI48709 position 247, AHI48699 position 247,
AHI48689 position 247, AHI48679 position 247, AHI48669 position 247, AHI48659
position 247, AHI48649 position 247, AHI48647 position 247, AHI48644 position 247,
AHI48641 position 247, AHI48638 position 247, AHI48635 position 247, AHI48633
position 247, AHI48623 position 247, AHI48612 position 247, AHI48601 position 247,
AHI48590 position 247, AHI48579 position 247, AHI48568 position 247, AHI48557
position 247, AHI48546 position 247, AHI48535 position 247, AHI48524 position 247,
AHL18098 position 247, AHC74105 position 247, AHC74095 position 247, AHB33333
position 247, K9N4V7 position 247, AGV08580 position 247, AGV08565 position 247,
AGV08553 position 247, AGV08542 position 247, AGV08531 position 247, AGV08517
position 247, AGV08512 position 247, AGV08502 position 247, AGV08499 position 247,
AGV08487 position 247, AGV08474 position 247, AGV08462 position 247, AGV08451
position 247, AGV08433 position 247, AGV08418 position 247, AGV08397 position 247,
AGN72646 position 247, AGN70980 position 247, AGN70970 position 247, AGN70958
position 247, AGN70936 position 247.
2014
AHY21476 position 247, AHX71953 position 247.
AHN10819 is a from human host in Saudi Arabia

### Example 8

### Analysis of Accession No. AGV08386 for Replikin Sequences Conserved in Human Host and Shared with Isolates in Camel Hosts

PubMed Code:
AGV08386
Description:
Spread, circulation, and evolution of the Middle East respiratory syndrome coronavirus
Direct Submission
Isolated:
2012 in Saudi Arabia
Source:
Middle East respiratory syndrome coronavirus (MERS-CoV)
Strain:
MERS-CoV
(SEQ ID NO: 223)
m¹ a² s³ p⁴ a⁵ a⁶ p⁷ r⁸ a⁹ v¹⁰ s¹¹ f¹² a¹³ y¹⁴ n¹⁵ n¹⁶ d¹⁷ i¹⁸ t¹⁹ n²⁰ t²¹ n²² l²³ s²⁴ r²⁵ g²⁶ r²⁷ g²⁸ r²⁹ n³⁰ p³¹
k³² p³³ r³⁴ a³⁵ a³⁶ p³⁷ n³⁸ n³⁹ t⁴⁰ v⁴¹ s⁴² w⁴³ y⁴⁴ t⁴⁵ g⁴⁶ l⁴⁷ t⁴⁸ q⁴⁹ h⁵⁰ g⁵¹ k⁵² v⁵³ p⁵⁴ l⁵⁵ t⁵⁶ f⁵⁷ p⁵⁸ p⁵⁹ g⁶⁰
q⁶¹ g⁶² v⁶³ p⁶⁴ l⁶⁵ n⁶⁶ a⁶⁷ n⁶⁸ s⁶⁹ t⁷⁰ p⁷¹ a⁷² q⁷³ n⁷⁴ a⁷⁵ g⁷⁶ y⁷⁷ w⁷⁸ r⁷⁹ r⁸⁰ q⁸¹ d⁸² r⁸³ k⁸⁴ i⁸⁵ n⁸⁶ t⁸⁷ g⁸⁸ n⁸⁹
g⁹⁰ i⁹¹ k⁹² q⁹³ l⁹⁴ a⁹⁵ p⁹⁶ r⁹⁷ w⁹⁸ y⁹⁹ f¹⁰⁰ y¹⁰¹ y¹⁰² t¹⁰³ g¹⁰⁴ t¹⁰⁵ g¹⁰⁶ p¹⁰⁷ e¹⁰⁸ a¹⁰⁹ a¹¹⁰ l¹¹¹ p¹¹² f¹¹³ r¹¹⁴ a¹¹⁵
v¹¹⁶ k¹¹⁷ d¹¹⁸ g¹¹⁹ i¹²⁰ v¹²¹ w¹²² v¹²³ h¹²⁴ e¹²⁵ d¹²⁶ g¹²⁷ a¹²⁸ t¹²⁹ d¹³⁰ a¹³¹ p¹³² s¹³³ t¹³⁴ f¹³⁵ g¹³⁶ t¹³⁷ r¹³⁸ n¹³⁹
p¹⁴⁰ n¹⁴¹ n¹⁴² d¹⁴³ s¹⁴⁴ a¹⁴⁵ i¹⁴⁶ v¹⁴⁷ t¹⁴⁸ q¹⁴⁹ f¹⁵⁰ a¹⁵¹ p¹⁵² g¹⁵³ t¹⁵⁴ k¹⁵⁵ l¹⁵⁶ p¹⁵⁷ k¹⁵⁸ n¹⁵⁹ f¹⁶⁰ h¹⁶¹ i¹⁶² e¹⁶³
g¹⁶⁴ t¹⁶⁵ g¹⁶⁶ g¹⁶⁷ n¹⁶⁸ s¹⁶⁹ q¹⁷⁰ s¹⁷¹ s¹⁷² s¹⁷³ r¹⁷⁴ a¹⁷⁵ s¹⁷⁶ s¹⁷⁷ v¹⁷⁸ s¹⁷⁹ r¹⁸⁰ n¹⁸¹ s¹⁸² s¹⁸³ r¹⁸⁴ s¹⁸⁵ s¹⁸⁶ s¹⁸⁷
q¹⁸⁸ g¹⁸⁹ s¹⁹⁰ r¹⁹¹ s¹⁹² g¹⁹³ n¹⁹⁴ s¹⁹⁵ t¹⁹⁶ r¹⁹⁷ g¹⁹⁸ t¹⁹⁹ s²⁰⁰ p²⁰¹ g²⁰² p²⁰³ s²⁰⁴ g²⁰⁵ i²⁰⁶ g²⁰⁷ a²⁰⁸ v²⁰⁹ g²¹⁰ g²¹¹
d²¹² l²¹³ l²¹⁴ y²¹⁵ l²¹⁶ d²¹⁷ l²¹⁸ l²¹⁹ n²²⁰ r²²¹ l²²² q²²³ a²²⁴ l²²⁵ e²²⁶ s²²⁷ g²²⁸ k²²⁹ v²³⁰ k²³¹ q²³² s²³³ q²³⁴ p²³⁵
k²³⁶ v²³⁷ i²³⁸ t²³⁹ k²⁴⁰ k²⁴¹ d²⁴² a²⁴³ a²⁴⁴ a²⁴⁵ a²⁴⁶ k²⁴⁷ n²⁴⁸ k²⁴⁹ m²⁵⁰ r²⁵¹ h²⁵² k²⁵³ r²⁵⁴ t²⁵⁵ s²⁵⁶ t²⁵⁷ k²⁵⁸ s²⁵⁹
f²⁶⁰ n²⁶¹ m²⁶² v²⁶³ q²⁶⁴ a²⁶⁵ f²⁶⁶ g²⁶⁷ l²⁶⁸ r²⁶⁹ g²⁷⁰ p²⁷¹ g²⁷² d²⁷³ l²⁷⁴ q²⁷⁵ g²⁷⁶ n²⁷⁷ f²⁷⁸ g²⁷⁹ d²⁸⁰ l²⁸¹ q²⁸² l²⁸³
n²⁸⁴ k²⁸⁵ l²⁸⁶ g²⁸⁷ t²⁸⁸ e²⁸⁹ d²⁹⁰ p²⁹¹ r²⁹² w²⁹³ p²⁹⁴ q²⁹⁵ i²⁹⁶ a²⁹⁷ e²⁹⁸ l²⁹⁹ a³⁰⁰ p³⁰¹ t³⁰² a³⁰³ s³⁰⁴ a³⁰⁵ f³⁰⁶ m³⁰⁷
g³⁰⁸ m³⁰⁹ s³¹⁰ q³¹¹ f³¹² k³¹³ l³¹⁴ t³¹⁵ h³¹⁶ q³¹⁷ n³¹⁸ n³¹⁹ d³²⁰ d³²¹ h³²² g³²³ n³²⁴ p³²⁵ v³²⁶ y³²⁷ f³²⁸ l³²⁹ r³³⁰ y³³¹
s³³² g³³³ a³³⁴ i³³⁵ k³³⁶ l³³⁷ d³³⁸ p³³⁹ k³⁴⁰ p³⁴¹ p³⁴² n³⁴³ y³⁴⁴ p³⁴⁵ k³⁴⁶ w³⁴⁷ l³⁴⁸ e³⁴⁹ l³⁵⁰ l³⁵¹ e³⁵² q³⁵³ n³⁵⁴ i³⁵⁵
d³⁵⁶ a³⁵⁷ y³⁵⁸ k³⁵⁹ t³⁶⁰ f³⁶¹ p³⁶² k³⁶³ k³⁶⁴ e³⁶⁵ k³⁶⁶ k³⁶⁷ q³⁶⁸ k³⁶⁹ a³⁷⁰ p³⁷¹ k³⁷² e³⁷³ e³⁷⁴ s³⁷⁵ t³⁷⁶ d³⁷⁷ q³⁷⁸ m³⁷⁹
s³⁸⁰ e³⁸¹ p³⁸² p³⁸³ k³⁸⁴ e³⁸⁵ q³⁸⁶ r³⁸⁷ v³⁸⁸ q³⁸⁹ g³⁹⁰ s³⁹¹ i³⁹² t³⁹³ q³⁹⁴ r³⁹⁵ t³⁹⁶ r³⁹⁷ p³⁹⁸ s³⁹⁹ V⁴⁰⁰ q⁴⁰¹ p⁴⁰² g⁴⁰³
p⁴⁰⁴ g⁴⁰⁵ p⁴⁰⁶ m⁴⁰⁷ i⁴⁰⁸ d⁴⁰⁹ v⁴¹⁰ n⁴¹¹ t⁴¹² d⁴¹³
(SEQ ID NO: 224)
h⁵⁰_g⁵¹_k⁵²_v⁵³_p⁵⁴_l⁵⁵_t⁵⁶_f⁵⁷_p⁵⁸_p⁵⁹_g⁶⁰_q⁶¹_g⁶²_v⁶³_p⁶⁴_l⁶⁵_n⁶⁶_a⁶⁷_n⁶⁸_s⁶⁹_t⁷⁰_p⁷¹_a⁷²_q⁷³_n⁷⁴_a⁷⁵_g⁷⁶_y⁷⁷
w⁷⁸_r⁷⁹_r⁸⁰_q⁸¹_d⁸²_r⁸³_k⁸⁴_i⁸⁵_n⁸⁶_t⁸⁷_g⁸⁸_n⁸⁹_g⁹⁰_i⁹¹_k⁹²
(SEQ ID NO: 225)
k⁸⁴_i⁸⁵_n⁸⁶_t⁸⁷_g⁸⁸_n⁸⁹_g⁹⁰_i⁹¹_k⁹²__l⁹⁴_a⁹⁵_p⁹⁶_r⁹⁷_w⁹⁸_y⁹⁹_f¹⁰⁰_y¹⁰¹_y¹⁰²_t¹⁰³_g¹⁰⁴_t¹⁰⁵_g¹⁰⁶_p¹⁰⁷_e¹⁰⁸_a¹⁰⁹
a¹¹⁰_l¹¹¹_p¹¹²_f¹¹³_r¹¹⁴_a¹¹⁵_v¹¹⁶_k¹¹⁷_d¹¹⁸_g¹¹⁹_i¹²⁰_v¹²¹_w¹²²_v¹²³_h¹²⁴
Mid-molecule
(SEQ ID NO: 226)
k²²⁹_v²³⁰_k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹
h²⁵²
(SEQ ID NO: 227)
k²³¹_q²³²_s²³³_q²³⁴_p²³⁵_k²³⁶_v²³⁷_i²³⁸_t²³⁹_k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 228)
k²⁴⁰_k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 24)
k²⁴¹_d²⁴²_a²⁴³_a²⁴⁴_a²⁴⁵_a²⁴⁶_k²⁴⁷_n²⁴⁸_k²⁴⁹_m²⁵⁰_r²⁵¹_h²⁵²
(SEQ ID NO: 22)
k²⁴⁷_a²⁴⁸_k²⁴⁹_m²⁵⁰_r²⁵¹_h²⁵²_k²⁵³
(SEQ ID NO: 25)
k²⁴⁹_m²⁵⁰_r²⁵¹_h²⁵²_k²⁵³_r²⁵⁴_t²⁵⁵_s²⁵⁶_t²⁵⁷_k²⁵⁸
Carboxy-terminal
(SEQ ID NO: 229)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p³⁴²_n³⁴³_y³⁴⁴_n³⁴⁵_k³⁴⁶
(SEQ ID NO: 230)
h³¹⁶_q³¹⁷_n³¹⁸_n³¹⁹_d³²⁰_d³²¹_h³²²_g³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸
p³³⁹_k³⁴⁰_n³⁴¹_p³⁴²_n³⁴³_y³⁴⁴_n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹
p³⁶²_k³⁶³_k³⁶⁴_e³⁶⁵_k³⁶⁶
(SEQ ID NO: 231)
h³²²_q³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶
(SEQ ID NO: 232)
h³²²_q³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶
(SEQ ID NO: 233)
h³²²_q³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
(SEQ ID NO: 234)
h³²²_q³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
q³⁶⁸_k³⁶⁹
(SEQ ID NO: 235)
h³²²_q³²³_n³²⁴_p³²⁵_v³²⁶_y³²⁷_f³²⁸_l³²⁹_r³³⁰_y³³¹_s³³²_g³³³_a³³⁴_i³³⁵_k³³⁶_l³³⁷_d³³⁸_p³³⁹_k³⁴⁰_n³⁴¹_p³⁴²_n³⁴³_y³⁴⁴
n³⁴⁵_k³⁴⁶_w³⁴⁷_l³⁴⁸_e³⁴⁹_l³⁵⁰_l³⁵¹_e³⁵²_q³⁵³_n³⁵⁴_i³⁵⁵_d³⁵⁶_a³⁵⁷_y³⁵⁸_k³⁵⁹_t³⁶⁰_f³⁶¹_p³⁶²_k³⁶³_k³⁶⁴_e³⁶⁵_k³⁶⁶_k³⁶⁷
q³⁶⁸_k³⁶⁹_a³⁷⁰_p³⁷¹_k³⁷²
Replikin Count in AGV08386 is the number of Replikin sequences per 100 amino acid
residues, which is 15/413 and equals 3.6.

### Example 9

### Analysis of Accession No. AGV08441 for Replikin Sequences Conserved in Human Host and Shared with Isolates in Camel Hosts

AGV08441
2013 Saudi Arabia
Source:
Middle East respiratory syndrome coronavirus (MERS-CoV)
Strain:
MERS-CoV
(SEQ ID NO: 236)
m¹ e² e³ s⁴ l⁵ m⁶ d⁷ v⁸ p⁹ s¹⁰ t¹¹ s¹² g¹³ t¹⁴ q¹⁵ v¹⁶ y¹⁷ s¹⁸ r¹⁹ k²⁰ a²¹ r²² k²³ r²⁴ s²⁵ h²⁶ s²⁷ p²⁸ t²⁹
k³⁰ k³¹ l³² r³³ y³⁴ v³⁵ k³⁶ r³⁷ r³⁸ f³⁹ s⁴⁰ l⁴¹ l⁴² r⁴³ p⁴⁴ e⁴⁵ d⁴⁶ l⁴⁷ s⁴⁸ v⁴⁹ i⁵⁰ v⁵¹ q⁵² p⁵³ t⁵⁴ h⁵⁵ y⁵⁶
v⁵⁷ r⁵⁸ v⁵⁹ t⁶⁰ f⁶¹ s⁶² d⁶³ p⁶⁴ a⁶⁵ m⁶⁶ w⁶⁷ y⁶⁸ l⁶⁹
Amino-terminal
(SEQ ID NO: 5)
k²⁰_a²¹_r²²_k²³_r²⁴_s²⁵_h²⁶_s²⁷_p²⁸_t²⁹_k³⁰
(SEQ ID NO: 237)
k²⁰_a²¹_r²²_k²³_r²⁴_s²⁵_h²⁶_s²⁷_p²⁸_t²⁹_k³⁰_k³¹_l³²_r³³_y³⁴_v³⁵_k³⁶_r³⁷_r³⁸_f³⁹_s⁴⁰_l⁴¹_l⁴²_r⁴³_p⁴⁴_e⁴⁵_d⁴⁶_l⁴⁷_
s⁴⁸_v⁴⁹_i⁵⁰_v⁵¹_q⁵²_p⁵³_t⁵⁴_h⁵⁵
(SEQ ID NO: 238)
k²³_r²⁴_s²⁵_h²⁶_s²⁷_p²⁸_t²⁹_k³⁰_k³¹_l³²_r³³_y³⁴_v³⁵_k³⁶_r³⁷_r³⁸_f³⁹_s⁴⁰_l⁴¹_l⁴²_r⁴³_p⁴⁴_e⁴⁵_d⁴⁶_l⁴⁷_s⁴⁸_v⁴⁹_i⁵⁰_v⁵¹_
q⁵²_p⁵³_t⁵⁴_h⁵⁵
(SEQ ID NO: 3)
k²³_r²⁴_s²⁵_h²⁶_s²⁷_p²⁸_t²⁹_k³⁰
(SEQ ID NO: 23)
k²³_r²⁴_s²⁵_h²⁶_s²⁷_p²⁸_t²⁹
Mid-molecule
(SEQ ID NO: 4)
h²⁶_s²⁷_p²⁸_t²⁹_k³⁰_k³¹_l³²_r³³_y³⁴_v³⁵
(SEQ ID NO: 239)
k³⁰_k³¹_l³²_r³³_y³⁴_v³⁵_k³⁶_r³⁷_r³⁸_f³⁹_s⁴⁰_l⁴¹_l⁴²_r⁴³_p⁴⁴_e⁴⁵_d⁴⁶_l⁴⁷_s⁴⁸_v⁴⁹_i⁵⁰_v⁵¹_q⁵²_p⁵³_t⁵⁴_h⁵⁵
Carboxy-terminal
Zero Replikins.
Replikin Count in AFY13314 is the number of Replikin sequences per 100 amino acid
residues, which is 7/69 and equals 10.1.
Sequence History by Year for Replikin Sequences Identified in Accession No.
AGV08441
(SEQ ID NO: 5)
KARKRSHSPTK
(identified in, for example, camel Qatar 2014 and in
human Saudi Arabia AHN10815 after exposure to infected camel;
(SEQ ID NO: 23)
KRSHSPTK
is another Replikin sequence identified within SEQ ID NO: 5)
All occurrences of the sequence by year:
2012
YP_007188582 position 20, AGV08382 position 20, AGV08587 position 20, AGV08411
position 20, AFY13310 position 20, AGG22545 position 20, AFS88939 position 20.
2013
AHY22567 position 20, AHY22557 position 20, AHY22547 position 20, AHY22537
position 20, AHY22527 position 20, AHY04437 position 20, AHE78113 position 20,
AHE78102 position 20, AHN10815 position 20, AHI48842 position 20, AHI48834
position 20, AHI48827 position 20, AHI48821 position 20, AHI48815 position 20,
AHI48714 position 20, AHI48705 position 20, AHI48695 position 20, AHI48685 position
20, AHI48675 position 20, AHI48665 position 20, AHI48655 position 20, AHI48629
position 20, AHI48619 position 20, AHI48608 position 20, AHI48597 position 20,
AHI48586 position 20, AHI48575 position 20, AHI48564 position 20, AHI48553 position
20, AHI48542 position 20, AHI48531 position 20, AHI48520 position 20, AHL18094
position 32, AHC74101 position 20, AHC74091 position 20, AHB33329 position 20,
AGV08576 position 20, AGV08561 position 20, AGV08549 position 20, AGV08538
position 20, AGV08527 position 20, AGV08508 position 20, AGV08495 position 20,
AGV08483 position 20, AGV08470 position 20, AGV08458 position 20, AGV08447
position 20, AGV08441 position 20, AGV08429 position 20, AGV08423 position 20,
AGV08393 position 20, K9N643 position 20, AGN72642 position 20, AGN70976 position
20, AGN70965 position 20, AGN70954 position 20, AGN70932 position 20.
2014
AHX71949 position 20 .
(SEQ ID NO: 3)
KRSHSPTKK
(identified in, for example, camel in Qatar 2014)
All occurrences of the sequence by year:
2012
YP_007188582 position 23, AGV08382 position 23, AGV08587 position 23, AGV08411
position 23, AFY13310 position 23, AGG22545 position 23, AFS88939 position 23.
2013
AHY22567 position 23, AHY22557 position 23, AHY22547 position 23, AHY22537
position 23, AHY2252 position 23, AHY04437 position 23, AHE78113 position 23,
AHE78102 position 23, AHN10815 position 23, AHI48842 position 23, AHI48834
position 23, AHI48827 position 23, AHI48821 position 23, AHI48815 position 23,
AHI48714 position 23, AHI48705 position 23, AHI48695 position 23, AHI48685 position
23, AHI48675 position 23, AHI48665 position 23, AHI48655 position 23, AHI48629
position 23, AHI48619 position 23, AHI48608 position 23, AHI48597 position 23,
AHI48586 position 23, AHI48575 position 23, AHI48564 position 23, AHI48553 position
23, AHI48542 position 23, AHI48531 position 23, AHI48520 position 23, AHL18094
position 35, AHC74101 position 23, AHC74091 position 23, AHC74084 position 23,
AHB33329 position 23, AGV08576 position 23, AGV08561 position 23, AGV08549
position 23, AGV08538 position 23, AGV08527 position 23, AGV08508 position 23,
AGV08495 position 23, AGV08483 position 23, AGV08470 position 23, AGV08458
position 23, AGV08447 position 23, AGV08441 position 23, AGV08429 position 23,
AGV08423 position 23, AGV08393 position 23, K9N643 position 23, AGN72642 position
23, AGN70976 position 23, AGN70965 position 23, AGN70954 position 23, AGN70932
position 23.
2014
AHX71949 position 23.
(SEQ ID NO: 4)
HSPTKKLRYVK
(identified in, for example, camel in Qatar 2014)
All occurrences of the sequence by year:
2012
YP_007188582 position 26, AGV08382 position 26, AGV08587 position 26, AGV08411
position 26, AFY13310 position 26, AGG22545 position 26, AGH58721 position 26,
AFS88939 position 26.
2013
AHY22567 position 26, AHY22557 position 26, AHY22547 position 26, AHY22537
position 26, AHY22527 position 26, AHY04437 position 26, AHE78113 position 26,
AHE78102 position 26, AHX00734 position 26, AHX00724 position 26, AHX00714
position 26, AHN10815 position 26, AHI48842 position 26, AHI48834 position 26,
AHI48827 position 26, AHI48821 position 26, AHI48815 position 26, AHI48714 position
26, AHI48705 position 26, AHI48695 position 26, AHI48685 position 26, AHI48675
position 26, AHI48665 position 26, AHI48655 position 26, AHI48629 position 26,
AHI48619 position 26, AHI48608 position 26, AHI48597 position 26, AHI48586 position
26, AHI48575 position 26, AHI48564 position 26, AHI48553 position 26, AHI48542
position 26, AHI48531 position 26, AHI48520 position 26, AHL18094 position 38,
AHC74101 position 26, AHC74091 position 26, AHC74084 position 26, AHB33329
position 26, AGV08576 position 26, AGV08561 position 26, AGV08549 position 26,
AGV08538 position 26, AGV08527 position 26, AGV08508 position 26, AGV08495
position 26, AGV08483 position 26, AGV08470 position 26, AGV08458 position 26,
AGV08447 position 26, AGV08441 position 26, AGV08429 position 26, AGV08423
position 26, AGV08393 position 26, K9N643 position 26, AGN72642 position 26,
AGN70976 position 26, AGN70965 position 26, AGN70954 position 26, AGN70932
position 26.
2014
AHY21472 position 26, AHX71949 position 26.

### Example 10

### Analysis of Accession No. K9N4V7 (Betacoronavirus England 1) for Replikin Sequences Conserved in Human Host and Shared with Isolates in Camel Hosts

(SEQ ID NO: 24)
KDAAAAKNKMRH
All occurrences of the sequence by year:
2012
YP_007188586 position 241, AGV08386 position 241, AGV08591 position 241,
AGV08415 position 241, AFY13314 position 241, AGG22549 position 241, AGH58724
position 241, AFS88943 position 241 .
2013
AHY22571 position 241, AHY22561 position 241, AHY22551 position 241, AHY22541
position 241, AHY22531 position 241, AHE78112 position 241, AHE78101 position 241,
AHX00738 position 241, AHX00728 position 241, AHX00718 position 241, AHN10819
position 241, AHI48838 position 241, AHI48819 position 241, AHI48812 position 241,
AHI48810 position 241, AHI48808 position 241, AHI48806 position 241, AHI48804
position 241, AHI48718 position 241, AHI48709 position 241, AHI48699 position 241,
AHI48689 position 241, AHI48679 position 241, AHI48669 position 241, AHI48659
position 241, AHI48649 position 241, AHI48647 position 241, AHI48644 position 241,
AHI48641 position 241, AHI48638 position 241, AHI48635 position 241, AHI48633
position 241, AHI48623 position 241, AHI48612 position 241, AHI48601 position 241,
AHI48590 position 241, AHI48579 position 241, AHI48568 position 241, AHI48557
position 241, AHI48546 position 241, AHI48535 position 241, AHI48524 position 241,
AHL18098 position 241, AHC74105 position 241, AHC74095 position 241, AHB33333
position 241, K9N4V7 position 241, AGV08580 position 241, AGV08565 position 241,
AGV08553 position 241, AGV08542 position 241, AGV08531 position 241, AGV08517
position 241, AGV08512 position 241, AGV08502 position 241, AGV08499 position 241,
AGV08487 position 241, AGV08474 position 241, AGV08462 position 241, AGV08451
position 241, AGV08433 position 241, AGV08418 position 241, AGV08397 position 241,
AGN72646 position 241, AGN70980 position 241, AGN70970 position 241, AGN70958
position 241, AGN70936 position 241.
2014
AHY21476 position 241, AHX71953 position 241.
(SEQ ID NO: 25)
KMRHKRTSTK
All occurrences of the sequence by year:
2012
YP_007188586 position 249, AGV08386 position 249, AGV08591 position 249,
AGV08415 position 249, AFY13314 position 249, AGG22549 position 249, AGH58724
position 249, AFS88943 position 249.
2013
AHY22571 position 249, AHY22561 position 249, AHY22551 position 249, AHY22541
position 249, AHY22531 position 249, AHE78112 position 249, AHE78101 position 249,
AHX00738 position 249, AHX00728 position 249, AHX00718 position 249, AHN10819
position 249, AHI48838 position 249, AHI48819 position 249, AHI48812 position 249,
AHI48810 position 249, AHI48808 position 249, AHI48806 position 249, AHI48804
position 249, AHI48718 position 249, AHI48709 position 249, AHI48699 position 249,
AHI48689 position 249, AHI48679 position 249, AHI48669 position 249, AHI48659
position 249, AHI48649 position 249, AHI48647 position 249, AHI48644 position 249,
AHI48641 position 249, AHI48638 position 249, AHI48635 position 249, AHI48633
position 249, AHI48623 position 249, AHI48612 position 249, AHI48601 position 249,
AHI48590 position 249, AHI48579 position 249, AHI48568 position 249, AHI48557
position 249, AHI48546 position 249, AHI48535 position 249, AHI48524 position 249,
AHL18098 position 249, AHC74105 position 249, AHC74095 position 249, AHB33333
position 249, K9N4V7 position 249, AGV08580 position 249, AGV08565 position 249,
AGV08553 position 249, AGV08542 position 249, AGV08531 position 249, AGV08517
position 249, AGV08512 position 249, AGV08502 position 249, AGV08499 position 249,
AGV08487 position 249, AGV08474 position 249, AGV08462 position 249, AGV08451
position 249, AGV08433 position 249, AGV08418 position 249, AGV08397 position 249,
AGN72646 position 249, AGN70980 position 249, AGN70970 position 249, AGN70958
position 249, AGN70936 position 249.
2014
AHY21476 position 249, AHX71953 position 249.

### Example 11

### Replikin Concentration by Year for Isolates of MERS-CoV from 2012 Through 2014

| | | | Mean | | |
|---|---|---|---|---|---|
| | | | Replikin | | |
| | | No. of | Count | | |
| | | Isolates | per | | |
| Year | PubMed Accession Number-Replikin Count | per year | year | S.D. | Significance |
| 2012 | YP_007188577 162 YP_007188586 21 YP_007188585 3 | 52 | 2.9 | 1.7 | low p > .50 |
| | YP_007188582 12 YP_007188579 13 YP_007188578 100 | | | | |
| | AGV08386 15 AGV08385 3 AGV08382 12 AGV08379 13 | | | | |
| | AGV08378 100 AGV08377 162 AGV08591 15 AGV08590 3 | | | | |
| | AGV08587 12 AGV08584 13 AGV08583 100 AGV08582 162 | | | | |
| | AGV08415 15 AGV08414 3 AGV08411 12 AGV08408 13 | | | | |
| | AGV08407 100 AGV08406 162 AFY13314 21 AFY13313 3 | | | | |
| | AFY13310 12 AFY13307 13 AFY13306 162 AFY13305 100 | | | | |
| | AGO06003 6 AGO06001 2 AGO06000 4 AGO05997 2 | | | | |
| | AGG22549 21 AGG22548 3 AGG22545 12 AGG22542 13 | | | | |
| | AGG22541 100 AGG22540 162 AGH58724 15 AGH58721 12 | | | | |
| | AGH58718 3 AGH58717 13 AGH58716 160 AGH58715 98 | | | | |
| | AFV09327 99 AFS88944 161 AFS88943 15 AFS88942 3 | | | | |
| | AFS88939 15 AFS88936 13 | | | | |
| 2013 | AHY22571 13 AHY22570 3 AHY22567 12 AHY22565 13 | 501 | 2.4 | 1.4 | low p > .50, |
| | AHY22564 100 AHY22563 162 AHY22561 13 AHY22560 3 | | | | prev p < .05 |
| | AHY22557 12 AHY22555 13 AHY22554 100 AHY22553 162 | | | | |
| | AHY22551 15 AHY22550 3 AHY22547 12 AHY22545 13 | | | | |
| | AHY22544 100 AHY22543 165 AHY22541 16 AHY22540 3 | | | | |
| | AHY22537 12 AHY22535 13 AHY22534 100 AHY22533 162 | | | | |
| | AHY22531 16 AHY22530 3 AHY22527 12 AHY22525 13 | | | | |
| | AHY22524 100 AHY22523 162 AHY04437 12 AHY04436 4 | | | | |
| | AHY04435 2 AHY04434 2 AHY04433 6 AHX71943 2 | | | | |
| | AHX71942 2 AHX71941 4 AHX71940 6 AHE78113 12 | | | | |
| | AHE78112 15 AHE78111 3 AHE78108 13 AHE78107 98 | | | | |
| | AHE78106 160 AHE78102 12 AHE78101 15 AHE78100 3 | | | | |
| | AHE78097 13 AHE78096 98 AHE78095 160 AHX00738 15 | | | | |
| | AHX00737 3 AHX00734 8 AHX00731 13 AHX00730 162 | | | | |
| | AHX00729 100 AHX00728 15 AHX00727 3 AHX00724 8 | | | | |
| | AHX00721 13 AHX00720 162 AHX00719 100 AHX00718 15 | | | | |
| | AHX00717 3 AHX00714 8 AHX00711 13 AHX00710 162 | | | | |
| | AHX00709 100 K9N638 100 K9N7C7 162 AHN10826 1 | | | | |
| | AHN10825 2 AHN10824 4 AHN10819 15 AHN10818 3 | | | | |
| | AHN10815 12 AHN10812 13 AHN10811 98 AHN10810 148 | | | | |
| | AHI48842 12 AHI48838 15 AHI48837 3 AHI48834 12 | | | | |
| | AHI48827 12 AHI48825 2 AHI48824 3 AHI48821 12 AHI48819 | | | | |
| | 15 AHI48818 3 AHI48815 12 AHI48812 15 AHI48810 16 | | | | |
| | AHI48808 15 AHI48806 15 AHI48804 15 AHI48803 6 | | | | |
| | AHI48802 1 AHI48801 6 AHI48800 6 AHI48799 2 AHI48798 69 | | | | |
| | AHI48797 25 AHI48796 47 AHI48795 53 AHI48794 67 | | | | |
| | AHI48793 28 AHI48792 61 AHI48791 75 AHI48790 4 | | | | |
| | AHI48789 61 AHI48788 5 AHI48787 36 AHI48786 15 | | | | |
| | AHI48785 14 AHI48784 59 AHI48783 25 AHI48782 25 | | | | |
| | AHI48781 22 AHI48780 4 AHI48779 30 AHI48778 35 | | | | |
| | AHI48777 63 AHI48776 133 AHI48775 16 AHI48774 18 | | | | |
| | AHI48773 9 AHI48772 18 AHI48771 10 AHI48770 11 | | | | |
| | AHI48769 100 AHI48768 148 AHI48767 100 AHI48766 143 | | | | |
| | AHI48765 100 AHI48764 148 AHI48763 100 AHI48762 145 | | | | |
| | AHI48761 100 AHI48760 144 AHI48759 100 AHI48758 142 | | | | |
| | AHI48757 100 AHI48756 148 AHI48755 100 AHI48754 146 | | | | |
| | AHI48753 4 AHI48752 23 AHI48751 4 AHI48750 23 AHI48749 | | | | |
| | 10 AHI48748 42 AHI48747 14 AHI48746 22 AHI48745 10 | | | | |
| | AHI48744 52 AHI48743 4 AHI48742 12 AHI48741 4 AHI48740 | | | | |
| | 23 AHI48739 13 AHI48738 12 AHI48737 13 AHI48736 16 | | | | |
| | AHI48735 7 AHI48734 12 AHI48733 13 AHI48732 12 | | | | |
| | AHI48731 7 AHI48730 12 AHI48729 7 AHI48728 12 AHI48727 | | | | |
| | 13 AHI48726 12 AHI48725 3 AHI48724 12 AHI48723 1 | | | | |
| | AHI48722 12 AHI48721 1 AHI48720 12 AHI48718 16 | | | | |
| | AHI48717 3 AHI48714 12 AHI48711 13 AHI48710 12 | | | | |
| | AHI48709 8 AHI48708 3 AHI48705 12 AHI48702 13 AHI48701 | | | | |
| | 12 AHI48699 8 AHI48698 3 AHI48695 12 AHI48692 13 | | | | |
| | AHI48691 14 AHI48689 15 AHI48688 3 AHI48685 12 | | | | |
| | AHI48682 13 AHI48681 12 AHI48679 15 AHI48678 3 | | | | |
| | AHI48675 12 AHI48672 13 AHI48671 12 AHI48669 8 | | | | |
| | AHI48668 3 AHI48665 12 AH148662 13 AHI48661 14 | | | | |
| | AHI48659 16 AHI48658 3 AHI48655 12 AH148652 13 | | | | |
| | AHI48651 3 AHI48649 15 AHI48648 3 AHI48647 15 AHI48646 | | | | |
| | 3 AHI48644 15 AHI48643 3 AHI48641 15 AHI48640 3 | | | | |
| | AHI48638 15 AHI48637 3 AHI48635 15 AHI48634 3 AHI48633 | | | | |
| | 15 AHI48632 3 AHI48629 12 AHI48626 13 AHI48625 72 | | | | |
| | AHI48624 134 AHI48623 15 AHI48622 3 AHI48619 12 | | | | |
| | AHI48616 13 AHI48615 75 AHI48614 137 AHI48612 15 | | | | |
| | AHI48611 3 AHI48608 12 AHI48605 13 AHI48604 100 | | | | |
| | AHI48603 162 AHI48601 15 AHI48600 3 AHI48597 12 | | | | |
| | AHI48594 13 AHI48593 98 AHI48592 160 AHI48590 15 | | | | |
| | AHI48589 3 AHI48586 12 AHI48583 13 AHI48582 98 | | | | |
| | AHI48581 160 AHI48579 15 AHI48578 3 AHI48575 12 | | | | |
| | AHI48572 13 AHI48571 100 AHI48570 162 AHI48568 15 | | | | |
| | AHI48567 3 AHI48564 12 AHI48561 13 AHI48560 100 | | | | |
| | AHI48559 162 AHI48557 15 AHI48556 3 AHI48553 12 | | | | |
| | AHI48550 13 AHI48549 100 AHI48548 162 AHI48546 15 | | | | |
| | AHI48545 3 AHI48542 12 AHI48539 13 AHI48538 100 | | | | |
| | AHI48537 162 AHI48535 15 AHI48534 3 AHI48531 12 | | | | |
| | AHI48528 13 AHI48527 100 AHI48526 162 AHI48524 15 | | | | |
| | AHI48523 3 AHI48520 12 AHI48517 13 AHI48516 100 | | | | |
| | AHI48515 162 AHL31380 1 AHL31379 12 AHL31378 1 | | | | |
| | AHL31377 9 AHL31376 1 AHL31375 9 AHL31374 1 | | | | |
| | AHL31373 9 AHL31372 1 AHL31371 9 AHL31370 1 | | | | |
| | AHL31369 9 AHL31368 1 AHL31367 1 AHL31366 1 | | | | |
| | AHL31365 1 AHL31364 1 AHL31363 1 AHL31362 1 | | | | |
| | AHL31361 1 AHL31360 1 AHL31359 1 AHL18098 13 | | | | |
| | AHL18097 3 AHL18094 12 AHL18091 160 AHL18090 13 | | | | |
| | AHL18089 101 K9N5Q8 13 K9N7A1 3 AHC74105 15 | | | | |
| | AHC74104 3 AHC74101 12 AHC74098 13 AHC74097 62 | | | | |
| | AHC74096 101 AHC74095 15 AHC74094 3 AHC74091 12 | | | | |
| | AHC74088 13 AHC74087 62 AHC74086 101 AHC74084 12 | | | | |
| | AHC74082 2 AHC74081 2 AHC74080 5 AHB33333 15 | | | | |
| | AHB33332 3 AHB33329 12 AHB33326 13 AHB33325 99 | | | | |
| | AHB33324 161 4MOD_B 6 4MOD_A 6 K9N4V7 21 4L3N_B 1 | | | | |
| | 4L3N_A 1 AGV08597 8 AGV08596 100 AGV08595 143 | | | | |
| | AGV08594 6 AGV08593 15 AGV08580 15 AGV08579 3 | | | | |
| | AGV08576 12 AGV08573 13 AGV08572 100 AGV08571 162 | | | | |
| | AGV08570 18 AGV08569 1 AGV08568 12 AGV08567 43 | | | | |
| | AGV08565 15 AGV08564 3 AGV08561 12 AGV08558 13 | | | | |
| | AGV08557 100 AGV08556 162 AGV08555 53 AGV08553 15 | | | | |
| | AGV08552 3 AGV08549 12 AGV08546 13 AGV08545 100 | | | | |
| | AGV08544 162 AGV08542 15 AGV08541 3 AGV08538 12 | | | | |
| | AGV08535 13 AGV08534 100 AGV08533 162 AGV08531 15 | | | | |
| | AGV08530 3 AGV08527 12 AGV08524 14 AGV08523 52 | | | | |
| | AGV08522 42 AGV08520 1 AGV08519 6 AGV08517 15 | | | | |
| | AGV08516 3 AGV08515 25 AGV08514 12 AGV08512 15 | | | | |
| | AGV08511 3 AGV08508 12 AGV08505 13 AGV08504 44 | | | | |
| | AGV08502 15 AGV08501 3 AGV08499 15 AGV08498 3 | | | | |
| | AGV08495 12 AGV08492 13 AGV08491 100 AGV08490 162 | | | | |
| | AGV08489 23 AGV08487 16 AGV08486 3 AGV08483 12 | | | | |
| | AGV08480 13 AGV08479 100 AGV08478 162 AGV08477 7 | | | | |
| | AGV08476 12 AGV08474 15 AGV08473 3 AGV08470 12 | | | | |
| | AGV08467 13 AGV08466 100 AGV08465 162 AGV08464 25 | | | | |
| | AGV08462 15 AGV08461 3 AGV08458 12 AGV08455 13 | | | | |
| | AGV08454 100 AGV08453 162 AGV08451 15 AGV08450 3 | | | | |
| | AGV08447 12 AGV08444 13 AGV08443 100 AGV08442 162 | | | | |
| | AGV08441 7 AGV08438 13 AGV08437 44 AGV08436 27 | | | | |
| | AGV08435 17 AGV08433 8 AGV08432 3 AGV08429 12 | | | | |
| | AGV08426 13 AGV08425 12 AGV08423 12 AGV08420 6 | | | | |
| | AGV08418 15 AGV08417 3 AGV08405 2 AGV08404 16 | | | | |
| | AGV08403 25 AGV08402 37 AGV08401 25 AGV08400 1 | | | | |
| | AGV08399 9 AGV08397 15 AGV08396 3 AGV08393 12 | | | | |
| | AGV08390 13 AGV08389 97 AGV08388 159 4L72_B 1 4L72_A | | | | |
| | 12 4KR0_B 7 4KR0_A 12 4KQZ_B 7 4KQZ_A 7 K9N643 12 | | | | |
| | AGN72646 15 AGN72645 3 AGN72642 12 AGN72639 162 | | | | |
| | AGN72638 100 AGN70980 15 AGN70979 3 AGN70976 12 | | | | |
| | AGN70973 13 AGN70972 100 AGN70971 162 AGN70970 15 | | | | |
| | AGN70968 3 AGN70965 12 AGN70962 13 AGN70961 100 | | | | |
| | AGN70960 162 AGN70958 15 AGN70957 3 AGN70954 12 | | | | |
| | AGN70951 13 AGN70950 100 AGN70949 162 AGN70936 15 | | | | |
| | AGN70935 3 AGN70932 12 AGN70929 13 AGN70928 100 | | | | |
| | AGN70927 162 AGN52936 13 | | | | |
| 2014 | AHY21476 15 AHY21475 3 AHY21472 12 AHY21469 13 | 12 | 2.6 | 1.4 | prev p > .50 |
| | AHY21468 160 AHY21467 98 AHX71953 16 AHX71952 3 | | | | |
| | AHX71949 12 AHX71946 13 AHX71945 100 AHX71944 162 | | | | |

### Example 12

### Conserved MERS CoV Sequences

The following sequences are provided as examples of sequences conserved in isolates of MERS CoV across time and in various regions. Each sequence is provided below with a listing of the accession number and position in the publicly-disclosed sequence wherein the peptide sequence begins.

(SEQ ID NO: 22)
KNKMRHK

2012

AGV08591 position 247, AGV08415 position 247, AGV08386 position 247, YP_007188586 position 247, AFY13314 position 247, AGG22549 position 247, AGH58724 position 247, AFS88943 position 247.

2013

AGV08580 position 247, AGV08565 position 247, AGV08553 position 247, AGV08542 position 247, AGV08531 position 247, AGV08517 position 247, AGV08512 position 247, AGV08502 position 247, AGV08499 position 247, AGV08487 position 247, AGV08474 position 247, AGV08462 position 247, AGV08451 position 247, AGV08433 position 247, AGV08418 position 247, AGV08397 position 247, K9N4V7 position 247, AGN72646 position 247, AGN70980 position 247, AGN70970 position 247, AGN70958 position 247, AGN70936 position 247.

(SEQ ID NO: 18)
KAAVHKWK

2012

AGV08583 position 1568, AGV08582 position 1568, AGV08407 position 1568, AGV08406 position 1568, AGV08378 position 1568, AGV08377 position 1568, YP 007188577 position 1568, YP 007188578 position 1568, AFY13306 position 1568, AFY13305 position 1568, AGG22541 position 1568, AGG22540 position 1568.

2013

K9N638 position 1568, K9N7C7 position 1568, AGV08596 position 1568, AGV08595 position 1568, AGV08572 position 1568, AGV08571 position 1568, AGV08557 position 1568, AGV08556 position 1568, AGV08555 position 155, AGV08545 position 1568, AGV08544 position 1568, AGV08534 position 1568, AGV08533 position 1568, AGV08522 position 241, AGV08491 position 1568, AGV08490 position 1568, AGV08479 position 1568, AGV08478 position 1568, AGV08466 position 1568, AGV08465 position 1568, AGV08454 position 1568, AGV08453 position 1568, AGV08443 position 1568, AGV08442 position 1568, AGV08436 position 525, AGV08402 position 156, AGV08389 position 1568, AGV08388 position 1568, AGN72639 position 1568, AGN72638 position 1568, AGN70972 position 1568, AGN70971 position 1568, AGN70961 position 1568, AGN70960 position 1568, AGN70950 position 1568, AGN70949 position 1568, AGN70928 position 1568, AGN70927 position 1568.

(SEQ ID NO: 23)
KRSHSPTK

2012

AGV08587 position 23, AGV08411 position 23, AGV08382 position 23, YP 007188582 position 23, AFY13310 position 23, AGG22545 position 23, AFS88939 position 23.

2013

AGV08576 position 23, AGV08561 position 23, AGV08549 position 23, AGV08538 position 23, AGV08527 position 23, AGV08508 position 23, AGV08495 position 23, AGV08483 position 23, AGV08470 position 23, AGV08458 position 23, AGV08447 position 23, AGV08441 position 23, AGV08429 position 23, AGV08423 position 23, AGV08393 position 23, K9N643 position 23, AGN72642 position 23, AGN70976 position 23, AGN70965 position 23, AGN70954 position 23, AGN70932 position 23.

(SEQ ID NO: 5)
KARKRSHSPTK

2012

AGV08587 position 20, AGV08411 position 20, AGV08382 position 20, YP 007188582 position 20, AFY13310 position 20, AGG22545 position 20, AFS88939 position 20.

2013 AGV08576 position 20, AGV08561 position 20, AGV08549 position 20, AGV08538 position 20, AGV08527 position 20, AGV08508 position 20, AGV08495 position 20, AGV08483 position 20, AGV08470 position 20, AGV08458 position 20, AGV08447 position 20, AGV08441 position 20, AGV08429 position 20, AGV08423 position 20, AGV08393 position 20, K9N643 position 20, AGN72642 position 20, AGN70976 position 20, AGN70965 position 20, AGN70954 position 20, AGN70932 position 20.

Conserved sequences are useful as targets for control of replication and lethality in vaccines. A vaccine combining conserved sequences or employing individual sequences or conserved sequences as vaccine targets may be used to prevent or control MERS CoV infection and outbreaks.

### Example 13

### MERS COV Replikin Concentrations Compared Between Time Periods

Gene and amino acid sequences publicly available for isolates of MERS CoV were queried at www.pubmed.com. Replikin concentrations were determined and numbers of isolates with Replikin concentrations greater than 4.0 were compared to numbers of isolates with Replikin concentrations 4.0 or less. A percentage was determined for each time period.

Table 1 provides the data from April 2012.

TABLE 1

| Date of Publication | | | |
|---|---|---|---|
| of Specimen | Replikin | Gene | Accession |
| YYYMMDD | Concentration | Segment | Number |
| 20120400 | 1 | S | AGH58717 |
| 20120400 | 1.4 | M | AGH58718 |
| 20120400 | 4.9 | NS3c | K9N643 |
| 20120400 | 2.2 | ORF1a | AGH58715 |
| 20120400 | 2.3 | ORF1ab | AGH58716 |
| 20120400 | 2.3 | ORF1a | K9N638 |
| 20120400 | 3.6 | N | AGH58724 |
| 20120400 | 4.9 | ORF4b | AGH58721 |
| 20120400 | 2.2 | ORF1a | AGH58715 |
| 20120400 | 2.2 | ORF1a | AGH58715 |
| 20120400 | 2.3 | ORF1ab | AGH58716 |
| 20120400 | 1 | S | AGH58717 |
| 20120400 | 1.4 | M | AGH58718 |
| 20120400 | 4.9 | ORF4b | AGH58721 |

The number of isolates from April 2012 was 15. The number of isolates with a Replikin concentration of greater than 4.0 was 3. The percentage of isolates with a Replikin concentration of greater than 4.0 is 20%.

Table 2 provides the data from May through June 2012.

TABLE 2

| Date of Publication | | | |
|---|---|---|---|
| of Specimen | Replikin | | |
| YYYMMDD | Concentration | Gene Segment | Accession Number |
| 20120513 | 3.6 | N | AFS88943 |
| 20120513 | 3.6 | N | AFS88943 |
| 20120513 | 3.6 | N | AGV08415 |
| 20120612 | 1.4 | M | AGV08414 |
| 20120613 | 1 | S | AFS88936 |
| 20120613 | 6.1 | ORF4b | AFS88939 |
| 20120613 | 2.3 | ORF1ab | AFS88944 |
| 20120613 | 2.3 | ORF1a | AFV09327 |

The number of isolates from May through June 2012 was 8. The number of isolates with a Replikin concentration of greater than 4.0 was 1. The percentage of isolates with a Replikin concentration of greater than 4.0 is 12.50%.

Table 3 provides the data from September 2012.

TABLE 3

| Date of Publication | | | |
|---|---|---|---|
| of Specimen | Replikin | | |
| YYYMMDD | Concentration | Gene Segment | Accession Number |
| 20120900 | 2.3 | ORF1ab | K9N7C7 |
| 20120900 | 1.4 | M | K9N7A1 |
| 20120900 | 1 | NS3C | K9N5Q8 |
| 20120900 | 5.1 | N | K9N4V7 |
| 20120911 | 2.3 | ORF1a | AFY13305 |
| 20120911 | 2.3 | ORF1b | AFY13306 |
| 20120911 | 1 | S | AFY13307 |
| 20120911 | 4.9 | NS3C | AFY13310 |
| 20120911 | 1.4 | M | AFY13313 |
| 20120911 | 5.1 | N | AFY13314 |
| 20120911 | 1.4 | M | AFY13313 |
| 20120911 | 1 | S | AFY13307 |
| 20120911 | 2.3 | ORF1a | AFY13305 |
| 20120911 | 2.3 | ORF1b | AFY13306 |
| 20120911 | 4.9 | NS3C | AFY13310 |
| 20120911 | 5.1 | N | AFY13314 |
| 20120919 | 2.3 | ORF1ab | AGG22540 |
| 20120919 | 2.3 | ORF1a | AGG22541 |
| 20120919 | 1 | S | AGG22542 |
| 20120919 | 4.9 | ORF4b | AGG22545 |
| 20120919 | 1.4 | M | AGG22548 |
| 20120919 | 5.1 | N | AGG22549 |
| 20120919 | 1.4 | M | AGG22548 |
| 20120919 | 1 | S | AGG22542 |
| 20120919 | 2.3 | ORF1ab | AGG22540 |
| 20120919 | 4.9 | ORF4b | AGG22545 |
| 20120919 | 5.1 | N | AGG22549 |

The number of isolates from September 2012 was 30. The number of isolates with a Replikin concentration of greater than 4.0 was 9. The percentage of isolates with a Replikin concentration of greater than 4.0 is 30%.

Table 4 provides the data from October 2012.

TABLE 4

| Date of Publication | | | |
|---|---|---|---|
| of Specimen | Replikin | | |
| YYYMMDD | Concentration | Gene Segment | Accession Number |
| 20121016 | 1.4 | M | AFS88942 |
| 20120000 | 4.4 | ORF1ab | AGO05997 |
| 20120000 | 2.7 | ORF1ab | AGO06000 |
| 20120000 | 3.3 | ORF1ab | AGO06001 |
| 20120000 | 4.3 | S | AGO06003 |
| 20121023 | 2.3 | ORF1ab | AGV08377 |
| 20121023 | 2.3 | ORF1a | AGV08378 |
| 20121023 | 1 | S | AGV08379 |
| 20121023 | 7.6 | ORF4b | AGV08382 |
| 20121023 | 1.4 | M | AGV08385 |
| 20121023 | 3.6 | N | AGV08386 |
| 20121016 | 1.4 | M | AFS88942 |
| 20120000 | 4.4 | ORF1ab | AGO05997 |

The number of isolates from October 2012 was 11. The number of isolates with a Replikin concentration of greater than 4.0 was 3. The percentage of isolates with a Replikin concentration of greater than 4.0 is 27%.

Table 5 provides the data from February 2013.

TABLE 5

| Date of Publication | | | |
|---|---|---|---|
| of Specimen | Replikin | | |
| YYYMMDD | Concentration | Gene Segment | Accession Number |
| 20130000 | 2.3 | ORF1ab | AGN72639 |
| 20130000 | 4.9 | ORF4b | AGN72642 |
| 20130000 | 2.3 | ORF1ab | AGN72639 |
| 20130000 | 2.3 | ORF1ab | AGN72638 |
| 20130000 | 1 | | AGN52936 |
| 20130000 | 1 | S | AGN52936 |
| 20130205 | 2.2 | ORF1ab | AGV08388 |
| 20130205 | 2.2 | ORF1a | AGV08389 |
| 20130205 | 1 | S | AGV08390 |
| 20130205 | 2.2 | ORF1ab | AGV08388 |
| 20130205 | 2.2 | ORF1a | AGV08389 |
| 20130205 | 1 | S | AGV08390 |
| 20130205 | 4.9 | ORF4b | AGV08393 |
| 20130205 | 1.4 | M | AGV08396 |
| 20130205 | 3.6 | N | AGV08397 |
| 20130000 | 2.3 | ORF1ab | AGN72639 |

The number of isolates from February 2013 was 15. The number of isolates with a Replikin concentration of greater than 4.0 was 2. The percentage of isolates with a Replikin concentration of greater than 4.0 is 13.30%.

Table 6 provides the data from April 2013.

TABLE 6

| Date of Publication | | | |
|---|---|---|---|
| of Specimen | Replikin | | |
| YYYMMDD | Concentration | Gene Segment | Accession Number |
| 20130421 | 1 | S | AGN70951 |
| 20130421 | 2.3 | ORF1a | AGN70950 |
| 20130421 | 2.3 | ORF1ab | AGN70949 |
| 20130421 | 4.9 | ORF4b | AGN70954 |
| 20130421 | 1.4 | M | AGN70957 |
| 20130421 | 3.6 | N | AGN70958 |
| 20130422 | 2.3 | ORF1ab | AGN70971 |
| 20130422 | 2.3 | ORF1a | AGN70972 |
| 20130422 | 1 | S | AGN70973 |
| 20130422 | 4.9 | ORF4b | AGN70976 |
| 20130422 | 1.4 | M | AGN70979 |
| 20130422 | 3.6 | N | AGN70980 |
| 20130421 | 1 | S | AGN70951 |

The number of isolates from April 2013 was 11. The number of isolates with a Replikin concentration of greater than 4.0 was 2. The percentage of isolates with a Replikin concentration of greater than 4.0 is 18.2%.

Table 7 provides the data from May 1, 2013.

TABLE 7

| Date of Publication | | | |
|---|---|---|---|
| of Specimen | Replikin | | |
| YYYMMDD | Concentration | Gene Segment | Accession Number |
| 20130501 | 2.3 | ORF1ab | AGN70927 |
| 20130501 | 2.3 | ORF1a | AGN70928 |
| 20130501 | 1 | S | AGN70929 |
| 20130501 | 4.9 | ORF4b | AGN70932 |
| 20130501 | 1.4 | M | AGN70935 |
| 20130501 | 3.6 | N | AGN70936 |
| 20130501 | 2.5 | ORF1ab | AGV08404 |
| 20130501 | 1.5 | S | AGV08420 |
| 20130501 | 4.9 | ORF4b | AGV08423 |
| 20130501 | 2.8 | ORF1ab | AGV08425 |
| 20130501 | 1 | S | AGV08426 |
| 20130501 | 4.9 | ORF4b | AGV08429 |
| 20130501 | 1.4 | M | AGV08432 |
| 20130501 | 2.9 | N | AGV08433 |
| 20130501 | 1.9 | ORF1ab | AGV08437 |
| 20130501 | 1 | S | AGV08438 |
| 20130501 | 10.1 | ORF4b | AGV08441 |
| 20130501 | 1.4 | M | AGV08501 |
| 20130501 | 3.6 | N | AGV08502 |

The number of isolates from May 1, 2013 was 19. The number of isolates with a Replikin concentration of greater than 4.0 was 4. The percentage of isolates with a Replikin concentration of greater than 4.0 is 21.1%.

Table 8 provides the data from May 2-30, 2013.

TABLE 8

| Date of Publication | | | |
|---|---|---|---|
| of Specimen | Replikin | | |
| YYYMMDD | Concentration | Gene Segment | Accession Number |
| 20130502 | 2.9 | ORF1ab | AGV08435 |
| 20130502 | 3.1 | ORF1ab | AGV08399 |
| 20130502 | 0.3 | S | AGV08400 |
| 20130503 | 3.9 | ORF1ab | AGV08464 |
| 20130503 | 3.9 | ORF1ab | AGV08464 |
| 20130507 | 2.9 | ORF1a | AGV08402 |
| 20130507 | 2.3 | ORF1ab | AGV08442 |
| 20130507 | 2.3 | ORF1a | AGV08443 |
| 20130507 | 1 | S | AGV08444 |
| 20130507 | 4.9 | ORF4b | AGV08447 |
| 20130507 | 1.4 | M | AGV08450 |
| 20130507 | 3.6 | N | AGV08451 |
| 20130508 | 1.1 | ORF1ab | AGV08401 |
| 20130509 | 4 | ORF1ab | AGV08403 |
| 20130509 | 0.7 | ORF1ab | AGV08405 |
| 20130509 | 2 | M | AGV08417 |
| 20130509 | 3.6 | N | AGV08418 |
| 20130509 | 3.9 | ORF1ab | AGV08436 |
| 20130509 | 3 | ORF1ab | AGV08476 |
| 20130509 | 0.7 | S | AGV08477 |
| 20130509 | 2.4 | ORF1ab | AGV08489 |
| 20130509 | 2.3 | ORF1ab | AGN70960 |
| 20130509 | 2.3 | ORF1a | AGN70961 |
| 20130509 | 1 | S | AGN70962 |
| 20130509 | 4.9 | ORF4b | AGN70965 |
| 20130509 | 1.4 | M | AGN70968 |
| 20130513 | 2.3 | ORF1ab | AGV08465 |
| 20130513 | 2.3 | ORF1a | AGV08466 |
| 20130513 | 1 | S | AGV08467 |
| 20130513 | 4.9 | ORF4b | AGV08470 |
| 20130513 | 1.4 | M | AGV08473 |
| 20130513 | 3.6 | N | AGV08474 |
| 20130515 | 2.8 | S | 4KQZ_B |
| 20130515 | 2.8 | S | 4KR0_B |
| 20130523 | 2.3 | ORF1ab | AGV08478 |
| 20130523 | 2.3 | Orf1a | AGV08479 |
| 20130523 | 1 | S | AGV08480 |
| 20130523 | 4.9 | ORF4b | AGV08483 |
| 20130523 | 1.4 | M | AGV08486 |
| 20130523 | 3.9 | N | AGV08487 |
| 20130530 | 2.3 | ORF1ab | AGV08490 |
| 20130530 | 2.3 | ORF1a | AGV08491 |
| 20130530 | 1 | S | AGV08492 |
| 20130530 | 4.9 | ORF4b | AGV08495 |
| 20130530 | 1.4 | M | AGV08498 |
| 20130530 | 3.6 | N | AGV08499 |

The number of isolates from May 2-30, 2013 was 48. The number of isolates with a Replikin concentration of greater than 4.0 was 7. The percentage of isolates with a Replikin concentration of greater than 4.0 is 14.6%.

Table 9 provides the data from June 2013.

TABLE 9

| Date of Publication | | | |
|---|---|---|---|
| of Specimen | Replikin | | |
| YYYMMDD | Concentration | Gene Segment | Accession Number |
| 20130604 | 2.3 | ORF1a | AGV08454 |
| 20130604 | 1 | S | AGV08455 |
| 20130604 | 4.9 | ORF4b | AGV08458 |
| 20130604 | 1.4 | M | AGV08461 |
| 20130604 | 3.6 | N | AGV08462 |
| 20130606 | 0.5 | N3 | 4L3N_A |
| 20130619 | 2.3 | ORF1ab | AGV08406 |
| 20130619 | 2.3 | ORF1a | AGV08407 |
| 20130619 | 1 | S | AGV08408 |
| 20130619 | 7.6 | ORF4b | AGV08411 |

The number of isolates from June 2013 was 10. The number of isolates with a Replikin concentration of greater than 4.0 was 2. The percentage of isolates with a Replikin concentration of greater than 4.0 is 20%.

### Example 14

### Analysis of AHY22523

Accession number AHY22523 was analyzed. The following sequences were identified and with further analysis were determined to be conserved among camels and humans. AHY22523 is entitled “Middle East Respiratory Syndrome Coronavirus Quasispecies That Include Homologues of Human Isolates Revealed through Whole-Genome Analysis and Virus Cultured from Dromedary Camels in Saudi Arabia Direct Submission.”

(SEQ ID NO: 26)
HVERKDVPYPK
(SEQ ID NO: 13)
KGDSCSSNCKH
(SEQ ID NO: 27)
KGMQLLHTK
(SEQ ID NO: 19)
KLNPSEDFIKH
(SEQ ID NO: 18)
KAAVHKWK
(SEQ ID NO: 6)
KHVEVFTDGK
(SEQ ID NO: 20)
KFCDHMVK
(SEQ ID NO: 28)
KEGSSVTLKH
(SEQ ID NO: 14)
HARLKGGLILK
(SEQ ID NO: 11)
KFYQHVINGCK
(SEQ ID NO: 12)
KQVHQVQLTDK.

A synthetic Replikin vaccine containing an approximately equal-parts-by-weight mixture of the eleven MERS CoV Replikin peptides was designed for use against relatively lethal isolates of MERS CoV. The vaccine was engineered from sequences confirmed to be conserved among camels and humans across time. Conservation was particularly noted in the key amino acid residues of the Replikin sequence, namely, lysine and histidine amino acid residues. The vaccine was engineered to inhibit the lethality of relatively lethal strains of MERS CoV.

The vaccine may be administered to an animal or human susceptible, exposed to, or suffering from infection of MERS CoV. The blocking mechanism of the vaccine provides a therapeutic and prophylactic effect. The immune response generated by the vaccine provides a prophylactic effect. Infection and replication are blocked by targeting these sequences.

### Example 15

### Analysis of all Isolates of MERS-CoV

All isolates of the virus genes reported to date in at the NCBI PubMed database from both humans and camels was undertaken and revealed specific shared Replikin structures which were not just homologous but identical in the virus genes infecting both human and camel hosts, conserved since 2012 in cases from all countries affected. A Replikins Synthetic Vaccine based on these identical shared structures was produced. This vaccine is an inhibitor of the progression of MERS-CoV. The Anti-MERS-CoV vaccine was prepared for testing in humans and camels.

Camels are understood to be a reservoir and source of human MERS infection mostly because of the detection in camels of antibodies that react with MERS (1,2). The present quantitative gene specific structural findings support this understanding.

SARS, a coronavirus related to MERS, produced approximately 8000 human cases and 800 deaths in 2003. MERS has a higher mortality rate than SARS. MERS started in 2012 with an approximate mortality rate of 40%, and now, perhaps with better care, is still approximately 27%. The duration of the outbreak is already greater. This is in contrast to SARS, who's abrupt rise and rapid decline in gene Replikin concentration signaled in 2002 that the 2003 outbreak would occur but would be over soon (and it was within one year). The outbreak of MERS in 2012, the increased Replikin concentrations reported in October 2013, and the upsurge in MERS clinical outbreaks in 2014 now indicate a more prolonged course and a greater risk of pandemic. In agreement with these differences, the highest individual gene Replikin concentrations observed to date in MERS is double that in SARS.

The Replikins Synthetic Anti-MERS Vaccine is unique because:
1. Prediction: The vaccine is based on gene technology which correctly predicts, one to two years in advance, a coming breakthrough or pandemic, as established retrospectively one year before the SARS outbreak of 2003; and prospectively i) in June 2013 for the current MERS outbreaks, ii) in 2006 for the 2007 H5N1 lethal outbreak in Indonesia, and iii) in 2008 for the 2009 H1N1 pandemic.
2. The concentration of gene Replikin sequences (Replikin Count=Number of Replikins per 100 amino acids) relates quantitatively to rapid replication of the pathogen, and to morbidity and mortality, whether the pathogen is a virus, bacterium, or cancer cell.
3. The prediction identifies the specific gene Replikin structures involved in the rapid replication and outbreaks of the attacking pathogen.
4. The prediction also defines the geographic areas that have the highest pathogen Replikins activity, from which global spread initiates, and to which public health and vaccine containment efforts can be directed (as with Indonesia for H5N1 in 2006, and Saudi Arabia for the current MERS outbreaks).
5. The specific gene Replikin structures identified are the basis of the synthetic vaccine designed;
6. Such Replikin vaccines have been produced freeze-dried by solid phase synthesis in 7 days, can be produced in unlimited quantities before the outbreak peaks, have been shipped without need of refrigeration, and have been found effective against Taura Syndrome virus in shrimp and influenza H5N1 virus in chickens.
7. The discovery of identical MERS gene Replikin structures in both infected humans and in camels provides a surprising defined parallel host relationship for vaccine testing.

Replikin sequences, therefore, provide a complete defense system, not previously available, beginning with prediction pre-outbreak, based on detection and specific identification of the quantitative gene changes associated with rapid replication. These gene changes can be addressed in advance of, or after, outbreaks occur, providing time to possibly prevent pandemics rather than just react to them.

While it would have been preferable to begin testing the Replikins vaccine when the upturn in gene Replikin Count was noted in October 2013, there still may be time to reduce or interrupt the present progression of MERS-CoV.

1. CIDRAP NEWS. Reports: Mers-CoV found in Saudi's patient's camel. Nov. 11, 2013.
2. Memish, Ziad. Promed Mail. Animal reservoirs, camels. http://www.isid.org Nov. 12, 2013.
3. Bogoch S and Bogoch ES. Nature Precedings doi: 10.1038/npre.2012.6952.1 Bogoch Replikins Pandemic Prevention: Increase of Strain-Specific Influenza Genomic Replikin Counts, Having Predicted Outbreaks and their Location Seven Times Consecutively, Up to Two Years in Advance, Provides Time for Prevention of Pandemics.

### Example 16

### October 2013 Identification of Increasing Replikin Concentration in MERS-CoV

In October 2013, the applicants identified an increase in Replikin concentration in isolates of MERS-CoV.

The BBC reported the inventor's analysis that Middle East Respiratory Syndrome corona virus (MERS) has undergone several fluctuations in the virus' ability to replicate and could be rising again. The MERS Replikin concentration peaked in virulence and infectivity in September of 2012 with a count of 27.7% and dropped sharply to a low of 14.3% by February of 2013. The count began to rise again and as of June 2013 (the last month for which data were available at the time of analysis in October 2013) had reached 20%. “MERS Replikin Count Increases; Updated Data Needed,” BBC News Blog, Monday, Oct. 21, 2013, Michelle Blowers 3:56 p.m.

1-2. (canceled)

3. An immunogenic or blocking composition comprising at least one isolated or synthesized peptide that is at least 80% homologous with at least one of SEQ ID NO(s): 1-28.

4-12. (canceled)

13. The immunogenic or blocking composition of claim 3, wherein said at least one isolated or synthesized peptide consists of at least one of SEQ ID NO(s): 1-28.

14. The immunogenic or blocking composition of claim 3, wherein said at least one isolated or synthesized peptide consists of at least one of SEQ ID NO(s): 1-9.

15. The immunogenic or blocking composition of claim 3, wherein said at least one isolated or synthesized peptide consists of at least one of SEQ ID NO(s): 5, 18, 22, and 23.

16. The immunogenic or blocking composition of claim 3, wherein said at least one isolated or synthesized peptide consists of at least one of SEQ ID NO(s): 6, 11, 12, 13, 14, 18, 19, 20, 26, 27, and 28.

17. (canceled)

18. The immunogenic or blocking composition of claim 3, wherein said at least one peptide comprises at least one Replikin sequence shared among at least one isolate of MERS CoV isolated from a human and at least one isolate of MERS CoV isolated from a camel.

19-24. (canceled)

25. An isolated or purified antibody, antibody fragment, or binding agent that specifically binds to at least a portion of a peptide that is at least 80% homologous with at least one of SEQ ID NO(s): 1-28.

26. (canceled)

27. The isolated or purified antibody, antibody fragment, or binding agent of claim 25 that specifically binds to at least a portion of any one of SEQ ID NO(s): 1-28.

28. The isolated or purified antibody, antibody fragment, or binding agent of claim 25 that specifically binds to at least a portion of any one of SEQ ID NO(s): 1-9.

29. A method of making a vaccine comprising: isolating or synthesizing a peptide that is at least 80% homologous with at least one Replikin peptide sequence identified in a MERS CoV as a component of a vaccine; and making said vaccine.

30. (canceled)

31. The immunogenic or blocking composition of claim 3 comprising a mixture of at least nine peptides having different amino acid sequences, wherein said mixture comprises at least one peptide having an amino acid sequence that is at least 80% homologous with SEQ ID NO: 1, at least one peptide having an amino acid sequence that is at least 80% homologous with SEQ ID NO: 2, at least one peptide having an amino acid sequence that is at least 80% homologous with SEQ ID NO: 3, at least one peptide having an amino acid sequence that is at least 80% homologous with SEQ ID NO: 4, at least one peptide having an amino acid sequence that is at least 80% homologous with SEQ ID NO: 5, at least one peptide having an amino acid sequence that is at least 80% homologous with SEQ ID NO: 6, at least one peptide having an amino acid sequence that is at least 80% homologous with SEQ ID NO: 7, at least one peptide having an amino acid sequence that is at least 80% homologous with SEQ ID NO: 8, and at least one peptide having an amino acid sequence that is at least 80% homologous with SEQ ID NO: 9.

32. The immunogenic or blocking composition of claim 31 comprising a mixture of at least one peptide of each of SEQ ID NO(s): 1-9.


SEQUENCE LISTING (239 sequences)

SEQ ID NO: 1 - protein, 7 aa, Artificial Sequence
  Description of Artificial Sequence Synthetic peptide
KQKAPKH

SEQ ID NO: 2 - protein, 9 aa, Artificial Sequence
  Description of Artificial Sequence Synthetic peptide
KSAGHPFNK

SEQ ID NO: 3 - protein, 9 aa, Artificial Sequence
  Description of Artificial Sequence Synthetic peptide
KRSHSPTKK

SEQ ID NO: 4 - protein, 11 aa, Artificial Sequence
  Description of Artificial Sequence Synthetic peptide
HSPTKKLRYVK

SEQ ID NO: 5 - protein, 11 aa, Artificial Sequence
  Description of Artificial Sequence Synthetic peptide
KARKRSHSPTK

SEQ ID NO: 6 - protein, 10 aa, Artificial Sequence
  Description of Artificial Sequence Synthetic peptide
KHVEVFTDGK

SEQ ID NO: 7 - protein, 10 aa, Artificial Sequence
  Description of Artificial Sequence Synthetic peptide
HKWKMVVCDK

SEQ ID NO: 8 - protein, 11 aa, Artificial Sequence
  Description of Artificial Sequence Synthetic peptide
KKHVEVFTDGK

SEQ ID NO: 9 - protein, 13 aa, Artificial Sequence
  Description of Artificial Sequence Synthetic peptide
KDAAAAKNKMRHK

SEQ ID NO: 10 - protein, 11 aa, Artificial Sequence
  Description of Artificial Sequence Synthetic peptide
KSVVRHLGVTK

SEQ ID NO: 11 - protein, 11 aa, Artificial Sequence
  Description of Artificial Sequence Synthetic peptide
KFYQHVINGCK

SEQ ID NO: 12 - protein, 11 aa, Artificial Sequence
  Description of Artificial Sequence Synthetic peptide
KQVHQVQLTDK

SEQ ID NO: 13 - protein, 11 aa, Artificial Sequence
  Description of Artificial Sequence Synthetic peptide
KGDSCSSNCKH

SEQ ID NO: 14 - protein, 11 aa, Artificial Sequence
  Description of Artificial Sequence Synthetic peptide
HARLKGGLILK

SEQ ID NO: 15 - protein, 20 aa, Artificial Sequence
  Description of Artificial Sequence Synthetic peptide
KAMLLKKEPLLYVPIRLAGH

SEQ ID NO: 16 - protein, 9 aa, Artificial Sequence
  Description of Artificial Sequence Synthetic peptide
KHLVPLMHK

SEQ ID NO: 17 - protein, 9 aa, Artificial Sequence
  Description of Artificial Sequence Synthetic peptide
KYYAFLNKH

SEQ ID NO: 18 - protein, 8 aa, Artificial Sequence
  Description of Artificial Sequence Synthetic peptide
KAAVHKWK

SEQ ID NO: 19 - protein, 11 aa, Artificial Sequence
  Description of Artificial Sequence Synthetic peptide
KLNPSEDFIKH

SEQ ID NO: 20 - protein, 8 aa, Artificial Sequence
  Description of Artificial Sequence Synthetic peptide
KFCDHMVK

SEQ ID NO: 21 - protein, 11 aa, Artificial Sequence
  Description of Artificial Sequence Synthetic peptide
KPGHAMPSLFK

SEQ ID NO: 22 - protein, 7 aa, Artificial Sequence
  Description of Artificial Sequence Synthetic peptide
KNKMRHK

SEQ ID NO: 23 - protein, 8 aa, Artificial Sequence
  Description of Artificial Sequence Synthetic peptide
KRSHSPTK

SEQ ID NO: 24 - protein, 12 aa, Artificial Sequence
  Description of Artificial Sequence Synthetic peptide
KDAAAAKNKMRH

SEQ ID NO: 25 - protein, 10 aa, Artificial Sequence
  Description of Artificial Sequence Synthetic peptide
KMRHKRTSTK

SEQ ID NO: 26 - protein, 11 aa, Artificial Sequence
  Description of Artificial Sequence Synthetic peptide
HVERKDVPYPK

SEQ ID NO: 27 - protein, 9 aa, Artificial Sequence
  Description of Artificial Sequence Synthetic peptide
KGMQLLHTK

SEQ ID NO: 28 - protein, 10 aa, Artificial Sequence
  Description of Artificial Sequence Synthetic peptide
KEGSSVTLKH

SEQ ID NO: 29 - protein, 7078 aa, Artificial Sequence
  Description of Artificial Sequence Synthetic polypeptide
MSFVAGVTAQGARGTYRAALNSEKHQDHVSLTVPLCGSGNLVEKLSPWFMDGENAYEVVKAMLLKKEPLLYVPIRLAGHTRHLPGPRVYLVERLIACENPFMVNQLAYSSSANGSLVGTTLQGKPIGMFFPYDIELVTGKQNILLRKYGRGGYHYTPFHYERDNTSCPEWMDDFEADPKGKYAQNLLKKLIGGDVTPVDQYMCGVDGKPISAYAFLMAKDGITKLADVEADVAARADDEGFITLKNNLYRLVWHVERKDVPYPKQSIFTINSVVQKDGVENTPPHYFTLGCKILTLTPRNKWSGVSDLSLKQKLLYTFYGKESLENPTYIYHSAFIECGSCGNDSWLTGNAIQGFACGCGASYTANDVEVQSSGMIKPNALLCATCPFAKGDSCSSNCKHSVAQLVSYLSERCNVIADSKSFTLIFGGVAYAYFGCEEGTMYFVPRAKSVVSRIGDSIFTGCTGSWNKVTQIANMFLEQTQHSLNFVGEFVVNDVVLAILSGTTTNVDKIRQLLKGVTLDKLRDYLADYDVAVTAGPFMDNAINVGGTGLQYAAITAPYVVLTGLGESFKKVATIPYKVCNSVKDTLTYYAHSVLYRVFPYDMDSGVSSFSELLFDCVDLSVASTYFLVRLLQDKTGDFMSTIITSCQTAVSKLLDTCFEATEATFNFLLDLAGLFRIFLRNAYVYTSQGFVVVNGKVSTLVKQVLDLLNKGMQLLHTKVSWAGSNISAVIYSGRESLIFPSGTYYCVTTKAKSVQQDLDVILPGEFSKKQLGLLQPTDNSTTVSVTVSSNMVETVVGQLEQTNMHSPDVIVGDYVIISEKLFVRSKEEDGFAFYPACTNGHAVPTLFRLKGGAPVKKVAFGGDQVHEVAAVRSVTVEYNIHAVLDTLLASSSLRTFVVDKSLSIEEFADVVKEQVSDLLVKLLRGMPIPDFDLDDFIDAPCYCFNAEGDASWSSTMIFSLHPVECDEECSEVEASDLEEGESECISETSTEQVDVSHEISDDEWAAAVDEAFPLDEAEDVTESVQEEAQPVEVPVEDIAQVVIADTLQETPVVSDTVEVPPQVVKLPSEPQTIQPEVKEVAPVYEADTEQTQSVTVKPKRLRKKRNVDPLSNFEHKVITECVTIVLGDAIQVAKCYGESVLVNAANTHLKHGGGIAGAINAASKGAVQKESDEYILAKGPLQVGDSVLLQGHSLAKNILHVVGPDARAKQDVSLLSKCYKAMNAYPLVVTPLVSAGIFGVKPAVSFDYLIREAKTRVLVVVNSQDVYKSLTIVDIPQSLTFSYDGLRGAIRKAKDYGFTVFVCTDNSANTKVLRNKGVDYTKKFLTVDGVQYYCYTSKDTLDDILQQANKSVGIISMPLGYVSHGLDLIQAGSVVRRVNVPYVCLLANKEQEAILMSEDVKLNPSEDFIKHVRTNGGYNSWHLVEGELLVQDLRLNKLLHWSDQTICYKDSVFYVVKNSTAFPFETLSACRAYLDSRTTQQLTIEVLVTVDGVNFRTVVLNNKNTYRSQLGCVFFNGADISDTIPDEKQNGHSLYLADNLTADETKALKELYGPVDPTFLHRFYSLKAAVHKWKMVVCDKVRSLKLSDNNCYLNAVIMTLDLLKDIKFVIPALQHAFMKHKGGDSTDFIALIMAYGNCTFGAPDDASRLLHTVLAKAELCCSARMVWREWCNVCGIKDVVLQGLKACCYVGVQTVEDLRARMTYVCQCGGERHRQIVEHTTPWLLLSGTPNEKLVTTSTAPDFVAFNVFQGIETAVGHYVHARLKGGLILKFDSGTVSKTSDWKCKVTDVLFSGQKYSSDCNVVRYSLDGNFRTEVDPDLSAFYVKDGKYFTSEPPVTYSPATILAGSVYTNSCLVSSDGQPGGDAISLSFNNLLGFDSSKPVTKKYTYSFLPKEDGDVLLAEFDTYDPIYKNGAMYKGKPILWVNKASYDTNLNKFNRASLRQIFDVAPIELENKFTPLSVESTPVEPPTVDVVALQQEMTIVKCKGLNKPFVKDNVSFVADDSGTPVVEYLSKEDLHTLYVDPKYQVIVLKDNVLSSMLRLHTVESGDINVVAASGSLTRKVKLLFRASFYFKEFATRTFTATTAVGSCIKSVVRHLGVTKGILTGCFSFVKMLFMLPLAYFSDSKLGTTEVKVSALKTAGVVTGNVVKQCCTAAVDLSMDKLRRVDWKSTLRLLLMLCTTMVLLSSVYHLYVFNQVLSSDVMFEDAQGLKKFYKEVRAYLGISSACDGLASAYRANSFDVPTFCANRSAMCNWCLISQDSITHYPALKMVQTHLSHYVLNIDWLWFAFETGLAYMLYTSAFNWLLLAGTLHYFFAQTSIFVDWRSYNYAVSSAFWLFTHIPMAGLVRMYNLLACLWLLRKFYQHVINGCKDTACLLCYKRNRLTRVEASTVVCGGKRTFYITANGGISFCRRHNWNCVDCDTAGVGNTFICEEVANDLTTALRRPINATDRSHYYVDSVTVKETVVQFNYRRDGQPFYERFPLCAFTNLDKLKFKEVCKTTTGIPEYNFIIYDSSDRGQESLARSACVYYSQVLCKSILLVDSSLVTSVGDSSEIATKMFDSFVNSFVSLYNVTRDKLEKLISTARDGVRRGDNFHSVLTTFIDAARGPAGVESDVETNEIVDSVQYAHKHDIQITNESYNNYVPSYVKPDSVSTSDLGSLIDCNAASVNQIVLRNSNGACIWNAAAYMKLSDALKRQIRIACRKCNLAFRLTTSKLRANDNILSVRFTANKIVGGAPTWFNALRDFTLKGYVLATIIVFLCAVLMYLCLPTFSMVPVEFYEDRILDFKVLDNGIIRDVNPDDKCFANKHRSFTQWYHEHVGGVYDNSITCPLTVAVIAGVAGARIPDVPTTLAWVNNQIIFFVSRVFANTGSVCYTPIDEIPYKSFSDSGCILPSECTMFRDAEGRMTPYCHDPTVLPGAFAYSQMRPHVRYDLYDGNMFIKFPEVVFESTLRITRTLSTQYCRFGSCEYAQEGVCITTNGSWAIFNDHHLNRPGVYCGSDFIDIVRRLAVSLFQPITYFQLTTSLVLGIGLCAFLTLLFYYINKVKRAFADYTQCAVIAVVAAVLNSLCICFVASIPLCIVPYTALYYYATFYFTNEPAFIMHVSWYIMFGPIVPIWMTCVYTVAMCFRHFFWVLAYFSKKHVEVFTDGKLNCSFQDAASNIFVINKDTYAALRNSLTNDAYSRFLGLFNKYKYFSGAMETAAYREAAACHLAKALQTYSETGSDLLYQPPNCSITSGVLQSGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQSGVRKVTYGTAHWLFATLVSTYVIILQATKFTLWNYLFETIPTQLFPLLFVTMAFVMLLVKHKHTFLTLFLLPVAICLTYANIVYEPTTPISSALIAVANWLAPTNAYMRTTHTDIGVYISMSLVLVIVVKRLYNPSLSNFALALCSGVMWLYTYSIGEASSPIAYLVFVTTLTSDYTITVFVTVNLAKVCTYAIFAYSPQLTLVFPEVKMILLLYTCLGFMCTCYFGVFSLLNLKLRAPMGVYDFKVSTQEFRFMTANNLTAPRNSWEAMALNFKLIGIGGTPCIKVAAMQSKLTDLKCTSVVLLSVLQQLHLEANSRAWAFCVKCHNDILAATDPSEAFEKFVSLFATLMTFSGNVDLDALASDIFDTPSVLQATLSEFSHLATFAELEAAQKAYQEAMDSGDTSPQVLKALQKAVNIAKNAYEKDKAVARKLERMADQAMTSMYKQARAEDKKAKIVSAMQTMLFGMIKKLDNDVLNGIISNARNGCIPLSVIPLCASNKLRVVIPDFTVWNQVVTYPSLNYAGALWDITVINNVDNEIVKSSDVVDSNENLTWPLVLECTRASTSAVKLQNNEIKPSGLKTMVVSAGQEQTNCNTSSLAYYEPVQGRKMLMALLSDNAYLKWARVEGKDGFVSVELQPPCKFLIAGPKGPEIRYLYFVKNLNNLHRGQVLGHIAATVRLQAGSNTEFASNSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQSKDSNFLNRVRGSIVNARIEPCSSGLSTDVVFRAFDICNYKAKVAGIGKYYKTNTCRFVELDDQGHHLDSYFVVKRHTMENYELEKHCYDLLRDCDAVAPHDFFIFDVDKVKTPHIVRQRLTEYTMMDLVYALRHFDQNSEVLKAILVKYGCCDVTYFENKLWFDFVENPSVIGVYHKLGERVRQAILNTVKFCDHMVKAGLVGVLTLDNQDLNGKWYDFGDFVITQPGSGVAIVDSYYSYLMPVLSMTDCLAAETHRDCDFNKPLIEWPLTEYDFTDYKVQLFEKYFKYWDQTYHANCVNCTDDRCVLHCANFNVLFAMTMPKTCFGPIVRKIFVDGVPFVVSCGYHYKELGLVMNMDVSLHRHRLSLKELMMYAADPAMHIASSNAFLDLRTSCFSVAALTTGLTFQTVRPGNFNQDFYDFVVSKGFFKEGSSVTLKHFFFAQDGNAAITDYNYYSYNLPTMCDIKQMLFCMEVVNKYFEIYDGGCLNASEVVVNNLDKSAGHPFNKFGKARVYYESMSYQEQDELFAMTKRNVIPTMTQMNLKYAISAKNRARTVAGVSILSTMTNRQYHQKMLKSMAATRGATCVIGTTKFYGGWDFMLKTLYKDVDNPHLMGWDYPKCDRAMPNMCRIFASLILARKHGTCCTTRDRFYRLANECAQVLSEYVLCGGGYYVKPGGTSSGDATTAYANSVFNILQATTANVSALMGANGNKIVDKEVKDMQFDLYVNVYRSTSPDPKFVDKYYAFLNKHFSMMILSDDGVVCYNSDYAAKGYIAGIQNFKETLYYQNNVFMSEAKCWVETDLKKGPHEFCSQHTLYIKDGDDGYFLPYPDPSRILSAGCFVDDIVKTDGTLMVERFVSLAIDAYPLTKHEDIEYQNVFWVYLQYIEKLYKDLTGHMLDSYSVMLCGDNSAKFWEEAFYRDLYSSPTTLQAVGSCVVCHSQTSLRCGTCIRRPFLCCKCCYDHVIATPHKMVLSVSPYVCNAPGCGVSDVTKLYLGGMSYFCVDHRPVCSFPLCANGLVFGLYKNMCTGSPSIVEFNRLATCDWTESGDYTLANTTTEPLKLFAAETLRATEEASKQSYAIATIKEIVGERQLLLVWEAGKSKPPLNRNYVFTGYHITKNSKVQLGEYIFERIDYSDAVSYKSSTTYKLTVGDIFVLTSHSVATLTAPTIVNQERYVKITGLYPTITVPEEFASHVANFQKSGYSKYVTVQGPPGTGKSHFAIGLAIYYPTARVVYTACSHAAVDALCEKAFKYLNIAKCSRIIPAKARVECYDRFKVNETNSQYLFSTINALPETSADILVVDEVSMCTNYDLSIINARIKAKHIVYVGDPAQLPAPRTLLTRGTLEPENFNSVTRLMCNLGPDIFLSMCYRCPKEIVSTVSALVYNNKLLAKKELSGQCFKILYKGNVTHDASSAINRPQLTFVKNFITANPAWSKAVFISPYNSQNAVARSMLGLTTQTVDSSQGSEYQYVIFCQTADTAHANNINRFNVAITRAQKGILCVMTSQALFESLEFTELSFTNYKLQSQIVTGLFKDCSRETSGLSPAYAPTYVSVDDKYKTSDELCVNLNLPANVPYSRVISRMGFKLDATVPGYPKLFITREEAVRQVRSWIGFDVEGAHASRNACGTNVPLQLGFSTGVNFVVQPVGVVDTEWGNMLTGIAARPPPGEQFKHLVPLMHKGAAWPIVRRRIVQMLSDTLDKLSDYCTFVCWAHGFELTSASYFCKIGKEQKCCMCNRRAAAYSSPLQSYACWTHSCGYDYVYNPFFVDVQQWGYVGNLATNHDRYCSVHQGAHVASNDAIMTRCLAIHSCFIERVDWDIEYPYISHEKKLNSCCRIVERNVVRAALLAGSFDKVYDIGNPKGIPIVDDPVVDWHYFDAQPLTRKVQQLFYTEDMASRFADGLCLFWNCNVPKYPNNAIVCRFDTRVHSEFNLPGCDGGSLYVNKHAFHTPAYDVSAFRDLKPLPFFYYSTTPCEVHGNGSMIEDIDYVPLKSAVCITACNLGGAVCRKHATEYREYMEAYNLVSASGFRLWCYKTFDIYNLWSTFTKVQGLENIAFNVVKQGHFIGVEGELPVAVVNDKIFTKSGVNDICMFENKTTLPTNIAFELYAKRAVRSHPDFKLLHNLQADICYKFVLWDYERSNIYGTATIGVCKYTDIDVNSALNICFDIRDNGSLEKFMSTPNAIFISDRKIKKYPCMVGPDYAYFNGAIIRDSDVVKQPVKFYLYKKVNNEFIDPTECIYTQSRSCSDFLPLSDMEKDFLSFDSDVFIKKYGLENYAFEHVVYGDFSHTTLGGLHLLIGLYKKQQEGHIIMEEMLKGSSTIHNYFITETNTAAFKAVCSVIDLKLDDFVMILKSQDLGVVSKVVKVPIDLTMIEFMLWCKDGQVQTFYPRLQASADWKPGHAMPSLFKVQNVNLERCELANYKQSIPMPRGVHMNIAKYMQLCQYLNTCTLAVPANMRVIHFGAGSDKGIAPGTSVLRQWLPTDAIIIDNDLNEFVSDADITLFGDCVTVRVGQQVDLVISDMYDPTTKNVTGSNESKALFFTYLCNLINNNLALGGSVAIKITEHSWSVELYELMGKFAWWTVFCTNANASSSEGFLLGINYLGTIKENIDGGAMHANYIFWRNSTPMNLSTYSLFDLSKFQLKLKGTPVLQLKESQINELVISLLSQGKLLIRDNDTLSVSTDVLVNTYRKLR

SEQ ID NO: 30 - protein, 42 aa, Artificial Sequence
  Description of Artificial Sequence Synthetic polypeptide
HQDHVSLTVPLCGSGNLVEKLSPWFMDGENAYEVVKAMLLKK

SEQ ID NO: 31 - protein, 39 aa, Artificial Sequence
  Description of Artificial Sequence Synthetic polypeptide
HVSLTVPLCGSGNLVEKLSPWFMDGENAYEVVKAMLLKK

SEQ ID NO: 32 - protein, 23 aa, Artificial Sequence
  Description of Artificial Sequence Synthetic peptide
KAMLLKKEPLLYVPIRLAGHTRH

SEQ ID NO: 33 - protein, 15 aa, Artificial Sequence
  Description of Artificial Sequence Synthetic peptide
KQNILLRKYGRGGYH

SEQ ID NO: 34 - protein, 20 aa, Artificial Sequence
  Description of Artificial Sequence Synthetic peptide
KQNILLRKYGRGGYHYTPFH

SEQ ID NO: 35 - protein, 35 aa, Artificial Sequence
  Description of Artificial Sequence Synthetic polypeptide
HYTPFHYERDNTSCPEWMDDFEADPKGKYAQNLLK

SEQ ID NO: 36 - protein, 36 aa, Artificial Sequence
  Description of Artificial Sequence Synthetic polypeptide
HYTPFHYERDNTSCPEWMDDFEADPKGKYAQNLLKK

SEQ ID NO: 37 - protein, 30 aa, Artificial Sequence
  Description of Artificial Sequence Synthetic polypeptide
HYERDNTSCPEWMDDFEADPKGKYAQNLLK

SEQ ID NO: 38 - protein, 31 aa, Artificial Sequence
  Description of Artificial Sequence Synthetic polypeptide
HYERDNTSCPEWMDDFEADPKGKYAQNLLKK

SEQ ID NO: 39 - protein, 48 aa, Artificial Sequence
  Description of Artificial Sequence Synthetic polypeptide
HVERKDVPYPKQSIFTINSVVQKDGVENTPPHYFTLGCKILTLTPRNK

SEQ ID NO: 40 - protein, 28 aa, Artificial Sequence
  Description of Artificial Sequence Synthetic peptide
KDVPYPKQSIFTINSVVQKDGVENTPPH

SEQ ID NO: 41 - protein, 17 aa, Artificial Sequence
  Description of Artificial Sequence Synthetic peptide
HYFTLGCKILTLTPRNK

SEQ ID NO: 42 - protein, 27 aa, Artificial Sequence
  Description of Artificial Sequence Synthetic peptide
HYFTLGCKILTLTPRNKWSGVSDLSLK

SEQ ID NO: 43 - protein, 37 aa, Artificial Sequence
  Description of Artificial Sequence Synthetic polypeptide
HYFTLGCKILTLTPRNKWSGVSDLSLKQKLLYTFYGK

SEQ ID NO: 44 - protein, 41 aa, Artificial Sequence
  Description of Artificial Sequence Synthetic polypeptide
KILTLTPRNKWSGVSDLSLKQKLLYTFYGKESLENPTYIYH

SEQ ID NO: 45 - protein, 32 aa, Artificial Sequence
  Description of Artificial Sequence Synthetic polypeptide
KWSGVSDLSLKQKLLYTFYGKESLENPTYIYH

SEQ ID NO: 46 - protein, 22 aa, Artificial Sequence
  Description of Artificial Sequence Synthetic peptide
KQKLLYTFYGKESLENPTYIYH

SEQ ID NO: 47 - protein, 20 aa, Artificial Sequence
  Description of Artificial Sequence Synthetic peptide
KLLYTFYGKESLENPTYIYH

SEQ ID NO: 48 - protein, 40 aa, Artificial Sequence
  Description of Artificial Sequence Synthetic polypeptide
HSLNFVGEFVVNDVVLAILSGTTTNVDKIRQLLKGVTLDK

SEQ ID NO: 49 - protein, 23 aa, Artificial Sequence
  Description of Artificial Sequence Synthetic peptide
KKVATIPYKVCNSVKDTLTYYAH

SEQ ID NO: 50 - protein, 22 aa, Artificial Sequence
  Description of Artificial Sequence Synthetic peptide
KVATIPYKVCNSVKDTLTYYAH

SEQ ID NO: 51 - protein, 15 aa, Artificial Sequence
  Description of Artificial Sequence Synthetic peptide
KVCNSVKDTLTYYAH

SEQ ID NO: 52 - protein, 21 aa, Artificial Sequence
  Description of Artificial Sequence Synthetic peptide
KVSTLVKQVLDLLNKGMQLLH

SEQ ID NO: 53 - protein, 15 aa, Artificial Sequence
  Description of Artificial Sequence Synthetic peptide
KQVLDLLNKGMQLLH

SEQ ID NO: 54 - protein, 22 aa, Artificial Sequence
  Description of Artificial Sequence Synthetic peptide
HSPDVIVGDYVIISEKLFVRSK

SEQ ID NO: 55 - protein, 22 aa, Artificial Sequence
  Description of Artificial Sequence Synthetic peptide
KLFVRSKEEDGFAFYPACTNGH

SEQ ID NO: 56 - protein, 47 aa, Artificial Sequence
  Description of Artificial Sequence Synthetic polypeptide
KLFVRSKEEDGFAFYPACTNGHAVPTLFRLKGGAPVKKVAFGGDQVH

SEQ ID NO: 57 - protein, 16 aa, Artificial Sequence
  Description of Artificial Sequence Synthetic peptide
HAVPTLFRLKGGAPVK

SEQ ID NO: 58 - protein, 17 aa, Artificial Sequence
  Description of Artificial Sequence Synthetic peptide
HAVPTLFRLKGGAPVKK

SEQ ID NO: 59 - protein, 17 aa, Artificial Sequence
  Description of Artificial Sequence Synthetic peptide
KGGAPVKKVAFGGDQVH

SEQ ID NO: 60 - protein, 32 aa, Artificial Sequence
  Description of Artificial Sequence Synthetic polypeptide
KGGAPVKKVAFGGDQVHEVAAVRSVTVEYNIH

SEQ ID NO: 61 - protein, 41 aa, Artificial Sequence
  Description of Artificial Sequence Synthetic polypeptide
HAVLDTLLASSSLRTFVVDKSLSIEEFADVVKEQVSDLLVK

SEQ ID NO: 62 - protein, 19 aa, Artificial Sequence
  Description of Artificial Sequence Synthetic peptide
KPKRLRKKRNVDPLSNFEH

SEQ ID NO: 63 - protein, 31 aa, Artificial Sequence
  Description of Artificial Sequence Synthetic polypeptide
HLKHGGGIAGAINAASKGAVQKESDEYILAK

SEQ ID NO: 64 - protein, 28 aa, Artificial Sequence
  Description of Artificial Sequence Synthetic peptide
HGGGIAGAINAASKGAVQKESDEYILAK

SEQ ID NO: 65 - protein, 32 aa, Artificial Sequence
  Description of Artificial Sequence Synthetic polypeptide
KESDEYILAKGPLQVGDSVLLQGHSLAKNILH

SEQ ID NO: 66 - protein, 24 aa, Artificial Sequence
  Description of Artificial Sequence Synthetic peptide
KESDEYILAKGPLQVGDSVLLQGH

SEQ ID NO: 67 - protein, 26 aa, Artificial Sequence
  Description of Artificial Sequence Synthetic peptide
HSLAKNILHVVGPDARAKQDVSLLSK

SEQ ID NO: 68 - protein, 18 aa, Artificial Sequence
  Description of Artificial Sequence Synthetic peptide
HVVGPDARAKQDVSLLSK

SEQ ID NO: 69 - protein, 49 aa, Artificial Sequence
  Description of Artificial Sequence Synthetic polypeptide
KGVDYTKKFLTVDGVQYYCYTSKDTLDDILQQANKSVGIISMPLGYVSH

SEQ ID NO: 70 - protein, 47 aa, Artificial Sequence
  Description of Artificial Sequence Synthetic polypeptide
HGLDLIQAGSVVRRVNVPYVCLLANKEQEAILMSEDVKLNPSEDFIK

SEQ ID NO: 71 - protein, 22 aa, Artificial Sequence
  Description of Artificial Sequence Synthetic peptide
KLNPSEDFIKHVRTNGGYNSWH

SEQ ID NO: 72 - protein, 40 aa, Artificial Sequence
  Description of Artificial Sequence Synthetic polypeptide
KLNPSEDFIKHVRTNGGYNSWHLVEGELLVQDLRLNKLLH

SEQ ID NO: 73 - protein, 47 aa, Artificial Sequence
  Description of Artificial Sequence Synthetic polypeptide
HVRTNGGYNSWHLVEGELLVQDLRLNKLLHWSDQTICYKDSVFYVVK

SEQ ID NO: 74 - protein, 36 aa, Artificial Sequence
  Description of Artificial Sequence Synthetic polypeptide
HLVEGELLVQDLRLNKLLHWSDQTICYKDSVFYVVK

SEQ ID NO: 75 - protein, 18 aa, Artificial Sequence
  Description of Artificial Sequence Synthetic peptide
HWSDQTICYKDSVFYVVK

SEQ ID NO: 76 - protein, 43 aa, Artificial Sequence
  Description of Artificial Sequence Synthetic polypeptide
HSLYLADNLTADETKALKELYGPVDPTFLHRFYSLKAAVHKWK

SEQ ID NO: 77 - protein, 49 aa, Artificial Sequence
  Description of Artificial Sequence Synthetic polypeptide
HSLYLADNLTADETKALKELYGPVDPTFLHRFYSLKAAVHKWKMVVCDK

SEQ ID NO: 78 - protein, 20 aa, Artificial Sequence
  Description of Artificial Sequence Synthetic peptide
HRFYSLKAAVHKWKMVVCDK

SEQ ID NO: 79 - protein, 14 aa, Artificial Sequence
  Description of Artificial Sequence Synthetic peptide
HRFYSLKAAVHKWK

SEQ ID NO: 80 - protein, 49 aa, Artificial Sequence
  Description of Artificial Sequence Synthetic polypeptide
KAAVHKWKMVVCDKVRSLKLSDNNCYLNAVIMTLDLLKDIKFVIPALQH

SEQ ID NO: 81 - protein, 44 aa, Artificial Sequence
  Description of Artificial Sequence Synthetic polypeptide
KWKMVVCDKVRSLKLSDNNCYLNAVIMTLDLLKDIKFVIPALQH

SEQ ID NO: 82 - protein, 49 aa, Artificial Sequence
  Description of Artificial Sequence Synthetic polypeptide
KWKMVVCDKVRSLKLSDNNCYLNAVIMTLDLLKDIKFVIPALQHAFMKH

SEQ ID NO: 83 - protein, 42 aa, Artificial Sequence
  Description of Artificial Sequence Synthetic polypeptide
KMVVCDKVRSLKLSDNNCYLNAVIMTLDLLKDIKFVIPALQH

SEQ ID NO: 84 - protein, 47 aa, Artificial Sequence
  Description of Artificial Sequence Synthetic polypeptide
KMVVCDKVRSLKLSDNNCYLNAVIMTLDLLKDIKFVIPALQHAFMKH

SEQ ID NO: 85 - protein, 35 aa, Artificial Sequence
  Description of Artificial Sequence Synthetic polypeptide
HTVLAKAELCCSARMVWREWCNVCGIKDVVLQGLK

SEQ ID NO: 86 - protein, 22 aa, Artificial Sequence
  Description of Artificial Sequence Synthetic peptide
HYVHARLKGGLILKFDSGTVSK

SEQ ID NO: 87 - protein, 29 aa, Artificial Sequence
  Description of Artificial Sequence Synthetic peptide
HYVHARLKGGLILKFDSGTVSKTSDWKCK

SEQ ID NO: 88 - protein, 39 aa, Artificial Sequence
  Description of Artificial Sequence Synthetic polypeptide
HYVHARLKGGLILKFDSGTVSKTSDWKCKVTDVLFSGQK

SEQ ID NO: 89 - protein, 14 aa, Artificial Sequence
  Description of Artificial Sequence Synthetic peptide
HYVHARLKGGLILK

SEQ ID NO: 90 - protein, 19 aa, Artificial Sequence
  Description of Artificial Sequence Synthetic peptide
HARLKGGLILKFDSGTVSK

SEQ ID NO: 91 - protein, 26 aa, Artificial Sequence
  Description of Artificial Sequence Synthetic peptide
HARLKGGLILKFDSGTVSKTSDWKCK

SEQ ID NO: 92 - protein, 36 aa, Artificial Sequence
  Description of Artificial Sequence Synthetic polypeptide
HARLKGGLILKFDSGTVSKTSDWKCKVTDVLFSGQK

SEQ ID NO: 93 - protein, 35 aa, Artificial Sequence
  Description of Artificial Sequence Synthetic polypeptide
KCKGLNKPFVKDNVSFVADDSGTPVVEYLSKEDLH

SEQ ID NO: 94 - protein, 33 aa, Artificial Sequence
  Description of Artificial Sequence Synthetic polypeptide
KGLNKPFVKDNVSFVADDSGTPVVEYLSKEDLH

SEQ ID NO: 95 - protein, 15 aa, Artificial Sequence
  Description of Artificial Sequence Synthetic peptide
HTLYVDPKYQVIVLK

SEQ ID NO: 96 - protein, 19 aa, Artificial Sequence
  Description of Artificial Sequence Synthetic peptide
KYQVIVLKDNVLSSMLRLH

SEQ ID NO: 97 - protein, 32 aa, Artificial Sequence
  Description of Artificial Sequence Synthetic polypeptide
HTVESGDINVVAASGSLTRKVKLLFRASFYFK

SEQ ID NO: 98 - protein, 34 aa, Artificial Sequence
  Description of Artificial Sequence Synthetic polypeptide
KLLFRASFYFKEFATRTFTATTAVGSCIKSVVRH

SEQ ID NO: 99 - protein, 38 aa, Artificial Sequence
  Description of Artificial Sequence Synthetic polypeptide
HLGVTKGILTGCFSFVKMLFMLPLAYFSDSKLGTTEVK

SEQ ID NO: 100 - protein, 29 aa, Artificial Sequence
  Description of Artificial Sequence Synthetic peptide
KLRRVDWKSTLRLLLMLCTTMVLLSSVYH

SEQ ID NO: 101 - protein, 32 aa, Artificial Sequence
  Description of Artificial Sequence Synthetic polypeptide
HIPMAGLVRMYNLLACLWLLRKFYQHVINGCK

SEQ ID NO: 102 - protein, 41 aa, Artificial Sequence
  Description of Artificial Sequence Synthetic polypeptide
HIPMAGLVRMYNLLACLWLLRKFYQHVINGCKDTACLLCYK

SEQ ID NO: 103 - protein, 16 aa, Artificial Sequence
  Description of Artificial Sequence Synthetic peptide
HVINGCKDTACLLCYK

SEQ ID NO: 104 - protein, 44 aa, Artificial Sequence
  Description of Artificial Sequence Synthetic polypeptide
KDTACLLCYKRNRLTRVEASTVVCGGKRTFYITANGGISFCRRH

SEQ ID NO: 105 - protein, 47 aa, Artificial Sequence
  Description of Artificial Sequence Synthetic polypeptide
HYYVDSVTVKETVVQFNYRRDGQPFYERFPLCAFTNLDKLKFKEVCK

SEQ ID NO: 106 - protein, 48 aa, Artificial Sequence
  Description of Artificial Sequence Synthetic polypeptide
HVSWYIMFGPIVPIWMTCVYTVAMCFRHFFWVLAYFSKKHVEVFTDGK

SEQ ID NO: 107 - protein, 21 aa, Artificial Sequence
  Description of Artificial Sequence Synthetic peptide
HFFWVLAYFSKKHVEVFTDGK

SEQ ID NO: 108 - protein, 27 aa, Artificial Sequence
  Description of Artificial Sequence Synthetic peptide
HTGSAFDGTMYGAFMDKQVHQVQLTDK

SEQ ID NO: 109 - protein, 42 aa, Artificial Sequence
  Description of Artificial Sequence Synthetic polypeptide
KVAAMQSKLTDLKCTSVVLLSVLQQLHLEANSRAWAFCVKCH

SEQ ID NO: 110 - protein, 27 aa, Artificial Sequence
  Description of Artificial Sequence Synthetic peptide
KVAAMQSKLTDLKCTSVVLLSVLQQLH

SEQ ID NO: 111 - protein, 40 aa, Artificial Sequence
  Description of Artificial Sequence Synthetic polypeptide
HLATFAELEAAQKAYQEAMDSGDTSPQVLKALQKAVNIAK

SEQ ID NO: 112 - protein, 47 aa, Artificial Sequence
  Description of Artificial Sequence Synthetic polypeptide
HLATFAELEAAQKAYQEAMDSGDTSPQVLKALQKAVNIAKNAYEKDK

SEQ ID NO: 113 - protein, 45 aa, Artificial Sequence
  Description of Artificial Sequence Synthetic polypeptide
KWARVEGKDGFVSVELQPPCKFLIAGPKGPEIRYLYFVKNLNNLH

SEQ ID NO: 114 - protein, 25 aa, Artificial Sequence
  Description of Artificial Sequence Synthetic peptide
KFLIAGPKGPEIRYLYFVKNLNNLH

SEQ ID NO: 115 - protein, 32 aa, Artificial Sequence
  Description of Artificial Sequence Synthetic polypeptide
KFLIAGPKGPEIRYLYFVKNLNNLHRGQVLGH

SEQ ID NO: 116 - protein, 33 aa, Artificial Sequence
  Description of Artificial Sequence Synthetic polypeptide
KTGTGIAISVKPESTADQETYGGASVCLYCRAH

SEQ ID NO: 117 - protein, 26 aa, Artificial Sequence
  Description of Artificial Sequence Synthetic peptide
KAKVAGIGKYYKTNTCRFVELDDQGH

SEQ ID NO: 118 - protein, 27 aa, Artificial Sequence
  Description of Artificial Sequence Synthetic peptide
KAKVAGIGKYYKTNTCRFVELDDQGHH

SEQ ID NO: 119 - protein, 37 aa, Artificial Sequence
  Description of Artificial Sequence Synthetic polypeptide
KAKVAGIGKYYKTNTCRFVELDDQGHHLDSYFVVKRH

SEQ ID NO: 120 - protein, 47 aa, Artificial Sequence
  Description of Artificial Sequence Synthetic polypeptide
KAKVAGIGKYYKTNTCRFVELDDQGHHLDSYFVVKRHTMENYELEKH

SEQ ID NO: 121 - protein, 35 aa, Artificial Sequence
  Description of Artificial Sequence Synthetic polypeptide
KVAGIGKYYKTNTCRFVELDDQGHHLDSYFVVKRH

SEQ ID NO: 122 - protein, 45 aa, Artificial Sequence
  Description of Artificial Sequence Synthetic polypeptide
KVAGIGKYYKTNTCRFVELDDQGHHLDSYFVVKRHTMENYELEKH

SEQ ID NO: 123 - protein, 24 aa, Artificial Sequence
  Description of Artificial Sequence Synthetic peptide
KVAGIGKYYKTNTCRFVELDDQGH

SEQ ID NO: 124 - protein, 25 aa, Artificial Sequence
  Description of Artificial Sequence Synthetic peptide
KVAGIGKYYKTNTCRFVELDDQGHH

SEQ ID NO: 125 - protein, 23 aa, Artificial Sequence
  Description of Artificial Sequence Synthetic peptide
HKLGERVRQAILNTVKFCDHMVK

SEQ ID NO: 126 - protein, 30 aa, Artificial Sequence
  Description of Artificial Sequence Synthetic polypeptide
HRDCDFNKPLIEWPLTEYDFTDYKVQLFEK

SEQ ID NO: 127 - protein, 33 aa, Artificial Sequence
  Description of Artificial Sequence Synthetic polypeptide
HRDCDFNKPLIEWPLTEYDFTDYKVQLFEKYFK

SEQ ID NO: 128 - protein, 17 aa, Artificial Sequence
  Description of Artificial Sequence Synthetic peptide
KVQLFEKYFKYWDQTYH

SEQ ID NO: 129 - protein, 31 aa, Artificial Sequence
  Description of Artificial Sequence Synthetic polypeptide
KVQLFEKYFKYWDQTYHANCVNCTDDRCVLH

SEQ ID NO: 130 - protein, 24 aa, Artificial Sequence
  Description of Artificial Sequence Synthetic peptide
HCANFNVLFAMTMPKTCFGPIVRK

SEQ ID NO: 131 - protein, 25 aa, Artificial Sequence
  Description of Artificial Sequence Synthetic peptide
KTCFGPIVRKIFVDGVPFVVSCGYH

SEQ ID NO: 132 - protein, 40 aa, Artificial Sequence
  Description of Artificial Sequence Synthetic polypeptide
KTCFGPIVRKIFVDGVPFVVSCGYHYKELGLVMNMDVSLH

SEQ ID NO: 133 - protein, 42 aa, Artificial Sequence
  Description of Artificial Sequence Synthetic polypeptide
KTCFGPIVRKIFVDGVPFVVSCGYHYKELGLVMNMDVSLHRH

SEQ ID NO: 134 - protein, 48 aa, Artificial Sequence
  Description of Artificial Sequence Synthetic polypeptide
HPFNKFGKARVYYESMSYQEQDELFAMTKRNVIPTMTQMNLKYAISAK

SEQ ID NO: 135 - protein, 28 aa, Artificial Sequence
  Description of Artificial Sequence Synthetic peptide
KYAISAKNRARTVAGVSILSTMTNRQYH

SEQ ID NO: 136 - protein, 32 aa, Artificial Sequence
  Description of Artificial Sequence Synthetic polypeptide
HQKMLKSMAATRGATCVIGTTKFYGGWDFMLK

SEQ ID NO: 137 - protein, 21 aa, Artificial Sequence
  Description of Artificial Sequence Synthetic peptide
KFYGGWDFMLKTLYKDVDNPH

SEQ ID NO: 138 - protein, 50 aa, Artificial Sequence
  Description of Artificial Sequence Synthetic polypeptide
KFYGGWDFMLKTLYKDVDNPHLMGWDYPKCDRAMPNMCRIFASLILARKH

SEQ ID NO: 139 - protein, 39 aa, Artificial Sequence
  Description of Artificial Sequence Synthetic polypeptide
KIVDKEVKDMQFDLYVNVYRSTSPDPKFVDKYYAFLNKH

SEQ ID NO: 140 - protein, 32 aa, Artificial Sequence
  Description of Artificial Sequence Synthetic polypeptide
HFSMMILSDDGVVCYNSDYAAKGYIAGIQNFK

SEQ ID NO: 141 - protein, 38 aa, Artificial Sequence
  Description of Artificial Sequence Synthetic polypeptide
KGYIAGIQNFKETLYYQNNVFMSEAKCWVETDLKKGPH

SEQ ID NO: 142 - protein, 44 aa, Artificial Sequence
  Description of Artificial Sequence Synthetic polypeptide
KGYIAGIQNFKETLYYQNNVFMSEAKCWVETDLKKGPHEFCSQH

SEQ ID NO: 143 - protein, 13 aa, Artificial Sequence
  Description of Artificial Sequence Synthetic peptide
KCWVETDLKKGPH

SEQ ID NO: 144 - protein, 19 aa, Artificial Sequence
  Description of Artificial Sequence Synthetic peptide
KCWVETDLKKGPHEFCSQH

SEQ ID NO: 145 - protein, 41 aa, Artificial Sequence
  Description of Artificial Sequence Synthetic polypeptide
KQSYAIATIKEIVGERQLLLVWEAGKSKPPLNRNYVFTGYH

SEQ ID NO: 146 - protein, 33 aa, Artificial Sequence
  Description of Artificial Sequence Synthetic polypeptide
HITKNSKVQLGEYIFERIDYSDAVSYKSSTTYK

SEQ ID NO: 147 - protein, 19 aa, Artificial Sequence
  Description of Artificial Sequence Synthetic peptide
KSSTTYKLTVGDIFVLTSH

SEQ ID NO: 148 - protein, 40 aa, Artificial Sequence
  Description of Artificial Sequence Synthetic polypeptide
HFAIGLAIYYPTARVVYTACSHAAVDALCEKAFKYLNIAK

SEQ ID NO: 149 - protein, 48 aa, Artificial Sequence
  Description of Artificial Sequence Synthetic polypeptide
HFAIGLAIYYPTARVVYTACSHAAVDALCEKAFKYLNIAKCSRIIPAK

SEQ ID NO: 150 - protein, 19 aa, Artificial Sequence
  Description of Artificial Sequence Synthetic peptide
HAAVDALCEKAFKYLNIAK

SEQ ID NO: 151 - protein, 27 aa, Artificial Sequence
  Description of Artificial Sequence Synthetic peptide
HAAVDALCEKAFKYLNIAKCSRIIPAK

SEQ ID NO: 152 - protein, 37 aa, Artificial Sequence
  Description of Artificial Sequence Synthetic polypeptide
HAAVDALCEKAFKYLNIAKCSRIIPAKARVECYDRFK

SEQ ID NO: 153 - protein, 19 aa, Artificial Sequence
  Description of Artificial Sequence Synthetic peptide
KKELSGQCFKILYKGNVTH

SEQ ID NO: 154 - protein, 18 aa, Artificial Sequence
  Description of Artificial Sequence Synthetic peptide
KELSGQCFKILYKGNVTH

SEQ ID NO: 155 - protein, 42 aa, Artificial Sequence
  Description of Artificial Sequence Synthetic polypeptide
KHLVPLMHKGAAWPIVRRRIVQMLSDTLDKLSDYCTFVCWAH

SEQ ID NO: 156 - protein, 19 aa, Artificial Sequence
  Description of Artificial Sequence Synthetic peptide
HGFELTSASYFCKIGKEQK

SEQ ID NO: 157 - protein, 30 aa, Artificial Sequence
  Description of Artificial Sequence Synthetic polypeptide
KIGKEQKCCMCNRRAAAYSSPLQSYACWTH

SEQ ID NO: 158 - protein, 36 aa, Artificial Sequence
  Description of Artificial Sequence Synthetic polypeptide
HEKKLNSCCRIVERNVVRAALLAGSFDKVYDIGNPK

SEQ ID NO: 159 - protein, 22 aa, Artificial Sequence
  Description of Artificial Sequence Synthetic peptide
KVYDIGNPKGIPIVDDPVVDWH

SEQ ID NO: 160 - protein, 11 aa, Artificial Sequence
  Description of Artificial Sequence Synthetic peptide
KRAVRSHPDFK

SEQ ID NO: 161 - protein, 14 aa, Artificial Sequence
  Description of Artificial Sequence Synthetic peptide
KRAVRSHPDFKLLH

SEQ ID NO: 162 - protein, 50 aa, Artificial Sequence
  Description of Artificial Sequence Synthetic polypeptide
HLLIGLYKKQQEGHIIMEEMLKGSSTIHNYFITETNTAAFKAVCSVIDLK

SEQ ID NO: 163 - protein, 37 aa, Artificial Sequence
  Description of Artificial Sequence Synthetic polypeptide
HIIMEEMLKGSSTIHNYFITETNTAAFKAVCSVIDLK

SEQ ID NO: 164 - protein, 46 aa, Artificial Sequence
  Description of Artificial Sequence Synthetic polypeptide
HIIMEEMLKGSSTIHNYFITETNTAAFKAVCSVIDLKLDDFVMILK

SEQ ID NO: 165 - protein, 32 aa, Artificial Sequence
  Description of Artificial Sequence Synthetic polypeptide
HNYFITETNTAAFKAVCSVIDLKLDDFVMILK

SEQ ID NO: 166 - protein, 41 aa, Artificial Sequence
  Description of Artificial Sequence Synthetic polypeptide
HNYFITETNTAAFKAVCSVIDLKLDDFVMILKSQDLGVVSK

SEQ ID NO: 167 - protein, 23 aa, Artificial Sequence
  Description of Artificial Sequence Synthetic peptide
HNYFITETNTAAFKAVCSVIDLK

SEQ ID NO: 168 - protein, 49 aa, Artificial Sequence
  Description of Artificial Sequence Synthetic polypeptide
KSQDLGVVSKVVKVPIDLTMIEFMLWCKDGQVQTFYPRLQASADWKPGH

SEQ ID NO: 169 - protein, 36 aa, Artificial Sequence
  Description of Artificial Sequence Synthetic polypeptide
KPGHAMPSLFKVQNVNLERCELANYKQSIPMPRGVH

SEQ ID NO: 170 - protein, 38 aa, Artificial Sequence
  Description of Artificial Sequence Synthetic polypeptide
KNVTGSNESKALFFTYLCNLINNNLALGGSVAIKITEH

SEQ ID NO: 171 - protein, 31 aa, Artificial Sequence
  Description of Artificial Sequence Synthetic polypeptide
HANYIFWRNSTPMNLSTYSLFDLSKFQLKLK

SEQ ID NO: 172 - protein, 39 aa, Artificial Sequence
  Description of Artificial Sequence Synthetic polypeptide
HANYIFWRNSTPMNLSTYSLFDLSKFQLKLKGTPVLQLK

SEQ ID NO: 173 - protein, 411 aa, Artificial Sequence [sequence omitted]

SEQ ID NO: 174 - protein, 43 aa, Artificial Sequence
  Description of Artificial Sequence Synthetic polypeptide
HGKVPLTFPPGQGVPLNANSTPAQNAGYWRRQDRKINTGNGIK

SEQ ID NO: 175 - protein, 41 aa, Artificial Sequence
  Description of Artificial Sequence Synthetic polypeptide
KINTGNGIKQLAPRWYFYYTGTGPEAALPFRAVKDGIVWVH

SEQ ID NO: 176 - protein, 24 aa, Artificial Sequence
  Description of Artificial Sequence Synthetic peptide
KVKQSQPKVITKKDAAAAKNKMRH

SEQ ID NO: 177 - protein, 22 aa, Artificial Sequence
  Description of Artificial Sequence Synthetic peptide
KQSQPKVITKKDAAAAKNKMRH

SEQ ID NO: 178 - protein, 31 aa, Artificial Sequence
  Description of Artificial Sequence Synthetic polypeptide
HQNNDDHGNPVYFLRYSGAIKLDPKNPNYNK

SEQ ID NO: 179 - protein, 51 aa, Artificial Sequence
  Description of Artificial Sequence Synthetic polypeptide
HQNNDDHGNPVYFLRYSGAIKLDPKNPNYNKWLELLEQNIDAYKTFPKKEK

SEQ ID NO: 180 - protein, 25 aa, Artificial Sequence
  Description of Artificial Sequence Synthetic peptide
HGNPVYFLRYSGAIKLDPKNPNYNK

SEQ ID NO: 181 - protein, 45 aa, Artificial Sequence
  Description of Artificial Sequence Synthetic polypeptide
HGNPVYFLRYSGAIKLDPKNPNYNKWLELLEQNIDAYKTFPKKEK

SEQ ID NO: 182 - protein, 46 aa, Artificial Sequence
  Description of Artificial Sequence Synthetic polypeptide
HGNPVYFLRYSGAIKLDPKNPNYNKWLELLEQNIDAYKTFPKKEKK

SEQ ID NO: 183 - protein, 48 aa, Artificial Sequence
  Description of Artificial Sequence Synthetic polypeptide
HGNPVYFLRYSGAIKLDPKNPNYNKWLELLEQNIDAYKTFPKKEKKQK

SEQ ID NO: 184 - protein, 51 aa, Artificial Sequence
  Description of Artificial Sequence Synthetic polypeptide
HGNPVYFLRYSGAIKLDPKNPNYNKWLELLEQNIDAYKTFPKKEKKQKAPK

SEQ ID NO: 185 - protein, 51 aa, Artificial Sequence
  Description of Artificial Sequence Synthetic polypeptide
KLDPKNPNYNKWLELLEQNIDAYKTFPKKEKKQKAPKEESTDQMSEPPKEH

SEQ ID NO: 186 - protein, 47 aa, Artificial Sequence
  Description of Artificial Sequence Synthetic polypeptide
KNPNYNKWLELLEQNIDAYKTFPKKEKKQKAPKEESTDQMSEPPKEH

SEQ ID NO: 187 - protein, 28 aa, Artificial Sequence
  Description of Artificial Sequence Synthetic peptide
KTFPKKEKKQKAPKEESTDQMSEPPKEH

SEQ ID NO: 188 - protein, 24 aa, Artificial Sequence
  Description of Artificial Sequence Synthetic peptide
KKEKKQKAPKEESTDQMSEPPKEH

SEQ ID NO: 189 - protein, 23 aa, Artificial Sequence
  Description of Artificial Sequence Synthetic peptide
KEKKQKAPKEESTDQMSEPPKEH

SEQ ID NO: 190 - protein, 21 aa, Artificial Sequence
  Description of Artificial Sequence Synthetic peptide
KKQKAPKEESTDQMSEPPKEH

SEQ ID NO: 191 - protein, 411 aa, Artificial Sequence [sequence omitted]

SEQ ID NO: 192 - protein, 43 aa, Artificial Sequence; identical to SEQ ID NO: 174
  Description of Artificial Sequence Synthetic polypeptide

SEQ ID NO: 193 - protein, 41 aa, Artificial Sequence; identical to SEQ ID NO: 175
  Description of Artificial Sequence Synthetic polypeptide

SEQ ID NO: 194 - protein, 24 aa, Artificial Sequence
  Description of Artificial Sequence Synthetic peptide
KVKQSQPKVITKKDAAAAKNKMRH

SEQ ID NO: 195 - protein, 22 aa, Artificial Sequence
  Description of Artificial Sequence Synthetic peptide
KQSQPKVITKKDAAAAKNKMRH

SEQ ID NO: 196 - protein, 13 aa, Artificial Sequence
  Description of Artificial Sequence Synthetic peptide
KKDAAAAKNKMRH

SEQ ID NO: 197 - protein, 31 aa, Artificial Sequence; identical to SEQ ID NO: 178
  Description of Artificial Sequence Synthetic polypeptide

SEQ ID NO: 198 - protein, 51 aa, Artificial Sequence; identical to SEQ ID NO: 179
  Description of Artificial Sequence Synthetic polypeptide

SEQ ID NO: 199 - protein, 25 aa, Artificial Sequence
  Description of Artificial Sequence Synthetic peptide
HGNPVYFLRYSGAIKLDPKNPNYNK

SEQ ID NO: 200 - protein, 45 aa, Artificial Sequence; identical to SEQ ID NO: 181
  Description of Artificial Sequence Synthetic polypeptide

SEQ ID NO: 201 - protein, 46 aa, Artificial Sequence; identical to SEQ ID NO: 182
  Description of Artificial Sequence Synthetic polypeptide

SEQ ID NO: 202 - protein, 48 aa, Artificial Sequence; identical to SEQ ID NO: 183
  Description of Artificial Sequence Synthetic polypeptide

SEQ ID NO: 203 - protein, 51 aa, Artificial Sequence; identical to SEQ ID NO: 184
  Description of Artificial Sequence Synthetic polypeptide

SEQ ID NO: 204 - protein, 51 aa, Artificial Sequence; identical to SEQ ID NO: 185
  Description of Artificial Sequence Synthetic polypeptide

SEQ ID NO: 205 - protein, 47 aa, Artificial Sequence; identical to SEQ ID NO: 186
  Description of Artificial Sequence Synthetic polypeptide

SEQ ID NO: 206 - protein, 28 aa, Artificial Sequence
  Description of Artificial Sequence Synthetic peptide
KTFPKKEKKQKAPKEESTDQMSEPPKEH

SEQ ID NO: 207 - protein, 24 aa, Artificial Sequence
  Description of Artificial Sequence Synthetic peptide
KKEKKQKAPKEESTDQMSEPPKEH

SEQ ID NO: 208 - protein, 23 aa, Artificial Sequence
  Description of Artificial Sequence Synthetic peptide
KEKKQKAPKEESTDQMSEPPKEH

SEQ ID NO: 209 - protein, 21 aa, Artificial Sequence
  Description of Artificial Sequence Synthetic peptide
KKQKAPKEESTDQMSEPPKEH

SEQ ID NO: 210 - protein, 413 aa, Artificial Sequence
  Description of Artificial Sequence Synthetic polypeptide
MASPAAPRAVSFAYNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 211 - protein, 43 aa, Artificial Sequence; identical to SEQ ID NO: 174
  Description of Artificial Sequence Synthetic polypeptide

SEQ ID NO: 212 - protein, 41 aa, Artificial Sequence; identical to SEQ ID NO: 175
  Description of Artificial Sequence Synthetic polypeptide

SEQ ID NO: 213 - protein, 24 aa, Artificial Sequence
  Description of Artificial Sequence Synthetic peptide
KVKQSQPKVITKKDAAAAKNKMRH

SEQ ID NO: 214 - protein, 22 aa, Artificial Sequence
  Description of Artificial Sequence Synthetic peptide
KQSQPKVITKKDAAAAKNKMRH

SEQ ID NO: 215 - protein, 13 aa, Artificial Sequence
  Description of Artificial Sequence Synthetic peptide
KKDAAAAKNKMRH

SEQ ID NO: 216 - protein, 31 aa, Artificial Sequence; identical to SEQ ID NO: 178
  Description of Artificial Sequence Synthetic polypeptide

SEQ ID NO: 217 - protein, 51 aa, Artificial Sequence; identical to SEQ ID NO: 179
  Description of Artificial Sequence Synthetic polypeptide

SEQ ID NO: 218 - protein, 25 aa, Artificial Sequence
  Description of Artificial Sequence Synthetic peptide
HGNPVYFLRYSGAIKLDPKNPNYNK

SEQ ID NO: 219 - protein, 45 aa, Artificial Sequence; identical to SEQ ID NO: 181
  Description of Artificial Sequence Synthetic polypeptide

SEQ ID NO: 220 - protein, 46 aa, Artificial Sequence; identical to SEQ ID NO: 182
  Description of Artificial Sequence Synthetic polypeptide

SEQ ID NO: 221 - protein, 48 aa, Artificial Sequence; identical to SEQ ID NO: 183
  Description of Artificial Sequence Synthetic polypeptide

SEQ ID NO: 222 - protein, 51 aa, Artificial Sequence; identical to SEQ ID NO: 184
  Description of Artificial Sequence Synthetic polypeptide

SEQ ID NO: 223 - protein, 413 aa, Artificial Sequence
  Description of Artificial Sequence Synthetic polypeptide
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 224 - protein, 43 aa, Artificial Sequence; identical to SEQ ID NO: 174
  Description of Artificial Sequence Synthetic polypeptide

SEQ ID NO: 225 - protein, 41 aa, Artificial Sequence; identical to SEQ ID NO: 175
  Description of Artificial Sequence Synthetic polypeptide

SEQ ID NO: 226 - protein, 24 aa, Artificial Sequence
  Description of Artificial Sequence Synthetic peptide
KVKQSQPKVITKKDAAAAKNKMRH

SEQ ID NO: 227 - protein, 22 aa, Artificial Sequence
  Description of Artificial Sequence Synthetic peptide
KQSQPKVITKKDAAAAKNKMRH

SEQ ID NO: 228 - protein, 13 aa, Artificial Sequence
  Description of Artificial Sequence Synthetic peptide
KKDAAAAKNKMRH

SEQ ID NO: 229 - protein, 31 aa, Artificial Sequence; identical to SEQ ID NO: 178
  Description of Artificial Sequence Synthetic polypeptide

SEQ ID NO: 230 - protein, 51 aa, Artificial Sequence; identical to SEQ ID NO: 179
  Description of Artificial Sequence Synthetic polypeptide

SEQ ID NO: 231 - protein, 25 aa, Artificial Sequence
  Description of Artificial Sequence Synthetic peptide
HGNPVYFLRYSGAIKLDPKNPNYNK

SEQ ID NO: 232 - protein, 45 aa, Artificial Sequence; identical to SEQ ID NO: 181
  Description of Artificial Sequence Synthetic polypeptide

SEQ ID NO: 233 - protein, 46 aa, Artificial Sequence; identical to SEQ ID NO: 182
  Description of Artificial Sequence Synthetic polypeptide

SEQ ID NO: 234 - protein, 48 aa, Artificial Sequence; identical to SEQ ID NO: 183
  Description of Artificial Sequence Synthetic polypeptide

SEQ ID NO: 235 - protein, 51 aa, Artificial Sequence; identical to SEQ ID NO: 184
  Description of Artificial Sequence Synthetic polypeptide

SEQ ID NO: 236 - protein, 69 aa, Artificial Sequence
  Description of Artificial Sequence Synthetic polypeptide
MEESLMDVPSTSGTQVYSRKARKRSHSPTKKLRYVKRRFSLLRPEDLSVIVQPTHYVRVTFSDPNMWYL

SEQ ID NO: 237 - protein, 36 aa, Artificial Sequence
  Description of Artificial Sequence Synthetic polypeptide
KARKRSHSPTKKLRYVKRRFSLLRPEDLSVIVQPTH

SEQ ID NO: 238 - protein, 33 aa, Artificial Sequence
  Description of Artificial Sequence Synthetic polypeptide
KRSHSPTKKLRYVKRRFSLLRPEDLSVIVQPTH

SEQ ID NO: 239 - protein, 26 aa, Artificial Sequence
  Description of Artificial Sequence Synthetic peptide
KKLRYVKRRFSLLRPEDLSVIVQPTH